(19) United States
(12) Patent Application Publication
(10) Pub. No.: US 2026/0071263 A1
(43) Pub. Date: Mar. 12, 2026

(54) METHODS FOR SPATIAL GENOMIC, EPIGENOMIC AND MULTI-OMIC PROFILING USING TRANSPOSASES AND LIGHT-ACTIVATED SPATIAL BARCODING

(71) Applicant: CAMBRIDGE ENTERPRISE LIMITED, Cambridge Cambridgeshire (GB)

(72) Inventors: Benjamin Nicholson, Cambridge Cambridgeshire (GB); Gregory Hannon, Cambridge Cambridgeshire (GB); Dario Bressan, Cambridge Cambridgeshire (GB); Sito Torres-Garcia, Cambridge Cambridgeshire (GB); Giorgia Battistoni, Cambridge Cambridgeshire (GB)

(73) Assignee: CAMBRIDGE ENTERPRISE LIMITED, Cambridge Cambridgeshire (GB)

(21) Appl. No.: 18/872,562

(22) PCT Filed: Jun. 13, 2023
(86) PCT No.: PCT/GB2023/051541

§ 371 Date: Dec. 6, 2024

(30) Foreign Application Priority Data
Jun. 14, 2022 (GB) 2208728.2

(51) Int. Cl. C12Q 1/6869; C12Q 1/6841

(52) U.S. Cl. CPC C12Q 1/6869; C12Q 1/6841

(57) ABSTRACT

The present invention relates to a method of spatially barcoding one or more biological molecules located on or within a substrate by using a transposase complex to label the biological molecule of interest with a detection probe which may then give rise to a spatial barcode. Such analysis may include determining the spatial profiling of one or more biological molecules, specifically the spatial analysis of DNA, open chromatin, chromatin features, proteins and/or RNA which may be spatially barcoded by methods of the invention, either alone or in various combinations. The invention further relates to a transposase complex for use in spatially barcoding one or more biological molecules and reagents kits for performing such methods.

### CROSS-REFERENCE TO RELATED APPLICATIONS

This application is a National Phase of International Application No. PCT/GB2023/051541, filed Jun. 13, 2023, which designated the U.S. and that International Application was published under PCT Article 21 (2) in English. This application also includes a claim of priority under 35 U.S.C. § 119 (a) and § 365 (b) to British patent application No. 2208728.2 filed Jun. 14, 2022, the entirety of which is hereby incorporated by reference.

### REFERENCE TO SEQUENCE LISTING

This application contains a Sequence Listing submitted as a computer readable form named “P336488US.xml”, having a size in bytes of 167,500 bytes, and created on Nov. 6, 2025 (production date). The information contained in this computer readable form is hereby incorporated by reference in its entirety.

### FIELD OF THE INVENTION

The present invention relates to a method of spatially barcoding one or more biological molecules located on or within a substrate by using a transposase complex to label the biological molecule of interest with a detection probe which may then give rise to a spatial barcode. Such analysis may include determining the spatial profiling of one or more biological molecules, specifically the spatial analysis of DNA, open chromatin, chromatin features, proteins and/or RNA which may be spatially barcoded by methods of the invention, either alone or in various combinations. The invention further relates to a transposase complex for use in spatially barcoding one or more biological molecules and reagents kits for performing such methods.

### INTRODUCTION

In situ analysis of the expression of biological molecules is an area of technology that has rapidly developed in recent years. In particular, the development of in situ transcriptomics and multiplexed histochemistry analysis techniques, that allow the determination of what genes are being expressed and/or what biological markers may be present, and to what level in any given location of a given tissue sample, have gained increasing popularity, and enabled a whole new range of biological investigations.

Historically, methods allowing the measurement of many biological molecules at once with high-throughput have provided data on average expression levels in a given sample, but without any context of where the molecules are being expressed. More recently, single-cell analysis techniques have been developed, in which cells from disaggregated tissues are analysed individually. While these methods provide more detailed information on the biological processes happening in the sample, and allow the identification of rare cell populations contributing to them, the absence of real spatial information is a significant hurdle to research. Since all of biology happens in space, the function of a given biological molecule in a process can only be fully understood by considering the spatial context in which the molecule itself is acting.

The field has started to address the need for spatial information by the provision of various in situ transcriptomics and proteomics methods, involving a means of spatial detection of gene or protein expression. There are also families of methods for spatial profiling of metabolites and other biological molecules. These methods can be broadly considered as two groups; image-based methods and image-free methods.

In image-based methods for gene expression measurement, the RNAs contained into the biological tissue are first contacted by DNA probes of complementary sequence. In some techniques, the probes are directly used for the detection and identification of each RNA molecule through fluorescence, using a variety of detection schemes which in some cases include signal amplification through branched DNA, hybridization chain reaction, or rolling circle amplification. In other techniques, the probes are used as primers for reverse transcription of the RNA, producing a complementary DNA molecule for each RNA transcript which can be amplified and detected through in-situ sequencing. Such methods include merFISH, seqFISH, starMAP, FISSEQ, ISS, and BARISTAseq. In all of these methods, the identification of multiple types of RNA molecules (corresponding to different genes) is achieved by repeated cycles of fluorescence imaging in which individual molecules are detected as fluorescent spots. These methods are limited in their ability to achieve single molecule imaging, since the image signals can have low intensity, be difficult to discriminate and suffer from auto-fluorescence or background noise. Furthermore, these methods do not allow the identification of very abundant RNA molecules, since these produce signals that overlap spatially and can't be decoded (crowding). The need for repeated imaging cycles is also a significant issue for many of these technologies, as the images from each cycle need to be exactly aligned to within a few nanometres of precision, which is technically challenging. Finally, these methods are time consuming, since they require a very high magnification in order to achieve single-molecule resolution, and can only image a very small area of the tissue each time. The time required for a full experiment scales both with the area of the tissue being analysed, and with the number of features (genes) being detected, which limits the amount of information that can be recovered.

Image-free gene expression measurement methods avoid the limitations that result from imaging the tissue sample, and rely on sequencing techniques to determine the location of a given RNA molecule. This requires the fusion of a spatial DNA barcode with the molecule to be detected, achieved by using the spatial barcode as primer for reverse transcription, which produces a spatially barcoded cDNA. These methods are much faster as the imaging time, which is relatively slow overall, is removed and data analysis is much simpler. Such methods include; 10× Visium, SlideSeq V1/V2, Seq-Scope, StereoSEQ, and HDST. However, these methods have a lower efficiency, and are commonly limited to capturing no more than 10% of the RNA content of a cell due to limitations in the reverse transcription step. In addition, they all require some sort of solid support on which the spatial DNA barcode is arrayed prior to adding the sample. This support is expensive to produce, fragile, and often results in low spatial resolution, which does not allow capture of information from single cells. Furthermore, the spatial barcoding does not follow the structure of the tissue, but the spatial addresses are arranged either in a regular square grid or randomly. This results in parts of the tissue not being analysed, and in some spatial barcodes overlapping multiple cells, producing imprecise information.

In situ proteomics methods use antibodies conjugated with probes that can be fluorescent molecules, heavy metal isotopes bound by a chemical polymer, or DNA molecules. The tissue is contacted with a library of antibodies so that multiple biological markers (typically protein or protein modifications) are bound by the antibody and linked to the probes. These methods include CODEX, Imaging mass cytometry, MIBI, 4i, cycIF, and Miltenyi MACSima. The probes are then detected by mass spectrometry or by fluorescence imaging (in the latter case, through subsequent imaging cycles as described above for the gene expression measurements). These methods suffer from many of the same issues described above for imaging-based gene expression measurements. Furthermore, in situ proteomics measurements exist mostly as a separate class of techniques, and have not been successfully integrated with gene expression measurements in a high-throughput way allowing measurements of hundreds of genes and proteins together in the same sample. Related antibody free methods can measure a variety of small molecules and potentially peptides and proteins by direct mass spectrometric imaging.

Current spatial profiling methods also have the limitation that they may only profile one or at most two types of biological molecules. For example, methods such as merFISH may be used to profile DNA and RNA whereas other methods such as merFISH, Nanostring geoMX, Nanostring cosMX, DBit-Seq, and seqFISH may profile a limited number of proteins. Other methods such as Spatial-CUT&Tag or Spatial-ATAC-seq may be used to profile chromatin features or open chromatin. However, the simultaneous profiling of DNA, open chromatin, chromatin marks (e.g., a histone mark, transcription factor and/or chromatin factor), protein and RNA (gene expression) at a single cell resolution has not been described in the art.

The present invention aims to solve one or more of the above-mentioned problems by the provision of a novel in situ spatial barcoding method that can be used for labelling several different types of biological molecule with spatial molecular information within the same method, such as: DNA, open chromatin, chromatin features of interest, protein and RNA (gene expression).

The methods of the present invention are based on a technique of using transposases to label or ‘tag’ biological molecules with detection probes which may then form the basis for a spatial barcode. The barcode may be built up through the use of light as a tool to guide spatial barcode assembly onto the labelled biological molecules, so as to allow the identification of the original spatial position of each molecule following high-throughput sequencing.

Advantageously, the methods of the present invention use simple commonly available instruments such as a light microscope and standard tissue slides to provide a method for spatially labelling biological molecules within an area of tissue, down to single cells or sub-cellular compartments, using a combinatorial barcode of arbitrary length. This can be achieved with a resolution equal to the diffraction limit of UV light, at high efficiency, and without many of the issues related to single-molecule imaging, while also bypassing many of the low-resolution and random assignment barcodes seen in spatial barcoding methods.

The method is advantageous in that it enables the quantification and spatial localization of effectively any biological molecule such as gene products, proteins and other biological markers such as chromatin features at the same time, and in the same sample, using high-throughput sequencing. This provides a so-called multi-omics approach to spatial sequencing which has not yet been achieved using many different types of biological molecule and different types of substrate. The method has single-molecule sensitivity, high throughput, and produces data that can be readily analysed using techniques available in the field. Furthermore, it incorporates features allowing the control of some significant sources of error such as off-target probe binding and background noise. The method of the invention is therefore cheaper, quicker, and more powerful, due to the higher sensitivity, the possibility of analysing multiple biological molecules at the same time, and the ease of analysis than existing methods, whilst still extracting detailed spatial information regarding the molecular make-up of a tissue.

The use of transposases also enables the use of custom detection probes containing for example, chemical modifications such as photo-cleavable groups. This enables the use of any transposase-based assay in conjunction with the methods disclosed herein. Furthermore, the methods disclosed herein are compatible with the use of for example, customisable grids of any shape and dimension which allows the selection and analysis of biological molecules in any area of a substrate.

### STATEMENTS OF INVENTION

According to a first aspect of the present invention, there is provided a method of spatially barcoding one or more biological molecules located on or within a substrate, comprising:
(a) Contacting the substrate with one or more transposase complexes, wherein each transposase complex comprises a transposase bound to at least one detection probe, to allow the or each transposase complex to attach the or each detection probe to a biological molecule of interest, optionally wherein the or each detection probe comprises a photocleavable group;
(b) Optionally, if the or each detection probe does not comprise a photocleavable group, adding a photocleavable group to the or each detection probe;
(c) Illuminating a location of interest within the substrate to be spatially barcoded, wherein the illumination cleaves or alters the photocleavable group of the or each detection probe within the location;
(d) Adding an index sequence of the spatial barcode to the or each detection probe within the location illuminated in step (c), wherein the index sequence comprises a photocleavable group;
(e) Repeating steps (c) and (d) until the desired index sequences are added to form a spatial barcode attached to the or each detection probe within the location of interest;
(f) Optionally sequencing the one or more spatially barcoded detection probes of step (e) or a derivative thereof.

According to one embodiment of the first aspect, there is provided a method of spatially barcoding (i) open chromatin and/or chromatin features, and (ii) RNA located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecules of step (a), wherein the or each detection probe optionally comprises a photocleavable group;
(c) Contacting the substrate with one or more detection probes to allow the or each detection probe to bind to an RNA of interest, wherein the or each detection probe optionally comprises a photocleavable group;
(d) Optionally, if the or each detection probe does not comprise a photocleavable group, adding a photocleavable group to the or each detection probe;
(e) Illuminating a location of interest within the substrate to be spatially barcoded, wherein the illumination cleaves or alters the photocleavable group of the or each detection probe within the location;
(f) Adding an index sequence of the spatial barcode to the or each detection probe within the location illuminated in step (e), wherein the index sequence comprises a photocleavable group;
(g) Repeating steps (e) and (f) until the desired index sequences are added to form a spatial barcode attached to the or each detection probe within the location of interest;
(h) Optionally sequencing the one or more spatially barcoded detection probes of step (g) or a derivative thereof.
In one embodiment, the method further comprises a step of performing in situ reverse transcription to generate RNA:DNA hybrids from the RNA molecules of interest as explained in relation to other methods herein. Suitably prior to step (e).

According to one embodiment of the first aspect, there is provided a method of spatially barcoding (i) open chromatin and/or chromatin features, and (ii) RNA located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecules of step (a), wherein the or each detection probe optionally comprises a photocleavable group;
(c) Contacting the substrate with a detection probe that targets one or more RNA molecules of interest.
(d) Performing in situ reverse transcription to generate RNA:DNA hybrids from the RNA molecules of interest;
(e) Optionally, contacting the substrate with one or more additional transposase complexes, wherein each additional transposase complex comprises a transposase bound to at least one detection probe and optionally comprises one or more secondary binding molecules, to allow the or each additional transposase complex to attach the detection probe to an RNA:DNA hybrid of interest, wherein the or each detection probe optionally comprises a photocleavable group; or optionally performing template switching;
(f) Optionally, if the or each detection probe does not comprise a photocleavable group, adding a photocleavable group to the or each detection probe;
(g) Illuminating a location of interest within the substrate to be spatially barcoded, wherein the illumination cleaves or alters the photocleavable group of the or each detection probe within the location;
(h) Adding an index sequence of the spatial barcode to the or each detection probe within the location illuminated in step (g), wherein the index sequence comprises a photocleavable group;
(i) Repeating steps (g) and (h) until the desired index sequences are added to form a spatial barcode attached to the or each detection probe within the location of interest;
(j) Optionally sequencing the one or more spatially barcoded detection probes of step (i) or a derivative thereof.

In one embodiment, in step (e) template switching takes place, suitably instead of the additional transposase step. Suitably in such an embodiment in step (d) a template switching oligonucleotide is annealed to the non-templated overhang of the RNA:DNA hybrid. Suitably the template switching oligonucleotide is complementary to, and anneals to, the non-templated overhang of DNA in the RNA:DNA hybrid. Preferably the template switching oligonucleotide allows the subsequent amplification of the RNA:DNA hybrid by enzymatic DNA synthesis. Suitably the template switching oligonucleotide comprises a PCR primer binding sequence and/or a T7 polymerase promoter sequence. Suitably the template switching oligonucleotide comprises a DNA oligonucleotide and optionally a chemical additive selected from Polyethylene glycol (PEG), betaine, Extreme Thermostable Single-Stranded DNA Binding Protein (ET-SSB), and manganese ions.

Suitably, the template switching oligonucleotide may comprise RNA bases or LNA (locked nucleic acid) modifications.

According to one embodiment of the first aspect, there is provided a method of spatially barcoding (i) open chromatin and/or chromatin features and/or DNA, and (ii) RNA located on or within a substrate, comprising:
(a) Optionally contacting the substrate with one or more chromatin feature binding molecules, wherein each chromatin feature binding molecule binds to a chromatin feature of interest;
(b) Contacting the substrate with a detection probe that targets one or more RNA molecules of interest;
(c) Performing in situ reverse transcription to generate RNA:DNA hybrids from the RNA molecules of interest;
(d) Contacting the substrate with one or more transposase complexes, wherein each transposase complex comprises a transposase bound to at least one detection probe and optionally comprises one or more secondary binding molecules, to allow the or each transposase complex to attach the detection probe to an RNA:DNA hybrid of interest, to DNA at an area of open chromatin, and/or to DNA in proximity of a chromatin binding molecules of step (a), wherein the or each detection probe optionally comprises a photocleavable group;
(e) Optionally, if the or each detection probe does not comprise a photocleavable group, adding a photocleavable group to the or each detection probe;
(f) Illuminating a location of interest within the substrate to be spatially barcoded, wherein the illumination cleaves or alters the photocleavable group of the or each detection probe within the location;
(g) Adding an index sequence of the spatial barcode to the or each detection probe within the location illuminated in step (f), wherein the index sequence comprises a photocleavable group;
(h) Repeating steps (f) and (g) until the desired index sequences are added to form a spatial barcode attached to the or each detection probe within the location of interest;
(i) Optionally sequencing the one or more spatially barcoded detection probes of step (h) or a derivative thereof.
Optionally, a step of template switching may take place, suitably after step (c). Suitably, in such an embodiment, in step (c), a template switching oligonucleotide is annealed to the non-templated overhang of the RNA:DNA hybrid. Suitably the template switching oligonucleotide is complementary to, and anneals to, the non-templated overhang of DNA in the RNA:DNA hybrid. Preferably the template switching oligonucleotide allows the subsequent amplification of the RNA:DNA hybrid by enzymatic DNA synthesis. Suitably the template switching oligonucleotide comprises a PCR primer binding sequence and/or a T7 polymerase promoter sequence. Suitably the template switching oligonucleotide comprises a DNA oligonucleotide and optionally a chemical additive selected from Polyethylene glycol (PEG), betaine, Extreme Thermostable Single-Stranded DNA Binding Protein (ET-SSB), and manganese ions.
Suitably, the template switching oligonucleotide may comprise RNA bases or LNA (locked nucleic acid) modifications.

According to one embodiment of the first aspect, there is provided a method of spatially barcoding open chromatin and/or chromatin features located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecule of step (a), wherein the or each detection probe optionally comprises a photocleavable group;
(c) Optionally, if the or each detection probe does not comprise a photocleavable group, adding a photocleavable group to the or each detection probe;
(d) Illuminating a location of interest within the substrate to be spatially barcoded, wherein the illumination cleaves or alters the photocleavable group of the or each detection probe within the location;
(e) Adding an index sequence of the spatial barcode to the or each detection probe within the location illuminated in step (d), wherein the index sequence comprises a photocleavable group;
(f) Repeating steps (d) and (e) until the desired index sequences are added to form a spatial barcode attached to the or each detection probe within the location of interest;
(g) Optionally sequencing the one or more spatially barcoded detection probes of step (f) or a derivative thereof.

According to one embodiment of the first aspect, there is provided a method of spatially barcoding RNA located on or within a substrate, comprising:
(a) Contacting the substrate with a detection probe that targets one or more RNA molecules of interest;
(b) Performing in situ reverse transcription to generate RNA:DNA hybrids from the RNA molecules of interest;
(c) Contacting the substrate with one or more transposase complexes, wherein each transposase complex comprises a transposase bound to at least one detection probe and optionally comprises one or more secondary binding molecules, to allow the or each transposase complex to attach the detection probe to an RNA:DNA hybrid of interest, wherein the or each detection probe optionally comprises a photocleavable group;
(d) Optionally, if the or each detection probe does not comprise a photocleavable group, adding a photocleavable group to the or each detection probe;
(e) Illuminating a location of interest within the substrate to be spatially barcoded, wherein the illumination cleaves or alters the photocleavable group of the or each detection probe within the location;
(f) Adding an index sequence of the spatial barcode to the or each detection probe within the location illuminated in step (e), wherein the index sequence comprises a photocleavable group;
(g) Repeating steps (e) and (f) until the desired index sequences are added to form a spatial barcode attached to the or each detection probe within the location of interest;
(h) Optionally sequencing the one or more spatially barcoded detection probes of step (g) or a derivative thereof.
Optionally, a step of template switching may take place, suitably after step (b). Suitably, in such an embodiment, in step (b), a template switching oligonucleotide is annealed to the non-templated overhang of the RNA:DNA hybrid. Suitably the template switching oligonucleotide is complementary to, and anneals to, the non-templated overhang of DNA in the RNA:DNA hybrid. Preferably the template switching oligonucleotide allows the subsequent amplification of the RNA:DNA hybrid by enzymatic DNA synthesis. Suitably the template switching oligonucleotide comprises a PCR primer binding sequence and/or a T7 polymerase promoter sequence. Suitably the template switching oligonucleotide comprises a DNA oligonucleotide and optionally a chemical additive selected from Polyethylene glycol (PEG), betaine, Extreme Thermostable Single-Stranded DNA Binding Protein (ET-SSB), and manganese ions.
Suitably, the template switching oligonucleotide may comprise RNA bases or LNA (locked nucleic acid) modifications.
In one embodiment of any of the methods, the transposase complex used is the transposase complex according to the second aspect of the invention.
In one embodiment of any of the methods, the transposase complex attaches the detection probe to the biological molecule to be spatially barcoded by cleaving and tagging the biological molecule with the detection probe. Suitably the transposase complex tagments the biological molecule with the or each detection probe.
In one embodiment of any of the above methods, the one or more chromatin features of interest is a histone mark, a transcription factor, a chromatin factor, a DNA modification site including DNA and RNA methylation sites and the like, a G-quadruplex, a protein that directly or indirectly associates with chromatin, or other molecules that bind to either the protein or chromatin.
In one embodiment of any of the above methods, the detection probe that targets one or more RNA molecules of interest is an RNA detection probe, suitably it comprises a nucleic acid comprising an extendable 3′-OH end, a modality barcode, and a photocleavable group.
In one embodiment of any of the above methods, the method may further comprises an amplification step. In one embodiment, the amplification step is performed after the initial tagmentation step and before the first index addition. Suitably, this amplification increases the pool of molecules available for index ligation and increase coverage for scarce detection targets. In some other embodiments, amplification is performed after all the indices have been added and before sequencing. This increases the abundance of the spatially barcoded products and reduces sample loss during library preparation prior to sequencing. In some embodiments, amplification can be performed twice, firstly after the initial tagmentation step and before index addition, and secondly after index addition. Suitably wherein the amplification step is performed using polymerase chain reaction, T7 RNA in situ transcription, or with loop-mediated isothermal amplification (LAMP).
In one embodiment of any of the above methods, the final index sequence added to the or each detection probe may lack a photocleavable group. Instead of a photocleavable group it may comprise a sequence allowing for example, sequencing library preparation.
In one embodiment, the RNA:DNA hybrid is a RNA:cDNA hybrid.
In one embodiment of any of the above aspects, the step of repeating the previous steps of illuminating and then adding index sequences may be optional, and may not occur. Suitably the method may relate to addition of only one index sequence. Therefore the desired index sequence is added in only one round, and there is no need to repeat the steps of illuminating and then adding index sequences. Therefore the methods encompass those in which the repeating step does not occur.

According to a second aspect of the invention, there is provided a transposase complex, comprising a dimer of transposases, each transposase comprising a detection probe, each detection probe having a first binding region for binding to the transposase, wherein at least one of the detection probes further comprises a second binding region for binding to a spatial barcode, and at least one of the detection probes optionally comprises a photocleavable group.
Suitably the first binding region is bound to the transposase. Suitably the second binding region is capable of binding to a spatial barcode. Suitably a spatial barcode is a nucleic acid sequence capable of identifying a location on or within a substrate, suitably a unique nucleic acid sequence capable of identifying a location on or within a substrate, suitably a unique nucleic acid sequence which has been assigned to a location on or within a substrate. Suitably any spatial barcode may be used with the transposase complex, however preferably the spatial barcode is formed of index sequences as described herein.
In one embodiment where the detection probe does not comprise a photocleavable group, a photocleavable group may be added to the detection probe. Suitably the detection probe may comprise a phosphate group (or a 5′-OH which may be enzymatically converted into a 5′ phosphate in situ). Suitably the photocleavable group is attached to the or each detection probe comprising a secondary binding region, suitably the photocleavable group is attached to the secondary binding region, suitably to enable the ligation of the spatial barcode.
In one embodiment, the dimer comprises a first transposase and a second transposase, each comprising a first detection probe and a second detection probe respectively, preferably wherein the first detection probe comprises the second binding region. In another embodiment, both the first and the second detection probes may comprise a second binding region.
In one embodiment, the first binding region binds to the transposase and comprises a double stranded region. Suitably the first binding region may be known as a ‘mosaic end’.
In one embodiment, the second binding region binds to an index sequence, suitably to multiple index sequences, suitably to multiple index sequences which may form a spatial barcode. Suitably therefore in the steps of the method in which an index sequence is added to the or each detection probe, the index sequence is added to the second binding region of the or each detection probe. Suitably the second binding region comprises a single stranded region. Suitably the second binding region may also be used for amplification, suitably therefore the second binding region may comprise a nucleic acid sequence for amplification.
In one embodiment, the transposase complex comprises one or more secondary binding molecules linked to the or each transposase by fusion or otherwise. Suitably the secondary binding molecule may be a protein-binding domain, a chromatin-binding domain, any domain that interacts with any protein in close proximity to chromatin, an antibody binding protein and/or an antibody or binding fragment thereof as described herein.
Therefore, in one embodiment of any of the above aspects, the one or more secondary binding molecules is selected from one or more of a protein-binding domain, a chromatin-binding domain, any domain that interacts with any protein in close proximity to chromatin an antibody binding protein, or an antibody.

According to a third aspect of the present invention, there is provided a kit comprising: a library of index sequences, one or more transposases, one or more detection probes, optionally a reverse transcriptase enzyme, optionally a ligase enzyme, and optionally one or more reagents.
In one embodiment of any of the above aspects, the or each detection probe further comprises a species barcode and/or a modality barcode.
In one embodiment of any of the above aspects, the detection probe or the RNA detection probe further comprises a nucleic acid sequence comprising a unique molecular identifier (UMI) and/or an amplification region.
In one embodiment of any of the above aspects, the detection probe further comprises one or more moieties or modifications for affinity purification, biotin, desthiobiotin, azides, alkynes, trans-cyclooctenes, strained alkynes such as dibenzylcyclooctyne (DBCO), chloroalkanes, benzyl-guanine. Suitably the detection probe further comprises one or more modifications for the library generation such as deoxyuracil.
In one embodiment of any of the above aspects, the RNA detection probe further comprises one or more moieties or modifications for affinity purification, biotin, desthiobiotin, azides, alkynes, trans-cyclooctenes, strained alkynes such as dibenzylcyclooctyne (DBCO), chloroalkanes, or benzyl-guanine.

Further features and embodiments of the above aspects will now be defined in the following sections. Each feature may be combined in any order or in any combination with any of the above aspects.

The term ‘nucleic acid’ as used herein refers to any polymer formed of a plurality of nucleotide bases, wherein the bases may be comprised of canonical or non-canonical bases, and wherein the backbone may be modified or unmodified, and wherein the nucleotides may be linked by conventional phosphodiester bonds, or non-conventional bonds such as phosphorothioate bonds or chemical bonds. The term ‘nucleic acid mimic’ as used herein refers to a nucleic acid which is non-natural in some manner, for example, wherein one or more of the nucleotide bases is non-canonical, or wherein the backbone is modified, or wherein the bases are non-conventionally linked. ‘nucleic acids’ and ‘nucleic acid mimics’ may include: bridged nucleic acids, locked nucleic acids, peptide nucleic acids, traditional DNA and RNA, for example. In some embodiments, a nucleic acid can be single-stranded, double-stranded, multi-stranded, or combinations thereof. Unless otherwise indicated, a particular nucleic acid sequence of the presently disclosed subject matter optionally comprises or encodes complementary sequences, in addition to any sequence explicitly indicated.

The term ‘a’ or ‘an’ as used herein may refer to the relevant feature in the singular or plural, and should be taken to mean at least one of the relevant feature, and may refer to one or more of the relevant feature.

As used herein, the term “plurality” refers to more than one. Thus, a “plurality of individuals” refers to at least two individuals. In some embodiments, the term plurality refers to more than half of the whole.

As used herein, the term “about” when referring to a value or to an amount of mass, weight, time, volume, concentration or percentage etc. is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate in the context of the invention.

“Probe” as used herein refers to a group of atoms or molecules which is capable of recognising and binding to a specific target molecule or cellular structure and thus allowing detection of the target molecule or structure. Particularly, “probe” refers to a DNA or RNA sequence which can be used to detect the presence of and to quantitate a complementary sequence by molecular hybridization.

The term “hybridize” as used herein refers to conventional hybridization conditions, preferably to hybridization conditions at which 5×SSPE, 1% SDS, 1×Denhardts solution is used as a solution and/or hybridization temperature is between 35° C. and 70° C., preferably 65° C. After hybridization, washing is preferably carried out first with 2×SSC, 1% SDS and subsequently with 0.2×SSC at temperatures between 35° C. and 75° C., particularly between 45° C. and 65° C., but especially at 59° C. (regarding the definition of SSPE, SSC and Denhardts solution see Sambrook et al. loc. cit.). High stringency hybridization conditions as for instance described in Sambrook et al, supra, are particularly preferred. Particularly preferred stringent hybridization conditions are for instance present if hybridization and washing occur at 65° C. as indicated above. Non-stringent hybridization conditions, for instance with hybridization and washing carried out at 45° C., are less preferred and at 35° C. even less.

“Sequence Identity”. The terms “identical” or “identity” in the context of two or more nucleic acid or protein sequences, refer to two or more sequences or sub-sequences that are the same or have a specified percentage of amino acid residues or nucleotides that are the same, when compared and aligned for maximum correspondence, as measured using one of the following sequence comparison algorithms or by visual inspection. If two sequences which are to be compared with each other differ in length, sequence identity preferably relates to the percentage of the nucleotide residues of the shorter sequence which are identical with the nucleotide residues of the longer sequence. As used herein, the percent identity/homology between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, WI 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

Another indication that two nucleic acid sequences are substantially identical is that the two molecules hybridize to each other under stringent conditions. The phrase: “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The “thermal melting point” is the temperature (under defined ionic strength and pH) at which 50% of the target sequence hybridizes to a perfectly matched probe. Very stringent conditions are selected to be equal to the melting temperature (T.sub.m) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2 times SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1 times SSC at 45° C. for 15 minutes. An example low stringency wash for a duplex of, e.g., more than 100 nucleotides, is 4-6 times SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g. when a copy of a nucleic acid is created using the maximum codon degeneracy permitted by the genetic code.

### Substrate or Tissue

In some aspects of the invention, the method involves spatially barcoding one or more biological molecules located on or within a substrate. Suitably the substrate may be an inert substrate such as glass, plastic, etc. Suitably the inert substrate may be a slide, plate, mount, tube, or other item for conducting an assay. Alternatively, the substrate may be living, suitably the substrate may be an or multiple organisms, suitably the substrate may be tissue. Suitably the tissue may be as defined herein. Any features defined herein in relation to an area of the tissue, may equally apply to a location on a substrate.

Some aspects of the present invention involve in situ analysis of gene expression, open chromatin, chromatin marks and/or protein/marker abundance on or within a substrate, which is preferably a biological tissue.

Suitably the tissue may be from any living source.

Suitably the tissue may be parts of an organism or multiple organisms. For example, the tissue may be a sectioned organism such as sections of C. elegans, embryos of the genus Drosophila, zebrafish embryos, or mouse embryos.

Suitably the tissue may be from a human or animal source.

Suitably the tissue may be diseased or healthy tissue.

Suitably the tissue is a sample of tissue. Suitably the sample of tissue is a section. Suitably the section may be obtained by any known means such as a microtome, cryostat, cryomicrotome or vibratome.

Suitably, the tissue section has a thickness ranging from 3 μm to 100 μm. In other embodiments, the tissue section may be thicker depending upon the ability to deliver the required amount of illumination in the area or location of interest with which to cleave or alter the photocleavable groups therein.

Suitably the tissue may be a monolayer of cells.

Suitably the tissue may be stained with one or more stains. Suitably the stains may be any stains known in the art of preparing tissue samples. Suitable stains may include nuclear and/or membrane stains. For example: eosin, DAPI, hematoxylin, phalloidin, WGA, and the like.

Suitably, the tissue may be subjected to one round of immunohistochemistry, or in situ hybridisation according to any method known in the art, for the purpose of visualizing the distribution of certain protein markers using fluorescence imaging. Suitably the methods may comprise a step of staining the tissue. Suitably the methods may comprise a step of immuno-staining the tissue. Suitably the round of immunohistochemistry may be carried out prior to step (a) of the methods of the invention.

Suitably, prior to the methods of the invention, the tissue or substrate is imaged. Suitably therefore the methods may comprise a step of imaging the tissue or substrate. Suitably the tissue is imaged by a camera. Suitably the camera captures one or more images of the tissue. Optionally the camera may be part of the instrument, suitably the microscope or slide scanner, used to image the tissue.

Suitably software is used to analyse the one or more images of the tissue. Suitably the software is operable to conduct image analysis. Suitably the software is operable to conduct mosaic imaging analysis. Suitably therefore the method may comprise a step of conducting mosaic imaging analysis of one or more images of the tissue or substrate. Suitably the software is operable to identify individual cells or sub-cellular regions within the or each image, suitably by automated object recognition. Suitably therefore the method may comprise identifying individual cells or sub-cellular regions in one or more images of the tissue. Suitably, the software allows a user to select any number of locations or areas of interest of any size, ranging from sub-cellular to the whole sample, for subsequent spatial barcoding. Suitably therefore the method may comprise a step in which one or more locations or areas of interest of the substrate are selected, suitably from the one or more images, for spatial barcoding.

In another embodiment, a grid may be applied to the tissue section or to an image of the tissue. Suitably this may be a fixed grid. Alternatively this may be a grid that is customisable. Suitably, areas of the grid may be assigned based on any regular pattern or any irregular pattern as desired. Suitably areas of the grid may be a polygon shape such as square, circle, rectangle, hexagon or the like. Alternatively, the regions can have a custom shape, such as an irregular polygon defined by a series of points, or could be drawn free-hand. Suitably, a series of identical or differently shaped regions might be arranged in a regular grid. Suitably the regions may be distributed according to a regular pattern with a defined X and Y pitch. Alternatively the grid might be irregular, with each region assigned a position as desired. Suitably the areas of the grid may be between 1 μm² and 1 cm², suitably between 1 μm² and 1 mm², suitably between 1 μm² and 100 μm². Suitably any number of arbitrarily defined regions, suitably ranging from one region to as many regions are allowed by the coding capacity of the indexing strategy, may be used to select one or more locations of interest of the substrate.

Optionally a grid approach may be combined with a mosaic imaging analysis as described above.

### Area or Location of Interest

The methods of the present invention comprise selecting and illuminating one or more locations or areas of interest, in which locations or areas detection probes are to be spatially barcoded.

Suitably, reference to ‘area’ or ‘location’ herein may refer to a two-dimensional region or a three-dimensional region. Suitably to a region of any size. Suitably the maximum size of the region may be determined by the properties of the illumination and/or the particular tissue or substrate used in the method.

Suitably a location of interest may be any region, suitably any region on a substrate. Suitably, an area of interest is a two-dimensional region.

Suitably an area of interest may be between 1 μm²-150 mm² in size, suitably between 1 μm²-1 mm² in size, suitably between 1 μm²-200,000 μm² in size, suitably between 1 μm²-20,000 μm² in size, suitably between 1 μm²-1000 μm² in size.

Suitably an area of interest may be any region within a tissue. Suitably, an area of interest may also be a three-dimensional region within the tissue.

Suitably an area of interest may be between 1 μm³-150 mm³ in size, suitably between 1 μm³-1 mm³ in size, suitably between 1 μm³-1,000,000 μm³ in size, suitably between 1 μm³-200,000 μm³ in size, suitably between 1 μm³-20,000 μm³ in size, suitably between 1 μm³-1000 μm³ in size, suitably between 1 μm³ to 1,000,000,000 μm³ in size, suitably 1 μm³ to 200,000,000 μm³ in size, suitably 1 μm³ to 20,000,000 μm³ in size, suitably 1 μm³ to 1,000,000 μm³ in size.

Suitably an area or location of interest may be a collection of cells, suitably an area or location of interest may comprise from 1 up to 100,000,000 cells, 1,000,000 cells, 1000 cells, 100 cells, 10 cells. Suitably an area or location of interest may comprise a single cell. Suitably an area or location of interest may comprise a sub-cellular region or compartment.

Suitably one or more locations or areas of interest are pre-selected, suitably prior to the methods of the invention. Suitably a user selects the locations or areas of interest, suitably from an image of the tissue. Suitably an area or location may be selected based on pixels or based on features of the image, or both. Suitably image processing aids selection of an area or location from an image. Suitably areas or locations may also be selected from an image using a grid approach as explained elsewhere herein.

Suitably software then assigns a unique spatial barcode to each selected location or area of interest. Suitably therefore, the methods of the invention may comprise a step of selecting one or more locations of interest of the substrate, or selecting one or more areas of interest of the tissue. Suitably therefore, the methods of the invention may comprise a step of assigning a spatial barcode to each selected location or area of interest.

Suitably multiple locations or areas of interest can be selected. Suitably the locations or areas of interest do not have to be contiguous.

Suitably the number of locations or areas that can be selected is determined by the number of possible unique spatial barcode sequences. The number of unique spatial barcode sequences is in turn determined by the number of different index sequences used and by the number of index sequences included in each spatial barcode.

Suitably, based on a method using 4 different index sequences and 10 index sequences per spatial barcode, up to around 1 million, specifically 1,048,576, locations or areas of interest can be selected.

### Transposase Complexes

The present invention makes use of transposase complexes which attach at least one detection probe to a biological molecule of interest.

Suitably, the transposase complex of the invention comprises a dimer of transposases, each transposase comprising a detection probe, each detection probe having a first binding region for binding to the transposase, wherein at least one of the detection probes further comprises a second binding region for binding to a spatial barcode, and at least one of the detection probes optionally further comprises a photocleavable group. Preferably the methods of the invention make use of the transposase complex of the invention, which is mostly described herein. Preferably therefore, the one or more transposase complexes used in any method of the invention are a transposase complex of the second aspect of the invention, which may suitably comprise any of the features described herein.

However, it is to be understood that any suitable transposase may be used in the methods of the invention. Suitably any transposase or complex thereof which is capable of being loaded with one or more detection probes, cleaving and tagging a biological molecule with said detection probes may be used. Suitable examples of various transposases that may be used are provided hereinbelow.

Suitably, where the detection probe does not comprise a photocleavable group, a photocleavable group may be added to the detection probe. Suitably the detection probe may comprise a phosphate group (or a 5′-OH which may be enzymatically converted into a 5′ phosphate in situ). Suitably the photocleavable group is attached to the or each detection probe, suitably to the secondary binding region, suitably to enable the ligation of the spatial barcode.

Suitably the spatial barcode may be any nucleic acid sequence with a unique sequence. Suitably a unique sequence which has been assigned to a given area of a substrate to be investigated. Suitably in the present invention, the spatial barcode is formed from a plurality of index sequences as described herein.

Suitably, a dimer of transposases refers to two transposase monomers or subunits that may be complexed together. Suitably this may be referred to a pair of transposases.

Suitably each transposase comprised in the dimer is the same transposase. Suitably the first and second transposases are the same, they have the same amino acid sequences. Even if, in some embodiments, one or both of the transposases comprises a fusion protein with another different protein, suitably the transposases comprise the same amino acid sequence.

Suitably, each of the detection probes, the first and second binding regions thereof, the index sequences of the spatial barcode and the photocleavable group of the transposase complex are as described elsewhere herein.

Suitably, the transposase complex comprises a transposase, suitably a first and a second transposase. Suitably each transposase comprises one or more detection probes as disclosed herein. Suitably each transposase comprises at least one detection probe. Suitably each transposase comprises one detection probe. Suitably, one transposase monomer comprises one detection probe. Suitably therefore the first transposase comprises the first detection probe and the second transposase comprises the second detection probe.

Suitably, one transposase monomer comprises one detection probe comprising two nucleic acids containing a first binding region to attach to the transposase, and a second binding region to attach to the spatial barcode. The or each detection probe may further comprise a photocleavable group, suitably attached to the second binding region, suitably to enable index sequences to be ligated thereto to form the spatial barcode. Suitably, the second binding region may also be used as an amplification region, suitably for library preparation. Suitably therefore the second binding region is for amplification.

In one embodiment, the dimer comprises a first transposase and a second transposase, each comprising a first detection probe and a second detection probe respectively, preferably wherein the first detection probe comprises the second binding region. In another embodiment, both the first and the second detection probes may comprise a second binding region.

Suitably each detection probe has a first binding region for binding to the transposase. Suitably the first binding region comprises a nucleic acid, suitably a double-stranded DNA sequence, that binds to a region of the transposase. In one embodiment, the first binding region binds to the transposase and comprises a double stranded region. Suitably the first binding region may be known as a ‘mosaic end’. Suitably, the first binding region comprises double-stranded DNA. Optionally the first binding region may further comprise one or more modifications to the nucleic acid sequence, suitably to the backbone of the sequence. For example, the first binding region may comprise phosphorothioate (PS) bonds. Suitably, the first binding region may also be used for adapter-switching reactions, suitably for library preparation. Suitable techniques of library preparation are known in the art, for example in Mulqueen et al. 2021.

Suitably the first binding region comprises a sequence recognised by the transposase, and bound by the transposase. Suitably the first binding region is between 10-40 nt in length, suitably between 15-30 nt in length, suitably between 15-20 nt in length, suitably around 19 nt in length. Suitably the first binding region comprises a DNA sequence having a sense strand according to SEQ ID NO: 76 (5′-P-CTGTCTCTTATACACATCT-3′) or a sequence having a limited number of modifications thereto, such as for example, up to 5, 4, 3, or 2 modifications.

Suitably either or both of the detection probes may further comprise a second binding region for binding to a spatial barcode. Suitably one of the detection probes may further comprise a second binding region for binding to a spatial barcode. Suitably the first detection probe comprises a second binding region for binding to a spatial barcode. Suitably for binding to an index sequence to build a spatial barcode. Suitably index sequences are ligated to the second binding region and subsequently to each other during each round of addition to build up a spatial barcode as described herein. Suitably the second binding region comprises a single stranded nucleic acid capable of binding to a spatial barcode, suitably single stranded DNA. Suitably the second binding region is attached to, and contiguous with, the first binding region to form the detection probe. Suitably the second binding region is attached to, and contiguous with, the antisense strand of the first binding region. Suitably the second binding region is attached to, and contiguous with, the 5′ end of the antisense strand of the first binding region.

In one embodiment, the second binding region binds to an index sequence, suitably to multiple index sequences, suitably to multiple index sequences which may form a spatial barcode. Suitably therefore, in the steps of the method in which an index sequence is added to the or each detection probe, the index sequence is added to the second binding region of the or each detection probe. Suitably by ligation.

Suitably either or both of the detection probes may further comprise a photocleavable group. Suitably one of the detection probes may further comprise a photocleavable group. Suitably the first detection probe comprises a photocleavable group. Suitably the photocleavable group is attached to the or each detection probe which comprises a second binding region, suitably to the second binding region, suitably to enable the attachment of spatial barcode thereto.

In one embodiment, therefore, the transposase complex comprises a first transposase and a second transposase, each comprising a first detection probe and a second detection probe respectively, wherein each detection probe comprises a first binding region for binding to the respective transposase, and wherein the first detection probe comprises a second binding region for binding to a spatial barcode and the photocleavable group.

Suitable further features of the detection probes are described herein below.

Suitably, the or each transposase of the transposase complex may further comprise one or more secondary binding molecules such as an antibody binding protein and/or an antibody, or other binding domain, as described further herein, to allow the transposase complex to attach the detection probe to a biological molecule of interest as disclosed herein. In one embodiment, the transposase complex comprises one or more secondary binding molecules such as a protein-binding domain, a chromatin-binding domain, a domain that interacts with any protein in close proximity to chromatin, an antibody binding protein and/or an antibody or binding fragment thereof as described herein.

Suitably, for example, the secondary binding molecule may bind to the chromatin feature binding molecule. Suitably this enables the transposase to attach the or each detection probe to the biological molecule at the position of the chromatin feature. Suitably, for example, in other situations, the secondary binding molecule may bind directly to the biological molecule. In such embodiments, the secondary biological molecule may comprise, for example, a binding domain specific for RNA:DNA or another nucleic acid, such as a transcription factor or Zinc finger, or may comprise a binding domain specific for modified nucleosides such as digoxigenin (DIG)-binding protein, which may be present within the biological molecule.

As such, the secondary binding molecule may be used as a tool to bring the transposase in proximity to the biological molecule so that it may cleave it and attach the or each detection probe thereto.

Suitably the secondary binding molecule is attached to the transposase complex, suitably by generating a modified transposase fused with the secondary binding molecule. Suitably such a fusion protein may be generated by genetic engineering and expressing the fusion protein recombinantly using any of the methods known in the art, or, alternatively, by crosslinking the secondary binding molecule to the transposase using any of the methods known in the art, suitably amino-reactive or thiol-reactive chemical crosslinkers. Suitably each transposase of the transposase dimer may comprise a secondary binding molecule. Suitably, a secondary binding molecule may be attached to the transposase by expression as fusion protein or by chemical conjugation (e.g., covalent linkage) to the transposase using for example, a sortase, or by a linker, suitably a peptide linker for example.

Suitably, in some embodiments of the methods, the transposase complex attaches one or more detection probes to an area of open chromatin and/or to DNA in proximity to a chromatin feature binding molecule bound to a chromatin feature of interest. Suitably, the transposase allows for the profiling of open (accessible areas of) chromatin and/or chromatin features. Suitably in embodiments where the transposase is used to profile chromatin features, the chromatin feature may first be bound by a chromatin feature binding molecule, which in turn is bound by a secondary binding molecule present in the transposase complex. In some embodiments, a further intermediate binding molecule may be present to link the chromatin feature binding molecule to the secondary binding molecule as explained elsewhere herein.

Suitably, in some embodiments of the methods, the transposase complex attaches one or more detection probes to DNA of interest. Suitably, the transposase allows for the profiling of DNA. Suitably in such embodiments, a secondary binding molecule may not be required.

Suitably, in some embodiments of the methods, the transposase complex attaches one or more detection probes to an RNA:DNA hybrid of interest. Suitably, the detection probe is attached to RNA by one transposase of the dimer of transposases and the detection probe is attached to DNA by the other transposase of the dimer of transposases. Suitably therefore, the transposase allows for the profiling of RNA. Suitably in some embodiments where the transposase is used to profile RNA:DNA hybrids then the RNA:DNA may be bound by a secondary binding molecule present in the transposase complex. Suitably the RNA:DNA hybrid may comprise modified nucleosides such as digoxigenin (DIG)-11-deoxyuridine to which the secondary binding molecule can bind. Suitably the RNA:DNA hybrid may comprise modified nucleosides by the use of modified dNTPs during the reverse transcription step of the methods.

Suitably the transposase complex cleaves and inserts one or more detection probes into the target biological molecule. Suitably the transposase complex cleaves and inserts one or more detection probes into open chromatin, into DNA, into an RNA:DNA hybrid, and/or into DNA in proximity of a chromatin feature binding molecule bound to a chromatin feature of interest.

Suitably by ‘in proximity’ of or to a chromatin feature binding molecule it is meant that the transposase complex cleaves within a short distance of the chromatin feature binding molecule, for example within 0.1 nm to 100 nm of the chromatin feature binding molecule, suitably within 2 nm to 50 nm, suitably within 4 nm and 40 nm, suitably within 5 nm to 20 nm of the chromatin feature binding molecule. Suitably this distance is determined by the specific transposase complex which is used. Suitably, the skilled person will understand that this distance is influenced by the way the secondary binding molecule is attached to the transposase complex, if this is achieved by a linker, then the length of the linker will affect the distance from the chromatin feature binding molecule to the point of cleavage. Suitably the distance between the chromatin feature binding molecule and the cleavage site may be as defined in the art for various transposase complexes. Suitably the exact point of cleavage of the DNA in proximity to the chromatin feature binding molecule is not critical to the invention.

Suitably the transposase complex cleaves the target biological molecule and inserts the one or more detection probes at the point of cleavage. Suitably the transposase complex cleaves the target biological molecule to generate a cleaved fragment. Suitably the transposase inserts the one or more detection probes into the cleaved fragment. Suitably at either end of the cleaved fragment. Suitably at the 5′ and the 3′ end of the cleaved fragment. Suitably therefore the transposase complex cleaves and ‘tags’ the biological molecule of interest, also known as ‘tagmentation’ in the art, with the one or more detection probes, suitably at either end thereof. Suitably in a process known as tagmentation. Suitably the transposase complex cleaves and tags open chromatin, DNA, RNA:DNA hybrids, and/or DNA in proximity of a chromatin feature binding molecule bound to a chromatin feature of interest with one or more detection probes. Suitably the transposase complex cleaves open chromatin, DNA, RNA:DNA hybrids, and/or DNA in proximity of a chromatin feature binding molecule bound to a chromatin feature of interest to generate a cleaved fragment thereof, and tags the cleaved fragment with one or more detection probes.

Suitably therefore the transposase complex tagments the biological molecule of interest. Suitably therefore the transposase complex tagments open chromatin, DNA, RNA:DNA hybrids, and/or DNA in proximity of a chromatin feature binding molecule bound to a chromatin feature of interest.

Suitably in some of the methods described herein, two steps of contacting with a transposase complex take place, suitably a first step with a first transposase complex and a second or additional step with a second transposase complex. Suitably the transposase complexes used in each step may be different. Suitably they may be directed to bind to different target biological molecules. Suitably this may be achieved by using transpose complexes with different secondary binding molecules specific for different targets. For example, in a method of spatially barcoding both a chromatin feature and RNA, the first transposase complex may comprise a secondary binding molecule which binds to the chromatin feature binding molecule to tagment DNA, and the additional transposase complex may comprise an secondary binding molecule which binds to RNA:DNA hybrids to tagment RNA.

Suitably, in such methods with two steps of contacting with a transposase complex, the steps may take place at the same time, suitably in a ‘one-pot’ reaction. Suitably, therefore the different biological molecules may be tagmented at the same time by both the first and second or additional transposase complexes. In such an embodiment, suitably the RNA:DNA hybrid may comprise modifications to distinguish it from the chromatin features, or open chromatin. Suitably such modifications may comprise modified nucleosides such as digoxigenin (DIG)-11-deoxyuridine in the sequence of the RNA:DNA hybrid to which the secondary binding molecule can bind. Suitably therefore in some methods of the invention, there may only be one step of contacting with a transposase complex, suitably contacting with a plurality of transposase complexes. Suitably the transposase complexes used in such a step may be targeted to a plurality of different biological molecules. Suitably therefore, they may comprise a plurality of different secondary binding molecules and additionally may comprise some transposase complexes with no secondary binding molecule for targeting open chromatin.

Suitably therefore, within one step of contacting with a transposase complex, different biological molecules may be tagmented. Suitably for example the first transposase complex may comprise a plurality of first transposase complexes, and the additional transposase complex may comprise a plurality of different transposase complexes. Suitably each transposase complex may comprise a different secondary binding molecule, but which may be directed to bind the same type of biological molecule. For example, in methods which spatially barcode chromatin features, suitably the first transposase complexes used in the first step of contacting with a transposase complex may each comprise a secondary binding molecule. Suitably however the secondary binding molecules may be directed to bind different chromatin features. Suitably some of the first transposase complexes may be directed to bind to histone methylation for example, and some may be directed to bind to a transcription factor of interest.

Therefore, the methods of the invention, through using a transposase complex which may be flexibly targeted to any biological molecule using secondary binding molecules, allows the spatial labelling and profiling of numerous different types of biological molecules at the same time, in a multiomics approach. In the methods which comprise a step of contacting a substrate with one or more transposase complexes, to allow the or each detection probe to be attached to a biological molecule, suitably the contact is under conditions sufficient to allow the transposase complex to attach the or each detection probe to a biological molecule. Suitably it is understood that the transposase complex may directly or indirectly contact the substrate, suitably the biological molecule to be spatially barcoded. Suitably the transposase complex may directly contact the biological molecule and ‘tagment’ it, for example DNA or RNA. Alternatively the transposase complex may indirectly contact the biological molecule and ‘tagment’ it, suitably via a chromatin feature binding molecule or another such binding molecule which may be bound to the biological molecule itself, for example histone modifications.

Suitable conditions may include contacting the substrate with the or each transposase complex for a sufficient length of time to allow the transposase complex to attach the detection probe to the biological molecule. Suitably a sufficient time is between 1 minute and 1 week, suitably between 5 minutes and 24 hours, suitably between 15 minutes and 6 hours, suitably around 30 minutes, depending on the abundance of the biological molecule, the substrate, and the size of the substrate or tissue sample.

Suitable conditions may include contacting the tissue with a sufficient amount of the transposase complex. Suitably, the transposase complex including the or each detection probe is supplied at a sufficient concentration to allow attachment to the biological molecule. Suitably a sufficient concentration is between 100 pM and 100 μM depending on the abundance of the biological molecule and the number of detection probes, suitably between 1 nM and 10 μM, suitably between 10 nM and 1 μM, suitably about 63 nM.

Suitable conditions to allow attachment of a detection probe to a given biological molecule of interest by a transposase complex will be known or determined by the skilled person.

Suitably, any transposase that cleaves and attaches a detection probe to a biological molecule as disclosed herein may be used in connection with the methods of the invention. Suitably, the transposases can be of prokaryotic or eukaryotic origin. Suitably, the transposases can be any naturally occurring or engineered, including mutated, transposase. Suitably, the transposase may include, but is not limited to, integrases, HERMES, and HIV integrases. These include Tn transposases (Tn5, Tn3, Tn7, Tn10, Tn552, Tn903) and its derivates (TnY), MuA transposases, Vibhar transposases, Ac-Ds, Ascot-1, Bsl, Cin4, Copia, En/Spm, F-element, hobo, Hsmar1, Hsmar2, IN(HIV), IS1, IS2, IS3, IS4, IS5, IS6, IS10, IS21, IS30, IS50, IS51, IS150, IS256, IS407, IS427, IS630, IS903, IS91, IS982, IS1031, ISL2, L1, Mariner, P-element, Tam3, Tc1, Tc3, Tel, THE-1, Tn/O, TnA, Toli, To12, Tyl, any prokaryotic transposase, or any transposase related to and/or derived from those listed here.

In some embodiments, each transposase is a Tn5 transposase. Suitably, each Tn5 transposase comprises the sequence of SEQ ID NO:1 or an active fragment thereof, or a sequence having at least 70%, 75%, 80%, 85%, 90%, 95%, 96%, 97%, 98%, 99% identity thereto. Suitably such levels of identity apply to each transposase only, and do not apply across entire fusion proteins of a transposase fused to another protein or protein domain.

Suitably, each transposase may be modified, or a derivative or fusion of a reference or wild type transposase. Suitably each transposase may be modified, suitably it may be modified to form a hyperactive transposase. Suitably a hyperactive transposase comprises an enzymatic activity which is higher than a reference or wild type transposase.

In some embodiments, each transposase is a modified transposase, suitably a transposase that has been modified to become hyperactive by one or more of the following mutations: E54K, M56A, L372P, Y41A, Y41C, S42A, W450C, E451C, E454C. Suitably each transposase may comprise a sequence according to any of SEQ ID NOs: 2 to 10, or an active fragment thereof, or a sequence having at least 70%, 75%, 80%, 85%, 90%, 95%, 96%, 97%, 98%, 99% identity thereto. Suitable modified transposes are be described in U.S. Pat. No. 7,608,434, Reznikoff and Gradman, for example.

### Binding Molecules/Chromatin Feature Binding Molecules/Secondary Binding Molecules

The present invention makes use of one or more binding molecules, suitably one or more chromatin feature binding molecules and one or more secondary binding molecules, and optionally one or more intermediate binding molecules.

Suitably, the chromatin feature binding molecule is operable to bind to a chromatin feature of interest as disclosed herein.

Suitably, the transposase complex may in some embodiments comprise one or more secondary binding molecules. Suitably, the secondary binding molecule is operable to bind to a chromatin feature binding molecule. Suitably the secondary binding molecule may also be operable to bind to a domain which binds to a biological molecule, such as a chromatin binding domain, a chromodomain, a transcription factor, etc. or to a feature of a biological molecule such as a tag such as a flourescent tag, or a modification, such as a modified nucleotide for example.

Suitably, the secondary binding molecule may also be operable to bind to an intermediate binding molecule. Suitably an intermediate binding molecule may link the chromatin feature binding molecule to the secondary binding molecule. For example, the chromatin feature binding molecule may be a primary antibody, the intermediate binding molecule may be a secondary antibody, and the secondary binding molecule may be an antibody binding protein.

Suitably, the chromatin feature binding molecule and/or intermediate binding molecule and/or secondary binding molecule is a protein. Suitably, the protein is an antibody binding protein (ABP), an antibody, a transcription factor (or a fragment thereof), a chromatin modifier protein (or a fragment thereof), or a protein interacting with a chromatin associated protein (or a fragment thereof). An antibody may be an antibody, an antigen-binding fragment thereof, a variable domain of a heavy chain camelid antibody (VHH), i.e., a nanobody, or an aptamer for example.

“Antibody” as employed herein refers to an immunoglobulin molecule as discussed below in more detail, in particular a full-length antibody or a molecule comprising a full-length antibody, for example a DVD-Ig molecule and the like. Suitably comprising at least one heavy or light chain CDR of a reference antibody molecule, and at least six CDRs from one or more reference antibody molecules. Suitably, the antibody as employed herein may refer to a primary antibody and/or a secondary antibody.

A “binding fragment thereof” is interchangeable with “antigen binding fragment thereof” and refers to an epitope/antigen binding fragment of an antibody fragment, for example comprising a binding region, in particular comprising 6 CDRs, such as 3 CDRs in heavy variable region and 3 CDRs in light variable region.

Suitably, the antibody or binding fragment thereof is selected from: naturally-occurring, polyclonal, monoclonal, multispecific, primate, mouse, camelid, shark, human, humanized, primatized, or chimeric. Suitably, the antibody or binding fragment thereof may be an epitope-binding fragment, e.g., Fab′ and F(ab′)2, Fd, Fvs, single-chain Fvs (scFv), disulfide-linked Fvs (sdFv), fragments comprising either a VL or VH domain, or fragments produced by a Fab expression library. Suitably, the antibody or binding fragment thereof may be a minibody, a mintbody (modification-specific intracellular antibody), a diabody, a triabody, a tetrabody, or a single chain antibody. Suitably, the antibody or binding fragment thereof is a monoclonal antibody. ScFv molecules are known in the art and are described, e.g., in U.S. Pat. No. 5,892,019.

Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

Suitably, the variable domain of a heavy chain (VHH) antibody can be a fragment of a camelid single-chain heavy chain antibody (also known as “nanobody”) including a binding region, suitably comprising 3 CDRs.

Suitably the antibody or binding fragment thereof may be derived from any organism. Suitably the antibody or binding fragment thereof may be produced endogenously within the organism or cell or may be heterologous and provided to the organism or cell.

The chromatin feature binding molecule may be a binding molecule which binds to a chromatin feature of interest such as a histone mark/modification, including but not limited to methylation, acetylation or ubiquitylation, transcription factor, chromatin associated protein such as a chromatin remodelling protein, chromatin factor, nucleic acid modifications, including DNA modification sites including DNA and RNA methylation sites, or a G-quadruplex.

Suitably the chromatin feature binding molecule is an antibody, or binding fragment thereof. Suitably the chromatin feature binding molecule is an antibody, or binding fragment thereof which is operable to bind to a chromatin feature of interest such as those listed above.

Suitably, the histone mark may be H3K4 mono-, di-, or tri-methylation, H3K4 acetylation, H3K9 mono-, di-, or tri-methylation, H3K9 acetylation, H3K27 mono-, di-, or tri-methylation, H3K27 acetylation, H3K36 mono-, di-, or tri-methylation, H3K36 acetylation, H4K20 mono-, di-, or tri-methylation, gamma H2A.X. Suitably, the antibody may be Rabbit polyclonal anti-H3K4me3 (Abcam, Cat #ab8580), Rabbit polyclonal anti-H3K9ac (Abcam, Cat #ab4441), Rabbit polyclonal anti-H3K9me3 (Abcam, Cat #ab8898), Rabbit polyclonal anti-H3K27me3 (Merck Millipore, Cat #07-449), Rabbit polyclonal anti-H3K36me3 (Active Motif, Cat #61902), Rabbit polyclonal anti-H4K20me1 (Abcam, Cat #ab9051), mouse anti-H3K27me3 (Abcam, cat #Ab6002), rabbit anti-H3K27ac (Abcam, cat #Ab177178), rabbit anti-H3K27me3 (Cell Signaling, cat #9733), mouse monoclonal anti-gammaH2A.X antibody (Abcam, cat #ab26350).

Suitably, the transcription factor may be, but are not limited to, TFIIA, TFIIB, TFIID, Hox family TF, HSF, SREBP, HIF, ZnF TF, Myc, basic-leucine zipper TF (bZIP), basic-helix-loop-helix TF, homeodomain proteins. In such case, the chromatin feature binding molecule may be antibody or VHH nanobody. The antibody may be Rabbit monoclonal Anti-TFIIB antibody (Abcam, cat #ab109106), Mouse monoclonal Anti-TATA binding protein TBP antibody (Abcam, cat #ab818), Rabbit monoclonal Anti-HSF1 antibody (Abcam, cat #ab52757), Rabbit polyclonal Anti-SREBP1 antibody (Abcam, cat #ab28481), Rabbit monoclonal Anti-HIF-1 alpha antibody (Abcam, cat #ab179483), Rabbit polyclonal Anti-Zfp281/ZNF281 antibody (Abcam, cat #ab101318), Rabbit monoclonal Anti-c-Myc antibody (Abcam, cat #ab32072), Rabbit polyclonal Anti-BHLHB5 antibody (Abcam, cat #ab204791).

Suitably, the chromatin associated protein or chromatin factor may be any chromatin associated protein or chromatin factor such as a chromodomain, bromodomain, zinc finger, TALE, or polymerase, or any of those listed below such as 14-3-3 domain, ADD domain, ankyrin, BAH domain, BIR domain, BRCT domain, tandem BRCT domain, bromodomain, double bromodomain, chromobarrel, chromodomain, double chromodomain, double PHD finger domain, MBT domain, PID domain, PHD domain, double PH domain, PWWP domain, royal family domain, Tudor domain, tandem Tudor domain, WD40 domain, or a zinc finger CW domain. Suitable examples are provided in U.S. Pat. No. 10,689,643 and EP3277710B1. Suitably the chromatin associated protein or chromatin factor may be, but is not limited to, CBX1, CBX7, Heterochromatin Protein 1a, Polycomb, TAF3, BRD4, MBD2, CTCF, ARID1B (or other ARID complex components), DNMT family protein, RNA Polymerase I, II or III subunits. In such case, the chromatin feature binding molecule may be antibody or VHH nanobody. The antibody may be Rabbit monoclonal Anti-CBX1 antibody (Abcam, cat #ab270988), Rabbit polyclonal Anti-cbx7 antibody (Abcam, cat #ab21873), Rabbit monoclonal Anti-TAF3 antibody (Abcam, cat #ab188332), Rabbit monoclonal Anti-HP1 alpha antibody (Abcam, cat #ab109028), Mouse monoclonal Anti-PCNA antibody (Abcam, cat #ab29), Rabbit monoclonal Anti-Brd4 antibody (Abcam, cat #ab128874), Rabbit monoclonal Anti-MBD2 antibody (Abcam, cat #ab188474), Rabbit monoclonal Anti-CTCF antibody (Abcam, cat #ab128873), mouse monoclonal Anti-ARID1B antibody (Abcam, cat #ab57461), rabbit monoclonal Anti-Dnmt1 antibody (Abcam, cat #ab188453), rabbit polyclonal Anti-RNA polymerase II CTD repeat YSPTSPS (phospho S5) antibody (Abcam, cat #ab5131), rabbit monoclonal Anti-RNA polymerase II CTD repeat YSPTSPS (phospho S2) antibody (Abcam, cat #ab238146), Rat monoclonal Anti-RNA polymerase II CTD repeat YSPTSPS (phospho S7) antibody (Abcam, cat #ab252853).

Suitably, the nucleic acid modification sites, including DNA methylation may be, for example, a 5-methylcytosine (5mC), 5-hydroxymethylcytosine (5hmC), 5-formylcytosine (5fC), and 5-carboxylcytosine (5caC), N4-methylcytosine, N6-methyladenine. In such case, the chromatin feature binding molecule may be antibody or VHH nanobody, or a domain that detects methylated DNA such as MBD [methyl-CpG-binding domain].

The antibody may be rabbit polyclonal anti-3-Methylcytosine (3-mC) antibody (Active Motif, cat #61112), mouse monoclonal Anti-5-methylcytosine (5-mC) antibody (Abcam, cat #ab10805), rat monoclonal Anti-5-hydroxymethylcytosine (5-hmC) antibody (Abcam, cat #ab106918), rabbit polyclonal Anti-5-formylcytosine (5-fC) antibody (Abcam, cat #ab231898), rabbit polyclonal Anti-5-carboxylcytosine (5-caC) antibody (Abcam, cat #ab231801), rabbit monoclonal anti-N6-Methyladenosine (m6A) antibody (ThermoFisher, cat #MA5-33030), rabbit polyclonal Anti-N6-methyladenosine (m6A) antibody (Abcam, cat #ab151230).

Suitably, a chromatin feature of interest may be a G-quadruplex. In such case, the chromatin feature binding molecule may be antibody or VHH nanobody. The antibody may be BG4 or 1H6.

Suitably the secondary binding molecule may be a further antibody or binding fragment thereof, or may be an antibody binding protein. Suitably the secondary binding molecule is an antibody binding protein which is operable to bind to an intermediate antibody, or to an antibody or binding fragment thereof which is operable to bind to a chromatin feature of interest.

Suitable antibody binding proteins may be: protein A from Staphylococcus aureus, protein G from Streptococcus, protein L from Peptostreptococcus magnus, or their variants.

Suitably, the chromatin feature binding molecule and/or secondary binding molecule comprised in the transposase complex may be a transcription factor, a chromatin associated protein or chromatin factor, or a fragment thereof, for instance a DNA or chromatin binding domain. Suitably, the DNA or chromatin binding domain has a structure providing specific binding to a particular chromatin modification or structure (as described above) or to a particular DNA sequence. Suitably, the DNA or chromatin binding domain could be a 14-3-3 domain, ADD domain, ankyrin, BAH domain, BIR domain, BRCT domain, tandem BRCT domain, bromodomain, double bromodomain, chromobarrel, chromodomain, double chromodomain, double PHD finger domain, MBT domain, PID domain, PHD domain, double PH domain, PWWP domain, royal family domain, Tudor domain, tandem Tudor domain, WD40 domain, or a zinc finger CW domain. Accordingly, the chromatin feature binding molecule and/or secondary binding molecule may bind to any other molecule which binds to the chromatin feature. Suitably, the chromatin feature may be tagged with a protein, for example a Halo tag, Flag tag, HA tag or V5 tag, or a fluorescent protein such as GFP, mCherry or RFP, and suitably the chromatin feature binding molecule and/or secondary binding molecule binds to said protein i.e., GFP, RFP, Halo tag, Flag tag, HA tag or V5 tag.

In an example, the chromatin feature binding molecule may be a rabbit primary antibody, which may be bound by an anti-rabbit-nanobody (secondary binding molecule)-Tn5 transposase fusion protein, or the chromatin feature may be a GFP-tagged transcription factor which may be bound by an anti-GFP-nanobody (secondary binding molecule) Tn5 transposase fusion protein.

Suitably, the chromatin feature binding molecule may be considered a primary antibody that may, for example, target and bind to a chromatin mark. Suitably, a further intermediate binding molecule such as a secondary antibody recognising the primary antibody may be employed for signal amplification purposes. Suitably, the secondary binding molecule (of the transposase) may then bind to the secondary antibody.

Suitably, one of more chromatin features of interest can be profiled simultaneously as explained above. In such a case, one transposase complex may target a chromatin feature of interest (e.g., DNA methylation) that is different to the chromatin feature of interest (e.g., histone mark) targeted by another transposase complex by using different secondary binding molecules within the transposase complexes. Accordingly, efficient multiplexing of several chromatin marks/features can be carried out simultaneously.

In the methods which comprise a step of contacting a substrate with one or more chromatin feature binding molecules or a transposase comprising one or more secondary binding molecules, suitably the contact is under conditions sufficient to allow binding of the or chromatin feature binding molecules to a chromatin feature of interest, and to allow any intermediate binding molecule to bind to the chromatin feature binding molecule if present, or to allow binding of the secondary binding molecules directly to the biological molecule such as a chromatin feature, or to the one or more chromatin feature binding molecules or indeed to the intermediate binding molecule if present.

Suitable conditions may include contacting the substrate with chromatin feature binding molecules, or with the transposase complex comprising a secondary binding molecule for a sufficient length of time to allow binding to chromatin feature of interest, or to the chromatin feature binding molecules respectively. Suitably a sufficient time is between 1 minute and 1 week, suitably between 10 minutes and 24 hours, suitably between 12 hours and 18 hours, suitably between 8 hours and 16 hours, depending on the abundance of the biological molecule, the substrate and the size of the substrate (e.g., a tissue sample). Suitably, a sufficient time is 10 minutes at room temperature, suitably, a sufficient time is 1 hour at room temperature, suitably, a sufficient time is 16 hours at 4° C., depending on the abundance of the biological molecule, the substrate and the size of the substrate.

Suitable conditions may include contacting the substrate with a sufficient concentration of the chromatin feature binding molecule to allow binding, or a sufficient concentration of intermediate binding molecule to allow binding, or a sufficient concentration of the transposase comprising a secondary binding molecule to allow binding. Suitably a sufficient concentration of chromatin feature binding molecule or intermediate binding molecule is between 0.1 ug/ml and 100 μg/ml depending on the abundance of the chromatin feature of interest. Suitably, a sufficient concentration of chromatin feature binding molecule or intermediate binding molecule is between 1 μg/ml and 10 μg/ml, suitably depending on the abundance of the chromatin feature of interest. Suitably a sufficient concentration of secondary binding molecule is between 1 pM and 100 mM. Suitably, a sufficient concentration of secondary binding molecule is between 1 nM and 100 μM.

Suitable conditions to allow binding of a chromatin feature binding molecule to bind to a chromatin feature of interest will be known or determined by the skilled person. Suitable conditions to allow binding of an intermediate binding molecule to a chromatin feature binding molecule, will be known or determined by the skilled person. Suitable conditions to allow binding of a secondary binding molecule to a chromatin feature binding molecule, or to an intermediate binding molecule, will be known or determined by the skilled person.

The transposase complexes disclosed herein may also be used in other methods of spatial profiling or spatial barcoding of biological molecules.

Therefore in a further aspect of the invention there is provided use of a transposase dimer of the second aspect of the invention in a method of spatial profiling or spatial barcoding of a biological molecule. Suitably, use of the transposase dimer of the second aspect of the invention in a method of 10× Visium, SlideSeq or HDST.

Advantageously this may make other known existing spatial profiling methods based on spatially barcoded sequencing (such as 10× Visium, SlideSeq or HDST) compatible with more biological molecules or features (such as DNA/chromatin marks) than is currently possible.

These methods broadly work by first lysing cells or tissue above a substrate bearing spatially barcoded poly-T oligonucleotides, which are used to reverse-transcribe cellular RNAs and assign them a spatial index. By way of example, a transposase such as Tn5 may be loaded with oligonucleotides, such as a detection probe, that carry, for example, a 3′ polyA overhang which the transposase then tagments into DNA (tethered to histone marks or open chromatin). Following tissue lysis and release of material from the section and onto the substrate, the resulting fragments that have been ‘tagmented’ by the transposase would bind to the poly-T spatially barcoded nucleotides on the substrate and be retro-transcribed and thus barcoded together with cellular mRNAs, optionally by adding a strand-displacing reverse transcriptase such as MMLV-RT to the reverse transcription mix. Cellular mRNAs would be discriminated from transposase-produced fragments by ways of a modality barcode incorporated in the detection probe as described elsewhere herein. Alternatively, a transposase such as Tn5 may be loaded with oligonucleotides, such as detection probes, that carry, for example, a phosphorylated 5′ end which the transposase then tagments into DNA as described above. Following tissue lysis and release of material from the section and onto the substrate, the resulting fragments that have been ‘tagmented’ by the transposase could be ligated to the spatially barcoded adapters on the substrate by adding T4 ligase, or another ligase, to the reverse transcription mix. Cellular mRNAs would be barcoded by the reverse transcription reaction, and chromatin features such as open chromatin or histone marks would be barcoded by ligation, and the two could be discriminated by means of a modality barcode incorporated in the detection probe loaded in the transposase, as described elsewhere herein. Following on from any of these approaches, gap filling, and other library construction steps (i.e., enzymatic fragmentation, end repair, A-tailing, ligation of read 2 primers and indexing PCR) can be performed. Alternatively, gap filling could be performed in situ.

### Detection Probe

The present invention makes use of detection probes which bind to biological molecules in or on the substate or tissue. Suitably the detection probes are comprised in the transposase complex as explained hereinabove.

Suitably the methods of the invention comprises a step of contacting the biological molecule with a transposase complex comprising one or more detection probes, so as to allow the transposases to attach the or each detection probe to the biological molecule of interest. In the methods which comprise a step of contacting a substrate with a transposase complex comprising one or more detection probes to allow the transposase complex to attach the or each detection probe to a biological molecule, suitably the contact is under conditions sufficient to allow attachment of the or each detection probe to a biological molecule. Suitably the attachment of the detection probe to the biological molecule may occur by tagmenting the biological molecule with the detection probe as is explained elsewhere herein.

Suitable conditions may include contacting the substrate with the transposase complex for a sufficient length of time to allow attachment of the or each detection probe to the biological molecule. Suitably a sufficient time is between 1 minute and 1 week, suitably between 5 minutes and 24 hours, suitably between 15 minutes and 6 hours, suitably 30 minutes, depending on the abundance of the biological molecule, the substrate, and the size of the substrate or tissue sample.

Suitable conditions may include contacting the substrate with a sufficient concentration of transposase complex to allow attachment of the or each detection probe to the biological molecule. Suitably a sufficient concentration is between 100 pM and 100 μM depending on the abundance of the biological molecule and the number of detection probes, suitably between 1 nM and 10 μM.

Suitable conditions to allow attachment of a given detection probe to a given biological molecule of interest will be known or determined by the skilled person.

Suitably the or each detection probe may comprise, for example, at least a first binding region, optionally a second binding region, optionally a modality barcode, and, optionally, any of the following elements: a photocleavable group, a species barcode, a unique molecular identifier (UMI), an amplification region, and a region allowing sequencing library preparation such as a binding region for a sequencing primer (sequencing element). Suitably therefore, any detection probe known in the art could be used, as long as it comprises the required binding region/s, and, optionally, any of the following elements: a species barcode, a UMI, a photocleavable group, a modality barcode, an amplification region, and/or a sequencing element. In some embodiments, the or each detection probe may comprise a binding region which also comprises a species barcode, or alternatively a binding region which also functions as a species barcode.

Suitably any of the optional features of any of the detection probes may be present in any order, with the exception that the first binding region is at the 3′ end of the detection probe, and the Photocleavable group is at the 5′ of the second binding region.

Suitably the detection probes used in transposase based steps of the methods and those used in non-transposase based steps of the methods are different, and have different required features.

Suitably the or each detection probe for use in steps of tagmentation using a transposases complex may be any probe which is suitable for tagmentation-based assays, for instance ATAC-Seq, CUT&Tag, ATAC-SEE, and similar. Suitably the or each detection probe in such embodiments may comprise, for example, at least a first binding region, optionally a second binding region, optionally a modality barcode, and, optionally, any of the following elements: a photocleavable group, a unique molecular identifier (UMI), an amplification region, and/or a sequencing element. Suitably therefore, any detection probe known in the art could be used, as long as it comprises the required binding region/s, and, optionally, any the following elements: a UMI, a photocleavable group, a modality barcode, an amplification region, and/or a sequencing element.

Suitably, the or each detection probe further comprises one or more moieties or modifications for affinity purification, such as: biotin, desthiobiotin, azides, alkynes, trans-cyclooctenes, strained alkynes such as dibenzylcyclooctyne (DBCO), chloroalkanes, or benzyl-guanine. Suitably the or each detection probe further comprises one or more modifications for the library generation such as deoxyuracil.

Suitably, the first and second binding regions may be as disclosed hereinabove. Suitably, the first binding region binds to a transposase as described above. Suitably, the second binding region binds to a spatial barcode which may be formed of a plurality of index sequences as explained hereinabove.

In a preferred embodiment, steps of tagmentation using a transposase complex comprise a first detection probe and second detection probe which both at least comprise a first binding region, and at least one of which also comprises a second binding region and a photocleavable group, and at least one of which also comprises a sequencing element. Other optional features listed above may also be present.

Suitably, it is noted that in methods wherein reverse transcription of RNA occurs for the spatial profiling of RNA without the use of a transposase complex, then suitably some detection probes are not comprised in the transposase complex. In such a case, suitably an RNA detection probe may be used. Suitably an RNA detection probe comprises at least a first binding region and a second binding region. Other optional features listed above may also be present. Suitably however the first binding region of said RNA detection probes does not bind to a transposase but binds to RNA directly, suitably the first binding region of said RNA detection probes binds to the polyA tail of RNA. Alternatively, the first binding region of said RNA detection probes may bind to a target RNA sequence, suitably via full or partial sequence complementarity. Suitably in such methods, there may be a step of adding RNA detection probes. Suitably contacting the substrate with one or more RNA detection probes to allow the or each RNA detection probe to bind to an RNA of interest.

Suitably the detection probes may not comprise photocleavable groups, in which case the methods may comprise a further step of adding a photocleavable group to the or each detection probe.

In one embodiment, one or more detection probes of the invention are used in the methods of the invention.

A detection probe suitably comprises:

A first, and optionally a second binding region.

In one embodiment, the detection probe further comprises a photocleavable group.

In one embodiment, the detection probe further comprises a species barcode.

In one embodiment, the detection probe further comprises a unique molecular identifier (UMI).

In one embodiment, the detection probe further comprises a modality barcode.

In one embodiment, the detection probe further comprises an amplification region.

In one embodiment, the detection probe further comprises a sequencing element.

Suitably the first binding region allows the detection probe to bind to a transposase, or to bind directly to a target biological molecule such as to RNA. Suitably the second binding region allows the detection probe to bind to a spatial barcode.

Suitably the features will each be described further below. Furthermore the features included in each form of a detection probe which may be used in the methods of the invention will now be described. Suitably, any of the features listed above may be present in a detection probe, suitably any of the features may be present in any order within the detection probe sequence, suitably the features may be present in any order from 5′ to 3′ of the detection probe sequence unless otherwise stated or dictated by function.

In one embodiment the or each detection probe is for use in a step of tagmentation using a transposase complex.

Suitably in such an embodiment, one detection probe may be used alone. In which case, suitably the detection probe comprises at least a first binding region and a second binding region, wherein the first binding region binds to the transposase. Suitably the detection probe may optionally further comprise a modality barcode, a UMI, and a photocleavable group. In one embodiment, the detection probe has the following structure:
3′-[first binding region]-[modality barcode]-[UMI]-[second binding region]-[photocleavable group]-5′

Suitably a sequencing element and/or amplification region may also be added to the detection probe by, for example, the spatial barcode or library generation step. Optionally the photocleavable group may be added via a bridge molecule, described hereinbelow.

Alternatively, in such an embodiment, two probes may be used as a pair, suitably a first detection probe and a second detection probe. Suitably the first detection probe comprises at least a first binding region and a second binding region. Suitably the second detection probe comprises at least a first binding region and a sequencing element. Wherein the first binding region binds to the transposase. Suitably the first detection probe may optionally comprise a a modality barcode, a UMI, and a photocleavable group. In one embodiment, the first detection probe has the following structure:
3′-[first binding region]-[modality barcode]-[UMI]-[second binding region]-[photocleavable group]-5′

Suitably the second detection probe may optionally comprise a modality barcode, a UMI, and an amplification region. In one embodiment, the second detection probe has the following structure:
3′-[first binding region]-[modality barcode]-[UMI]-[sequencing element]-[amplification region]-5′

Optionally the photocleavable group may be added via a bridge molecule, described hereinbelow.

Suitably, of course, the first and second detection probes may be interchangeable.

In another embodiment, the or each detection probe is for use in a step of spatially profiling a target biological molecule such as RNA.

Suitably in such an embodiment, the spatial profiling step may take place by hybridisation of the detection probe to the target biological molecule such as RNA. Suitably, in such an embodiment, the detection probe comprises a first binding region, a sequencing element and a second binding region. Suitably wherein the first binding region binds to target a sequence within the RNA by hybridisation thereto. Suitably the detection probe may optionally further comprise an amplification region, a UMI, a species barcode, and a photocleavable group. In one embodiment, the detection probe has the following structure:
3′-[first binding region]-[amplification region]-[sequencing element]-[UMI]-[species barcode]-[second binding region]-[photocleavable group]-5′

Optionally the photocleavable group may be added via a bridge molecule, described hereinbelow.

Alternatively in such an embodiment, the spatial profiling step may take place by reverse transcription of the RNA. Suitably, in such an embodiment, the detection probe comprises a first binding region, and a second binding region. Suitably wherein the first binding region binds to a target sequence within the RNA, suitably the first binding region is complementary to a sequence within the RNA or to the polyA region. Suitably the detection probe may optionally further comprise a modality barcode, a UMI, and a photocleavable group. In one embodiment, the detection probe has the following structure:
3′-[first binding region]-[modality barcode]-[UMI]-[second binding region]-[photocleavable group]-5′

Optionally the photocleavable group may be added via a bridge molecule, described hereinbelow.

In a preferred embodiment of the invention, a pair of detection probes is used in any step of tagmentation of the biological molecule using a transposase complex as described herein.

Suitably in such an embodiment the first binding region allows each detection probe to bind to a transposase as part of the transposase complex. Suitably the first binding region is as described hereinabove.

Suitably in such an embodiment the second binding region allows the detection probe to bind to a spatial barcode. Suitably the second binding region is as described hereinabove.

In one such embodiment, the or each detection probe comprises a first binding region, and a further nucleic acid sequence comprising at least a photocleavable group. In one embodiment, the or each detection probe comprises a first binding region linked to a further nucleic acid sequence comprising at least a photocleavable group. Suitably the further nucleic acid may further comprise a second binding region, a UMI, an amplification region, and/or a sequencing element.

In one such embodiment, the or each detection probe comprises a first binding region and a nucleic acid sequence linked thereto, wherein the nucleic acid sequence comprises: a UMI, a modality barcode, and a photocleavable group.

In one such embodiment, the or each detection probe comprises a first binding region and a nucleic acid sequence linked thereto, wherein the nucleic acid sequence comprises: a UMI, a modality barcode, an amplification region, and a photocleavable group.

In one such embodiment, the or each detection probe comprises a first binding region, a second binding region, and a nucleic acid sequence linked thereto, wherein the nucleic acid sequence comprises: a species barcode, a UMI, a modality barcode, an amplification region, and a photocleavable group.

In one such embodiment, the or each detection probe comprises a first binding region, a second binding region, and a nucleic acid sequence linked thereto, wherein the nucleic acid sequence comprises: a species barcode, a UMI, a modality barcode, an amplification region, a sequencing element, and a photocleavable group.

Suitably the order of the features within the detection probe is not critical, the features may be present in any order unless otherwise stated herein or dictated by function.

Suitably, the detection probe further comprises a modality barcode.

In one particularly preferred embodiment, the first detection probe comprises a first binding region, and a nucleic acid sequence comprising at least a sequencing element, and optionally a UMI, modality barcode, and amplification region. In one preferred embodiment, the second detection probe comprises a first binding region and a second binding region, and a nucleic acid sequence comprising at least a photocleavable group, and optionally a UMI, modality barcode, and amplification region.

In one embodiment, as explained above, in a preferred embodiment of the invention some of the methods include a step of spatially profiling RNA without using a transposase. In such embodiments, the detection probe for use in methods having a step of reverse transcription of RNA comprises a first binding region complementary to a polyA region in the RNA transcript or to another region of RNA specific to the one or more target RNA sequences, a nucleic acid sequence comprising a modality barcode, and a photocleavable group. Optional elements include a unique molecular identifier (random DNA region), a species barcode, an amplification region such as T7 promoter sequence, and a sequencing element as explained above. In the case that the detection probe is used for, for example, reverse transcription, the detection probe is not attached to a transposase complex. In such case, the first binding region may be a polyA binding region, a gene-specific primer region, or a degenerated sequence binding to multiple genes.

Suitably in such an embodiment, the detection probe may be an RNA detection probe. Suitably for use in the methods of analysing RNA.

In one embodiment, the modified detection probe may be elongated during the method of the invention, and may then further comprise a nucleic acid sequence which is complementary to a transcript of interest, suitably at the 3′ end. Suitably this additional nucleic acid sequence may be termed an elongation region, and is present at the 3′ end of the binding region of the detection probe after a step of elongation.

Suitably the or each binding region may be linked to the remaining features or components of the detection probe, which may comprise a nucleic acid sequence, by a covalent bond. Suitably, when the or each binding region comprises a nucleic acid itself, it is linked to the remaining features or components of the detection probe, which may comprise a nucleic acid sequence, by a phosphodiester bond.

Suitably the amplification region is a nucleic acid or nucleic acid mimic. Suitably the amplification region is DNA.

Suitably the amplification region comprises a promoter for a polymerase. Suitably the promoter is for an RNA polymerase. In one embodiment, the promoter is the T7 RNA polymerase promoter or that of another single subunit polymerase.

Suitably the species barcode is also a nucleic acid or nucleic acid mimic. Suitably the species barcode is DNA. Suitably, the species barcode is separate from the spatial barcode of the invention. Suitably the species barcode allows identification of the biological molecule that the detection probe binds to. Suitably, during sequencing, the species barcode identifies the biological molecule that the detection probe was bound to in the tissue. Suitably in the context of the present invention, a species barcode is used for spatial profiling of RNA, suitably by hybridisation as explained above. Suitably the species barcode indicates the origin of the profiled RNA sequence, such as which gene is encoded in the RNA sequence.

Suitably the UMI is also a nucleic acid or nucleic acid mimic. Suitably the UMI is DNA. Suitably the UMI is unique to each detection probe. Suitably the combination of the individual UMI and each detection probe molecule is unique. Suitably therefore, the UMI allows quantification of the detection probes by counting the number of different UMI sequences. Suitably the UMI thereby facilitates quantification of the biological molecule that the probe binds to. Suitably, during sequence analysis, the UMI identifies the detection probe and allows collapsing of reads that represent a single event of a detection probe binding to its target biological molecule. Suitably the number of different detection probe molecules bound to a biological molecule gives an indication of the expression of that biological molecule.

Suitably the photocleavable group is defined elsewhere herein.

Suitably the or each detection probe may further comprise one or more sequencing elements. Suitably the or each sequencing element aids later sequencing of the detection probe. Suitably at least one of the sequencing elements is a primer binding site. Suitably a primer binding site for sequencing library amplification. Suitably the primer binding site is for a forward primer, suitably a forward primer used for a sequencing library amplification.

Suitably, the or each detection probe comprises a modality barcode as disclosed herein that allows distinguishing between different biological molecules for profiling. Suitably, the modality barcode may be used instead of a species barcode or in addition to a species barcode.

The invention further makes use of one or more modality barcodes. In particular, the or each detection probe may comprise a modality barcode. Furthermore, the RNA detection probe may comprise a modality barcode, and the template switching oligonucleotides may also comprise a modality barcode.

Suitably, the modality barcode allows distinguishing between different biological molecules for profiling. For example, the modality barcode may identify a biological molecule as being of DNA, RNA, protein, chromatin, chromatin feature origin.

Suitably therefore each type of biological molecule to be spatially barcoded in a given method of the invention may be provided with a different modality barcode. Suitably therefore a different modality barcode may be provided for DNA, RNA, protein, chromatin, or chromatin features. Suitably a different modality barcode may be provided for different chromatin features.

Suitably the modality barcode may be used to separate the one or more spatially barcoded detection probes which derive from different biological molecules present in the substrate. Accordingly, the modality barcode will allow the user to distinguish between for example, RNA and chromatin when preparing libraries for sequencing.

Suitably, the modality barcode is a nucleic acid or nucleic acid mimic. Suitably the modality barcode is DNA. Suitably the modality barcode is unique to each category of biological molecules being profiled, for instance genomic DNA, RNA, and proteins. Suitably, probes directly binding to target biological molecules, probes loaded into a transposase complex, and probes binding to polyA RNA and extended by reverse transcription bear different modality barcodes. Suitably, the modality barcode allows discrimination, upon sequencing and data analysis, of data derived from direct binding, data derived from transposase integration, and data derived from reverse transcription.

Suitably the methods of the invention may comprise a step of sorting the spatially barcoded detection probes by the biological molecule from which they are derived, suitably sorting the spatially barcoded detection probes by modality barcode. Suitably this step takes place after any sequencing step.

### Bridge Molecule

The methods of the invention require the presence of a photocleavable group on the or each of the detection probe bound to a substrate that is subjected to the method. The photocleavable group allows control of which detection probes, and later which index sequences, are available for further index sequences to be added. In this way, the photocleavable groups allow control of where and when spatial barcodes are formed.

Suitably, the photocleavable group can either be a component of the or each detection probe, or it can be added onto the or each detection probe.

Suitably, a photocleavable group may be added to the or each detection probe by the addition of a molecule defined as a bridge. The use of a bridge molecule is advantageous in that it allows a large diversity of detection probes to be used on the specimen, without the need to modify each different molecule with a photocleavable group during chemical synthesis. This reduces the cost and complexity involved in the production of a library of detection probes which may be used in the methods of the invention.

Suitably, the bridge molecule is a nucleic acid or mimic. Suitably the bridge molecule is DNA. Suitably, the DNA bridge molecule is a double stranded DNA molecule. Suitably the bridge molecule is between 5 and 40 nucleotides in length and comprises a photocleavable group at the 5′ end or the 3′ end of the molecule, or both.

Suitably, in the cases in which a photocleavable group is not already present on the detection probe, a photocleavable group is added to the or each detection probe. Suitably this may comprise step (b) of the methods, suitably after contacting the substrate with the transposase complex. Alternatively, this may comprise a step during preparation of the transposase complex, suitably prior to performing the methods of the invention.

Suitably the bridge molecule is added to the or each detection probe by ligation. Suitably ligation of the bridge molecule is carried out by the same process of ligation as for the index sequences. Suitably by a ligase enzyme. Suitable ligases are described elsewhere herein.

Suitably, the bridge molecule may further comprise one or more sequencing elements, or purification elements to aid purification of the or each detection probe. Suitably the or each sequencing element aids later sequencing of the or each detection probe. Suitably at least one of the sequencing elements is a primer. Suitably the primer is a forward primer used for a sequencing library amplification.

### Biological Molecule

The methods of the invention allow the situ analysis of the expression of biological molecules in or on a substrate. In particular, the methods allow the spatial analysis of the expression of biological molecules in or on a tissue and of the distribution of open chromatin and chromatin modifications (such as histone marks, methylation, transcription factor binding, or DNA modifier protein binding) on the genome of a tissue.

Suitably the one or more biological molecules can be any molecule indicative of gene expression.

Suitably the one or more biological molecules can be any molecule indicative of chromatin structure or of the epigenetic status of a tissue.

Suitably the or each biological molecule may be selected from: a nucleic acid, a protein, a covalently modified nucleic acid, a covalently modified protein, a post-transcriptional protein modification, a metabolite, a small bioactive molecule, a nucleotide, and a drug.

Suitably the or each biological molecule may be a transcript, suitably a mRNA molecule, large or small non-coding RNA, circular RNA, or other expressed transcript, including alternatively spiced forms of mRNAs. Suitably, the or each biological molecule may be a covalently modified transcript bearing a modifying chemical group.

In one embodiment, the or each biological molecule is an RNA transcript.

Suitably, the or each biological molecule may be a DNA molecule, suitably a genomic DNA molecule or a heterologous DNA molecule. Suitably the or each biological molecule may be a circular DNA molecule or a DNA concatemer. Suitably, the or each biological molecule may be a covalently modified DNA molecule or a covalently modified histone bearing a modifying chemical group, suitably a methyl, hydroxymethyl or formyl group.

In one embodiment, the biological molecule is open chromatin (DNA). In one embodiment, the biological molecule is a chromatin feature such as a histone mark, transcription factor, chromatin associated protein or chromatin factor, DNA modification site including DNA and RNA methylation sites and the like, or a G-quadruplex.

Suitably, the or each biological molecule may be a protein, suitably a polypeptide.

Suitably, the or each biological molecule may be a post-translationally modified protein bearing a post-transcriptional modification known in the art, for instance a glycosylation, phosphorylation, acetylation, or the like.

Suitably, the or each biological molecule may be a metabolite, a small bioactive molecule, a nucleotide or nucleoside, a chemically modified nucleotide or nucleoside, or a drug.

Suitably the methods of the invention may allow analysis of one or more transcripts in a tissue, suitably any number of transcripts of interest are analysed in the method, suitably one or more transcripts of interest are analysed in the method. In some cases, the entire transcriptome in a tissue may be analysed. Suitably in such methods the or each biological molecule is a nucleic acid, suitably a transcript, suitably mRNA.

Suitably the methods of the invention may allow analysis of one or more proteins in a tissue, suitably any number of proteins of interest are analysed in the method, suitably one or more proteins of interest are analysed in the method. In some cases, the entire proteome in a tissue may be analysed. Suitably in such methods the or each biological molecule is a protein or a post-translationally modified protein, suitably a polypeptide or covalently modified polypeptide.

Suitably the methods of the invention may allow analysis of open chromatin in a tissue, suitably any number of DNA molecules of interest may be analysed in the method for areas of open chromatin. In some cases, the entire genome in a tissue may be analysed. Suitably in such methods the biological molecule is DNA, suitably open chromatin in DNA.

Suitably the methods of the invention may allow analysis of chromatin features in a tissue, suitably any number of chromatin features of interest are analysed in the method, suitably one or more chromatin features are analysed in the method. Suitably one or several chromatin feature landscapes may be analysed, suitably simultaneously. Suitably in such methods, the or each biological molecule is a histone mark, transcription factor, chromatin associated protein or chromatin factor, DNA modification site including DNA and RNA methylation sites and the like, or a G-quadruplex.

Suitably in such methods a plurality of biological molecules are bound by the detection probes, suitably the plurality of biological molecules comprise both nucleic acids and one or more other type of marker.

Suitably the methods of the invention may also allow analysis of the transcriptome, chromatin feature landscape, and proteome of a tissue, suitably in such methods a plurality of biological molecules are bound by detection probes, suitably the plurality of biological molecules comprise both nucleic acids and proteins, suitably both transcripts, DNA molecules and polypeptides, or covalently modified transcripts, DNA molecules and polypeptides.

Suitably in the methods of the invention a plurality of biological molecules are bound by the detection probes, suitably the plurality of biological molecules comprise both nucleic acids and one or more other types of biological molecule. Suitably in the methods, any of RNA, DNA, open chromatin, and chromatin features may be bound by detection probes. In one embodiment, both RNA and open chromatin may be bound by detection probes. In one embodiment, both RNA and chromatin features may be bound by detection probes. In one embodiment, RNA, open chromatin, and chromatin features may be bound by detection probes.

Suitably the methods of the invention may also allow the detection of DNA molecules, their copy number, and the presence or absence of single nucleotide variants or the length of simple repeats.

### Photocleavable Group

The present invention utilises bridge molecules, detection probes and index sequences that each may comprise a photocleavable group. The photocleavable group allows control of which detection probes, and later which index sequences, are available for further index sequences to be added. In this way, the photocleavable groups allow control of where and when spatial barcodes are formed.

Suitably, as discussed hereinabove, the or each detection probe may comprise a photocleavable group. Suitably, the or each bridge molecule comprises a photocleavable group. Suitably each index sequence comprises a photocleavable group. In one embodiment, the or each detection probe comprises a photocleavable group.

Alternatively, a photocleavable group may be added to the or each detection molecule by using a bridge molecule as described elsewhere herein.

Suitably the photocleavable group may be located at the 5′ end of the or each bridge molecule, detection probe, or index sequence. Suitably, the photocleavable group may alternatively be located at the 3′ end of the or each bridge molecule, detection probe, and index sequence.

Suitably the photocleavable group may be bound to the 5′ phosphate of the or each bridge molecule, detection probe, and index sequence.

Suitably, the photocleavable group may be bound to the 3′ hydroxyl of the or each bridge molecule, detection probe and index sequence.

Alternatively, the photocleavable group may be present within the bridge molecule, detection probe or index sequence. Suitably the photocleavable group is not present at the 5′ or 3′ end of the molecule, but internally within the sequence of the bridge molecule, detection probe or index sequence. Suitably, upon exposure to light, the photocleavable group is converted into 5′ phosphate termini.

Suitably the photo-cleavable group is a light-sensitive group which protects the 5′ or 3′ end of a nucleic acid sequence. Suitably the photo-cleavable group protects the 5′ or 3′ end of a nucleic acid sequence from addition of further nucleic acid sequences, suitably in the context of the present invention, the photocleavable group prevents the addition of an index sequence.

In one embodiment of the invention, the photocleavable groups when present, prevent a reaction from occurring, and when removed or altered permit a reaction to occur.

Suitably the photocleavable group prevents any hybridisation or ligation of nucleic acids to a bridge molecule, detection probe or index sequence. Suitably, in the case of a bridge molecule, or detection probe, the photocleavable group prevents ligation of an index sequence thereto. Suitably, in the case of an index sequence, the photocleavable group prevents hybridisation or ligation of a further index sequence thereto.

Suitably the photocleavable group comprises a cage. Suitably the cage protects the 5′ phosphate or the 3′ hydroxyl of a nucleic acid.

Suitably the photocleavable group is further attached to a fluorescent moiety. Suitably the fluorescent moiety allows detection of the photocleavable group and is suitably removed after removal or alteration of the photocleavable group.

Suitably, the photocleavable group may include a nitrobenzyl group, dimethoxy-nitrobenzyl group, nitrophenyl group, or nitroveratryl group.

Suitably the photocleavable group may be a PC-spacer or photocleavable spacer. Suitably the photocleavable spacer may comprise a structure according to formula I as noted in the examples.

Suitably the photocleavable group may be cleaved or altered by illumination. Suitably cleavage or alteration of the photocleavable group in response to illumination exposes the 5′ or 3′ end of the relevant nucleic acid. Suitably the cleavage or alteration of the photocleavable group allows the addition of further nucleic acid sequences, suitably index sequences, to the exposed 5′ or 3′ end of the nucleic acid; which may be a detection probe, a bridge molecule or an index sequence.

Suitably the photocleavable group may be altered by changing conformation in response to illumination, suitably by changing three-dimensional conformation in response to illumination.

Alternatively, the photocleavable group may be cleaved in response to illumination.

Suitably, the photocleavable group may be cleaved through a one-photon or two-photon mechanism. Suitably, in the one-photon mechanism, one single photon of light is on average absorbed by each photocleavable molecule resulting in photorelease. Suitably, illumination needed for this reaction is the range from 300 nm to 600 nm. Suitably, in the two-photon mechanism, two distinct photons of light are on average absorbed by each photocleavable molecule resulting in photorelease. Suitably, the two photons of light are absorbed within a femtosecond time period. Suitably, illumination needed for this reaction is in the range from 680 nm to 900 nm.

Suitable illumination which will act to cleave the photocleavable group is discussed elsewhere herein.

### Illumination

The methods of the invention rely on illumination of selected locations or areas of interest in a sequential manner to control the order in which index sequences are added to detection probes bound in those areas. The order in which index sequences are added to the detection probes forms a unique spatial barcode corresponding to each location or area of interest.

Suitably illuminating a location or area of interest comprises illuminating a location or area of interest that has been selected by a user. Suitably the or each location or area of interest is selected by a user using software. Suitably this selection of locations or areas takes place prior to the illuminating step of the methods.

Suitably illumination cleaves or alters photocleavable groups. Suitably illuminating a location or area of interest cleaves or alters the photocleavable groups present in that location or area. Suitably illuminating a location or area of interest cleaves or alters the photocleavable groups on the detection probes and/or the index sequences in that location or area. Suitably, in the first cycle of the methods, illuminating a location or area of interest cleaves or alters the photocleavable groups from each of the detection probes in that location or area. Suitably, in subsequent cycles of the methods, illuminating a location or area of interest cleaves or alters the photocleavable groups from each of the bound index sequences in that location or area.

Suitably illumination cleaves or alters photocleavable groups from the bridge molecules, detection probes and/or index sequences such that the 5′ end or 3′ end is exposed, and optionally available for reaction. Suitably illumination cleaves or alters photocleavable groups from the bridge molecules, detection probes and/or index sequences such that the 5′ phosphate or 3′ hydroxyl is exposed, and optionally available for reaction. Suitably illumination allows the addition of an index sequence to the 5′ end or the 3′ end of the bridge molecules, detection probes and/or index sequences. Suitably, illumination cleaves photocleavable groups which are internally positioned within the detection probe (i.e., not at the 5′ or 3′ end of the detection probe) so that they are converted into 5′ phosphate termini.

Suitably illuminating an area of interest allows index sequences to be added to the bridge molecules, detection probes and/or bound index sequences in that location or area. Suitably, in the first cycle of the methods, illuminating a location or area of interest allows an index sequence to be added to each of the bridge molecules, or detection probes in the location or area. Suitably, in subsequent cycles of the methods, illuminating a location or area of interest allows a further index sequence to be added to each of the bound index sequences in the location or area.

Suitably, illumination determines in which locations or areas of interest a given index sequence will be added.

Suitably, multiple locations or areas of interest may be illuminated at once. Suitably, the relevant step of the methods may comprise illuminating multiple locations or areas of interest.

Suitably therefore said illumination step may comprise creating a pattern of illumination. Suitably therefore said illumination step may comprise creating a pattern of illumination on the substrate or tissue, wherein the pattern of illumination comprises multiple locations or areas of interest. Suitably the same index sequence is added to each location or area of interest within a given pattern of illumination.

Suitably, the locations or areas of interest that are illuminated in said illumination step change in each round of said illumination steps. Suitably therefore the pattern of illumination changes in each round of said illumination steps.

Suitably, in each ‘round’ of said illumination steps, all of the areas/locations of interest that have the same index sequence for that position are illuminated and the relevant index sequence is contacted and suitably added.

Suitably the methods comprise multiple rounds of said illumination step until each of the different index sequences is contacted to the areas/locations of interest, suitably added to the areas/locations, to fulfil the relevant position of the spatial barcode.

Suitably, a cycle is complete after one round has been performed for each of the different index sequences used in the spatial barcodes. Suitably a method using 4 different index sequences will have 4 rounds per cycle.

Suitably therefore a ‘cycle’ corresponds to completing a position of the spatial barcode for each area/location of interest. Suitably a ‘cycle’ corresponds to contacting the locations/areas with each of the index sequences to be used in the method.

Suitably the first cycle comprises a plurality of rounds of said illumination steps to contact, suitably to add, the relevant index sequence corresponding to a first position in the spatial barcodes, to bound bridge molecules and/or detection probes in the selected locations/areas.

Suitably after the first cycle, all index sequences in the first position of the allocated spatial barcodes have been contacted, suitably added.

Suitably the second cycle comprises a plurality of rounds of said illumination steps to contact, suitably to add, the relevant index sequence, corresponding to a second position in the spatial barcodes, to bound index sequences in the selected locations/areas.

Suitably after the second cycle, all index sequences in the second position of the spatial barcodes have been contacted, suitably added.

Suitably any number of rounds per cycle may occur depending on the number of different index sequences to be used. Suitably any number of cycles may occur depending on the length of the spatial barcode to be added and therefore the number of index sequences comprised in each spatial barcode.

For example, each spatial barcode may comprise 10 positions and therefore 10 index sequences, and 4 different index sequences may be used in the method. Therefore the methods of the invention would comprise 4 rounds per cycle and 10 cycles in order to form the complete spatial barcodes.

Suitably, when referring to addition of ‘all’ index sequences in each cycle, and to ‘each’ of the different index sequences being added in a round, it will be appreciated that not every index sequence will always be added to every bound bridge molecules and/or detection probes, or every bound index sequence. Some index sequences may not be added due to expected inefficiencies in the method, for example ligase enzymes are not 100% efficient.

Suitably in some cases, only some of the index sequences are added. Suitably, only some index sequences are added to the bound bridge molecules and/or detection probes, or bound index sequences. Suitably, the index sequences are at least contacted with the relevant areas/positions for addition. Suitably a round or cycle is regarded as complete when all the required index sequences have been contacted with the relevant areas/locations.

Suitably illumination is not restricted to visible light, suitably use of the term ‘illumination’ of ‘illuminating’ herein refers to any wavelength of light, either visible or non-visible.

Suitably illumination of the or each location or area of interest is achieved by using a light source, suitably a light source of a constant wavelength, suitably by using a LED or a laser.

Suitably, illumination may be directed to each location or area of interest. Suitably by using a refractive or reflective optical system. Suitably the refractive or reflective optical system may have a resolution of 200 nm or above. Suitably the optical system may be comprised within a microscope, such as any microscope described in the art. Suitably the light source may also be comprised within a microscope. Suitably, the optical system includes an element to direct illumination to the or each location or area of interest. Suitably, the optical system includes an element to direct illumination from the light source to the or each location or area of interest.

In some embodiments, the element is a movable mirror, for example a galvanometric mirror. In some embodiments, the element is a digital micromirror device (DMD chip). In some embodiments, the element is a spatial light modulator.

Suitably the or each location or area of interest may be illuminated by light having a wavelength between 300-600 nm, suitably between 310 nm-570 nm, suitably between 320 nm-550 nm, suitably between 330 nm-520 nm, suitably between 340 nm-480 nm, suitably between 350 nm-450 nm, suitably between 360 nm-420 nm. Suitably, these wavelengths of light result in a one-photon photorelease process.

Alternatively, the or each location or area of interest may be illuminated by light having a wavelength between 680 nm and 900 nm, suitably between 700 and 850 nm, suitably between 720 and 800 nm. Suitably, these wavelengths of light result in a two-photon photorelease process.

Suitably the light may be UV or violet light or infrared light

In one embodiment, the or each location or area of interest is illuminated by light having a wavelength of between 350 nm-410 nm, for the one photon process, or 710 to 800 nm for the two-photon process. In one embodiment, the or each location or area of interest is illuminated with the same wavelength of light. Suitably the same wavelength of light is used throughout the methods of the invention.

Alternatively, a first location/area of interest may be illuminated by a first wavelength of light and a second location/area of interest may be illuminated by a second wavelength of light. Suitably, in this case one wavelength of light is in the 300 nm-450 nm range and a second wavelength of light is in the 500-600 nm range, using the one-photon photorelease process. Suitably the first and second locations/areas may be illuminated at the same time but by different wavelengths of light. Suitably, this may apply to multiple locations/areas of interest, which may be illuminated at the same time, but with different wavelengths of light.

Suitably each location/area of interest is illuminated with light of a sufficient power to cleave or alter the photocleavable groups in the given location or area. Suitably, each location/area of interest is illuminated with a light with an average power ranging from 10 mW/cm² to 30 W/cm², suitably from 20 mW/cm² to 20 W/cm², suitably from 50 mW/cm² to 10 W/cm², suitably from 100 mW/cm² to 5 W/cm², suitably from 200 mW/cm² to 1 W/cm². Suitably each location/area of interest is illuminated for a sufficient period of time to cleave or alter the photocleavable groups in that location/area. Suitably each location/area of interest is illuminated for between 1 seconds and 10 minutes, suitably between 5 seconds and 5 minutes, suitably between 10 seconds and 3 minutes, suitably between 30 seconds and 2 minutes. The time of illumination is dependent of the intensity of illumination. The skilled person will know how to adjust the time of illumination to achieve sufficient cleavage or alteration of the photocleavable groups.

In one embodiment, each location/area of interest is illuminated for 5 minutes. Suitably, therefore, step (c) comprises illuminating a location/area of interest for 5 minutes.

In one embodiment, each location/area of interest is illuminated for 30 seconds. Suitably, therefore, step (c) comprises illuminating a location/area of interest for 30 seconds.

### Addition of Index Sequences

The methods of the invention comprise the addition of index sequences in order to form the spatial barcode attached to the or each bridge molecule, or detection probe. Index sequences are added to a location or area that has been illuminated, and which therefore comprises detection probes, bridge molecules or bound index sequences with exposed 5′ or 3′ ends. Suitably, exposed 5′ or 3′ ends are reactive.

Suitably an index sequence is added to any exposed, or reactive, 5′ or 3′ end present in the location or area illuminated in said illumination step. Suitably, in a first cycle of the methods, an index sequence is added to any exposed, or reactive, 5′ or 3′ end of a bridge molecule, or detection probe present in the location or area illuminated in said illumination step. Suitably, in a subsequence cycle of the methods, an index sequence is added to any exposed, or reactive, 5′ or 3′ end of a bound index sequence present in the location or area illuminated said illumination step.

Suitably the or each index sequence is added by ligation, which may be chemical or enzymatic. Suitably by ligation onto the 5′ or 3′ end of a bridge molecule, or detection probe present in the location or area illuminated in said illumination step. Suitably in a first cycle of the methods. Suitably by ligation onto the 5′ or 3′ end of a bound index sequence present in the location or area illuminated in said illumination step. Suitably in a subsequent cycle of the methods. Suitably the or each index sequence is ligated by a ligase enzyme. Suitably the ligase enzyme may be selected from any ligase, such as: T4 ligase, T3 ligase, Taq ligase.

In one embodiment, the or each index sequence is ligated by T4 DNA ligase.

Suitably the or each bridge molecule is ligated to a detection probe by the same means.

Suitably, the ligase may be added to the methods of the invention during the step of adding the index sequences to ligate the or each index sequence. Suitably therefore the step of adding the index sequences may comprise ligating an index sequence of the spatial barcode to the or each detection probe or bridge molecule within the location or area illuminated in step said illumination step.

Alternatively, the ligase may be added to the methods of the invention after all of the index sequences have been added to ligate all of the index sequences that have been added to the or each detection probe or bridge molecule. Suitably, in this embodiment, the step of adding index sequences may comprise hybridising an index sequence of the spatial barcode to the or each detection probe or bridge molecule within the location or area illuminated in said illumination step. Suitably, the method further comprises a step after the step of adding the index sequences of ligating the index sequences to the or each detection probe or bridge molecule.

### Index Sequence

The methods of the invention employ index sequences which when added together in various different orders form spatial barcodes. These spatial barcodes indicate where in or on a substrate a given detection probe was bound, and therefore where a relevant biological molecule is present.

Suitably a spatial barcode is formed of a plurality of index sequences. Suitably, a spatial barcode comprises a plurality of index sequences. Suitably the index sequences are sequentially added together to form a spatial barcode, suitably by repeating steps said illumination steps of the method. Suitably during each cycle of the methods, an index sequence is added to each detection probe or bound index sequence. Suitably during the first cycle of the methods, a first index sequence is added to each detection probe or bound index sequence, during a second cycle of the methods, a second index sequence is added to each detection probe or bound index sequence and during subsequent cycles of the method, a third, fourth, etc. index sequence is added to each detection probe or bound index sequence.

Suitably during the first cycle of the methods, a first index sequence is added to each detection probe. Suitably during subsequent cycles of the methods, subsequent index sequences are added to each bound index sequence.

Each index sequence comprises:
a total length of between 5 and 50 nucleotides; and
a photocleavable group bound to one or both of the 5′ or 3′ ends of the molecule.

In one embodiment, the index sequences are nucleic acid sequences or nucleic acid mimics. Suitably comprising a 5′ and a 3′ end. Suitably, the index sequences may be RNA, DNA, or modified backbone nucleic acid sequences, comprised of canonical or non-canonical bases. In one embodiment, the index sequences are DNA. In one embodiment, each index sequence is a double stranded DNA. Suitably, each index sequence has a total length of between 10-40 nucleotides, suitably between 14-30 nucleotides, suitably between 15-25 nucleotides.

In one embodiment, each index sequence has a total length of 19-20 nucleotides.

Suitably the total length is the total length of the double stranded portion of the index sequence, suitably excluding any overhangs if present.

Suitably, each index sequence is produced by the annealing of nucleic acid strands having a total length of between 10-40 nucleotides, suitably between 14-30 nucleotides, suitably between 15-25 nucleotides.

In one embodiment, each index sequence is produced by the annealing of nucleic acid strands having a total length of 19-20 nucleotides.

Suitably each index sequence may comprise blunt ends.

Alternatively, each index sequence may comprise overhangs, suitably at both the 5′ and 3′ ends. Suitably the overhangs are complementary, suitably, the overhangs are complementary to overhangs on other index sequences. Suitably each overhang is partly or fully complementary to an overhang on another index sequence.

Suitably each overhang comprises a length of between 1-100 nucleotides, suitably 1-75, suitably, 1-50 nucleotides, suitably, 1-20 nucleotides, 1-15 nucleotides, suitably 1-10 nucleotides, suitably 3-10 nucleotides, suitably 3-9 nucleotides. Suitably each overhang comprises a length selected from 3, 4, 5, 6, 7, 8, 9, and 10 nucleotides. Suitably each overhang is 8 or 10 nucleotides in length. Suitably each overhang is 6 or 7 nucleotides in length.

Suitably each index sequence comprises a first overhang and a second overhang. Suitably the first and second overhangs may be independently located at the 5′ or 3′ ends of each index sequence.

In one embodiment, the overhangs located at the 5′ and 3′ end of the or each index sequence have the same length.

In one embodiment, the overhangs located at the 5′ and 3′ end of the or each index sequence have different lengths. Suitably each index sequence comprises a longer and a shorter overhang, located at either end of the molecule. Suitably a first longer overhang and a second shorter overhang. Suitably a longer overhang is located at a first end of the index sequence and a shorter overhang is located at a second end of the index sequence.

Suitably each index sequence comprises a first overhang of 6 nucleotides in length and a second overhang of 7 nucleotides in length. Suitably when the index sequences are added together to form a spatial barcode, the overhangs of the index sequences alternate. Suitably the overhangs alternate between 6 nucleotides in length and 7 nucleotides in length.

Suitably each index sequence comprises one or more photocleavable groups. The or each photocleavable group is as defined elsewhere herein.

Suitably, each index sequence comprises a central region having a unique nucleotide sequence distinct from that of all other index molecules.

Suitably each index sequence comprises a high GC content. Suitably each index sequence comprises a GC content of between 30% and 80%.

Suitably, each index sequence does not form any AA or TT dimers. Suitably when an index sequence is a double stranded DNA, it does not comprise any AA or TT dimers.

The present invention further provides a library of index sequences.

Suitably the library of index sequences comprises index sequences to be used in the methods of the invention. Suitably the library of index sequences comprises all of the index sequences to be used in the methods of the invention.

Suitably there are at least 4 different index sequences used in the method of the present invention. Suitably between 1-100 different index sequences may be used in the methods of the present invention. In one embodiment, 4 different index sequences are used in the present invention. Suitably a higher number of index sequences allows more diverse spatial barcodes to be generated, and therefore a higher number of unique barcodes to be generated, and therefore more locations/areas of interest to be labelled.

Suitably the index sequences may be classified into groups. Suitably the index sequences in each group have the same nucleotide sequence. Suitably the library may comprise a plurality of groups of index sequences.

Suitably therefore the library may comprise a plurality of index sequences, suitably a plurality of groups of index sequences. Suitably the library may comprise at least 2 groups of index sequences, wherein the index sequences in each group share the same nucleotide sequence. Suitably the library may comprise up to 100 groups of index sequences, wherein the index sequences in each group share the same nucleotide sequence.

For example, the library of the invention may comprise 4 groups of index sequences; group A, group B, group C, group D, wherein the index sequences in each group share the same nucleotide sequence.

In one embodiment, an index sequence may comprise a sequence according to any of those listed in the examples herein, or any combination thereof, or indeed any of those index sequences listed in related patent application WO2021/116715.

For example, an index sequence may comprise the sequence set out in SEQ ID NO:54, 55, 64 or 65.

### Spatial Barcode

The present invention provides methods of spatial barcoding. These methods comprise the addition of a spatial barcode to detection probes, optionally through a bridge molecule, in order to label where each detection probe is bound in or on a substrate.

The invention further provides a spatial barcode comprising a plurality of index sequences, wherein the index sequences are selected from the library as defined elsewhere herein.

As described above, each spatial barcode is formed of a plurality of index sequences. Suitably the index sequences in each spatial barcode are arranged in a unique order. Suitably, therefore, each spatial barcode is unique.

Suitably, the individual index sequences forming a spatial barcode are linked by a covalent chemical bond. Suitably the covalent chemical bond is compatible with polymerase enzymes, and compatible with high-throughput sequencing chemistry. Suitably the covalent chemical bond is compatible with polymerase enzymes.

Suitably, the individual index sequences forming a spatial barcode are linked by a phosphodiester bond.

Suitably one spatial barcode is allocated per each location or area of interest. Suitably a spatial barcode is unique to a selected location or area. Suitably, the same spatial barcode is added to each bridge molecule or detection probe within the same location or area of interest. Suitably therefore each spatial barcode indicates a given location or area of interest.

Suitably the or each spatial barcode comprises at least one index sequence. Suitably the or each spatial barcode comprises between 4-50 index sequences. Spatial barcodes comprising a higher number of index sequences have a higher encoding capacity and can label more unique locations/areas of interest. Suitably each index sequence within a spatial barcode may be the same or different.

Suitably the index sequences are added to the or each bridge molecule or detection probe in a specific order to build up the spatial barcode. Suitably one index sequence is added to the or each bridge molecule or detection probe in a first cycle of steps. Suitably one index sequence is then added to the or each detection probe per subsequent cycle of steps. Suitably by adding to the bound index sequences. Suitably these steps are repeated in cycles until the spatial barcode is fully formed and attached to the or each detection probe. Suitably therefore, the number of cycles of steps is determined by the length of the or each spatial barcode.

Suitably, the order of index sequences in each spatial barcode is optimised to reduce errors during sequencing.

The present invention further provides a library of spatial barcodes.

Suitably, each spatial barcode in the library comprises a plurality of index sequences, wherein the index sequences are selected from the library of index sequences as defined elsewhere herein. Suitably, each spatial barcode in the library is unique. Suitably, each spatial barcode in the library comprises a unique combination of index sequences.

Suitably, the library of spatial barcodes may be designed in order to reduce mis-identification errors after sequencing. Suitably, the library of spatial barcodes forms an error-correcting code. Many methods of producing error-correcting codes are known in the art

Suitably, the combination of index sequences in each spatial barcode included in the library may be chosen so that each spatial barcode has a Hamming distance of 1 from all other spatial barcodes included in the library. Suitably each spatial barcode has a Hamming distance of 1 from all other spatial barcodes used in a method of the invention.

Suitably, the Hamming distance between a pair of spatial barcodes is defined as the number of elements (in this case index sequences) in the first spatial barcode that have to be replaced with other index sequences in order to transform the first spatial barcode into a copy of the second spatial barcode.

Suitably, the combination of index sequences in each spatial barcode included in the library of spatial barcodes may be chosen so that each spatial barcode has a Hamming distance of 3, 5, or 7 from all other spatial barcodes included in the library. Suitably each spatial barcode has a Hamming distance of 3, 5, or 7 from all other spatial barcodes used in a method of the invention.

Suitably, the combination of index sequences in each spatial barcode included in the library of spatial barcodes may be chosen according to an error-correcting encoding scheme capable of correcting at least one, at least two or at least three substitution, deletion or insertion errors. Suitably the methods of the invention may comprise a step of assigning a spatial barcode to each location or area of interest within the tissue. Suitably this step occurs prior to the addition of index sequences in the methods. Suitably assigning a spatial barcode to each location or area of interest is carried out using software. Suitably assigning a spatial barcode to each location or area of interest is automatically carried out by software, suitably when a location or area of interest is selected by a user.

Suitably an assigned spatial barcode comprises a plurality of units. Suitably each unit corresponds to an index sequence. Suitable units may be any form of code, for example numbers or letters wherein each index sequence has a corresponding unit. For example, in an embodiment where 4 different index sequences are being used and the spatial barcode has a length of 4 units, units A, B, C and D may each correspond to a different index sequence. In such an embodiment, examples of assigned spatial barcodes may be: ABCD, ACBD, ADBC and the like.

### Sequencing

After the complete spatial barcodes are added to the bridge molecules or detection probes, a step of sequencing may then take place.

Suitably, sequencing may not take place immediately after the spatial barcodes are added. In some embodiments, the substrate comprising the spatial barcodes attached to bridge molecules, or detection probes may be stored prior to sequencing. The present invention therefore provides a substrate comprising spatially barcoded detection probes.

Suitably, when the complete spatial barcode has been added to a detection probe, the detection probe is then known as a spatially barcoded detection probe.

Suitably the or each spatially barcoded detection probe is sequenced. Suitably therefore, the or each detection probe and the attached spatial barcode are sequenced as a single nucleic acid, optionally further comprising a bridge molecule.

Suitably the detection probes provide information on what biological molecules are present and to what level in the tissue.

Suitably the spatial barcodes provide information on where the biological molecules are expressed in the tissue. Suitably, in which areas of interest the biological molecules are present.

Suitably therefore, in sequencing a single nucleic acid produced by the methods of the invention, identification, quantification and spatial information is provided for each biological molecule of interest.

Suitably the methods of the invention may further comprise a step of preparing the one or more spatially barcoded detection probes or root molecules for sequencing. Suitably this step occurs prior to the sequencing step.

In one embodiment, this includes removing the spatially barcoded detection probes from the substrate. In another embodiment, a portion or all of the spatially barcoded detection probes are amplified in situ, prior to preparation for sequencing.

Suitably, preparing the one or more spatially barcoded detection probes for sequencing may comprise adding modifiers to the or each spatially barcoded detection probe. Suitable modifiers may be those required to conduct sequencing, for example a primer or a PCR handle.

Suitably, a sequencing element, such as a sequencing primer required for sequencing library preparation, may be added to the end of each spatial barcode. Suitably to the 5′ end, or suitably to the 3′ end. Suitably the sequencing elements are added by PCR, enzymatic ligation, or by template switching of reverse transcription. Suitably in the case of adding to the 5′ end, the sequencing elements are added by ligation. Suitably in the case of adding to the 3′ end, the sequencing elements are added by template switching of reverse transcription, or by PCR or by ligation. Suitably addition of sequencing elements by PCR may comprise using random hexamer oligonucleotides comprising the sequence element at the 5′ end thereof. Suitably addition of sequencing elements by ligation may comprise a step of fragmentation of an elongated detection probe, suitably prior to ligation. Suitably using any ligase enzyme known in the art, or by using any reverse transcriptase known in the art, or by using any DNA polymerase enzyme known in the art. Suitably, the ligase enzyme used may one of the ligase enzymes described elsewhere herein. Suitably, the addition of a sequencing element is performed before the sequencing step of the methods of this invention. Alternatively, if the sequencing element is a 3′ primer, then the addition of the sequencing element can be performed at the same time as the detection probe is elongated.

Suitably, one or more spatially barcoded detection probes may be extracted from the tissue or specimen by any DNA extraction method known in the art, and the resulting pool of molecules may be stored prior to sequencing.

Suitably, preparing the one or more spatially barcoded detection probes for sequencing may comprise a step of transcription. Suitably transcribing the or each spatially barcoded detection probe or root molecule into RNA.

Suitably, preparing the one or more spatially barcoded detection probes or root molecules for sequencing may comprise a step of isolating the or each spatially barcoded detection probe. Suitably a step of isolating the or each spatially barcoded detection probe RNA.

Suitably, preparing the one or more spatially barcoded detection probes for sequencing may comprise a step of reverse transcription. Suitably reverse transcribing the or each spatially barcoded detection probe RNA.

Suitably, preparing the one or more spatially barcoded detection probes for sequencing may comprise a step of amplifying the one or more spatially barcoded detection probes. Suitably, amplifying the or each reverse transcribed spatially barcoded detection probe.

Suitably, the one or more spatially barcoded detection probes may be amplified by an enzymatic process using the amplification region included in each detection probe. Suitably, this amplification can happen while the spatially barcoded detection probes are still embedded in the tissue, or after they have been extracted as described above. In one embodiment, the amplification is performed by RNA transcription, in one embodiment, the enzyme used for amplification is T7 RNA polymerase. Alternatively, the amplification may be carried out by any other known amplification processes, for example rolling circle amplification or Loop-mediated isothermal amplification (LAMP). Suitably in such embodiments, the spatially barcoded detection probe is first circularised, suitably by a telomerase enzyme, suitably telN polymerase. Suitably the circularised spatially barcoded detection probe is then amplified, suitably by a strand-displacement polymerase, suitably by Phi29 DNA polymerase.

Suitably, the amplification process produces multiple copies of each spatially barcoded detection probe, replicating the sequence of the detection probe and of the spatial barcode. In one embodiment, such copies are RNA molecules.

Suitably therefore, preparing the one or more spatially barcoded detection probes for sequencing may comprise: adding modifiers to the or each spatially barcoded detection probe, transcribing the or each spatially barcoded detection probe into RNA, isolating the or each spatially barcoded detection probe RNA, reverse transcribing the or each spatially barcoded detection probe RNA into DNA, and amplifying the or each reverse transcribed spatially barcoded detection probe DNA.

Suitably after reverse transcription, the spatially barcoded detection probes form a sequencing library ready for sequencing.

Suitably, one or more further amplification steps may be employed in the methods disclosed herein. Suitably, the amplification step may be performed before or after performing in situ reverse transcription to generate RNA:cDNA hybrids from the RNA molecules of interest.

Alternatively the amplification step, may be performed after a step of contacting the substrate with one or more transposase complexes as disclosed herein. Suitably an amplification step is performed at the end of the methods described herein, suitably just prior to any sequencing step. Suitably to produce sequencing libraries.

Suitably, the amplification step may be performed using polymerase chain reaction, T7 RNA polymerase, or with loop-mediated isothermal amplification (LAMP).

### Reverse Transcription Step and Probe

The present invention may also make use of detection probes for spatially barcoding RNA, suitably termed an RNA detection probe, which may in some instances be used interchangeably with ‘Reverse transcriptase probe’ herein. An RNA detection probe that targets one of more RNA molecules of interest may be used in the methods disclosed herein relating to spatial barcoding of RNA. In particular, the RNA detection probe may be employed prior to an in situ reverse transcription step to generate RNA:cDNA hybrids from the RNA molecules of interest.

Suitably, the RNA detection probe further comprises a nucleic acid sequence comprising a unique molecular identifier (UMI) and/or an amplification region.

Suitably, the RNA detection probe comprises a nucleic acid comprising an extendable 3′-OH end, a modality barcode, and a photocleavable group.

Suitably, the RNA detection probe further comprises one or more moieties or modifications for affinity purification, biotin, desthiobiotin, azides, alkynes, trans-cyclooctenes, strained alkynes such as dibenzylcyclooctyne (DBCO), chloroalkanes, or benzyl-guanine. Suitably the detection probe further comprises one or more modifications for the library generation.

Suitably the RNA detection probe is capable of hybridizing to the 3′ end of RNA molecules. Suitably the RNA detection probe comprises a sequence which is complementary to the 3′ end of RNA molecules. Suitably the RNA detection probe may comprise a sequence which is complementary to a specific RNA sequence, or may comprise a random sequence such as random hexamers or random hexamers depleted for ribosomal RNA sequences, or may comprise a polyT sequence which is suitably complementary to polyA RNA features.

Suitably, in methods disclosed herein, a step of performing in situ reverse transcription is performed to generate RNA:cDNA hybrids. Suitably prior to the step of contacting of the substrate with one or more additional transposase complexes, wherein each additional transposase complex comprises a transposase bound to at least one detection probe, to allow the or each additional transposase complex to attach the detection probe to an RNA:cDNA hybrid of interest, wherein the or each detection probe comprises a photocleavable group.

Suitably the reverse transcription step is performed by a reverse transcriptase enzyme. Suitable reverse transcriptase enzymes include but are not limited to AML, MMLV, Superscript II, Superscript III, Superscript IV, Maxima H minus. Suitably therefore, the RNA molecules of interest are contacted with the reverse transcriptase under suitable conditions for the reverse transcriptase to generate RNA:cDNA hybrid molecules.

Suitably, the reverse transcription step may be performed using modified dNTPs such that the RNA:DNA hybrid comprises dNTPs. Suitably the modified dNTPs are digoxigenin (DIG)-11-deoxyuridine triphosphate (dUTP). Suitably this may facilitate the transposase complex distinguishing between the RNA:DNA hybrid and DNA as explained above.

Suitably, an enzymatic reaction as is known in the art may be used to generate a 3′ handle that allows amplification of RNA:cDNA generated by in situ reverse transcription. Template switching methods may include but are not limited to: template switching using a reverse transcription enzyme and a template switching oligonucleotide. Suitably the structure of a template switching oligonucleotide is known in the art and suitably the template switching oligonucleotide comprises a DNA sequence with a plurality of LNA (locked nucleic acid) or RNA bases, suitably at one end of the oligonucleotide forming a 3′ handle. Suitably the 3′ handle is complementary to a cDNA overhang created by the reverse transcriptase. Alternatively, using untemplated terminal extension by terminal deoxynucleotidyl transferase, pol-theta, or any other enzymes known in the art, followed by ligation of a 3′ handle, or untemplated terminal extension followed by PCR with an oligonucleotide including a 3′ handle.

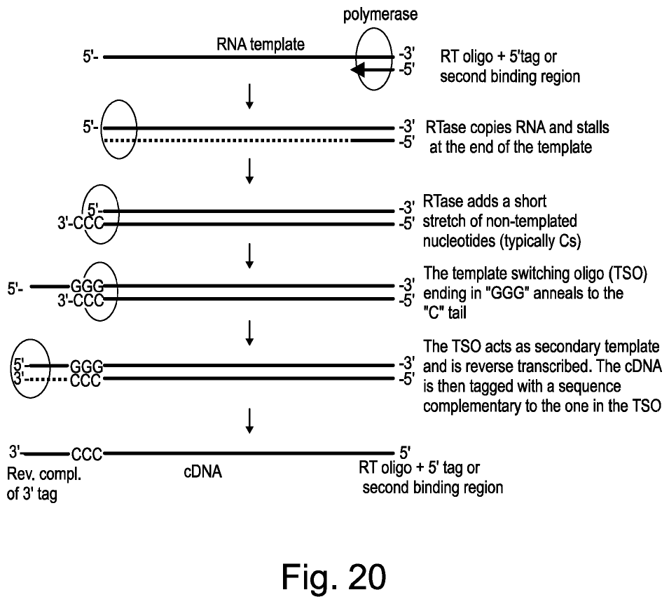

Suitably therefore methods which involve a step of reverse transcription may further comprise a step of template switching (see FIG. 20). Suitably the steps of template switching showing in FIG. 20 may be incorporated into any method described herein. Suitably, template switching may be used in conjunction with methods involving transposases as disclosed herein. Suitably, template switching may be used in situ. Suitably the step of template switching takes place prior to an amplification step, suitably prior to the final amplification step. Suitably, template switching is aided by supplementing additives to the reaction. Suitably, the additives may include agents capable of reducing the secondary structure of nucleic acids, suitably betaine. Suitably, the additives may include agents increasing density of the reaction buffer, suitably FICOLL. Suitably, the additives may include single-strand DNA binding proteins, suitably ET-SSB.

### Kit

A kit of the present invention comprises:
a library of index sequences, one or more transposases, one or more detection probes, optionally a ligase enzyme, and optionally one or more reagents.

Suitably the kit may comprise one or more transposase dimers, or transposase complexes as described herein. Suitably wherein the transposases are dimerised and each comprise a detection probe.

Suitably the library of index sequences, the transposases or transposase complex and the detection probes are as described herein.

Suitable reagents may be buffers, nucleotides, dilution agents, chelating agents, and the like.

Suitably the kit may further comprise a reverse transcriptase enzyme and optionally RNA detection probes and optionally, template switching oligonucleotides. Suitably the kit may further comprise a DNA polymerase enzyme, and optionally primers.

Suitably the kit may further comprise components and reagents for performing sequencing. Suitably the kit may further comprise instructions for performing a method of the invention.

In one embodiment, the kit comprises a library of index sequences, one or more transposases, one or more detection probes, a ligase enzyme, a reverse transcriptase enzyme and one or more RNA detection probes, and optionally one or more reagents.

In one embodiment, the kit comprises a library of index sequences, one or more transposases, one or more detection probes, a ligase enzyme, a reverse transcriptase enzyme, one or more RNA detection probes, one or more template switching oligonucleotides, and optionally one or more reagents.

### Additional Steps

One or more additional steps can be performed in conjunction with any one or more methods disclosed herein. For example, one or more of the following additional steps may be formed: (A) immobilization, (B) fixation, (C) permeabilization, (D) post-fixation, (E) protease/lipid clean-up, and (F) lysis and purification.

### (A) Immobilization

Suitably immobilization of substrates such as cells, tissues (or sections thereof), or small organisms (or sections thereof) and the like may be performed. Suitably, the substrate may be immobilized onto an item, suitably onto a slide, slide chamber, plate, tube, or other item suitable for immobilization. Suitably said a slide, slide chamber, plate, tube, or other item may be made from plastic, glass etc. and any other inert materials suitable for immobilization of substrates. Suitably said slide, slide chamber, plate, tube, or other item may also be treated with chemicals (including but not limited to) poly-L-lysine, fibronectin, concanavalin-A, allyltrichlorosilate, BIND-silane, or 3-aminopropyl-triethoxysilane) to improve adhesion of substrates such as tissues thereto. Suitably the substrate may be immobilized prior to the methods of the invention.

### (B) Fixation

Suitably, the substrates disclosed herein may further be fixed by any means known in the art. Suitably the substrate may be fixed to an item, suitably after immobilization of the substrate onto the item as explained above. For example, fixation may be performed using aldehydes, alcohols, or other methods in the art such as Paxgene, HOPE, UmFix, or other so called “molecular fixatives”. For example, suitably the sample may be fixated using 0.1-4% formaldehyde. Suitably fixation may be performed at room temperature or at 4° C. Suitably fixation may be performed for a time between 1 second and 1 hour, suitably between 1 minute and 15 minutes. Suitably the substrate may be fixed prior to the methods of the invention. Suitably the substrate may be immobilized and fixed prior to the methods of the invention.

### (C) Permeabilization

Suitably, the substrates disclosed herein may further be permeabilised by any means known in the art. For example, permeabilization may be performed using detergent incubation or alcohol precipitation. Suitably, permeabilization may be performed using digitonin. Suitably, permeabilization may be performed using Triton X-100 in concentrations ranging from 0.1% w/v to 1% w/v for an incubation time ranging between 5 minutes and 30 minutes. Suitably, permeabilization may be performed using Nonidet P40, Igepal CA-630, or Tergitol type NP-40, in concentrations ranging from 0.01% to 1% for incubation times ranging between 5 minutes and 1 hour. Suitably, the agents above might be used in combination.

Permeabilization may be performed after fixation, and suitably after immobilization of the substrate. Suitably the substrate may be permeabilized prior to the methods of the invention. Suitably the substrate may be immobilized, fixed and permeabilized prior to the methods of the invention.

### (D) Post-Fixation

Suitably, the substrates disclosed herein may undergo a further step of fixation, suitably termed ‘post-fixation’ by any means known in the art. For example, post-fixation may be performed using aldehydes, alcohols, or other methods in the art. For example, suitably the sample may be post-fixated using 0.5%-4% formaldehyde. In addition or as an alternative, acrylate-based hydrogels such as polyacrylamide or sodium acrylate may be used, with or without anchoring of DNA and RNA to the gel can be employed. Such steps may be performed after an initial fixation step as outlined in (B) and may be performed after permeabilization as outlined in (C). Suitably the substrate may undergo post-fixation after each step of contacting the substrate with a transposase complex. Suitably after the transposase complex has attached the or each detection probe to the biological molecules of interest. Suitably therefore the methods may comprise more than one post-fixation step. Suitably if the method comprises two transposase contacting steps, then two post-fixation steps are present.

### (E) Protease/Lipid Clean-Up

Suitably, the substrates disclosed herein may undergo a clean-up step. Suitably a step of protein and lipid removal. Suitably comprising protease digestion and/or incubation with detergents to remove proteins and lipids respectively and increase tissue permeability and optical clarity. Such steps may be performed after an initial fixation step as outlined in (B) after permeabilization as outlined in (C) or after a post-fixation step as outlined in (D). Suitably the clean-up step may take place after the last post-fixation step. Suitably therefore a step of protein and lipid removal takes place after contacting the substrate with a transposase complex, and after post-fixation of said substrate.

### (F) Lysis and Purification

Suitably, the substrates disclosed herein may also undergo a step of lysis followed by purification of the biological molecules bearing spatial barcodes using any methods known in the art. For example, one or more chemical moieties allowing subsequent purification may be used as disclosed herein. Suitably such steps take place after the formation of a spatial barcode attached to the or each detection probe within the location of interest. Suitably prior to any sequencing steps. Suitably prior to any template switching steps.

Further features and embodiments of the present invention will now be described by reference to the following figures in which:

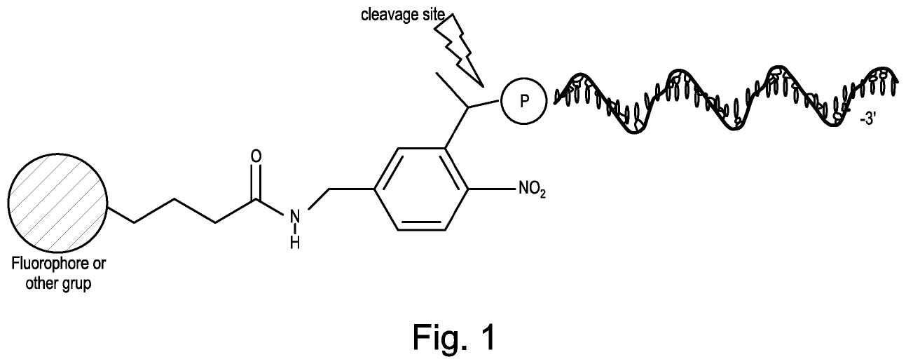

FIG. 1 shows: The structure of an embodiment of a photocleavable 5′ block on an oligonucleotide. The photocleavable group is released by illumination in the UV or violet range (340 nm to 410 nm) and yields an accessible 5′ phosphate group on the oligonucleotide, which can be targeted by ligation. The photocleavable group can be linked, on the side opposite to the protected phosphate, to a fluorophore group or to another nucleotide. This allows detection of the 5′ block and its release via fluorescence microscopy;

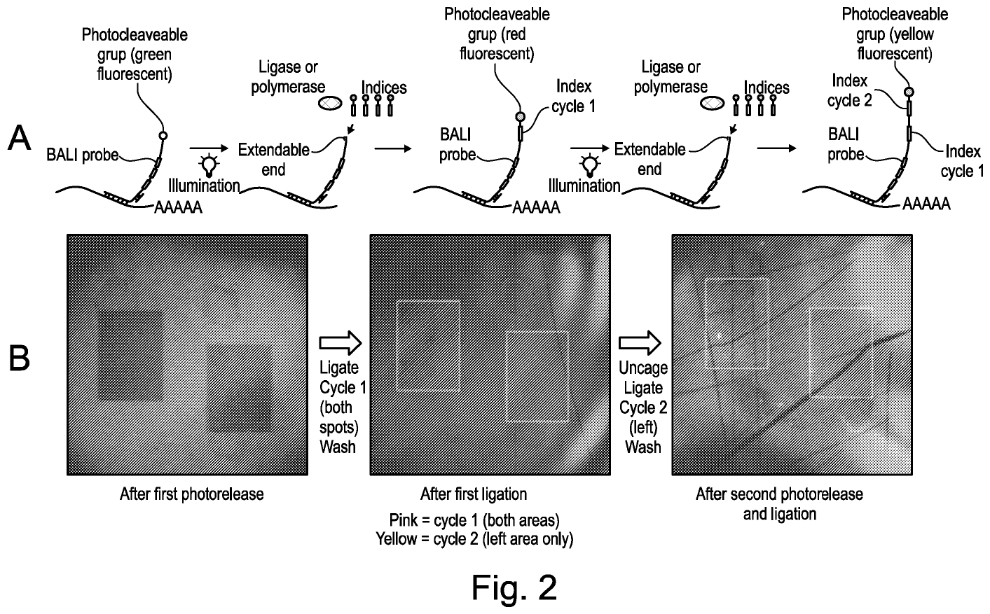

FIG. 2 shows: Proof of concept of spatial barcoding on a solid surface with two cycles of index ligation plus DNA bridge ligation. The schematics on top (A) indicate the process happening during the experiment. The detection probe (BALI probe) is ligated to a DNA bridge molecule bearing a photocleavable group labelled with the Alexa-488 fluorophore (cyan). The photocleavable group is cleaved by illumination, and a first index is ligated which bears a second photocleavable group labelled with the cy5 fluorophore (violet). The second photocleavable group is again removed by illumination, and a third index, labelled with Atto-568, is ligated (yellow). The images on the bottom (B) show the slide surface after the first photorelease step, after the ligation of the first index, and after the ligation of the second index. Two areas with reduced Alexa-488 signal (cyan) are visible on the left image, corresponding to the areas that have lost the first photocleavable group on the DNA bridge molecule. In the middle image, both areas show cy5 signal (pink), indicating successful ligation of the first index. In the right image, the leftmost area shows a reduced cy5 signal, due to the removal of the second photocleavable group, and an Atto-568 signal (yellow), indicating successful ligation of the second index;

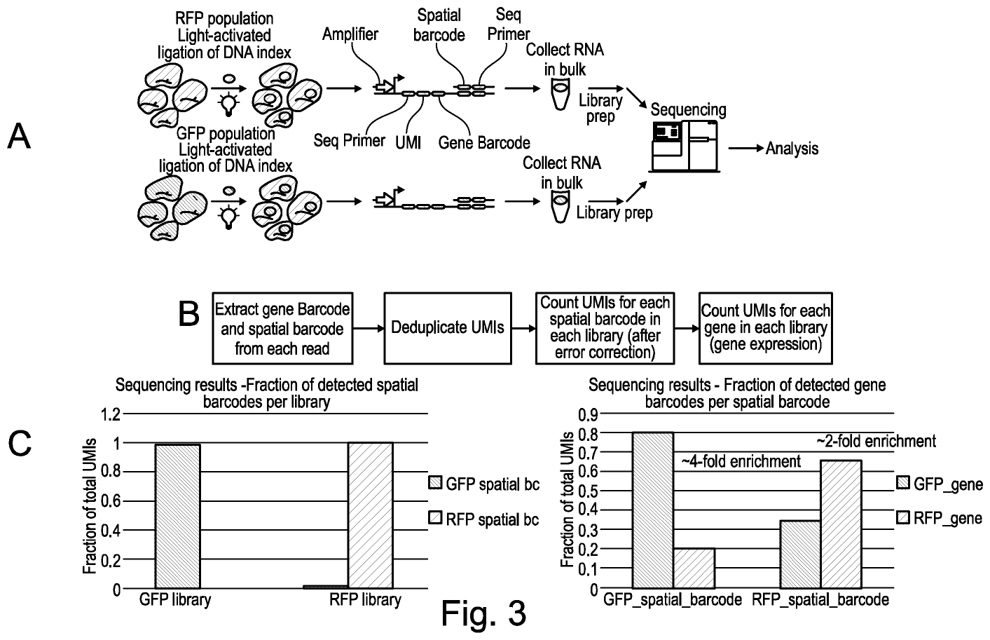

FIG. 3 shows: results of a proof of concept experiment (example 2) aimed at measuring gene expression in cultured cells using the light-based spatial barcoding steps of the invention and illumina DNA sequencing as quantification tool. For this experiment we used a pool of detection probes targeting two genes, green fluorescent protein and red fluorescent protein, expressed in two separate cell populations. Each population was barcoded with a different index sequence. The two populations were deconvolved after sequencing by matching the spatial barcodes assigned by the methods of this invention, and the abundance of the detection probes targeting each gene quantified. In a successful experiment, the gene abundance measured by the experiment should correspond to the fluorescent gene expressed in each cell population. (A) scheme of the experiment (B) computational analysis pipeline used to calculate results. (C) left: fraction of reads mapped to each spatial barcode (“GFP population” or “RFP population”) detected into the library produced from GFP or RFP cells. Essentially all reads bear the correct spatial barcode. Right: gene abundance measured for the GFP and RFP detection probes in each cell population. The GFP detection probes are predominantly detected in the GFP population and vice versa, indicating that the protocol can detect gene expression. Detection of RFP proves in the GFP population and vice versa is due to a specific binding of the detection probes

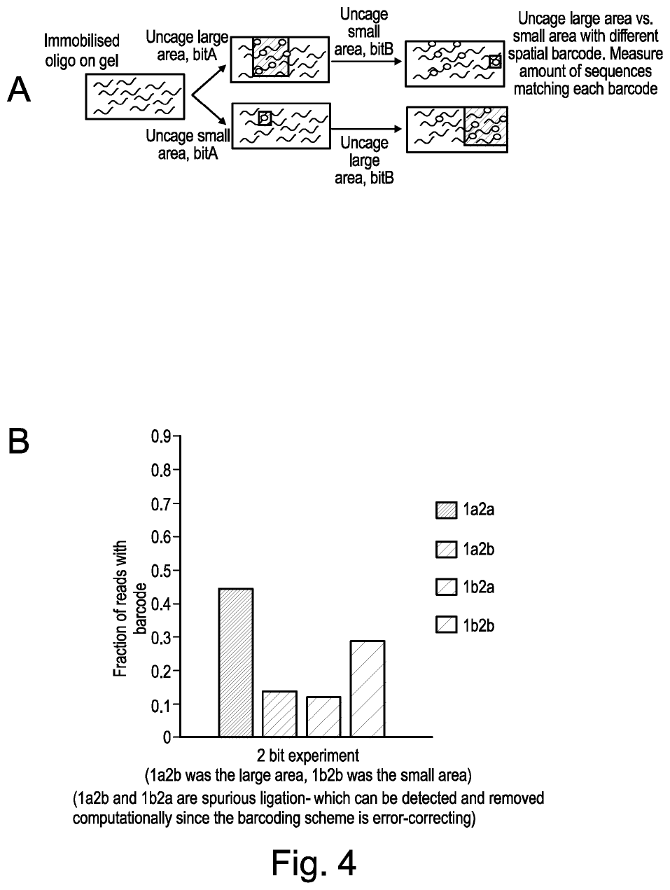

FIG. 4 shows: results on an experiment (example 3) aimed at showing that spatial barcoding of the invention can successfully measure the abundance of detection probes bound to different areas of the same tissue (example 9 was done on separate cell populations). A functionalised hydrogel bearing root molecules designed to resemble detection probes is used for this experiment. Areas of different sizes are uncaged and barcoded with a 2-bit spatial barcode using the methods of this invention. The abundance of detection probes in each spatially barcoded area is measured by illumina sequencing by mapping the spatial barcode present in each read. In a successful experiment, the abundance of detection probes measured in each spatially barcoded area should match the area size. (A) scheme of the experiment. (B) results of the quantification. The 2-bit spatial barcodes assigned to the “large” and “small” area were “1a2a” and “1b2b”. The experiment correctly measures more molecules for 1a2a. The other combinations (“1a2b” and “1b2a”) are presumably products of spontaneous uncaging and ligation produced by stray light in the experiment (since the proof of concept experiment could not be done in completely light-proof conditions)

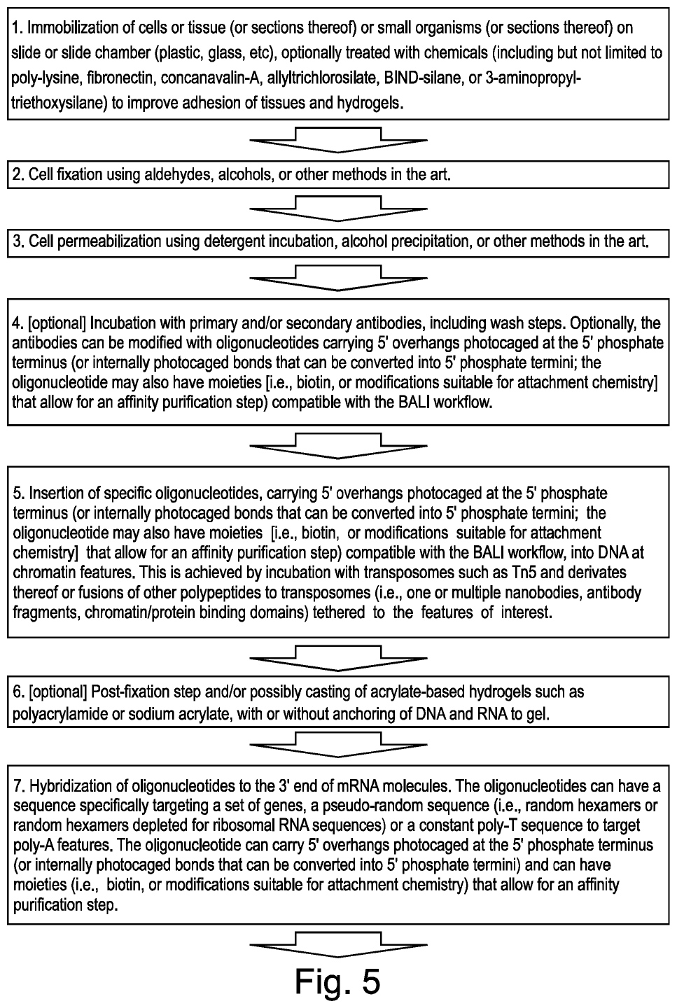

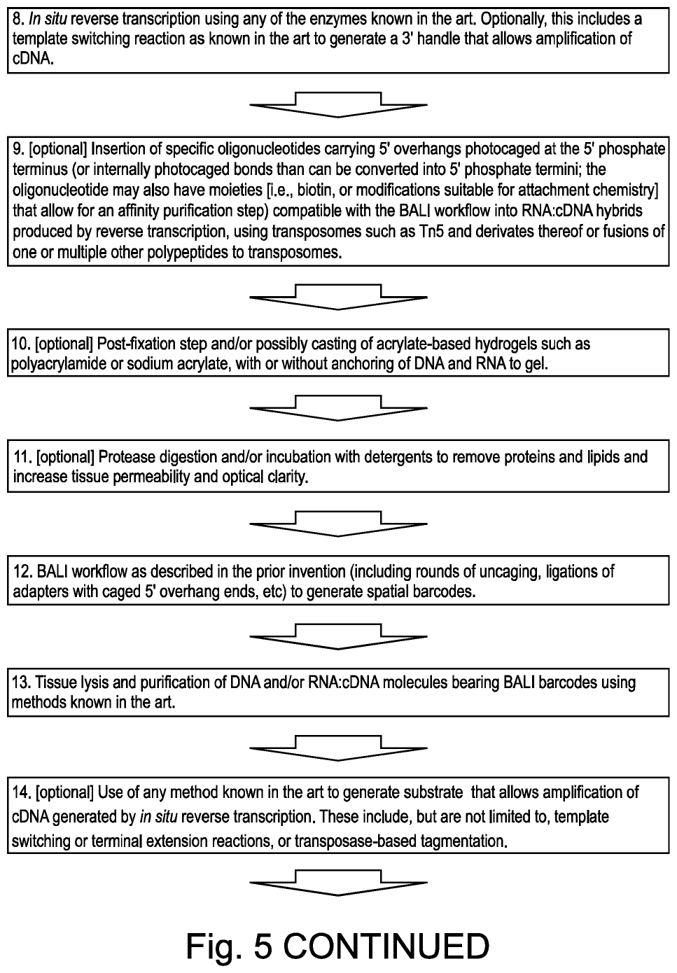

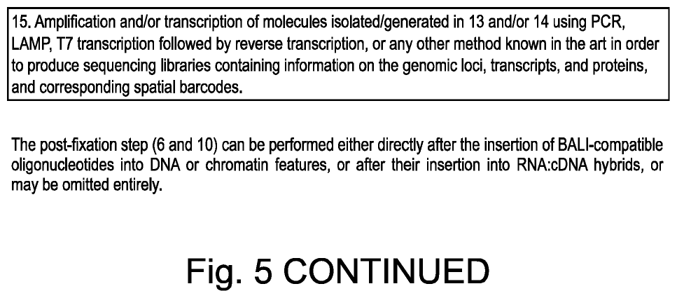

FIG. 5 shows: all steps that may be performed in connection with the method of the invention (referred to in the figure as “Spatial multi-omics”) involving the generation of substrates for spatial barcoding of multiple features simultaneously.

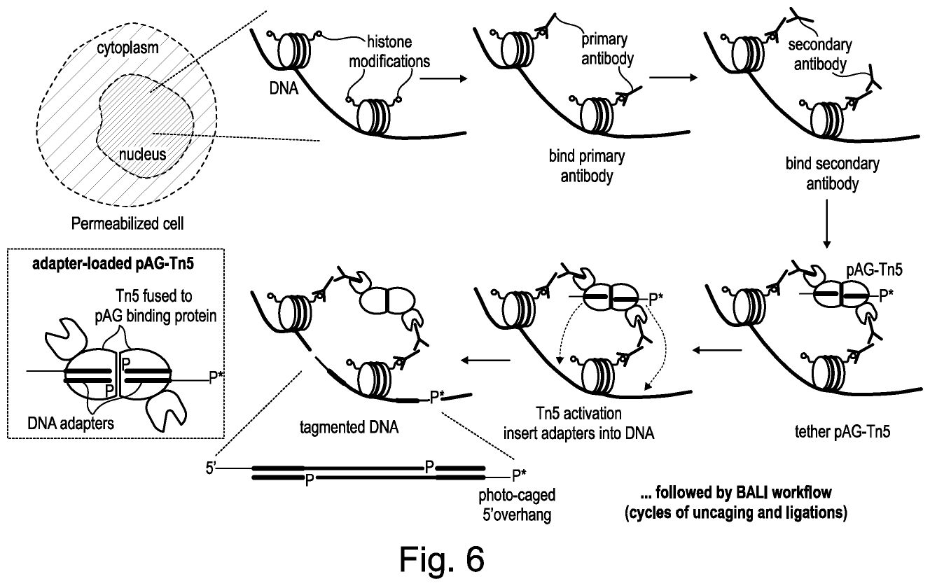

FIG. 6 shows: a schematic diagram for the method of the invention applied to chromatin features, such as histone modifications.

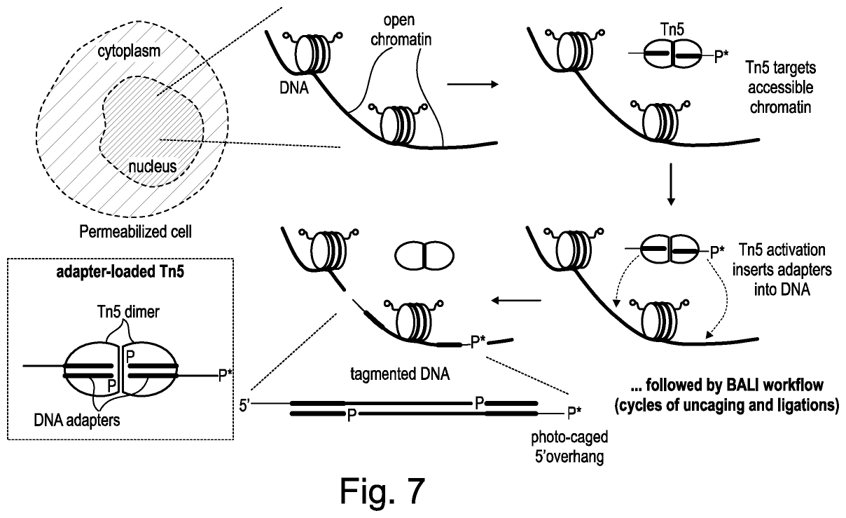

FIG. 7 shows: a schematic diagram for the method of the invention applied to open chromatin.

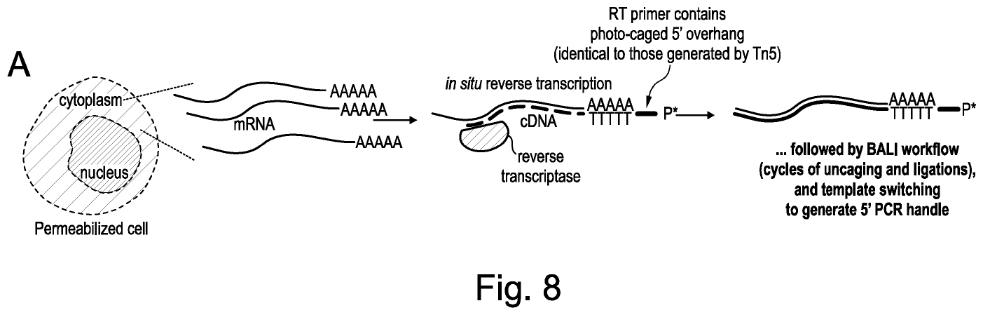

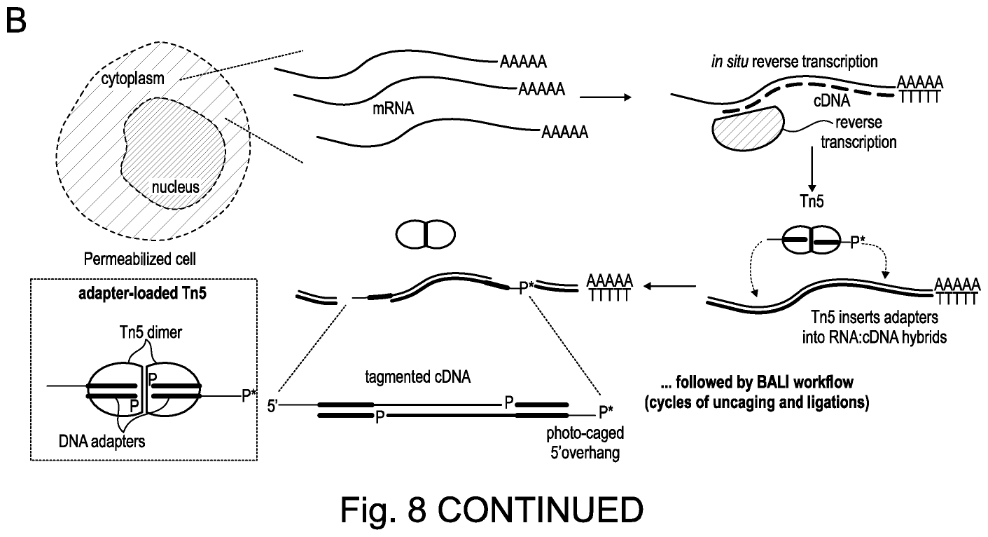

FIG. 8 shows: a schematic diagram for the method of the invention applied to RNA either (A) a schematic diagram of in situ reverse transcription to generate RNA:cDNA hybrids for the addition of a spatial barcode using a non-transposase based method (e.g., template switching—see also FIG. 20), or (B) a schematic diagram of in situ reverse transcription to generate RNA:cDNA hybrids followed by the insertion of photocaged detection probes for subsequent addition of a spatial barcode using a transposase complex.

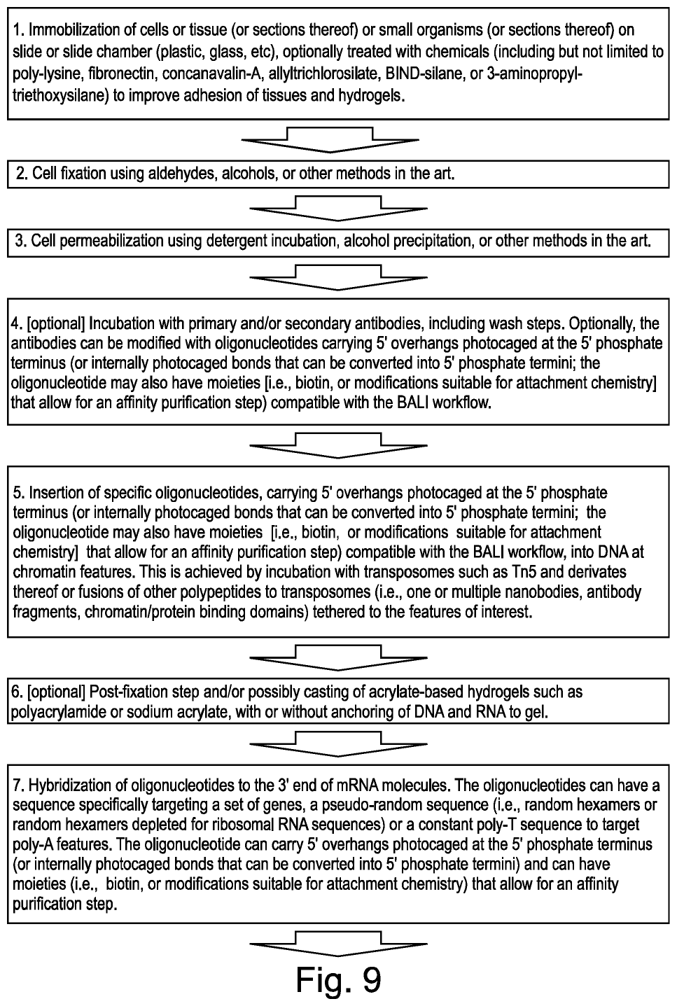

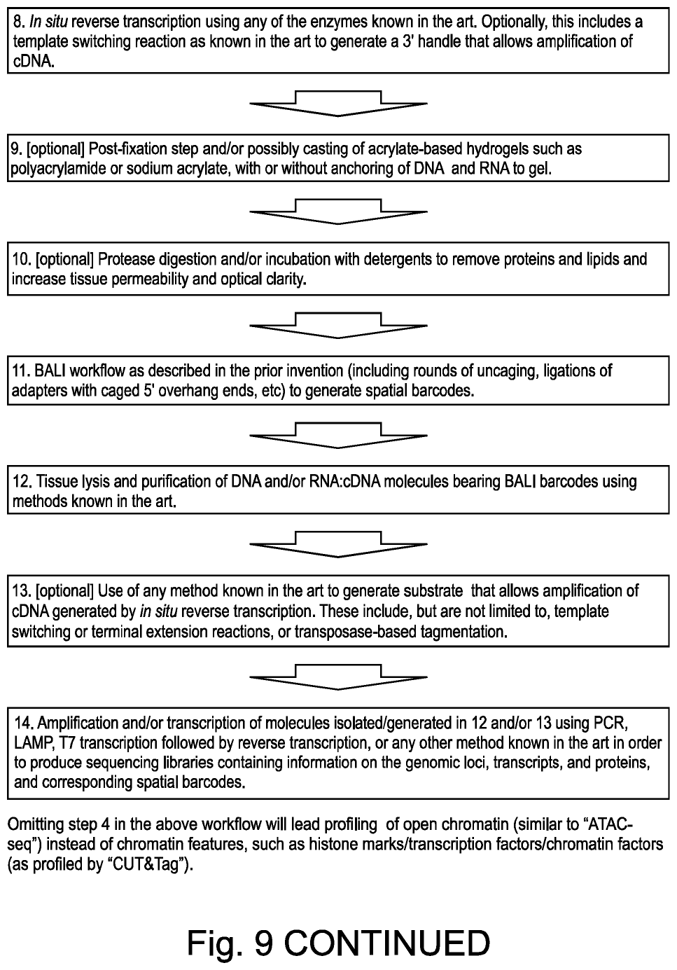

FIG. 9 shows: all possible steps that may be performed for profiling chromatin features such as histone marks, transcription factors, chromatin factors using a transposase complex and RNA (option 1 using Reverse-transcription).

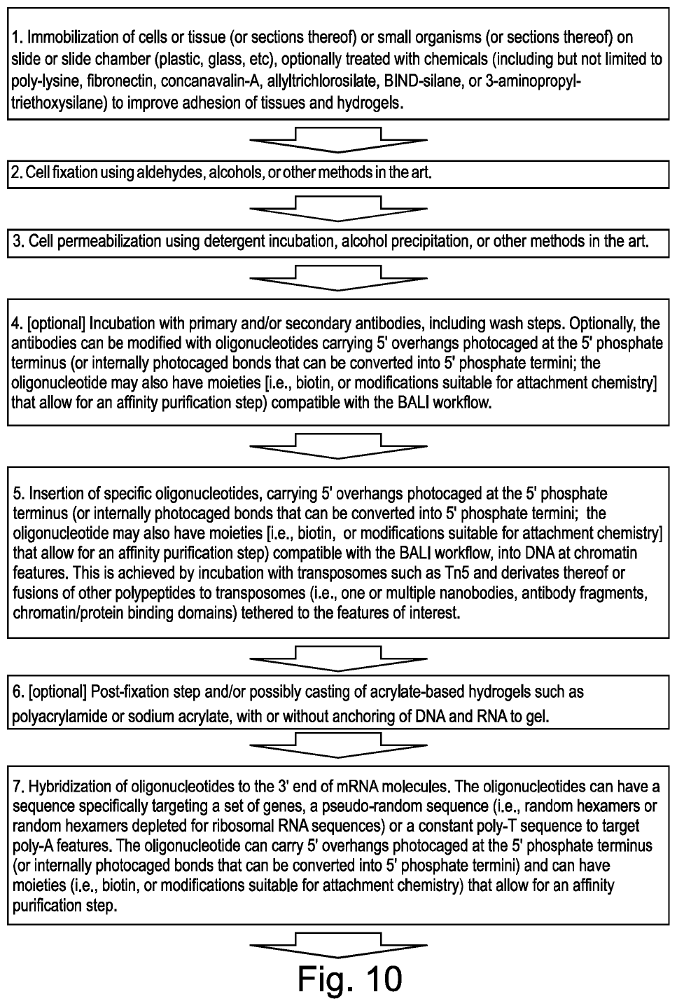

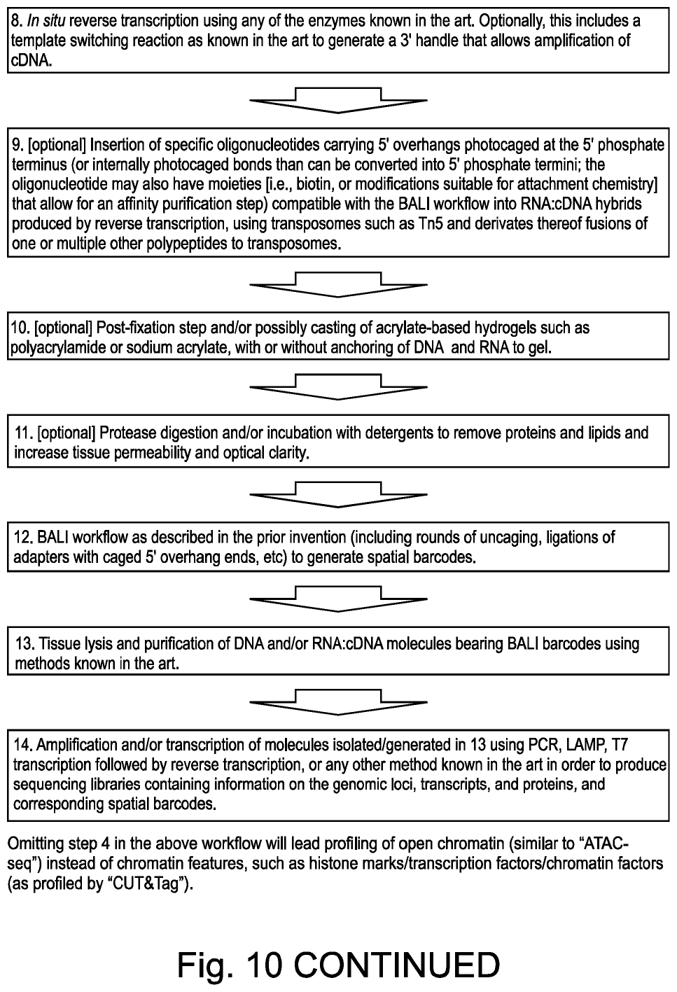

FIG. 10 shows: all possible steps that may be performed for profiling chromatin features such as histone marks, transcription factors, chromatin factors using a transposase complex and RNA (option 2 using a transposase complex).

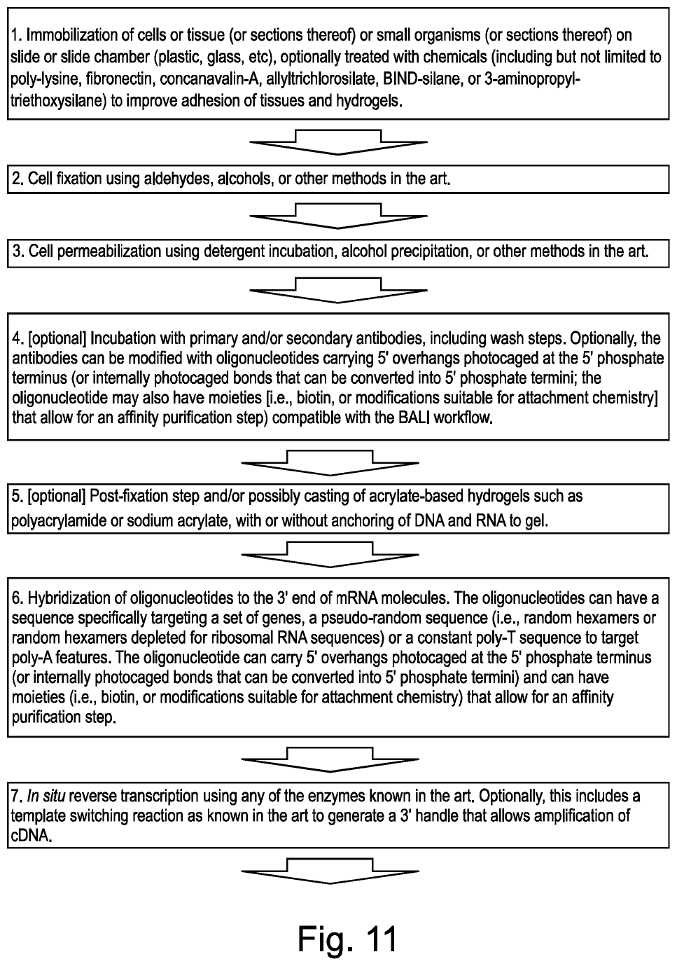

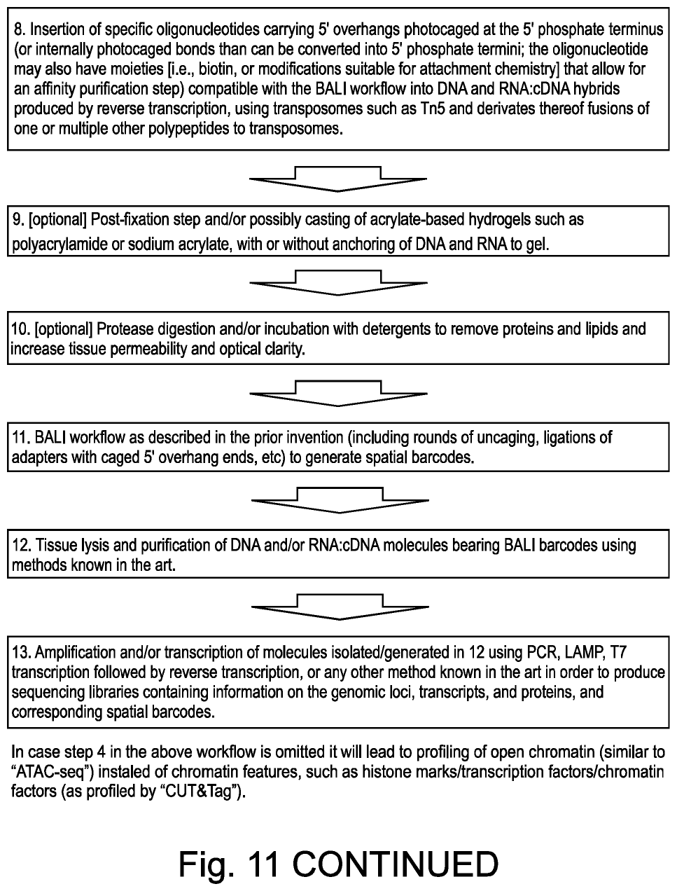

FIG. 11 shows: all the steps that may be performed for profiling of chromatin/DNA using a transposase complex and RNA (using a transposase complex)

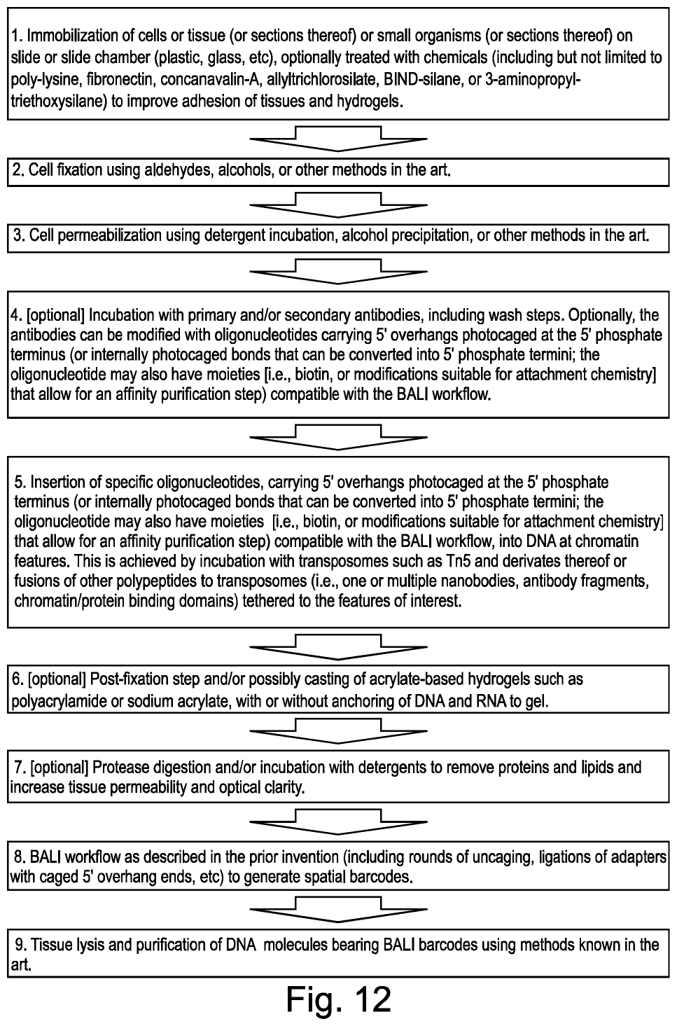

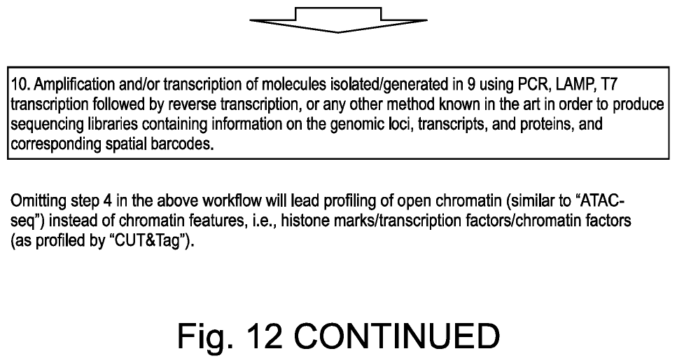

FIG. 12 shows: all the steps that may be performed for profiling of chromatin features only using a transposase complex.

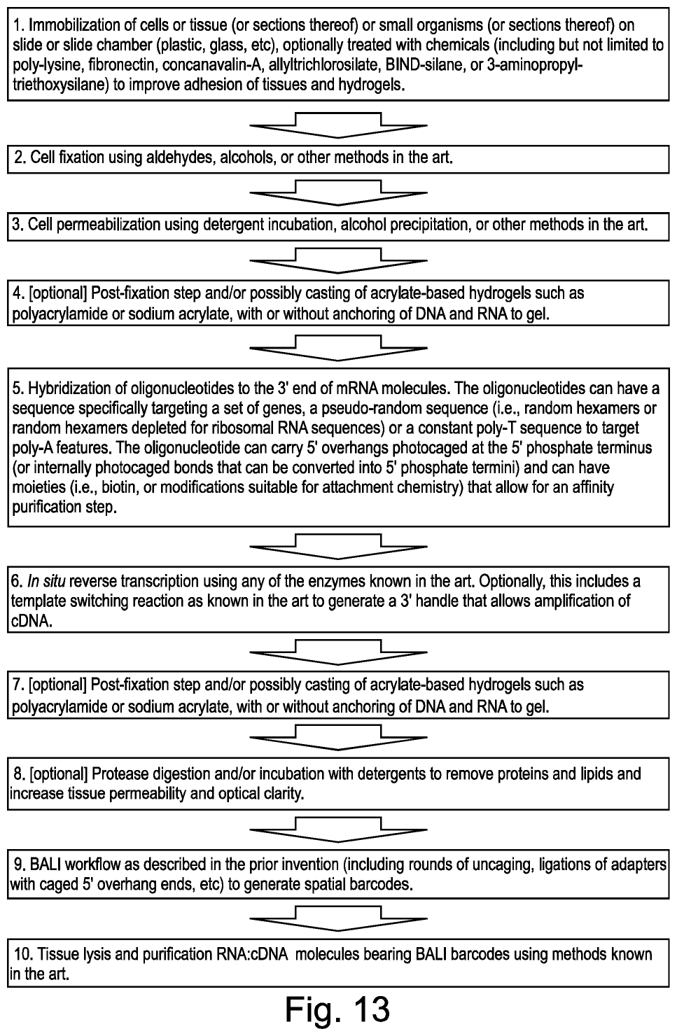

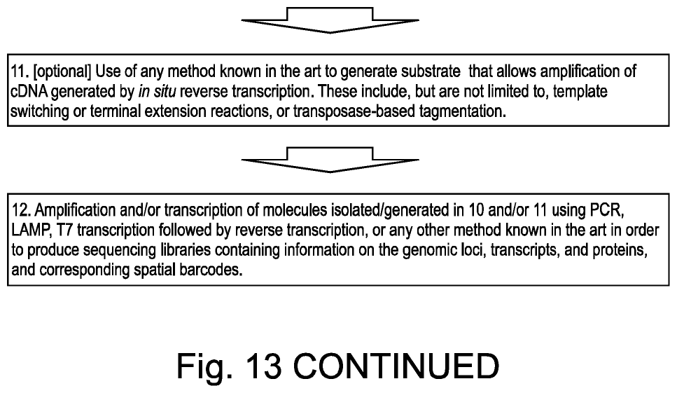

FIG. 13 shows: all the steps that may be performed for profiling of RNA only using reverse transcription.

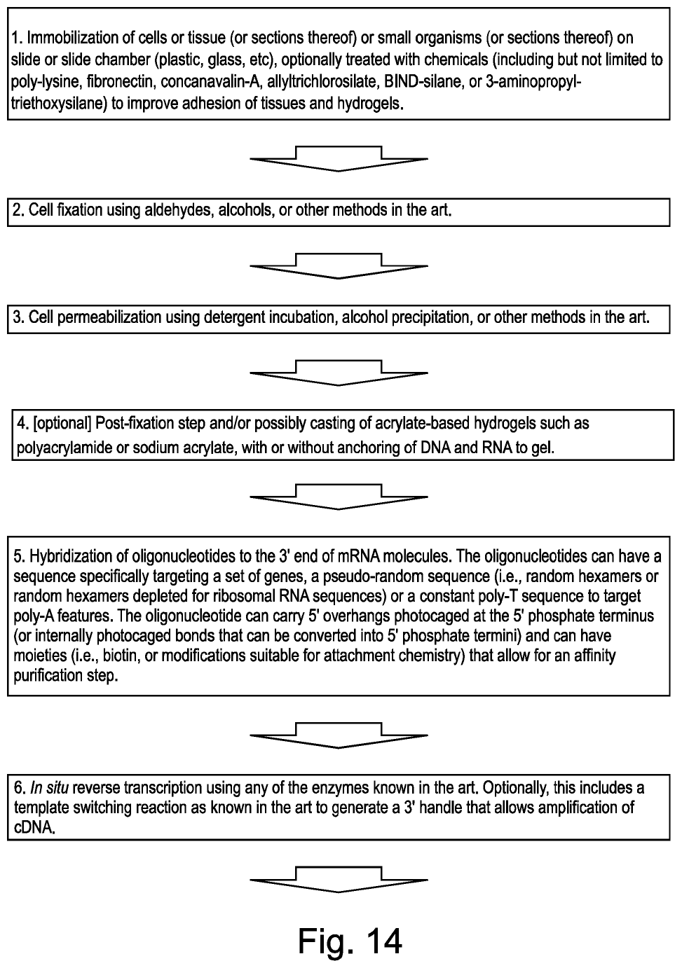

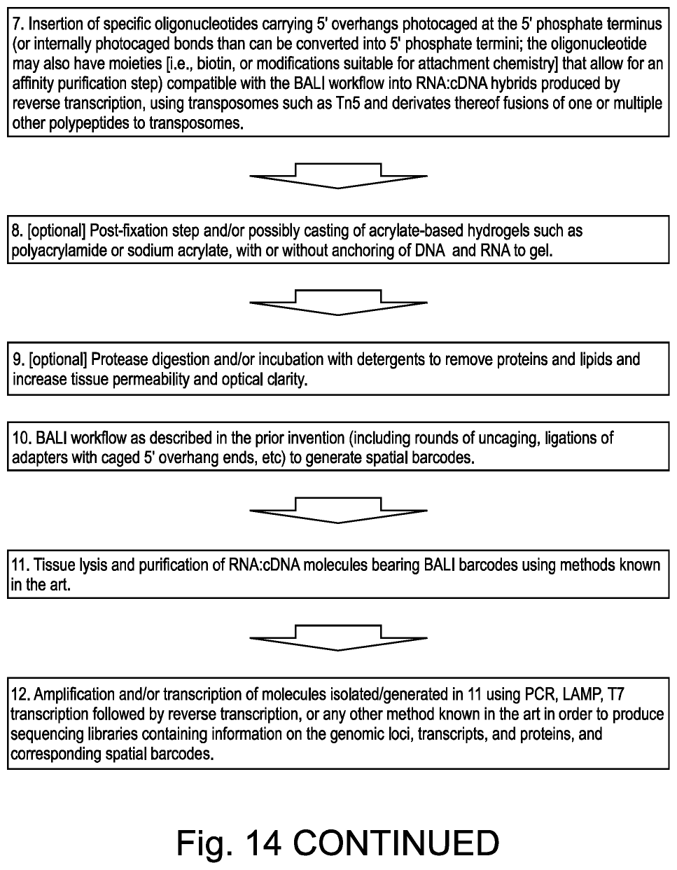

FIG. 14 shows: the steps that may be performed for profiling of RNA only using a transposase complex.

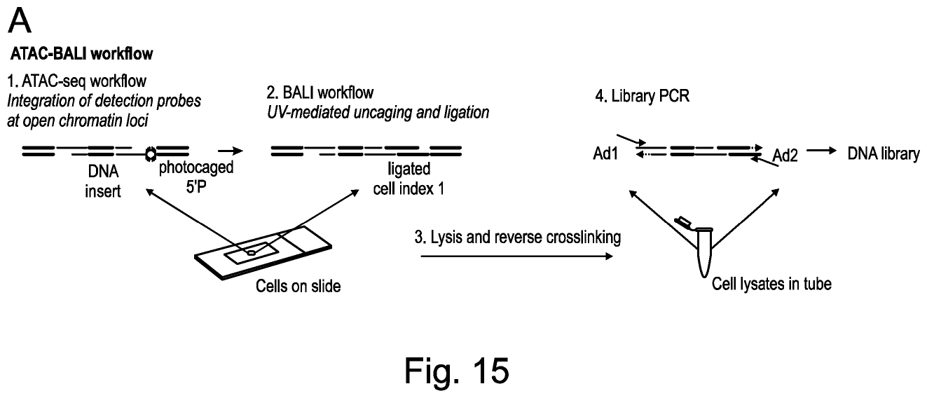

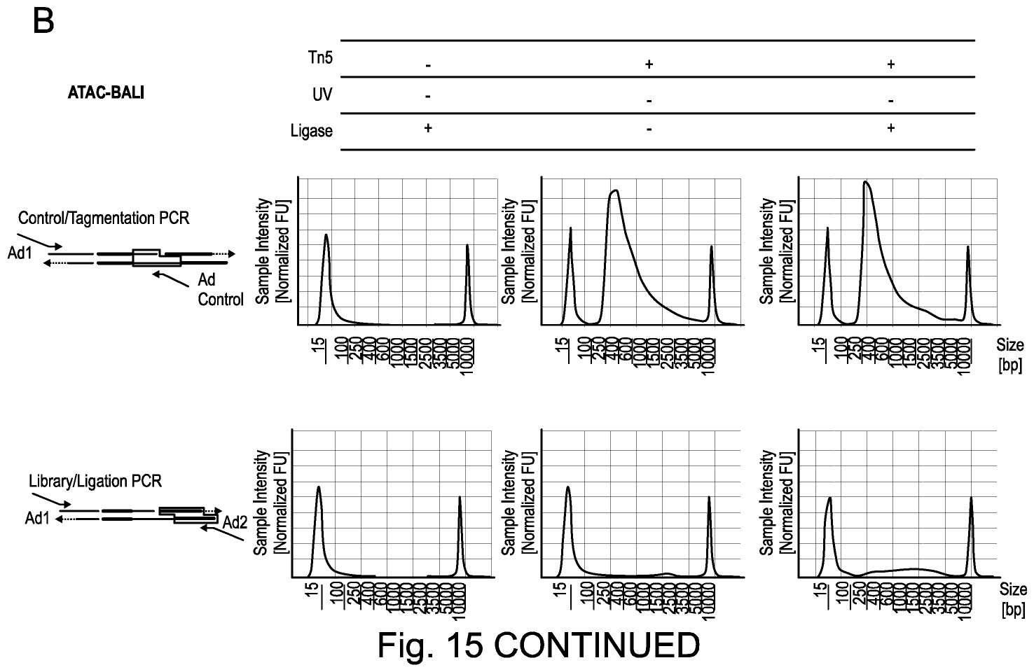

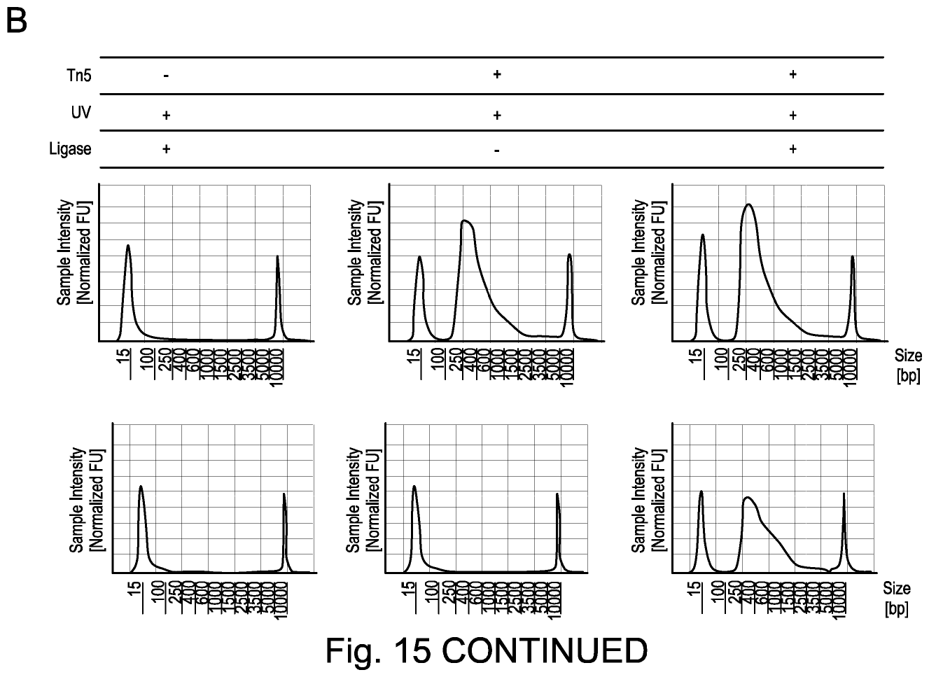

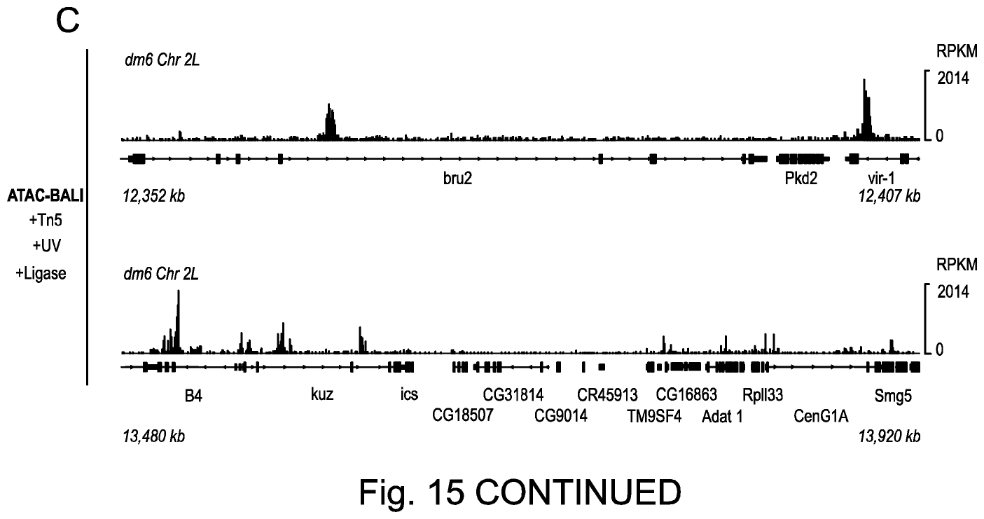

FIG. 15 shows: the procedure encompassing profiling of chromatin only, such steps being referred to ATAC-BALI in the figure. (A) Schematic of the procedure that was carried out on cells grown on slides. A transposase complex (Tn5) is used to attach detection probes harbouring a photocleavable group to DNA at open chromatin areas (1, ATAC-seq workflow). One round of illumination-mediated photocleavable group cleavage followed by one ligation allows the construction of a spatial barcode within the area of interest (2, BALI workflow). Following lysis and reverse crosslinking (3), library construction is carried out in vitro (4). (B) TapeStation D5000 ScreenTape profiles of samples obtained at the end of the procedure shown in (A). Positive Control PCR indicates successful integration of detection probes into DNA. Positive Library/Ligation PCR indicates first successful integration of detection probes into DNA and second successful construction of a spatial barcode by ligation of an index sequence after illumination-mediated photocleavable group cleavage. Positive Library/Ligation PCR products were only observed when UV light was used to uncage the detection probe and when T4 DNA ligase was present during the ligation reaction step. (C) ATAC-BALI profiles of positive Library/Ligation PCR sample shown in (B) following sequencing, spatial barcode trimming and mapping. Reads per kilobase per million mapped reads are shown for two genomic regions (chromosomal coordinates are indicated).

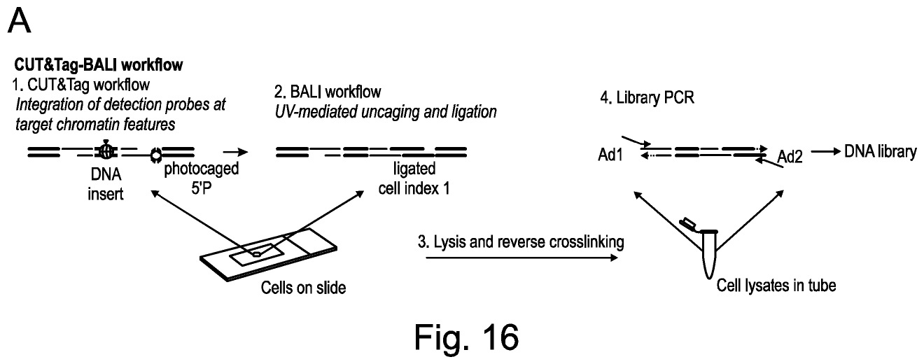

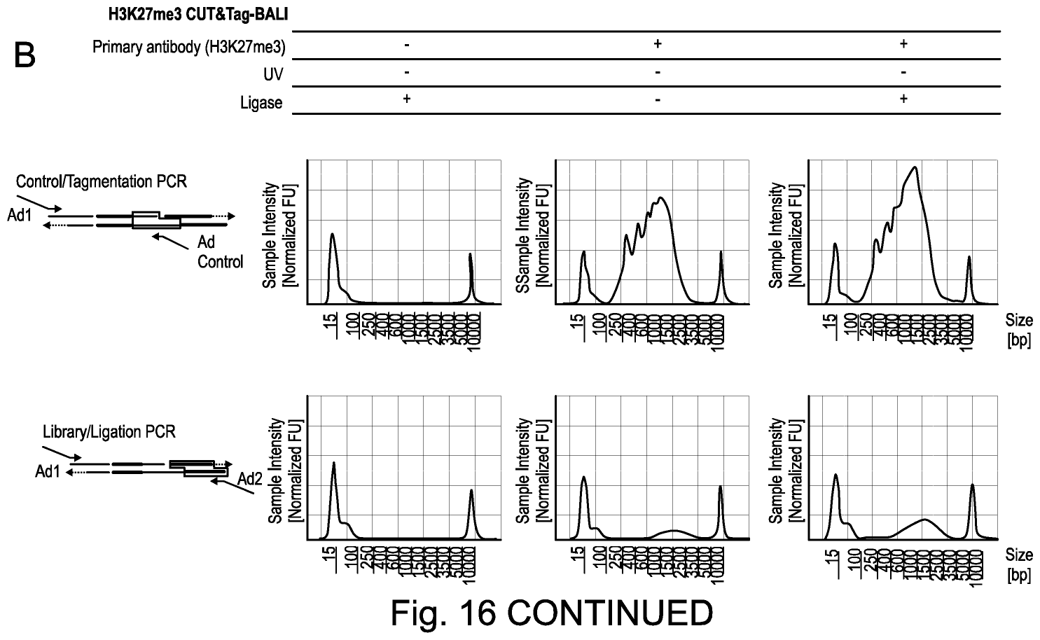

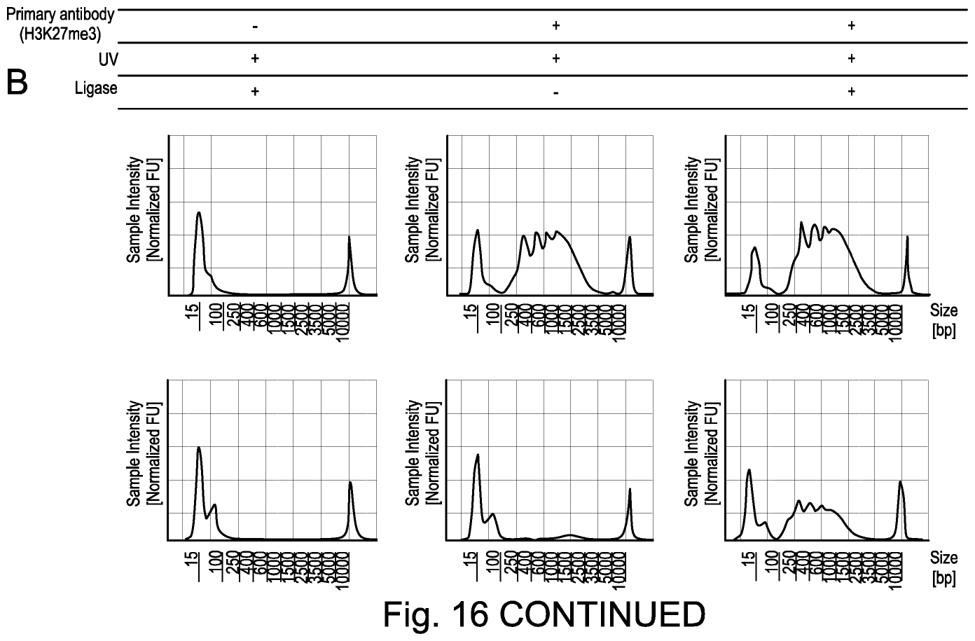

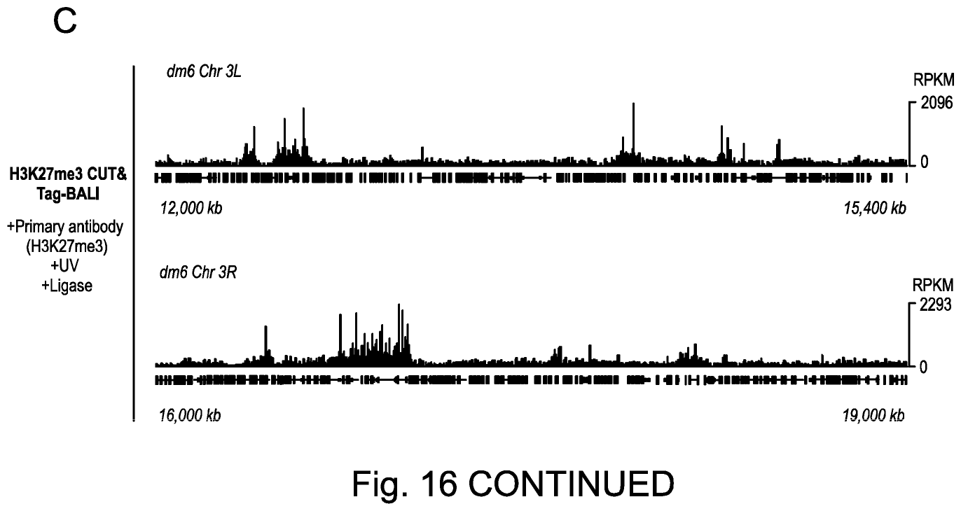

FIG. 16 shows: the procedure encompassing profiling of chromatin features only-such steps being referred to as CUT&Tag-BALI (CUT&Tag followed by BALI workflow) in the figure. (A) Schematic of the procedure that was carried out on cells grown on slides. A transposase complex (pA-Tn5) is used to attach detection probes harbouring a photocleavable group to DNA at target chromatin features (H3K27me3) by tethering via a primary antibody specific to this histone mark followed by a secondary antibody (1, CUT&Tag workflow). One round of illumination-mediated photocleavable group cleavage followed by one ligation allows the construction of a spatial barcode within the area of interest (2, BALI workflow). Following lysis and reverse crosslinking (3), library construction is carried out in vitro (4). (B) TapeStation High Sensitivity D5000 ScreenTape profiles of samples obtained at the end of the procedure shown in (A). Positive Control PCR indicates successful integration of detection probes into DNA. Positive Library/Ligation PCR indicates first successful integration of detection probes into DNA and second successful construction of a spatial barcode by ligation of an index sequence after illumination-mediated photocleavable group cleavage. Positive Library/Ligation PCR products were only observed when UV light was used to uncage the detection probe and when T4 DNA ligase was present during the ligation reaction step. (C) CUT&Tag-BALI profiles of positive Library/Ligation PCR samples shown in (B) following sequencing, spatial barcode trimming and mapping. Reads per kilobase per million mapped reads are shown for two genomic regions (chromosomal coordinates are indicated).

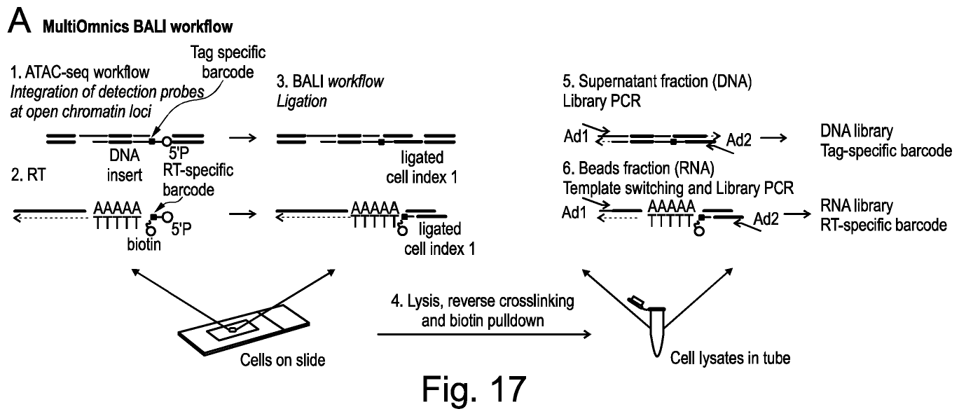

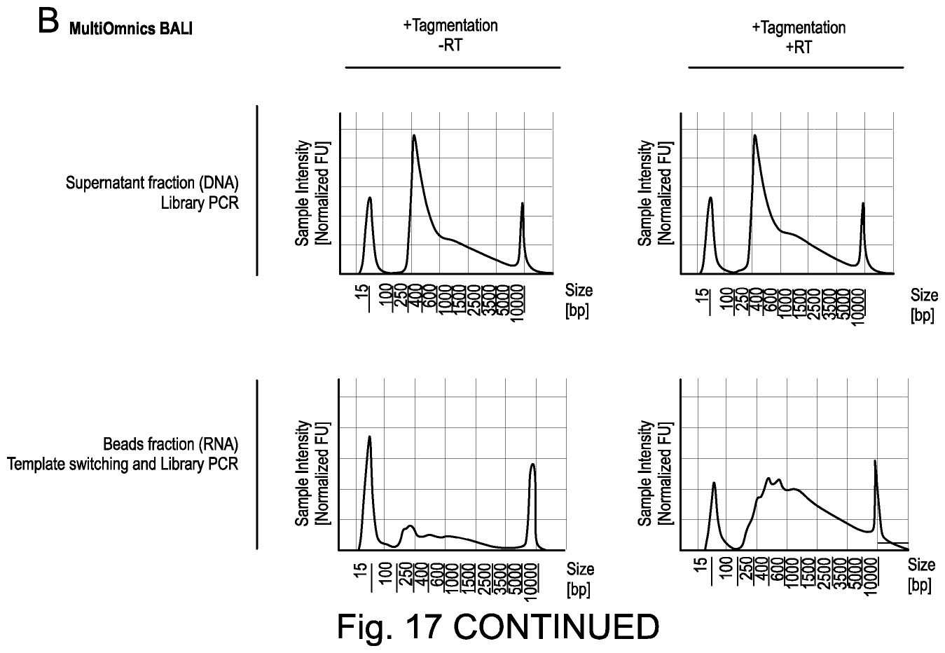

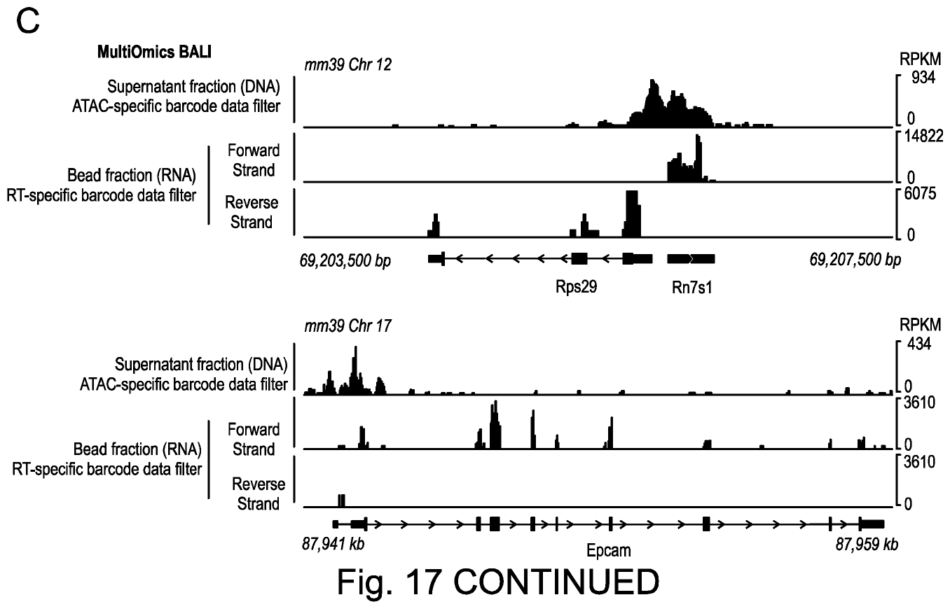

FIG. 17 shows: the procedure encompassing profiling of open chromatin and RNA together, such steps being referred to as MultiOmics BALI in the figure. (A) Schematic of the procedure that was carried out on cells grown on slides. First, a transposase complex (Tn5) is used to attach detection probes harbouring a 5′ phosphate group and a specimen-specific barcode to DNA at open chromatin areas (1, ATAC-seq workflow). Subsequently, a reverse transcription step is used to attach detection probes harbouring biotin, a 5′ phosphate group and a specimen-specific barcode to (polyA-containing) mRNAs (2, RT workflow). One round of ligation allows the construction of a spatial barcode within the area of interest (3, BALI workflow). Following lysis and reverse crosslinking, RNA and DNA fractions are physically separated using streptavidin magnetic beads and processed independently as indicated (4). DNA library construction (5) and RNA library construction (6) are carried out separately in vitro (B) TapeStation High Sensitivity D5000 ScreenTape profiles of DNA and RNA libraries obtained at the end of the procedure shown in (A). Positive Library PCR indicates successful integration of detection probes into DNA (top) or RNA (bottom) as well as successful construction of a spatial barcode by ligation of an index sequence (C) MultiOmics BALI profiles of positive Library PCR samples shown in (B) following sequencing, specimen-barcode filtering, spatial barcode trimming and mapping. Reads per kilobase per million mapped reads are shown for two genomic regions (chromosomal coordinates are indicated). RNA libraries are stranded with both forward and reverse strands displayed in separate tracks.

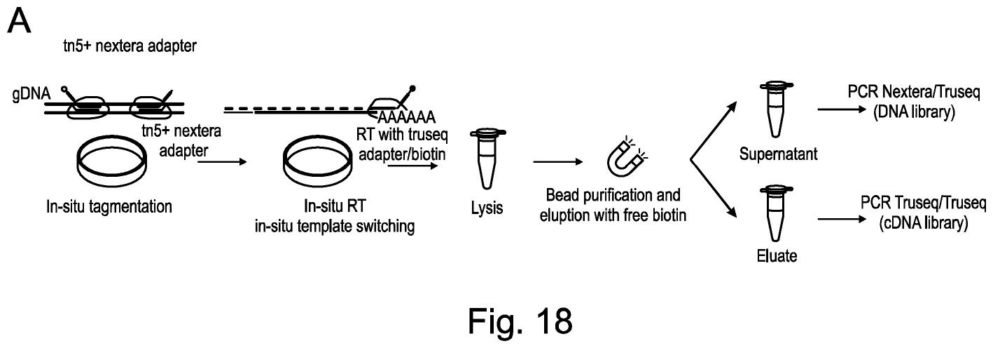

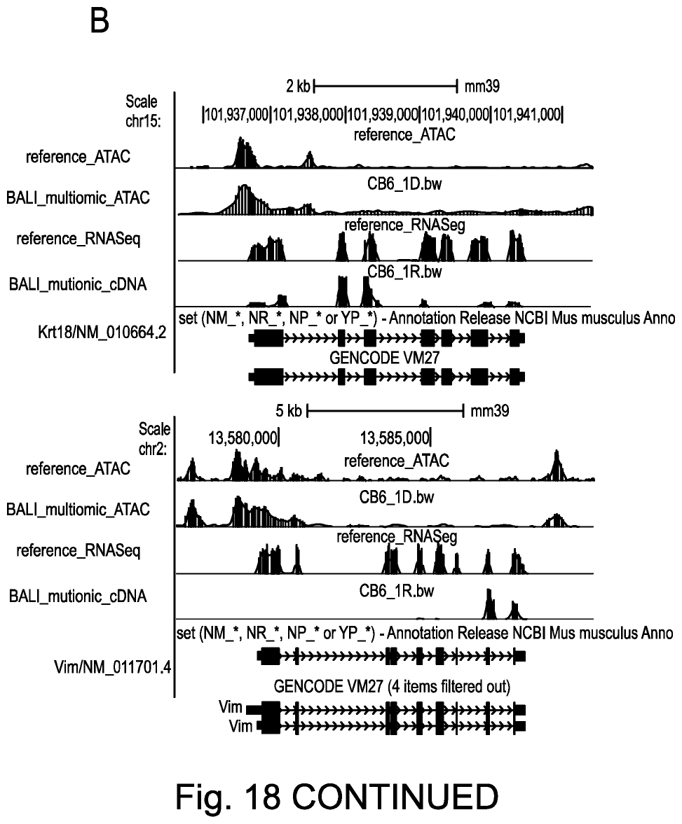

FIG. 18 shows: An alternative procedure encompassing profiling of open chromatin and RNA together, such steps being referred to as MultiOmics BALI in the figure. (A) Schematic of the procedure that was carried out on cells grown on slides. First, a transposase complex (Tn5) is used to attach detection probes harbouring a modality-specific barcode and sequencing primer to DNA at open chromatin areas (ATAC-Seq). Subsequently, a reverse transcription step is used to attach detection probes harbouring biotin, a sequencing primer and a modality-specific barcode to (polyA-containing) mRNAs. In this same step, a 3′ handle is added by means of in-situ template switching, bearing a second modality-specific sequencing primer. Following lysis and reverse crosslinking, RNA and DNA fractions are physically separated using streptavidin magnetic beads and processed independently. DNA library construction and RNA library construction are carried out separately in vitro using the modality-specific sequencing primers, and the resulting libraries are multiplexed on an Illumina NextSeq sequencing run. (B) MultiOmics BALI profiles following sequencing, modality-barcode filtering, and mapping. Reads per kilobase per million mapped reads are shown for two genomic regions (chromosomal coordinates are indicated). Both ATAC-Seq profiles and RNA expression profiles are shown. In both cases, results from this experiment are compared with reference data from the art (non-spatial ATAC-Seq and RNA-Seq).

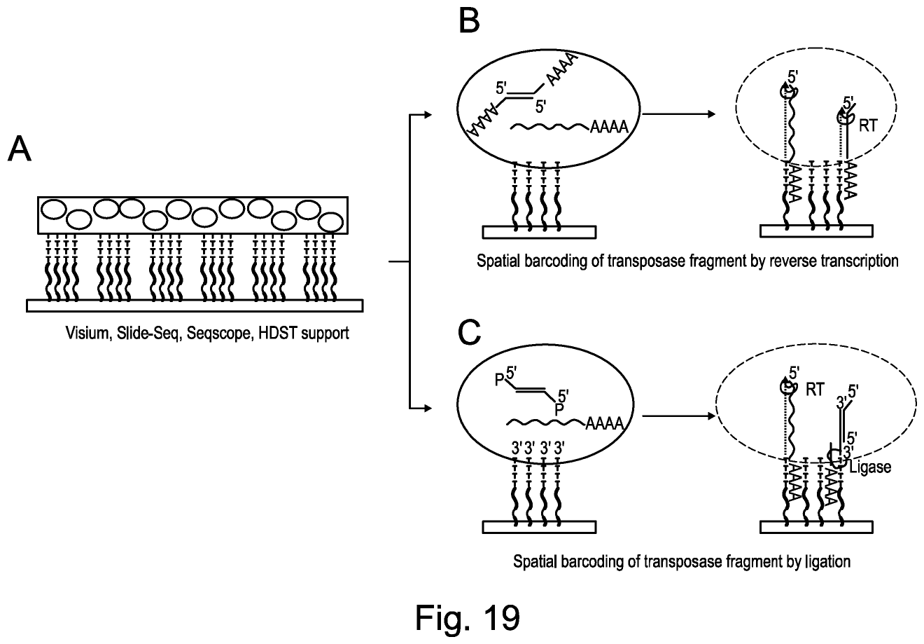

FIG. 19 shows: an application of the technology disclosed herein to existing spatially barcoded solid supports. (A) scheme of existing spatially barcoded supports, such as the slides used for 10× Visium, Slide-Seq V1/2, HDST or Seq-Scope. A tissue section is deposed over a support which is covalently modified with clusters of oligonucleotides exposing a poly-T 3′ terminus. All oligonucleotides within each cluster encode a cluster-specific and space-specific barcode sequence. PolyA modified mRNA from the tissue diffuses to the oligonucleotide clusters, is captured by the poly-T sequences, and is retro-transcribed by a reverse transcriptase producing spatially barcoded cDNA. (B) In one embodiment of the technology here described, chromatin/DNA and/or RNA:DNA duplexes are labelled with probes bearing a 3′ polyA sequence. This sequence can hybridize to the poly-T sequence exposed on the spatially barcoded support used by the existing method, so that the chromatin/DNA or RNA:cDNA fragments can be retro-transcribed together with mRNAs in the appropriate step. Optionally, the reverse transcription mix of the existing method is supplemented with a strand-displacing polymerase (suitably a strand displacing reverse transcriptase) capable of displacing one of the strands of the duplex. This produces copies of the chromatin/DNA or RNA:DNA duplexes which are spatially barcoded. (C) In a separate embodiment of the technology here described, chromatin/DNA and/or RNA:DNA duplexes are labelled with probes bearing a 5′ phosphate group. The poly-T oligonucleotides present on the spatially barcoded support are hybridized to a bridge oligonucleotide comprising a polyA sequence and a sequence complementary to the 5′ region of the probes. A DNA ligase (suitably T4 DNA ligase) is added to the reverse transcription mix of the existing method, and mediates the ligation of the chromatin/DNA or RNA:DNA duplexes to the spatially barcoded oligonucleotides linked to the support. This produces spatially barcoded chromatin/DNA or RNA:DNA fragments.

FIG. 20 shows: a template switching schematic which can be used in conjunction with the disclosed invention.

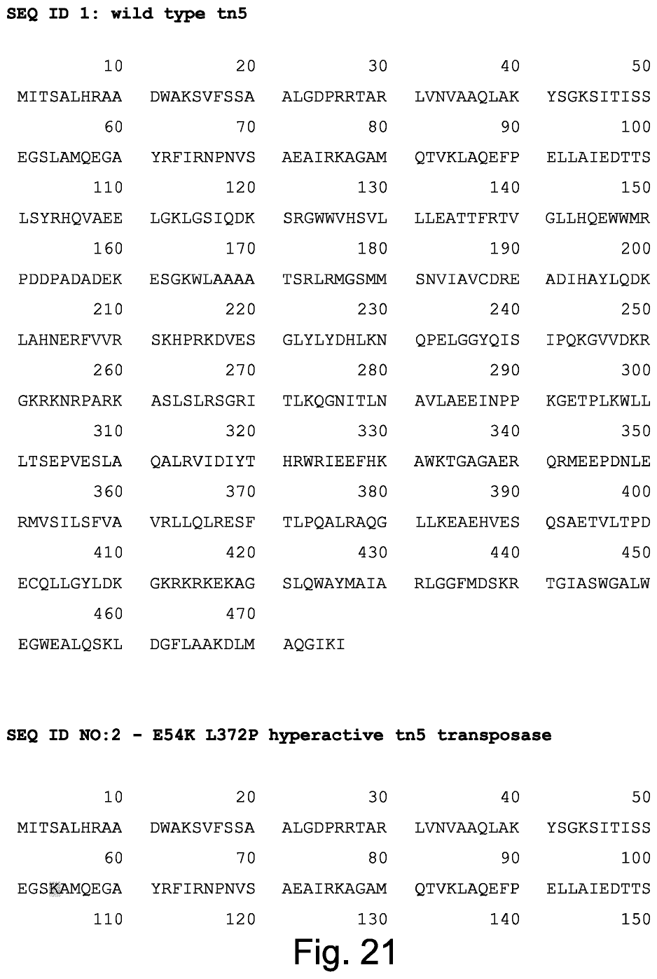

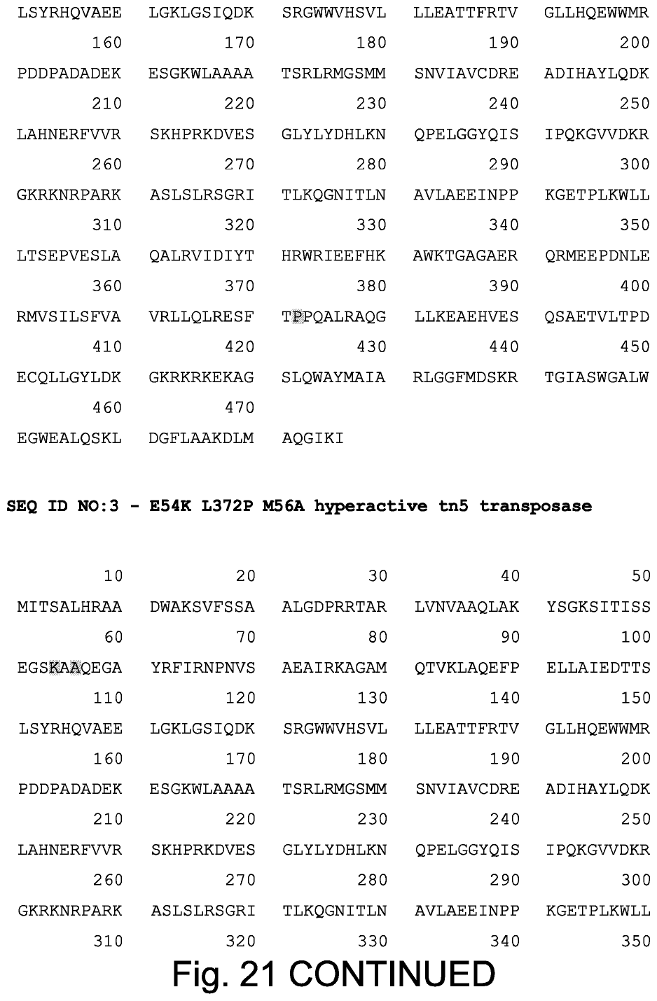

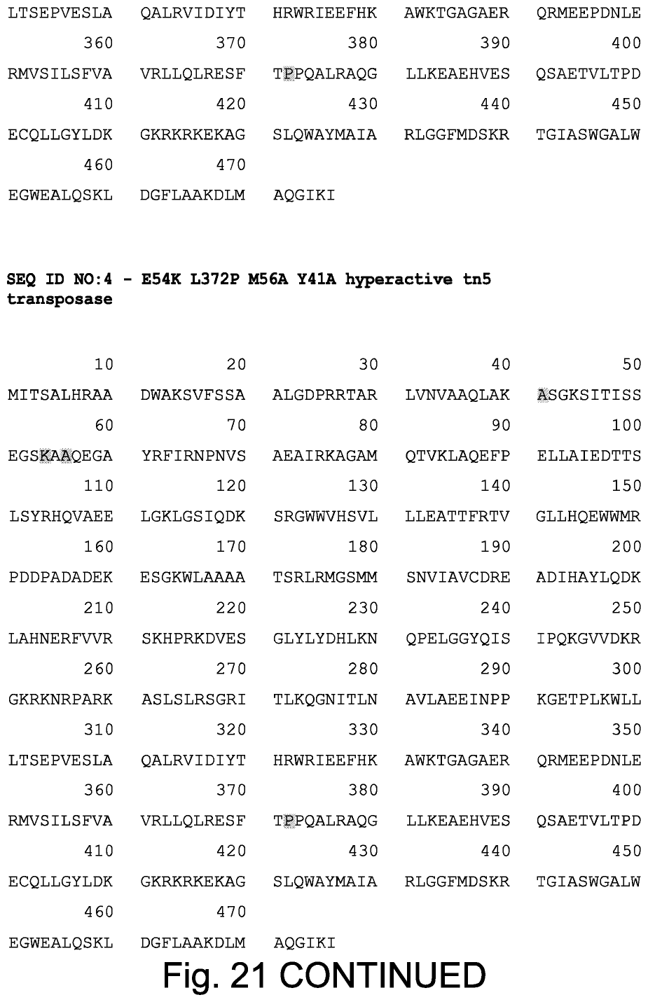

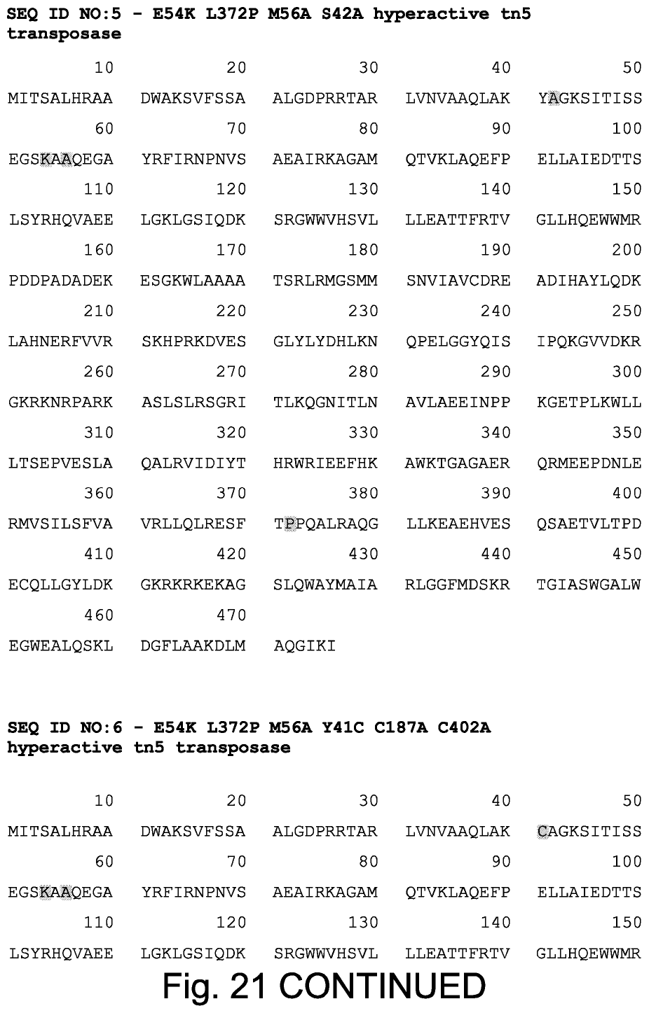

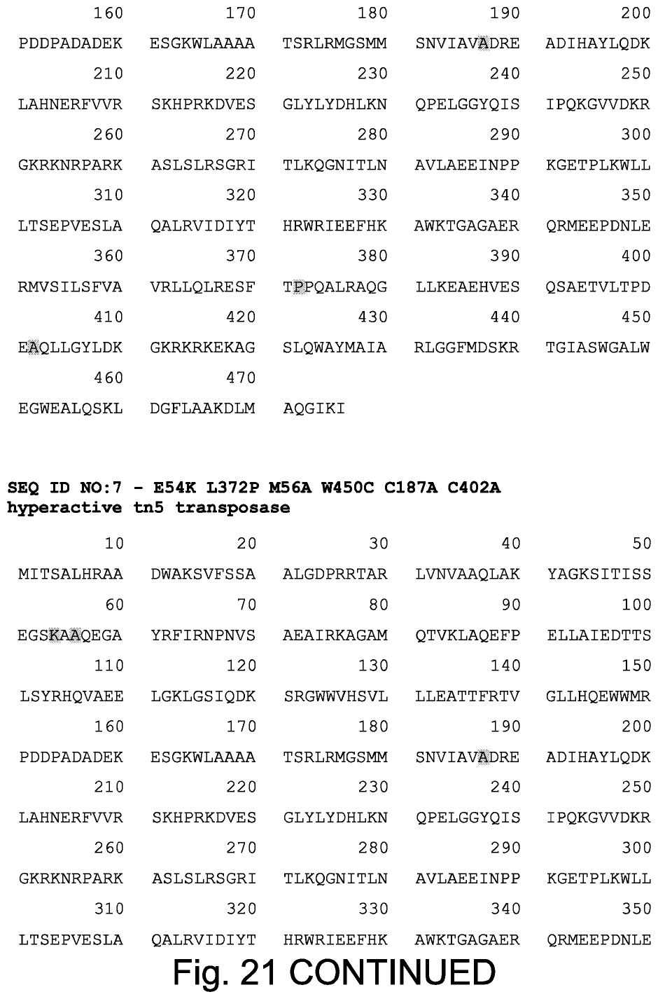

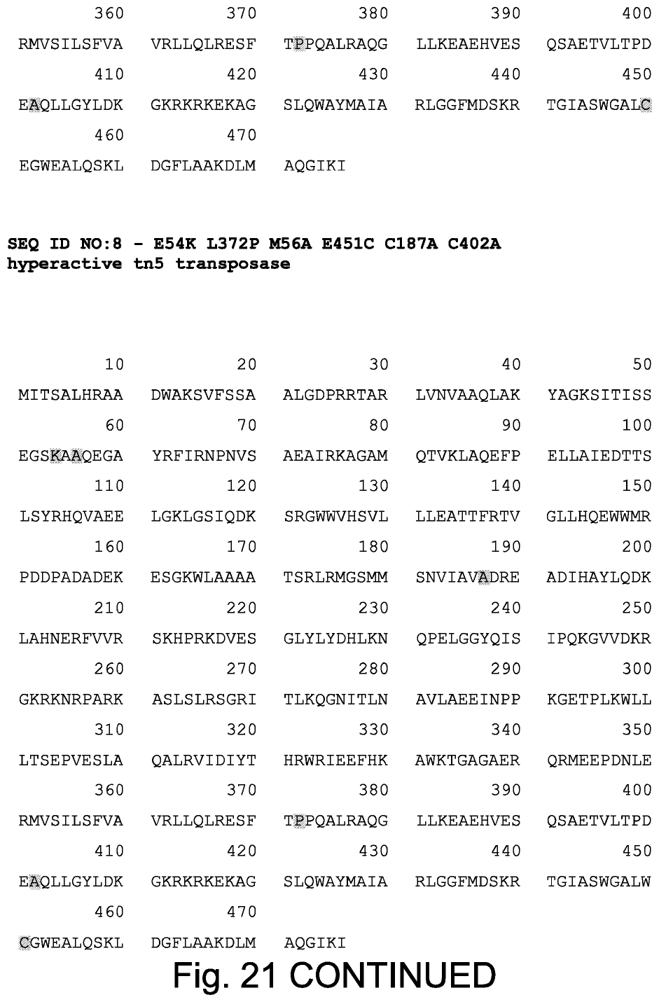

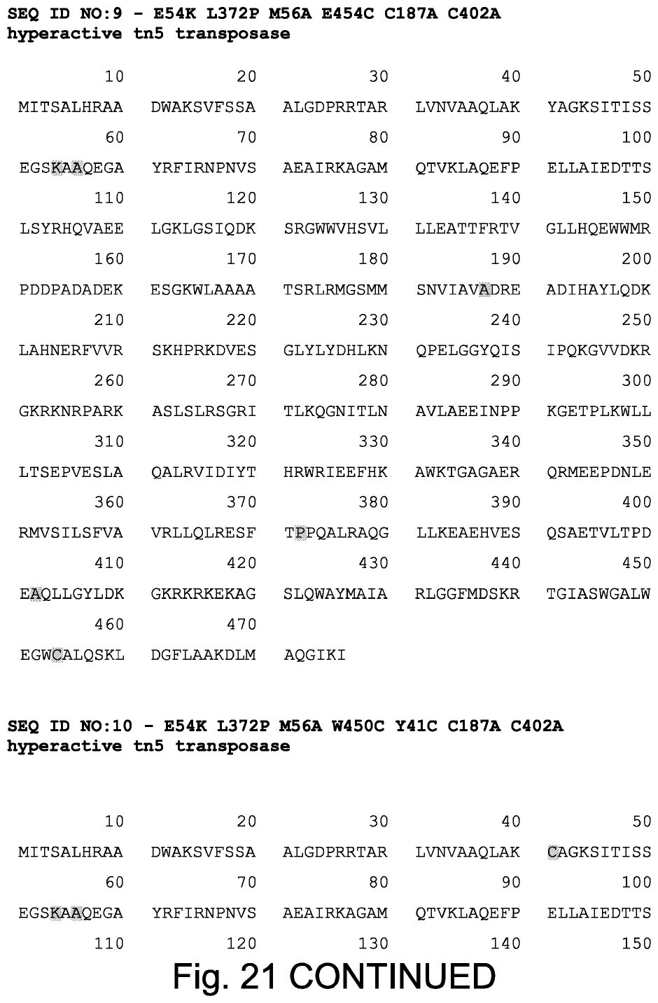

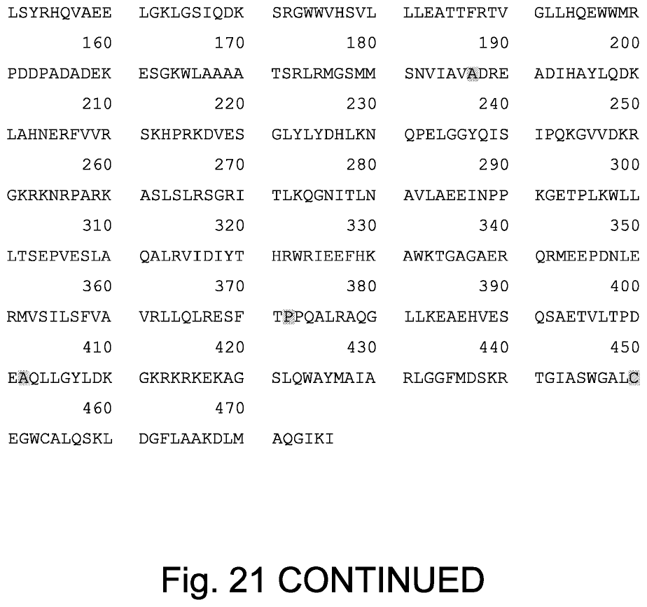

FIG. 21 shows: the amino acid sequences for wild type tn5 (SEQ ID NO:1), E54K L372P hyperactive tn5 transposase (SEQ ID NO:2), E54K L372P M56A hyperactive tn5 transposase (SEQ ID NO:3), E54K L372P M56A Y41A hyperactive tn5 transposase (SEQ ID NO:4), E54K L372P M56A S42A hyperactive tn5 transposase (SEQ ID NO:5), E54K L372P M56A Y41C C187A C402A hyperactive tn5 transposase (SEQ ID NO:6), E54K L372P M56A W450C C187A C402A hyperactive tn5 transposase (SEQ ID NO:7), E54K L372P M56A E451C C187A C402A hyperactive tn5 transposase (SEQ ID NO:8), E54K L372P M56A E454C C187A C402A hyperactive tn5 transposase (SEQ ID NO:9), and E54K L372P M56A W450C Y41C C187A C402A hyperactive tn5 transposase (SEQ ID NO: 10). Amino acid changes relative to wild type tn5 are highlighted in grey.

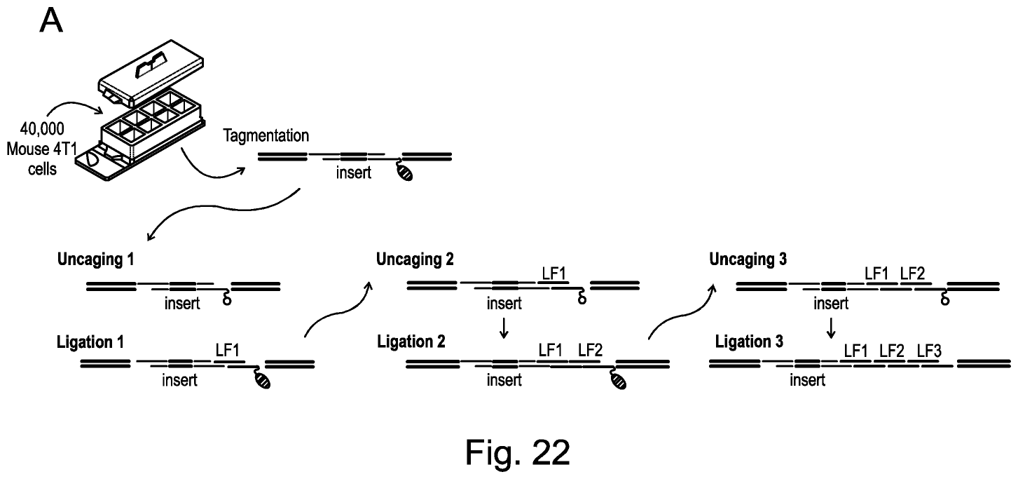

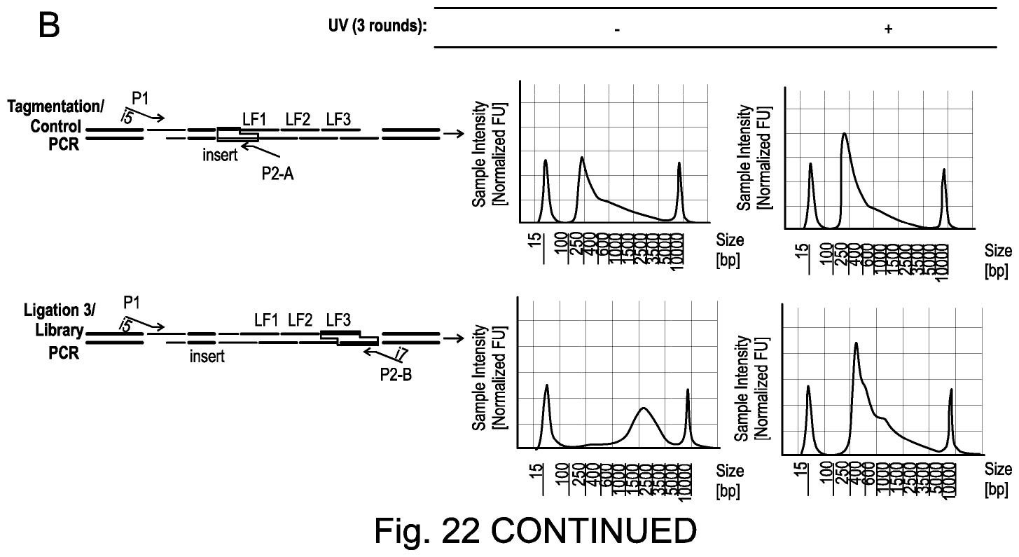

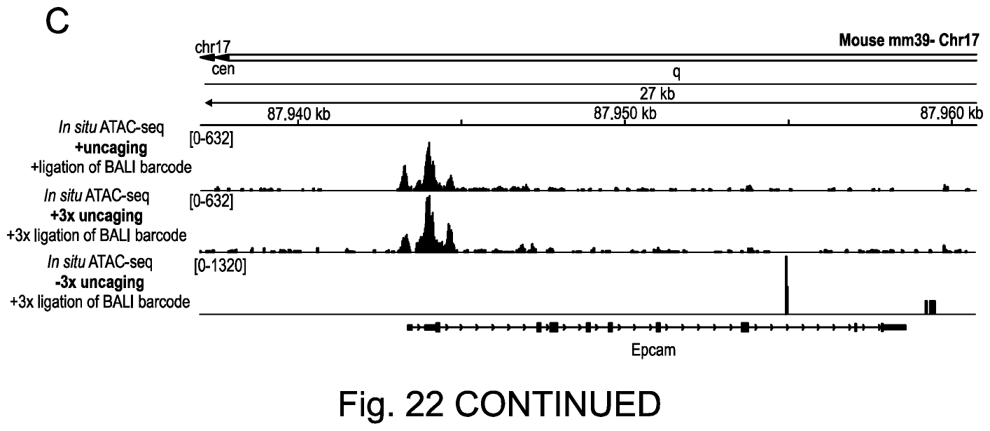

FIG. 22 shows: the procedure encompassing profiling of chromatin only, such steps being referred to ATAC-BALI in the figure. (A) Schematic of the procedure that was carried out on cells grown on slides. A transposase complex (Tn5) is used to attach detection probes harbouring a photocleavable group to DNA at open chromatin areas. Three rounds of illumination-mediated photocleavable group cleavage followed each by a ligation allows the construction of a spatial barcode within the area of interest. Following lysis and reverse crosslinking, library construction is carried out in vitro (not shown). (B) Schematic of the PCR strategy (left) is shown next to TapeStation D5000 ScreenTape profiles of samples obtained at the end of the procedure shown in (A). Tagmentation Control PCR indicates successful integration of detection probes into DNA (top two ScreenTape profiles). Ligation Library PCR indicates the successful integration of detection probes into DNA as well as successful construction of a spatial barcode by three ligation reactions of an index sequence after illumination-mediated photocleavable group cleavage. (C) ATAC-BALI profiles of positive (middle) and negative (bottom) Ligation Library PCR sample shown in (B) following sequencing, spatial barcode trimming and mapping. Ligation Library PCR sample with 1× ligation is shown as control (top). Reads per kilobase per million mapped reads are shown for the indicated genomic region on mouse chromosome 17 (coordinates are indicated).

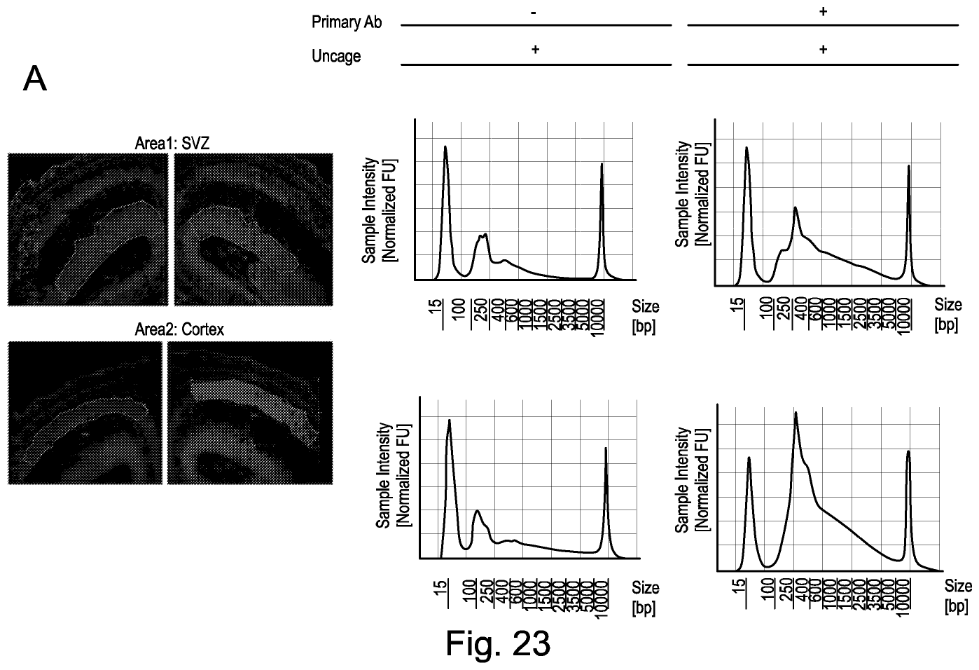

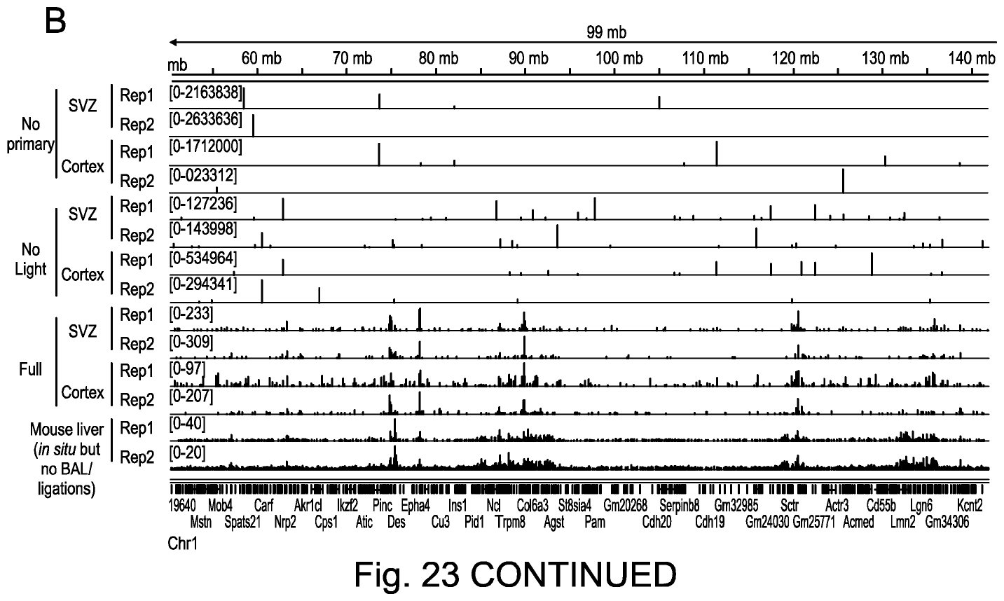

FIG. 23 shows: the procedure encompassing profiling of chromatin features only-such steps being referred to as CUT&Tag-BALI (CUT&Tag followed by BALI workflow) in the figure. (A) Left: Confocal images showing the regions that were photo-activated to allow the construction of a spatial barcode within the area of interest. Right: TapeStation High Sensitivity D5000 ScreenTape profiles of samples obtained at the end of the procedure. Samples that were exposed to primary antibody and illumination-mediated photocleavable group cleavage showed successful library construction (right), while samples not exposed to the primary antibody showed different library shape. (B) CUT&Tag-BALI profiles of positive Ligation Library PCR samples (“full”) along with no primary antibody or no illumination controls are shown following sequencing, spatial barcode trimming and mapping. Data from mouse liver are shown for comparison. Reads per kilobase per million mapped reads are shown for the indicated genomic region on mouse chromosome 1.

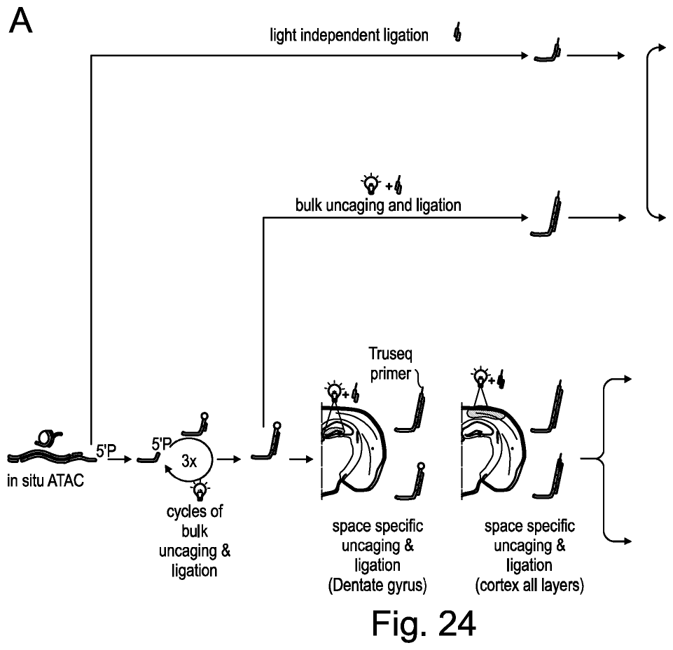

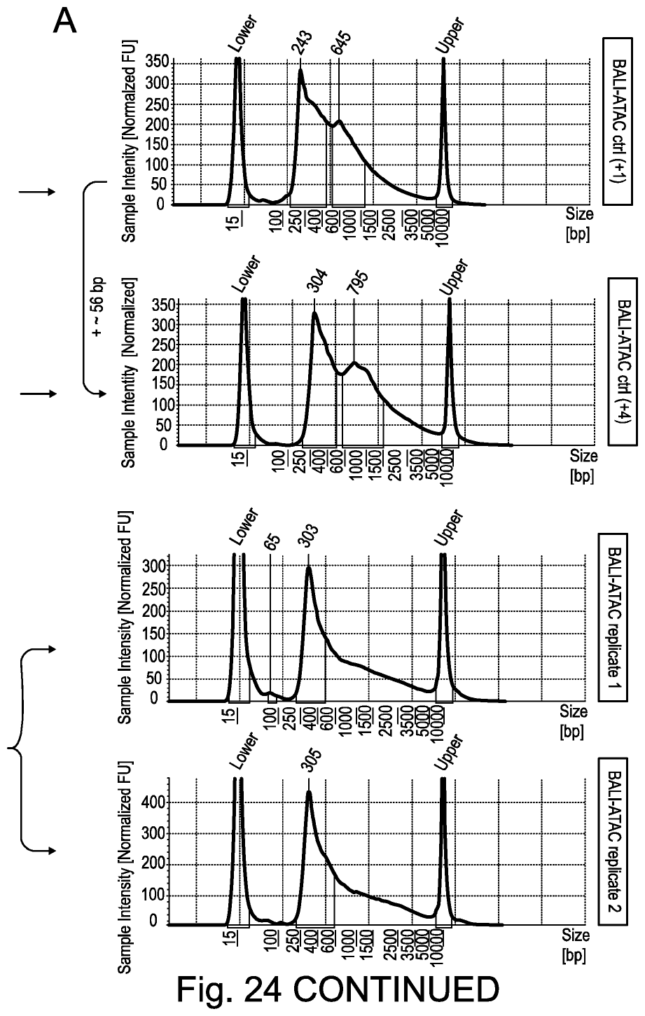

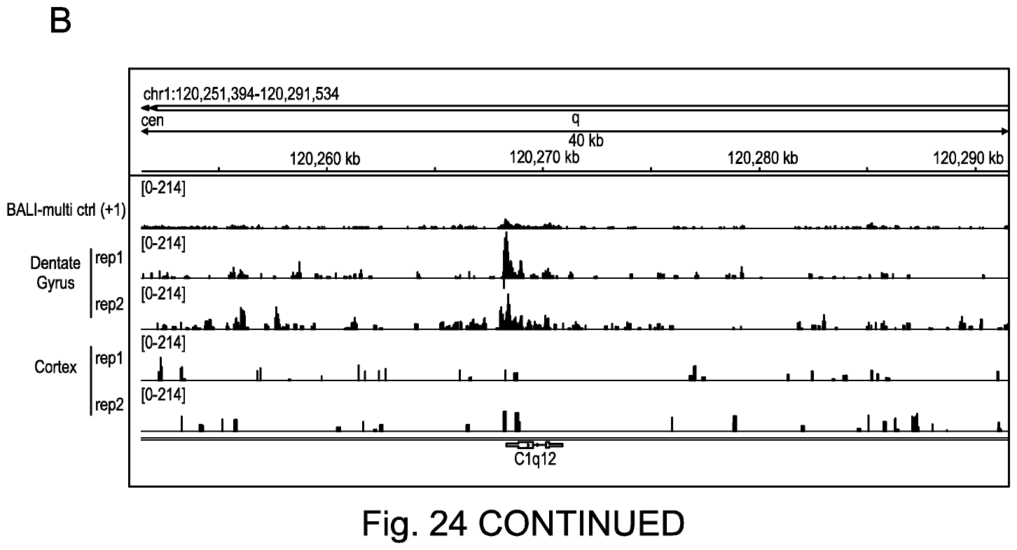

FIG. 24 shows: the procedure encompassing profiling of chromatin only, such steps being referred to ATAC-BALI in the figure. (A) Schematic of the procedure that was carried out on thin sections of flash frozen brain tissue from an adult mouse. A transposase complex (Tn5) is used to attach detection probes harbouring a 5′ phosphate to DNA at open chromatin areas. Four rounds of ligation followed by illumination-mediated photocleavable group cleavage allows the construction of a spatial barcode within the area of interest. In this experiment, illumination is performed across the tissue (referred as ‘bulk’) after the first and second ligation, whereas is directed to areas of interests after the third cycle to direct spatial specific barcoding of molecules in each of the two targeted areas (i.e. Dentate Gyrus and cortex). Following lysis and reverse crosslinking, library construction is carried out in vitro (not shown). Schematic of the expected barcodes are shown next to TapeStation D5000 ScreenTape profiles of samples obtained at the end of the procedure. The peak of the libraries shifts in accordance to the length of the barcode assigned to the samples with a difference of ˜56 bp between 1× Ligation and the 4× Ligation control and two BALI-ATAC replicates. (B) ATAC-BALI profiles of the Dentate Gyrus (lane 2 and 3) and cortex (lanes 4 and 5). The sample with 1× ligation is shown as control (lane 1). Reads per kilobase per million mapped reads are shown for a representative genomic locus (coordinates are indicated).

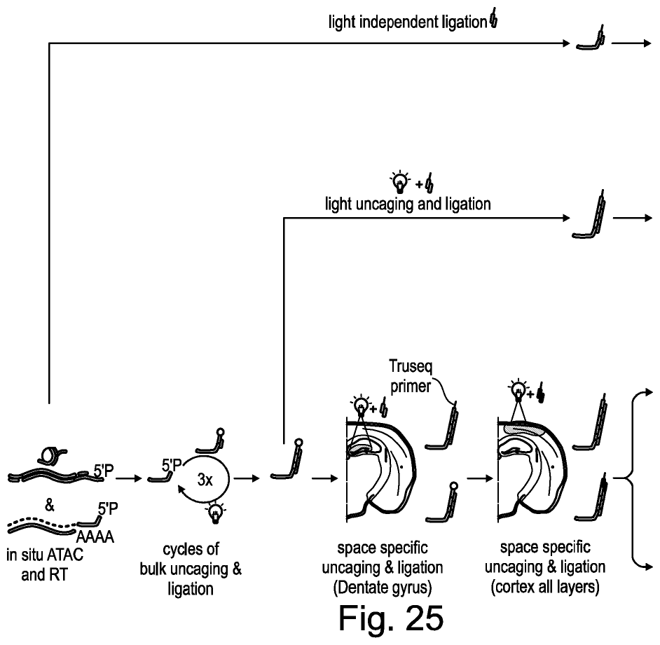

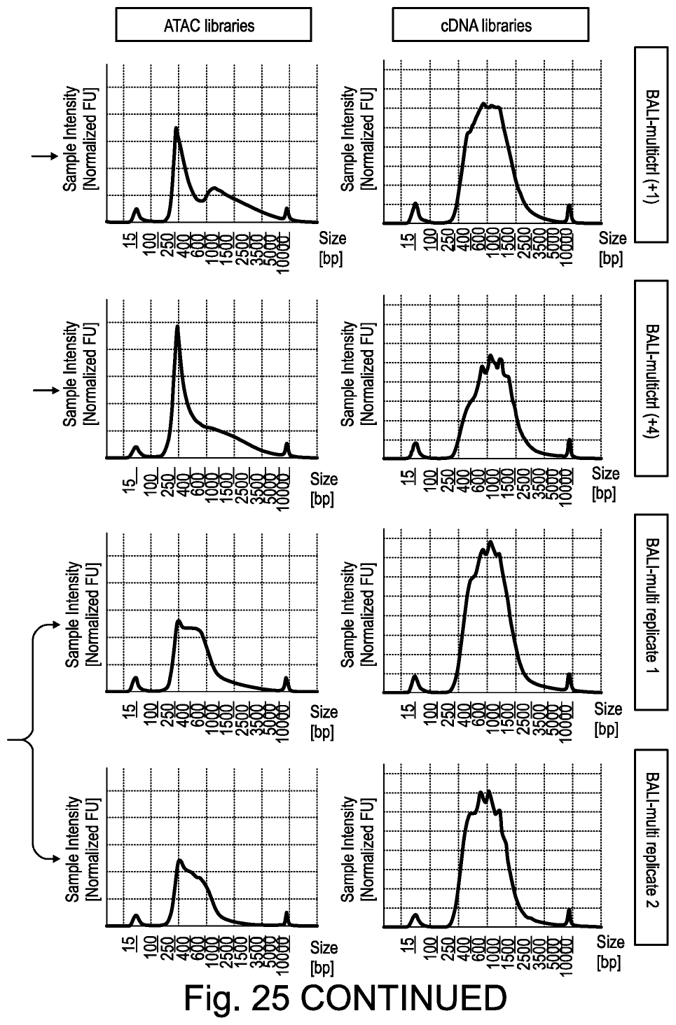

FIG. 25 shows: the procedure encompassing profiling of chromatin accessibility and gene expression, such steps being referred to BALI-multi in the figure. Schematic of the procedure that was carried out on thin sections of flash frozen brain tissue from an adult mouse. A transposase complex (Tn5) is used to attach detection probes harbouring a 5′ phosphate to DNA at open chromatin areas. Following tagmentation, a reverse transcriptase enzyme retrotranscribes mRNA in cDNA from a primer composed of a region annealing on the mRNA poly-A tail and a region harboring a 5′ phosphate. Compatible ligation overhangs are attached to both chromatin and cDNA. Four rounds of ligations followed by illumination-mediated photocleavable group cleavage allows the construction of a spatial barcode within the area of interest. In this experiment, illumination is performed across the tissue (referred as ‘bulk’) after the first and second ligation, whereas is directed to areas of interests after the third cycle to direct spatial specific barcoding of molecules in each of the two targeted areas (i.e., Dentate Gyrus and cortex). Following lysis and reverse crosslinking, library construction is carried out in vitro to generate cDNA libraries and ATAC libraries separately (not shown). Schematic of the expected barcodes are shown next to TapeStation D5000 ScreenTape profiles of samples obtained at the end of the procedure. The peak of the ATAC libraries shifts in accordance with the length of the barcode assigned to the samples with a difference of ˜56 bp between 1× Ligation and the 4× Ligation control and BALI-multi replicates. The cDNA libraries profiles are shown after the initial amplification of the whole cDNA molecules, prior to fragmentation to generate Illumina-compatible libraries.

### EXAMPLES

Methods are specified below when used. All oligonucleotides sequences were obtained from Integrated DNA technologies, AtdBio or Biomers, and all the chemicals and equipment are from the providers listed below in tables 1 and 2.

TABLE 1

| Consumable | Catalog # | Vendor |
|---|---|---|
| Nunc ™ Lab-Tek ™ II Chamber Slide ™ System | 154534 | Thermo Scientific |
| 8 × 0.2 mL PCR Tube Strip w/ individual attached Flat Cap | 781332 | Brand |
| Eppendorf Tubes ® 5.0 mL with snap cap | 0030119460 | Eppendorf |
| Eppendorf Protein LoBind tubes 1.5 ml PCR clean | 22431021 | Eppendorf |
| Falcon tube 50 ml | 430829 | Corning |
| Falcon tube 15 ml | 430791 | Corning |
| 100 mm TC-treated Culture Dish | 430167 | Corning |
| Parafilm PM 996 | N/A | Bemis |
| 0.2 ml 8-Strip PCR Tubes, Natural | I1402-3500 | Starlab |
| twin.tec ™ 96 Well LoBind PCR Plates, Skirted | 951020401 | Eppendorf |
| 75 cm² Cell Culture Flask | 430641U | Corning |
| Qubit ™ Assay Tubes | Q32856 | Thermo Fisher Scientific |
| Drosophila S2 cells | R69007 | Thermo Fisher Scientific |
| Drosophila zsGreen S2 cells | N/A | N/A |
| Schneider's Drosophila Medium (1X) | 21720001 | Thermo Fisher Scientific |
| | | (Gibco) |
| Fetal Bovine Serum, Heat Inactivated | F9665 | Sigma-Aldrich |
| Penicillin-Streptomycin (10,000 U/mL) | 15070063 | Thermo Fisher Scientific |
| | | (Gibco) |
| 4T1 cells | CRL-2539 | ATCC |
| Dulbecco's Modified Eagle Medium (1X) | 41966029 | Thermo Fisher Scientific |
| | | (Gibco) |
| Trypsin-EDTA (0.25%), phenol red | 25200056 | Thermo Fisher Scientific |
| | | (Gibco) |
| One Shot Fetal Bovine Serum (FBS) | A3840402 | Thermo Fisher Scientific |
| | | (Gibco) |
| L-Glutamine (200 mM) | 25030024 | Thermo Fisher Scientific |
| | | (Gibco) |
| Nuclease-Free Water | AM9937 | Ambion |
| Concanavalin A from Canavalia ensiformis (Jack bean) | L7647 | Sigma-Aldrich |
| Formaldehyde solution, for molecular biology, 36.5-38% in | F8775-4X25ML | Sigma-Aldrich |
| H2O | | |
| Milli-Q water | N/A | Media kitchen |
| PBS | N/A | Media kitchen |
| Glycine | G/800/60 | Fisher chemicals, UK |
| Digitonin, high purity | 300410-1 | VWR |
| Dimethyl sulfoxide, for molecular biology, ≥99.9% | D8418 | Sigma-Aldrich |
| Invitrogen UltraPure 1M Tris HCl Buffer, pH 7.5 | 15-567-027 | Thermo Fisher Scientific |
| NaCl 5M | N/A | Media kitchen |
| MgCl2 1M | N/A | Media kitchen |
| Tween ® 20 (Polysorbate) | 663684B | VWR chemicals |
| Nonidet ™ P 40 Substitute | 74385 | Sigma-Aldrich |
| Bovine Serum Albumin, heat shock fraction, pH 7, ≥98% | A9647 | Sigma-Aldrich |
| Tagmentase (Tn5 transposase) - unloaded | C01070010-20 | Diagenode |
| EDTA 0.5M | N/A | Media kitchen |
| Invitrogen UltraPure 1M Tris HCl Buffer, pH 8.0 | 15-568-025 | Thermo Fisher Scientific |
| SDS 20% | N/A | Media kitchen |
| Proteinase K, Molecular Biology Grade | P8107S | New England Biolabs (NEB) |
| NEBuffer 3.1 | B7203S | New England Biolabs (NEB) |
| NEBuffer ™ Set (r1.1, r2.1, r3.1 and rCutSmart ™) - new | B7030S | New England Biolabs (NEB) |
| T4 DNA Ligase - High Conc (2,000 U/ul) | M0202M | New England Biolabs (NEB) |
| Triton X-100 | T8787-100ML | Sigma-Aldrich |
| DNA Clean & Concentrator-5 with Zymo-Spin I Columns, | D4004 | Zymo Research |
| Uncapped | | |
| NEBNext ® High-Fidelity 2X PCR Master Mix | M0541L | New England Biolabs (NEB) |
| Ethyl alcohol, Pure | E7023 | Merck (Sigma-Aldrich) |
| Ampure XP Beads | N/A | Beckman coulter |
| Buffer EB | 19086 | Qiagen |
| Tapestation - D5000 ScreenTape | 5067-5588 | Agilent |
| Tapestation - D5000 Reagents | 5067-5589 | Agilent |
| Qubit dsDNA HS Assay Kit | Q32854 | Thermo Fisher Scientific |
| Tapestation - High Sensitivity D5000 ScreenTape | 5067-5592 | Agilent |
| Tapestation - High Sensitivity D5000 Reagents | 5067-5593 | Agilent |
| Alfa Aesar HEPES, 1.0M buffer soln., pH 7.5 | J60712-AK | Thermo Fisher Scientific |
| Spermidine, ≥99% (GC) | S2626-5G | Sigma-Aldrich |
| cOmplete ™, EDTA-free Protease Inhibitor Cocktail | 11873580001 | Roche |
| RNasin Plus RNase Inhibitor | N2615 | Promega |
| RNase Inhibitor, Murine | M0314L | New England Biolabs (NEB) |
| Invitrogen Ambion MgCl | AM9530G | Thermo Fisher Scientific |
| pA-Tn5 Transposase - unloaded | C01070002-30 | Diagenode |
| Tri Methyl Histone H3 (Lys27) (C36B11) Rabbit mAb | 9733S | Cell Signaling Technology |
| Guinea Pig anti-Rabbit IgG (Heavy & Light Chain) antibody | ABIN101961 | Antibodies-Online |
| Dynabeads MyOne Streptavidin C1 | 65001 | Thermo Fisher Scientific |
| dNTP Mix | R0192 | Thermo Fisher Scientific |
| POLY(ETHYLENE GLYCOL) BIOULTRA 6,000 | 81255-250G | Sigma-Aldrich |
| Thermo Scientific Maxima H Minus Reverse Transcriptase | EP0753 | Thermo Scientific |
| 20x SSC | AM9765 | Thermo Fisher Scientific |
| Ethylene carbonate, 98% | E26258-3KG | Sigma-Aldrich |
| DRAQ5 | 564902 | BD Biosciences |

TABLE 2

| Equipment | Vendor |
|---|---|
| Heracell 150 CO2 Incubator | Thermo Fisher Scientific |
| Heracell ™ VIOS 160i CO2 Incubator | Thermo Fisher Scientific |
| ZEISS Axio Vert.A1 | Zeiss |
| LUNA-FL ™ Dual Fluorescence Cell Counter | Logos Biosystems |
| LUNA ™ Cell Counting Slides | LabTech |
| Centrifuge 5810 R | Eppendorf |
| Vortex-Genie ™ 2 | Scientific Industries SI |
| Microcentrifuge, MiniStar | VWR |
| Analytical balance | Sartorius |
| HB-1D Hybridisation oven | Techne |
| U: Genius 3 UV transilluminator | Syngene |
| Capex 8C, Charles Austen Vacuum and | Charles Austen |
| pressure Laboratory Pump X37-950 | |
| Eppendorf ThermoMixer ® C | Eppendorf |
| Fresco ™ 17 Microcentrifuge | Thermo Fisher Scientific |
| Bio Rad DNA Engine Tetrad 2 | Bio-Rad |
| Thermal Cycler | |
| TapeStation System 4150/4200 | Agilent |
| Qubit 3/4 Fluorometer | Thermo Fisher Scientific |
| SSM3 3D Gyratory Rocker Mini | Stuart |
| DynaMag ™-2 Magnet | Thermo Fisher Scientific |
| DiaMag 0.2 ml - magnetic rack | B04000001 |
| Rotator SB3 | Stuart |
| Leica SP5 confocal microscope | Leica Microsystems |
| Bio-Rad CFX96 Real Time PCR | Bio-Rad |
| Detection System | |

The term ‘cage’ or ‘PC spacer’ throughout refers to a photocleavable spacer modification with the following structure (formula I) as is shown in FIG. 1:

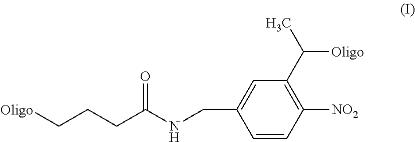

Suitable examples demonstrating the steps of the methods relating to addition of index sequences to detection probes using sequential illumination of locations or areas on a substrate in order to build up the spatial barcode have already been provided and explained in the previously published application by the same inventors WO2021/116715 which is hereby incorporated by reference. Examples 1 to 3 herein are exemplary proof of concept experiments presented in the previous application, repeated here for reference to demonstrate these steps of the methods. In particular to demonstrate the steps comprising illuminating a location of interest within the substrate to be spatially barcoded, wherein the illumination cleaves or alters the photocleavable group of the or each detection probe within the location; adding an index sequence of the spatial barcode to the or each detection probe within the location illuminated and repeating said steps until the desired index sequences are added to form a spatial barcode attached to the or each detection probe within the location of interest.

Examples 4 onwards relate to the experiments conducted herein to demonstrate the use of a transposase complex such methods in order to provide a multi-omics approach.

### Example 1

Unless otherwise specified all the chemical were obtained from Sigma-Aldrich. A solid surface labelled with a detection probe was produced as follows: the BALI_09 oligonucleotide was diluted to 1 μM final concentration in PBS buffer (250 ul per slide). A 1:100 dilution of a 10 mM solution of BS(PEG) 9 crosslinker (Pierce) in DMSO was added to the mix, and the resulting solution was spread on a glass slide coated with aminoalkylsilane (Sigma, Silane-Prep) using a coverslip. The slide was incubated for 2 h at 30° C. in a humid chamber, washed for 10 minutes with 0.1% glycine in PBS, and washed several times in PBS.

In order to produce a double-stranded end on the detection probe, the BALI_09 oligonucleotide was diluted to a final 1 μM concentration in hybridization buffer (10% ethylene carbonate in 2×SSC) and incubated on the slide surface for 15 minutes at room temperature, followed by two 5′ washes in hybridization solution at room temperature and three washes in 2×SSC at room temperature.

The detection probe bound to the slide was extended by a DNA bridge molecule bearing a photocleavable group and the Alexa-488 fluorophore as follows: the BALI_10 and BALI_11 primers were diluted to a final concentration of 5 μM in 5×SSC buffer, heated at 95 C for 5 minutes, and gradually cooled down to 30° C. on a PCR cycler using a temperature gradient of −1° C./30″. A ligation solution was prepared by mixing: 107.5 μl of ultra-pure water, 125 ul 2× quick ligation mix (NEB), 12.5 μl T4 ligase, high concentration (NEB), and 5 μl (final 100 μM) of BALI_10/11 oligos. The ligation solution was incubated on the slide for 30 minutes at room temperature, followed by three 5 minute washes in 2×SSC.

The slide bearing the detection probe extended by the photocleaved DNA bridge molecule was imaged on a Leica SP5 confocal microscope equipped with a 30 mW 405 nm solid state laser, an argon laser line at 488 and 514 nm, a He—Ne laser at 543 nm, and a solid state 647 nm laser. Alexa 488 was excited using the 488 nm laser, and the relative fluorescence signal captured by a PMT after a 510-540 nm bandpass filter. Atto-568 was excited by the 543 nm laser line and the relative fluorescence signal captured by a PMT after a 560-600 nm bandpass filter. Cy5 was excited by the 647 nm laser and the relative fluorescence signal captured by a PMT tube after a 660-750 nm bandpass filter. Once the surface of the slide was identified by detecting the plane of maximum Alexa-488 signal, photorelease was produced by illuminating two rectangular regions of interest with 100% power of the 405 nm laser for 5 minutes each. After photorelease, the slide was washed three times for 5′ in 2×SSC.

For the first spatial barcoding step, a double-stranded index composed of the BALI_12 and BALI_13 primers was produced by annealing the two oligonucleotides at a final concentration of 5 μM. This was performed by combining the two oligonucleotides at the indicated concentration, heating them up to 95° C. for 5 minutes in a thermal cycler, and cooling them down to 12° C. with a cooling rate of 0.1° C./second (again in a thermal cycler). A second ligation reaction was prepared as described before and incubated on the slide for 30′ at room temperature. After the ligation, the slide was washed for three times in 2×SSC at room temperature. The slide was imaged as above. Light was used to photorelease the photocleavable group only on one of the two barcoded areas for the same time and using the same power described above.

For the second spatial barcoding step, a double-stranded index composed of the BALI_13 and BALI_14 primers was produced by annealing the two oligonucleotides at a final concentration of 5 UM as described before. A third ligation reaction was prepared as described before and incubated on the slide for 30′ at room temperature. After the ligation, the slide was washed for three times in 2×SSC at room temperature and for three times for 5′ in 0.2×SSC at 50° C. The slide was imaged as above for a third time with the same settings.

Results are shown in FIG. 2.

TABLE 3

| Name | Sequence | SEQ ID NO: |
|---|---|---|
| BALI_09 | GGTAGTAT ATGTCGAGAGCTAGC CGCGCCTTAAGATAC CAATTAGAGT | 11 |
| | AGATCGGAAGAGCGTCGTGTAG CTCCCTATAGTGAGTCGTATTA | |
| | CTAGCTAGCG [Aminolink C6] | |
| BALI_10 | GCTAGCTCTCGACAT | 12 |
| BALI_11 | [Atto488] [cage] GTACCTGT CAGCTACCATG | 13 |
| BALI_12 | [5′ PHOS] ATACTACC CATGGTAGCTG | 14 |
| BALI_13 | [Cy5] [cage] GTATCGAG CTCTATACAC | 15 |
| BALI_14 | [5′ PHOS] ACAGGTAC GTGTATAGAG | 16 |
| BALI_15 | [Atto565] [cage] GTGAGCGT CGGACACCTAC | 17 |
| BALI_16 | [5′ PHOS] CTCGATAC GTAGGTGTCCG | 18 |

(“Cage” refers to the photocleavable spacer oligo modification as shown in FIG. 1,
“Atto488” refers to a the atto488 green fluorescent group bound to the 5′ of the
molecule, “Atto565” refers to the atto565 red fluorescent group bound to the 5′
end of a molecule, “cy5” refers to a cyanine 5 fluorescent group bound to the 5′
end of a molecule, ′aminolink C6′ refers to an NH2 group, 5′PHOS refers to
phosphate).

### Example 2

Unless otherwise specified all the chemical were obtained from Sigma-Aldrich.

Light-dependent barcoding gene expression measurements on cells.

In this experiment, cultured cells expressing either green fluorescent protein (GFP) or red fluorescent protein (RFP) were plated on two separate coverslips, and subjected to our protocol for light-dependent barcoding and gene expression measurement. This was done with a library of detection probes including sequences targeting both the GFP and RFP genes (BALI_77 to BALI_84), and using light to barcode such probes with one of two different spatial barcodes. Spatial barcode 1 was used to label GFP cells, whereas spatial barcode 2 was used to label RFP cells. Illumina sequencing was then used to measure how many detection probes targeting GFP/RFP were present in each spatially barcoded population.

4t1 mouse tumour cells expressing GFP or RFP were cultured on #1.5 thickness glass coverslips functionalised first with BIND-silane (GE Healthcare), and then overnight with 0.01% poly-L-lysine in complete culture medium (DMEM, 10% fetal bovine serum). Prior to the experiment, cells were fixed in 4% paraformaldehyde for 15 minutes, washed in PBS, and permeabilised in 0.5% Triton X-100 in phosphate-buffered saline (PBS) for 10 minutes.

The detection probes were diluted in encoding hybridization buffer (2×SSC buffer, 30% formamide, 10% dextran sulphate, 1 mg/ml yeast tRNA, 1:100 NEB murine ribonuclease inhibitor) at a final concentration of 1 μM, and the sample was diluted in the resulting mix for 48 h at 37° C. in a humidified chamber. After the hybridization, the sample was washed twice at 47° C. for 30 minutes in encoding wash buffer (2×SSC, 30% formamide), and twice at room temperature for 5 minutes in 2×SSC.

A thin hydrogel was cast over the cells by coating the coverslips with a 80 ul drop of degassed hydrogel buffer (4% 19:1 acrylamide: bis-acrylamide mix, 0.3M NaCl, 60 mM Tris-HCl pH 8, 0.05% TEMED, 0.05% Ammonium persulfate) and incubating for 1 h at room temperature. The samples were then digested in digestion buffer (2% SDS, 50 mM tris-HCl pH 8, 0.5% Triton X-100, 1:100 NEB Proteinase K enzyme) overnight at 37° C. in a humidified chamber. After the clearing, the coverslips were washed three times for 1 h in 2×SSC, then washed in secondary hybridization buffer (10% Ethylene Carbonate, 2×SSC) for 5 minutes, and hybridized with the BALI_85 oligo (10 nM final concentration, diluted in secondary hybridization buffer) for 15 minutes at room temperature. Finally, samples were washed once in secondary hybridization buffer and once in SSC for 5 minutes each.

Uncaging of the detection probes was performed on a leica SP5 confocal microscope equipped with a 30 mW 405 nm laser, using a 10× objective and 100% laser power. Uncaging was done for 5 minutes on 5 field of views (approx. 1 mm² each) per sample. Following uncaging, samples were ligated with either spatial barcode 1 or spatial barcode 2 by first annealing the BALI_86 and BALI_87 barcodes or BALI_88 and BALI_89 barcodes (by diluting them in 5×SSC at 5 μM concentration, heating at 95° C. for 5 minutes and cooling down slowly to room temperature over 30 minutes), and then incubating them for 30 minutes at room temperature in a ligation mix composed by 1×NEB quick ligation buffer, 100 U/ul T4 DNA ligase, and 100 nM annealed spatial barcode.

Following the ligation step, the hydrogel including the cells was scraped from the coverslips, transferred to a 1.5 ml tube, and diluted in 500 ul 0.4M NaCl. DNA was released by vortexing for 1 h at high speed and purified by ethanol precipitation.

The precipitated DNA (including the barcoded detection probes) was used to produce an illumina sequencing library by two successive rounds of PCR, first using the BALI_90 and BALI_91 primer and the Q5 enzyme from NEB (standard protocol) and the using the Illumina universal forward truseq primer and indexed DNA LT reverse truseq primers (indexes 006 and 012) and the NEB phusion enzyme (standard protocol).

The libraries were sequenced using an Illumina MiSeq sequencer (paired end 150 reads) and analysed through a bioinformatic pipeline developed in the python programming language, which is briefly schematised in additional FIG. 3B. Results are show in FIG. 3.

TABLE 4

| Name | Sequence | SEQ ID NO: |
|---|---|---|
| BALI_77 | [CAGE] GTGACC ATGTCGAGAGCTAGC NNNNNNNNNNNNNNN CATCGTGAGT | 19 |
| | AGATCGGAAGAGCGTCGTGTAG CTCCCTATAGTGAGTCGTATTA CTAGCTAGCG | |
| | TTGAAGAAGATGGTGCGCTCCTGGACGTAGCCTTCG | |
| BALI_78 | [CAGE] GTGACC ATGTCGAGAGCTAGC NNNNNNNNNNNNNNN CATCGTGAGT | 20 |
| | AGATCGGAAGAGCGTCGTGTAG CTCCCTATAGTGAGTCGTATTA CTAGCTAGCG | |
| | CTTGAAGTCGATGCCCTTCAGCTCGATGCGGTTCAC | |
| BALI_79 | [CAGE] GTGACC ATGTCGAGAGCTAGC NNNNNNNNNNNNNNN CATCGTGAGT | 21 |
| | AGATCGGAAGAGCGTCGTGTAG CTCCCTATAGTGAGTCGTATTA CTAGCTAGCG | |
| | TGCTTGTCGGCCATGATATAGACGTTGTGGCTGTTG | |
| BALI_80 | [CAGE] GTGACC ATGTCGAGAGCTAGC NNNNNNNNNNNNNNN CATCGTGAGT | 22 |
| | AGATCGGAAGAGCGTCGTGTAG CTCCCTATAGTGAGTCGTATTA CTAGCTAGCG | |
| | CATGTGATCGCGCTTCTCGTTGGGGTCTTTGCTCAG | |
| BALI_81 | [CAGE] GTGACC ATGTCGAGAGCTAGC NNNNNNNNNNNNNNN GGTACAGATG | 23 |
| | AGATCGGAAGAGCGTCGTGTAG CTCCCTATAGTGAGTCGTATTA CTAGCTAGCG | |
| | GATGGCCATGTTATCCTCCTCGCCCTTGCTCACCAT | |
| BALI_82 | [CAGE] GTGACC ATGTCGAGAGCTAGC NNNNNNNNNNNNNNN GGTACAGATG | 24 |
| | AGATCGGAAGAGCGTCGTGTAG CTCCCTATAGTGAGTCGTATTA CTAGCTAGCG | |
| | CTCGGGGAAGGACAGCTTCAAGTAGTCGGGGATGTC | |
| BALI_83 | [CAGE] GTGACC ATGTCGAGAGCTAGC NNNNNNNNNNNNNNN GGTACAGATG | 25 |
| | AGATCGGAAGAGCGTCGTGTAG CTCCCTATAGTGAGTCGTATTA CTAGCTAGCG | |
| | CTTGTAGGTGGTCTTGACCTCAGCGTCGTAGTGGCC | |
| BALI_84 | [CAGE] GTGACC ATGTCGAGAGCTAGC NNNNNNNNNNNNNNN GGTACAGATG | 26 |
| | AGATCGGAAGAGCGTCGTGTAG CTCCCTATAGTGAGTCGTATTA CTAGCTAGCG | |
| | CTGTTCCACGATGGTGTAGTCCTCGTTGTGGGAGGT | |
| BALI_85 | GCTAGCTCTCGACAT | 27 |
| BALI_86 | AGACGTGTGCTCTTCCGATCTCAGCTACCATG | 28 |
| BALI_87 | GGTCACCATGGTAGCTGAGATCGGAAGAGCACACGTCT | 29 |
| BALI_88 | AGACGTGTGCTCTTCCGATCTGACAATGAGGC | 30 |
| BALI_89 | GGTCACGCCTCATTGTCAGATCGGAAGAGCACACGTCT | 31 |
| BALI_90 | AGACGTGTGCTCTTCCGATCT | 32 |
| BALI_91 | TAATACGACTCACTATAGGGAGCTACAC | 33 |
| Illumina | AATGATACGGCGACCACCGAGATCTACACTCTTTCCCTACACGACGCTCTTCCGATCT | 34 |
| universal | | |
| FW | | |
| Truseq | | |
| Illumina | CAAGCAGAAGACGGCATACGAGATATTGGCGTGACTGGAGTTCAGACGTGTGCTCTTCCGATC | 35 |
| Truseq LT | | |
| rev. index | | |
| 006 | | |
| Illumina | CAAGCAGAAGACGGCATACGAGATTACAAGGTGACTGGAGTTCAGACGTGTGCTCTTCCGATC | 36 |
| Truseq LT | | |
| rev. index | | |
| 012 | | |

(“cage” refers to the Photocleavable spacer modification as shown in FIG. 1, ′N′ refers to any
nucleotide of A, T, G, or C)

### Example 3

Unless otherwise specified all the chemical were obtained from Sigma-Aldrich. Spatial indexing and assessment of quantification capability on functionalised hydrogel.

TABLE 5

| Name | SEQ ID NO: | Sequence | Notes |
|---|---|---|---|
| BALI_92 | 37 | [acrylate] cgacatcggcaagCTACTACGGCTAGCC | |
| | | TAATACGACTCACTATAGGGAG ACTCTAATTG GTATAGAATTCCGCG | |
| | | GCTAGCTCTCGACAT | |
| BALI_93 | 38 | [phosphate] GTGACGT ATGTCGAGAGCTAGC CGCGGAATTCTATAC | |
| 3 | | CAATTAGAGT CTCCCTATAGTGAGTCGTATTA GGC | |
| BALI_94 | 39 | [cy5] [cage] GaGCGTgcaccacagtcg | Bit0 |
| 4 | | | |
| BALI_95 | 40 | ACGTCACcgactgtggtgc | Bit0 |
| 5 | | | |
| BALI_96 | 41 | [cy3] [cage] GTGGACCgcacgtatggcg | 1a |
| 6 | | | |
| BALI_97 | 42 | ACGCtCcgccatacgtgc | 1a |
| 7 | | | |
| BALI_98 | 43 | [cy3] [cage] GTGGACCgcacgagtcccg | 1b |
| 8 | | | |
| BALI_99 | 44 | ACGCtCcgggactcgtgc | 1b |
| 9 | | | |
| BALI 100 | 45 | AGACGTGTGCTCTTCCGATCTACTGAGGTGAGC | 2a |
| 00 | | | |
| BALI 102 | 46 | GGTCCACGCTCACCTCAGTAGATCGGAAGAGCACACGTCT | 2a |
| 01 | | | |
| BALI 102 | 47 | AGACGTGTGCTCTTCCGATCTCGACGATCTAGC | 2b |
| 02 | | | |
| BALI_103 | 48 | GGTCCACGCTAGATCGTCGAGATCGGAAGAGCACACGTCT | 2b |
| 03 | | | |
| BALI_104 | 49 | AGACGTGTGCTCTTCCGATCT | |
| 04 | | | |
| BALI_105 | 50 | AATGATACGGCGACCACCGAGATCTACACTCTTTCCCTACACGACGCTCTTC | |
| | | CGATCTNNNNNNGCGGCTAGCTCTCGACAT | |

(“cage” refers to the Photocleavable spacer modification as shown in FIG. 1, “acrylate” to a 5′
acrydite group, “cy3” refers to a cyanine 3 fluorescent group bound to the 5′ of the molecule,
“cy5” refers to a cyanine 5 fluorescent group bound to the 5′ end of a molecule, ′N′ refers to
any nucleotide of A, T, G, or C).

This experiment is designed to measure whether the amount of detection probes bound to a spatial region of a sample (in this case a functionalised hydrogel), and spatially indexed by our technology, can be measured by sequencing. Detection probes are homogeneously distributed on a functionalised coverslip, and two areas (a large one and a small one) are functionalised using different 2-bit spatial barcodes (two indexes each). Sequencing is then used to validate that the barcode assigned to the “large” area is more abundant than the barcode assigned to the “small” area.

An oligo-functionalised hydrogel was prepared by first pre-annealing oligos BALI_92 and BALI_93 by combining them to a final concentration of 15 μM in 2×SSC, heating to 95° C. for 2 minutes and cooling down to room temperature for 30 minutes, and then diluting the annealed oligos to a final concentration of 1 μM in degassed gel buffer (4% 19:1 acrylamide: bisacrylamide, 0.3 M NaCl, 60 mM Tris-HCl pH 8). A 80 ul drop of the gel solution was used to coat coverslips functionalised in BIND-Silane (GE healthcare) by incubation for 1 h at room temperature. BALI_92 and BALI_93 are designed to mimic a detection probe with an annealed stabiliser region.

The functionalised gel was first washed 3 times for 5 minutes at in 2×SSC (room temperature). A first ligation was then performed to attach a caged “bridge” molecule to the detection probes. Oligos BALI_94 and BALI_95 were annealed by combining them to a final concentration of 5 μM in 2×SSC, heating to 95° C. for 2 minutes and cooling down to room temperature for 30 minutes, and then further diluted to a final concentration of 500 nM in a ligation mix including 1×Quick ligation buffer (NEB) and 100 U/ul T4 DNA ligase. The functionalised coverslips were incubated with the ligation mix for 30 minutes at room temperature, and washed 3 times for 3 minutes at room temperature in 2×SSC.

Following the ligation, a dephosphorylation reaction was performed to remove any phosphate group produced by spontaneous unspecific uncaging of the photocage group. This was done by incubating the samples for 30 minutes at 37° C. in a mixture including 1× Cutsmart buffer (NEB) and 0.05 U/ul shrimp alkaline phosphatase, followed by three washes at room temperature for 5 minutes in 2×SSC.

Uncaging of the first “large” area was then performed on a leica SP5 confocal microscope equipped with a 30 mW 405 nm laser, using a 10× objective and 100% laser power. Uncaging was done for 5 minutes on 20 fields of view (approx 1 mm² each). Following this, the first bit of the spatial barcode was ligated to this area by incubating the sample for 30 minutes at room temperature in a ligation mix including 1× Quick ligation buffer (NEB), 100 U/ul T4 DNA ligase and 500 nM of oligos BALI_96 and BALI_97 annealed as described above. Ligation was followed by 3 washes at room temperature for 5 minutes is 2×SSC.

A second “small” area was then uncaged (as above, 4 fields of view), followed by ligation using annealed oligos BALI_98 and BALI_99 and by another round of washes.

The first “large” area was then localized again on the microscope using the loss of cy5 fluorescence and the acquisition of cy3 fluorescence as guide, and uncaged again with the same parameters, followed by ligation with oligos BALI_100 and BALI_101. The same was done for the “small” area, with oligos BALI_102 and BALI_103. In between ligation/uncaging steps the sample was washed three times at room temperature for 5 minutes in 2×SSC.

After completion of the spatial barcoding, the signal from the barcoded detection probes was amplified by in situ RNA transcription by incubating the sample in a transcription mixture containing 130 ul ultrapure H2O, 72 ul NTP mix (from the NEB Hiscribe T7 quick kit) and 14.4 ul of T7 RNA polymerase. Transcription was performed for 2 h at 37 C, after which the gel and transcription mixture were collected, diluted with 130 ul ultrapure H2O, and purified via ethanol precipitation in presence of 0.3 M Sodium acetate.

The recovered RNA was reverse transcribed using the superscript III kit (thermo scientific) according to standard protocols, using BALI_104 as a gene-specific primer. The resulting cDNA was then converted in an Illumina sequencing library using primers BALI_105 and the standard reverse indexed Truseq LT primer (index 006)

The libraries were sequenced using an Illumina MiSeq sequencer (paired end 150 reads) and analysed through a custom bioinformatics pipeline to quantify the abundance of each spatial index combination. Results are shown in FIG. 4.

### Example 4 (ATAC-BALI)

### Assembling Transposomes (Tn5)

In a PCR tube, adapter pair (detection probe) Tn5ME-A was produced by mixing 9.5 ul of 100 uM oBC3620 (Tn5MEfwd-A) and 9.5 ul of 100 uM oBC3622 (Tn5MErev) oligonucleotides (re-suspended in nuclease-free water) with 1 ul of 20× annealing buffer (800 mM Tris-HCl PH 8.0, 1 M NaCl). In a separate PCR tube, adapter pair (detection probe) Tn5ME-OL1 was generated by mixing 9.5 ul of 100 μM oBC3656 (Tn5MEfwd-OL1) and 9.5 ul of 100 uM oBC3622 (Tn5MErev) oligonucleotides (re-suspended in nuclease-free water) were mixed with 1 ul of 20× annealing buffer (800 mM Tris-HCl pH 8.0, 1 M NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed oligos were stored at −20° C. until further use.

Transposomes were assembled with equal amounts of separately pre-annealed Tn5ME-A and Tn5ME-OL1 adapters (detection probes). In a PCR tube, 5 ul of annealed Tn5ME-A adapters (oBC3620/oBC3622) and 5 ul of annealed Tn5ME-OL1 adapters (oBC3656/oBC3622) were mixed before adding 10 ul of 2 mg/ml Tagmentase (Tn5 Transposase, unloaded) and incubation for 30 minutes at 23° C. in a thermocycler. 20 ul of loaded transposome complexes were mixed with 10 ul of 100% glycerol and stored at −20° C. until further use.

TABLE 6
(detection probes)

| | | SEQ |
|---|---|---|
| Oligo ID | Oligo sequence (5′ −> 3′) | ID NO: |
| oBC3620 | TCGTCGGCAGCGTCAGATGTGTATAAGAGACAG | 51 |
| oBC3622 | /5Phos/CTGTCTCTTATACACATCT | 52 |
| oBC3656 | /5SpPC/gtcactccgcctcgtAGATGTGTATA | 53 |
| | AGAGACAG | |

/5Phos/ indicates 5′ phosphorylated group.
/5SpPC/ indicates 5′ photocaged group that can be cleaved
under UV light into a 5′
phosphorylated group. This oligonucleotide was purified
using “HPLC Purification”.

### Preparation of Slides and Plating of Cells on Slide

A chamber slide (8-well glass slide) was used, and 500 ul ConA was added if using fly cells (0.5 mg/ml in sterile H₂O, stored at ° 4C). This was incubated overnight at 26° C. in a tissue culture incubator. Slide chambers were washed once with 500 ul 1×PBS (137 mM NaCl, 2.7 mM KCl, 10 mM Na₂HPO₄, 1.8 mM KH₂PO₄). Per chamber, 50,000 Drosophila S2 cells in 200 ul growth media (Schneider media Gibco, 10% FBS, 1% Pen-Strep) from a 2.5×10\hat{ }5 cells/ml dilution were plated. The cells were incubated for 4 hours at 26° C. in a tissue culture incubator to allow cells to attach to ConA (cells flatten out and change morphology). Cells were checked under a microscope.

### Fixation

Cells were removed from a tissue culture incubator and the culture media removed. To each chamber, 500 ul of 0.5% formaldehyde diluted in PBS (27 ul of 37% PFA stock diluted in 1973 ul PBS) was added. This was incubated for 5 min at room temperature. Fixation was quenched with glycine (25 ul of 2.5M stock per chamber, corresponding to 1/20th of total sample volume). This was then incubated at room temperature for 5 minutes. The PFA and glycine solution was removed and the cell chambers were washed twice with 500 ul of 1×PBS. Cells were checked under a microscope.

### Permeabilization

Cell chambers were rinsed once in nuclease-free water. 500 ul lysis buffer (10 mM Tris-HCl pH7.4, 10 mM NaCl, 3 mM MgCl₂, 0.01% Tween-20, 0.01% NP-40, 0.001% Digitonin, 1% BSA) were added for permeabilization and then incubated for 15 minutes at room temperature. Lysis buffer was removed and 500 ul wash buffer (10 mM Tris-HCl PH7.4, 10 mM NaCl, 3 mM MgCl2, 0.1% Tween-20, 1% BSA) was added followed by incubation for 5 minutes at room temperature.

### Tagmentation

Wash buffer was removed and 100 μl of transposition mix (0.5 ul of home-loaded Tn5 transposase complex with Tn5ME-A and Tn5ME-OL1 adapters (detection probes) in 10 mM Tris-HCl PH 7.6, 5 mM MgCl2, 10% Dimethyl Formamide, 0.33×PBS, 0.1% Tween-20, 0.01% Digitonin) were added before incubation for 30 minutes at 37° C. in a hybridisation oven. Transposition mix was removed and 200 ul of 40 mM EDTA was added and incubated at room temperature for 5 minutes to stop transposition/inactivate the transposase. EDTA was removed and 200 ul NEBuffer 3.1, was added and then incubated at room temperature for 5 minutes. Cells were checked under a microscope.

### Uncaging

Chambers that should not be illuminated were covered with aluminium foil. Slides were placed under a UV transilluminator (with a mid-wavelength of 302 nm, suitable to uncage the photo-cleavable [5SpPC] modifications containing a photo-liable functional group that is cleavable by UV light of specific wavelength between 300-350 nm) and irradiated for exactly 120 seconds.

### Ligation

Ligation fragments (index sequences) were annealed: 10 ul of fragment 1/oBC3631 (100 uM) and 10 ul of fragment 2/oBC3650 (100 uM) oligonucleotides were mixed with 20 ul 2× annealing buffer (20 mM Tris, pH7.5-8.0, 100 mM NaCl, 2 mM EDTA) in a PCR tube. Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed ligation fragments were stored at −20° C. until use.

NEBuffer 3.1 was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed ligation fragments at 25 μM concentration) was added, then incubated for 30 minutes at 37° C. in a hybridisation oven. Ligation reaction solution was added along with 500 ul 1×PBS. This was incubated for 5 min at room temperature. Cells were checked under a microscope.

TABLE 7
(index sequences)

| | | SEQ |
|---|---|---|
| Oligo ID | Oligo sequence (5′ > 3′) | ID NO: |
| oBC3631 | GTCTCGTGGGCTCGGAGATGTGTATAAGAGA | 54 |
| | CAG | |
| oBC3650 | acgaggcggagtgacCTGTCTCTTATACACA | 55 |
| | TCTC | |

### Lysis and De-Crosslinking

Chambers were quickly rinsed in 500 ul nuclease-free water. Ensured to remove all of the liquid. 200 ul of reverse crosslinking solution (50 mM Tris-HCl pH8.0, 1 mM EDTA, 1% SDS, 150 mM NaCl, 0.4 mg/ml proteinase K, and 0.2 M glucose if using fly cells) were added and incubated at room temperature for 20 minutes, carefully shaking the chambers every 5 minutes. Subsequently, the solution was transferred to an Eppendorf tube and incubated with the samples overnight at 60° C. with shaking (1,000 rpm) in a thermomixer (Eppendorf). Slides were checked under a microscope for residual cells indicative of unsuccessful lysis.

### Library Preparation

DNA was purified using Zymo DNA Clean & Concentrator-5 columns. 1 ml of DNA Binding buffer was added to 200 ul lysate, mixed by pipetting up and down and the mixture added to a DNA column. This was followed by spinning at 16,000×g for 30 sec and discarding the flow-through. Column was washed twice with 200 ul Wash buffer, spinning at 16,000×g for 30 sec each time. DNA was eluted in 20 ul elution buffer.

Two PCRs were setup per sample in PCR tubes: PCR A controls for integration while PCR B generated the actual sequencing library. For PCR A, mixed 9.5 ul eluted DNA with either 10.5 ul water, 2.5 ul of 25 uM oBC3657 oligonucleotide, 2.5 ul of 25 uM oBC3716 oligonucleotide, and 25 ul of NEBNext High-Fidelity 2× Master Mix. For PCR B, mixed 9.5 ul eluted DNA with 10.5 ul water, 2.5 ul of 25 uM oBC3657 oligonucleotide, 2.5 ul of 25 uM P7-containing Ad2.xx oligonucleotide (see Table 6), and 25 ul of NEBNext High-Fidelity 2× Master Mix. The reactions were incubated in a PCR cycler for 5 minutes at 72° C. (gap repair), 2 minutes at 98° C., followed by 17 cycles of 10 seconds at 98° C., 10 seconds at 60° C., and 20 seconds at 72° C. Finish reaction by incubation for 1 minute at 72° C., then held at 4° C.

TABLE 8
(amplification sequences)

| Oligo ID | Oligo sequence (5′ > 3′) | SEQ ID NO: |
|---|---|---|
| oBC3657 | AATGATACGGCGACCACCGAGATCTACACTCGTCGGCAGCGTCAG | 56 |
| oBC3716 | CAAGCAGAAGACGGCATACGAGATgtcactccgcctcgtAG | 57 |
| Ad2.12 | CAAGCAGAAGACGGCATACGAGATTCCTCTACGTCTCGTGGGCTCGGAGATGTG | 58 |
| Ad2.13 | CAAGCAGAAGACGGCATACGAGATCAGATCCAGTCTCGTGGGCTCGGAGATGTG | 59 |

Adxx oligonucleotides for the library preparation were adapted with slight modifications from Buenrostro et al. 2015 (https://doi.org/10.1038/nature14590).

PCR clean up reactions were performed using 1.2× volume of Ampure XP beads. 60 ul of Ampure XP beads was added to a 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to the sample, vortexed to mix and incubated at room temperature for 2 minutes. Beads were bound to a magnet at room temperature for 5 minutes and the eluate containing library DNA fragments was collected.

Resulting libraries were checked on a DNA D5000 TapeStation instrument.

Results are shown in FIGS. 15A-C.

### Example 5 (CUT&Tag-BALI)

### Assembling Transposomes (Tn5)

In a PCR tube, adapter pair (detection probe) Tn5ME-A was produced by mixing 13 ul of 100 uM oBC3620 (Tn5MEfwd-A) and 6.5 ul of 100 uM oBC3622 (Tn5MErev) oligonucleotides (re-suspended in nuclease-free water) with 1 ul of 20× annealing buffer (800 mM Tris-HCl PH 8.0, 1 M NaCl). In a separate PCR tube, adapter pair (detection probe) Tn5ME-OL1 was generated by mixing 13 ul of 100 uM oBC3656 (Tn5MEfwd-OL1) and 6.5 ul of 100 uM oBC3622 (Tn5MErev) oligonucleotides (re-suspended in nuclease-free water) were mixed with 1 ul of 20× annealing buffer (800 mM Tris-HCl pH 8.0, 1 M NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed oligos were stored at −20° C. until further use.

Transposomes were assembled with equal amounts of separately pre-annealed Tn5ME-A and Tn5ME-OL1 adapters (detection probes). In a PCR tube, 3.13 ul of annealed Tn5ME-A adapters (oBC3620/oBC3622) and 3.13 ul of annealed Tn5ME-B adapters (oBC3656/oBC3622) were mixed before adding 5 ul of 3.68 mg/ml pA-Tn5 (protein A-Tn5) Transposase (unloaded) and incubation for 30 minutes at 23° C. in a thermocycler. The loaded transposome complexes were mixed with 6.25 ul of 100% glycerol and stored at −20° C. until further use.

TABLE 9
(detection probes)

| | | SEQ |
|---|---|---|
| Oligo ID | Oligo sequence (5′ −> 3′) | ID NO: |
| oBC3620 | TCGTCGGCAGCGTCAGATGTGTATAAGAGA | 51 |
| | CAG | |
| oBC3622 | /5Phos/CTGTCTCTTATACACATCT | 52 |
| oBC3656 | /5SpPC/gtcactccgcctcgtAGATGTGT | 53 |
| | ATAAGAGACAG | |

/5Phos/ indicates 5′ phosphorylated group.
/5SpPC/ indicates 5′ photocaged group that can be cleaved
under UV light into a 5′ phosphorylated group. This
oligonucleotide was purified using “HPLC Purification”.

### Preparation of Slides and Plating of Cells on Slide

Using chamber slides (8-well glass slide), 500 ul ConA was added if fly cells were used (0.5 mg/ml in sterile H2O, stored at ° 4C). This was incubated overnight at 26° C. in a tissue culture incubator. The slide chamber was washed once with 500 ul 1×PBS (137 mM NaCl, 2.7 mM KCl, 10 mM Na₂HPO₄, 1.8 mM KH₂PO₄). Per chamber, 50,000 Drosophila S2 cells in 200 ul growth media (Schneider media Gibco, 10% FBS, 1% Pen-Strep) from a 2.5×10\hat{ }5 cells/ml dilution were plated. This was incubated for 4 hours at 26° C. in a tissue culture incubator to allow cells to attach to ConA (cells flatten out and change morphology). Cells were checked under a microscope.

### Fixation

Cells from tissue culture incubator were removed and the culture media removed. To each chamber, 500 ul of 0.5% formaldehyde diluted in PBS (27 ul of 37% PFA stock diluted in 1973 ul PBS) was added. This was incubated for 5 min at room temperature. Fixation was quenched with glycine (25 ul of 2.5M stock per chamber, corresponding to 1/20^{th} of total sample volume). This was then incubated at room temperature for 5 minutes. PFA and glycine solution were removed and cell chambers were washed twice with 500 ul of 1×PBS. Cells were checked under a microscope.

### Permeabilization and Antibody Incubation

500 ul C&T wash buffer (20 mM HEPES pH 7.5, 150 mM NaCl, 0.5 mM spermidine) containing 1× Roche Complete Protease Inhibitor EDTA-free tablet per 50 ml was added and incubated for 5 minutes at room temperature. 100 ul antibody buffer (2 mM EDTA, 0.1% BSA, 0.05% Digitonin, 20 mM HEPES pH 7.5, 150 mM NaCl, 0.5 mM spermidine) containing 1× Roche Complete Protease Inhibitor EDTA-free tablet per 50 ml and 1 ul of primary antibody (1:100 for rabbit anti-H3K27me3 antibody which detects tri-methylated histone 3 lysine 27, a repressive mark) or no antibody as control was added. This was rocked at room temperature for 2 hours. 100 ul Dig-wash buffer (0.05% Digitonin, 20 mM HEPES pH 7.5, 150 mM NaCl, 0.5 mM spermidine) containing 1× Roche Complete Protease Inhibitor EDTA-free tablet per 50 ml and 1 ul of secondary antibody (1:100 for anti-rabbit secondary guinea pig antibody) which binds to the primary antibody was added. This was rocked at room temperature for 45 minutes. The liquid was removed and sample washed thrice with 500 ul Dig-wash buffer.

### Tagmentation

Wash buffer was removed and 100 μl of transposition mix (0.4 ul of home-loaded pA-Tn5 transposase complex with Tn5ME-A and Tn5ME-OL1 adapters in 0.01% Digitonin, 20 mM HEPES pH 7.5, 300 mM NaCl, 0.5 mM spermidine) containing 1× Roche Complete Protease Inhibitor EDTA-free tablet per 50 ml was added before incubation with rocking at room temperature for 1 hour. Transposition mix was removed and sample washed thrice with 500 ul Dig-300 buffer (0.01% Digitonin, 20 mM HEPES PH 7.5, 300 mM NaCl, 0.5 mM spermidine) containing 1× Roche Complete Protease Inhibitor EDTA-free tablet per 50 ml. The liquid was removed and 200 ul Tagmentation buffer (10 mM MgCl2, 0.01% Digitonin, 20 mM HEPES pH 7.5, 300 mM NaCl, 0.5 mM spermidine) containing 1× Roche Complete Protease Inhibitor EDTA-free tablet per 50 ml was added and incubated 30 minutes at 37° C. in a hybridisation oven. Tagmentation buffer was removed, and 200 ul of 40 mM EDTA was added before incubating at room temperature for 5 minutes to stop transposition/inactivate the transposase. EDTA was removed and 200 ul NEBuffer 3.1 was added, then incubated at room temperature for 5 minutes. Cells were checked under a microscope.

### Uncaging

Chambers that should not be illuminated were covered with aluminium foil. Slides were placed under UV transilluminator (with a mid-wavelength of 302 nm, suitable to uncage the photo-cleavable [5SpPC] modifications containing a photo-liable functional group that is cleavable by UV light of specific wavelength between 300-350 nm) and irradiated for exactly 150 seconds.

### Ligation

Ligation fragments (index sequences) were annealed: 10 ul of fragment 1/oBC3631 (100 uM) and 10 ul of fragment 2/oBC3650 (100 uM) oligonucleotides were mixed with 20 ul 2× annealing buffer (20 mM Tris, pH7.5-8.0, 100 mM NaCl, 2 mM EDTA) in a PCR tube. Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed ligation fragments were stored at −20° C. until use.

NEBuffer 3.1 was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed ligation fragments at 25 uM concentration) was added, then incubated for 30 minutes at 37° C. in a hybridisation oven. Ligation reaction solution was removed and 500 ul 1×PBS added. This was incubated for 5 min at room temperature. Cells were checked under a microscope.

TABLE 10
(index sequences)

| | | SEQ |
|---|---|---|
| Oligo ID | Oligo sequence (5′ > 3′) | ID NO: |
| oBC3631 | GTCTCGTGGGCTCGGAGATGTGTATAAGAG | 54 |
| | ACAG | |
| oBC3650 | acgaggcggagtgacCTGTCTCTTATACAC | 55 |
| | ATCTC | |

### Lysis and De-Crosslinking

Chambers were quickly rinsed in 500 ul nuclease-free water. Ensured to remove all of the liquid. 200 ul of reverse crosslinking solution (50 mM Tris-HCl PH8.0, 1 mM EDTA, 1% SDS, 150 mM NaCl, 0.4 mg/ml proteinase K, and 0.2 M glucose if fly cells are used) was added and incubated at room temperature for 20 minutes, carefully shaking the chambers every 5 minutes. Subsequently, the solution was transferred to an Eppendorf tube and the samples incubated overnight at 60° C. with shaking (1,000 rpm) in a thermomixer (Eppendorf). Slides were checked under a microscope for residual cells indicative of unsuccessful lysis.

### Library Preparation

DNA was purified using Zymo DNA Clean & Concentrator-5 columns. 1 ml of DNA Binding buffer was added to 200 ul lysate, mixed by pipetting up and down and added mixture to a DNA column. This was spun at 16,000×g for 30 sec and the flow-through discarded. Column was washed twice with 200 ul Wash buffer, spinning at 16,000×g for 30 sec each time. DNA was eluted in 20 ul elution buffer.

Two PCRs were set up per sample in PCR tubes: PCR A controls for integration while PCR B generated the actual sequencing library. For PCR A, mixed 9.5 ul eluted DNA with either 10.5 ul water, 2.5 ul of 25 uM oBC3657 oligonucleotide, 2.5 ul of 25 uM oBC3716 oligonucleotide, and 25 ul of NEBNext High-Fidelity 2× Master Mix. For PCR B, mixed 9.5 ul eluted DNA with 10.5 ul water, 2.5 ul of 25 uM oBC3657 oligonucleotide, 2.5 ul of 25 uM P7-containing Ad2.xx oligonucleotide (see Table 9), and 25 ul of NEBNext High-Fidelity 2× Master Mix. The reactions were incubated in a PCR cycler for 5 minutes at 72° C. (gap repair), 2 minutes at 98° C., followed by 17 cycles of 10 seconds at 98° C., 10 seconds at 60° C., and 20 seconds at 72° C. Finish reaction by incubation for 1 minute at 72° C., then held at 4° C.

TABLE 11
(amplification sequences)

| Oligo ID | Oligo sequence (5′ > 3′) | SEQ ID NO: |
|---|---|---|
| oBC3657 | AATGATACGGCGACCACCGAGATCTACACTCGTCGGCAGCGTCAG | 56 |
| oBC3716 | CAAGCAGAAGACGGCATACGAGATgtcactccgcctcgtAG | 57 |
| Ad2.14 | CAAGCAGAAGACGGCATACGAGATACAAACGGGTCTCGTGGGCTCGGAGATGTG | 60 |
| Ad2.15 | CAAGCAGAAGACGGCATACGAGATACCCAGCAGTCTCGTGGGCTCGGAGATGTG | 61 |

Adxx oligonucleotides for the library preparation were adapted with slight modifications from Buenrostro et al. 2015 (https://doi.org/10.1038/nature14590).

Clean-up PCR reactions were performed using 1.2× volume of Ampure XP beads. 60 ul of Ampure XP beads were added to 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to sample, vortexed to mix and incubated at room temperature for 2 minutes. Beads were bound to a magnet at room temperature for 5 minutes and the eluate containing library DNA fragments was collected.

Resulting libraries were checked on a High Sensitivity DNA D5000 TapeStation instrument. Results are shown in FIGS. 16A-C.

### Example 6 (MultiOmics BALI Showing ATAC-Seq and RNA Via RT and In Vitro Template Switching)

### Assembling Transposomes (Tn5)

In a PCR tube, adapter pair (detection probe) Tn5ME-A was produced by mixing 9.5 ul of 100 uM oBC3620 (Tn5MEfwd-A) and 9.5 ul of 100 uM oBC3622 (Tn5MErev) oligonucleotides (re-suspended in nuclease-free water) with 1 ul of 20× annealing buffer (800 mM Tris-HCl pH 8.0, 1 M NaCl). In a separate PCR tube, adapter pair (detection probe) Tn5ME-OL1 was generated by mixing 9.5 ul of 100 μM oBC3729 (Tn5MEfwd-OL1-P) and 9.5 ul of 100 uM oBC3622 (Tn5MErev) oligonucleotides (re-suspended in nuclease-free water) were mixed with 1 ul of 20× annealing buffer (800 mM Tris-HCl pH 8.0, 1 M NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed oligos were stored at −20° C. until further use.

Transposomes were assembled with equal amounts of separately pre-annealed Tn5ME-A and Tn5ME-OL1 adapters. In a PCR tube, 5 ul of annealed Tn5ME-A adapters (oBC3620/oBC3622) and 5 ul of annealed Tn5ME-OL1 adapters (oBC3729/oBC3622) were mixed before adding 10 ul of 2 mg/ml Tagmentase (Tn5 Transposase, unloaded) and incubation for 30 minutes at 23° C. in a thermocycler. 20 ul of loaded transposome complexes were mixed with 10 ul of 100% glycerol and stored at −20° C. until further use.

TABLE 12
(detection probes)

| Oligo ID | Oligo sequence (5′ −> 3′) | SEQ ID NO: |
|---|---|---|
| oBC3620 | TCGTCGGCAGCGTCAGATGTGTATAAGAGACAG | 51 |
| oBC3622 | /5Phos/CTGTCTCTTATACACATCT | 52 |
| oBC3729 | /5Phos/gtcactccgcctcgtCGTGATAGATGTGTATAAGAGACAG | 62 |
| oBC3751 | /5Phos/gtcactccgcctcgtACATCG/iBiodT/TTTTTTTTTTTTTTTTTVN | 63 |

/5Phos/ indicates 5′ phosphorylated group.
V indicates any base that is not T (i.e., A, C, G), N indicates any base.
/iBiodT/ indicates internal biotin group that can be used to separate RNA/cDNA fractions
from DNA maternal. This oligonucleotide was purified using “HPLC Purification”.

### Preparation of Cells on Slide

Using chamber slide (8-well glass slide), per chamber 20,000 mouse 4T1 cells in 300 ul growth media (DMEM supplemented with 10% FBS, 1% Pen-Strep, 2 mM L-Glutamine) were added. Cells were incubated over night at 37° C. in a tissue culture incubator with 5% CO2, yielding ˜40,000 cells the next morning when the experiment was continued. Cells were checked under a microscope.

### Fixation

Cells were removed from the tissue culture incubator and the culture media removed. To each chamber, 500 ul of 0.5% formaldehyde diluted in PBS (27 ul of 37% PFA stock diluted in 1973 ul PBS) was added. This was incubated for 5 min at room temperature. Fixation was quenched with glycine (25 ul of 2.5M stock per chamber, corresponding to 1/20th of total sample volume). This was then incubated at room temperature for 5 minutes. The PFA and glycine solution was removed and the cell chambers were twice with 500 ul of 1×PBS.

### Permeabilization

The cell chamber was rinsed once in nuclease-free water. 500 ul lysis buffer (10 mM Tris-HCl pH7.4, 10 mM NaCl, 3 mM MgCl2, 0.01% Tween-20, 0.01% NP-40, 0.001% Digitonin, 1% BSA, 0.1 U/ul RNasin RNase Inhibitor) was added for permeabilization and incubated for 15 minutes at room temperature. Lysis buffer was removed and 400 ul wash buffer (10 mM Tris-HCl pH7.4, 10 mM NaCl, 3 mM MgCl2, 0.1% Tween-20, 1% BSA, 0.1 U/ul RNasin RNase Inhibitor) added followed by incubation for 5 minutes at room temperature.

### Tagmentation

Wash buffer was removed and 100 ul of transposition mix (0.5 ul of home-loaded Tn5 transposase with Tn5ME-A and Tn5ME-OL1 adapters in 10 mM Tris-HCl PH 7.6, 5 mM MgCl2, 10% Dimethyl Formamide, 0.33×PBS, 0.1% Tween-20, 0.01% Digitonin, 0.4 U/ul RNasin RNase Inhibitor) was added before incubation for 30 minutes at 37° C. in a hybridisation oven. Transposition mix was removed, 200 ul of 40 mM EDTA was added and incubate at room temperature for 5 minutes to stop transposition/inactivate the transposase. EDTA was removed and sample was washed twice with 300 ul wash buffer (10 mM Tris-HCl PH7.4, 10 mM NaCl, 3 mM MgCl2, 0.1% Tween-20, 1% BSA, 0.4 U/ul RNasin RNase Inhibitor).

### Reverse Transcription

Wash buffer was removed and 100 ul of RT mix (1× Maxima RT buffer, 0.5 mM dNTPs, 5 uM oBC3751 [anchored oligo-d (T)-OL1-P] oligonucleotide], 15% PEG 6000, 15 U/ul Maxima H Minus Reverse Transcriptase, 0.01% Digitonin, 0.4 U/ul RNasin RNase Inhibitor) was added before incubation for 35 minutes at 42° C. in a hybridisation oven. RT mix was removed, 200 ul of 40 mM EDTA was added and incubated at room temperature for 5 minutes to stop reverse transcription. EDTA was removed, followed by rinsing once with 200 ul NEBuffer 3.1, then incubating for 5 minutes at room temperature with 200 ul NEBuffer 3.1.

### Ligation

Ligation fragments (index sequences) were annealed: 10 ul of fragment 1/oBC3736 (100 μM) and 10 ul of fragment 2/oBC3758 (100 μM) oligonucleotides were mixed with 20 ul 2× annealing buffer (20 mM Tris, pH7.5-8.0, 100 mM NaCl, 2 mM EDTA) in a PCR tube. Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed ligation fragments were stored at −20° C. until use.

NEBuffer 3.1 was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed ligation fragments at 25 μM concentration) were added, then incubated for 30 minutes at 37° C. in a hybridisation oven. The ligation reaction solution was removed and 500 ul of 1×PBS was added. This was incubated for 5 min at room temperature.

TABLE 13
(index sequences)

| Oligo ID | Oligo sequence (5′ > 3′) | SEQ ID NO: |
|---|---|---|
| oBC3736 | GTCTCGTGGGCTCGGAGATGTGTATAAGAGACAGGTCACACCCGcg | 64 |
| oBC3758 | acgaggcggagtgacCGCGGGTGTGACCTGTCTCTTATACACATCTCC | 65 |

### Lysis and De-Crosslinking

Ensured to remove all of the liquid. 200 ul of reverse crosslinking solution (50 mM Tris-HCl pH8.0, 1 mM EDTA, 1% SDS, 150 mM NaCl, 0.4 mg/ml proteinase K, 0.2 U/ul RNasin RNase Inhibitor) was added and incubated at room temperature for 20 minutes, carefully shaking the chambers every 5 minutes. Subsequently, the solution was transferred to an Eppendorf tube and the samples incubated for 1 hour at 55° C. with shaking (1,000 rpm) in a thermomixer (Eppendorf). Slides were checked under a microscope for residual cells indicative of unsuccessful lysis. 10 ul of 100 mM PMSF was added to each sample in 200 ul reverse crosslinking solution, mixed and incubated at room temperature for 10 minutes.

Separate DNA Fraction from RNA/cDNA Material

For each sample, 10 ul of MyOne C1 Dynabeads was washed thrice with 60 ul 1× B&W-T buffer (5 mM Tris pH 8.0, 1 M NaCl, 0.5 mM EDTA, 0.05% Tween-20, 0.25 U/ul RNasin RNase Inhibitor) in Eppendorf tube. For each sample, resuspend beads in 200 ul of 2× B&W buffer (10 mM Tris pH 8.0, 2 M NaCl, 1 mM EDTA, 0.5 U/ul RNasin RNase Inhibitor) and mixed with 200 μl of the reverse crosslinked material, then incubated the mixture on a rotator at 10 rpm for 1 hour at room temperature. Lysate was put on a magnetic stand to separate the supernatant (with DNA fraction) from beads (containing RNA/cDNA). The supernatant (˜400 ul) was transferred to a new Eppendorf tube and DNA purification was performed.

### Library Preparation DNA

DNA was purified using Zymo DNA Clean & Concentrator-5 columns. 1 ml of DNA Binding buffer was added to 200 ul lysate, mixed by pipetting up and down and added mixture to a DNA column. This was spun at 16,000×g for 30 sec and the flow-through discarded. Column was washed twice with 200 ul Wash buffer, spinning at 16,000×g for 30 sec each time. DNA was eluted in 20 ul elution buffer.

For each sample, a PCR was set up by mixing 9.5 ul eluted DNA with either 10.5 ul water, 2.5 ul of 25 uM oBC3657 oligonucleotide, 2.5 ul of 25 uM P7-containing Ad2.xx oligonucleotide (see Table 12), and 25 ul of NEBNext High-Fidelity 2× Master Mix. The reactions were incubated in a PCR cycler for 5 minutes at 72° C. (gap repair), 2 minutes at 98° C., followed by 17 cycles of 10 seconds at 98° C., 10 seconds at 60° C., and 20 seconds at 72° C. The reaction was finished by incubation for 1 minute at 72° C., then held at 4° C.

TABLE 14
(amplification sequences)

| Oligo ID | Oligo sequence (5′ > 3′) | SEQ ID NO: |
|---|---|---|
| oBC3657 | AATGATACGGCGACCACCGAGATCTACACTCGTCGGCAGCGTCAG | 56 |
| Ad2.5 | CAAGCAGAAGACGGCATACGAGATAGGAGTCCGTCTCGTGGGCTCGGAGATGTG | 66 |
| Ad2.6 | CAAGCAGAAGACGGCATACGAGATCATGCCTAGTCTCGTGGGCTCGGAGATGTG | 67 |
| Ad2.13 | CAAGCAGAAGACGGCATACGAGATCAGATCCAGTCTCGTGGGCTCGGAGATGTG | 59 |
| Ad2.14 | CAAGCAGAAGACGGCATACGAGATACAAACGGGTCTCGTGGGCTCGGAGATGTG | 60 |
| oBC3763 | TCGTCGGCAGCGTCAGATGTGTATAAGAGACAGrGrG+G | 68 |

rG indicates RNA base, while +G indicates LNA base.

Adxx oligonucleotides for the library preparation were adapted with slight modifications from Buenrostro et al. 2015 (https://doi.org/10.1038/nature 14590).

Clean-up PCR reactions were performed using 1.2× volume of Ampure XP beads. 60 ul of Ampure XP beads was added to 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to the sample, vortexed to mix and incubated at room temperature for 2 minutes. Beads were bound to a magnet at room temperature for 5 minutes and the eluate containing library DNA fragments was collected.

### In Vitro Template Switching and RNA Library Preparation

Beads containing RNA/cDNA were washed thrice with 100 ul of 1× B&W-T buffer (5 mM Tris pH 8.0, 1 M NaCl, 0.5 mM EDTA, 0.05% Tween-20, 0.25 U/ul RNasin RNase Inhibitor), followed by one wash with 1×STE (10 mM Tris pH 8.0, 50 mM NaCl, 1 mM EDTA, 0.25 U/ul RNasin RNase Inhibitor). Beads were moved to a PCR tube following the first resuspension step.

For in vitro template switching, beads were bound to a magnet, 1×STE was removed and beads resuspended in 50 μl of template switching mix (1× Maxima RT buffer, 1 mM dNTPs, 2.5 uM oBC3763 template-switching oligonucleotide, 15% PEG 6000, 10 U/ul Maxima H Minus Reverse Transcriptase, 4 U/ul RNasin RNase Inhibitor). Beads were rotated at 10 rpm for 30 minutes at room temperature, then the beads were shaken at 300 rpm for 90 minutes at 42° C., while resuspending the beads every 30 minutes by flicking. The supernatant was removed and beads washed twice with 100 ul of 1×STE without disturbing the beads pellet.

The liquid was removed and beads resuspended in 50 ul PCR mix containing 25 ul of NEBNext High-Fidelity 2× Master Mix, 23.4 ul water, 0.8 ul of 25 uM oBC3657 oligonucleotide, and 0.8 ul of 25 uM P7-containing Ad2.xx oligonucleotide (see Table 12). The reactions were incubated in a PCR cycler for 3 minutes at 95° C., followed by 24 cycles of 20 seconds at 98° C., 20 seconds at 65° C., and 20 seconds at 72° C. The reaction was finished by incubation for 1 minute at 72° C., then held at 4° C. Tubes were placed on a magnet and the supernatant transferred to a new tube.

Clean-up PCR reactions were performed using 0.85× volume of Ampure XP beads. 42.5 ul of Ampure XP beads were added to 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to the sample, vortexed to mix and incubated at room temperature for 2 minutes. Beads were bound to a magnet at room temperature for 5 minutes and the eluate containing library DNA fragments was collected.

Resulting libraries were checked on a Tapestation platform using DNA D5000 Screentape (DNA) and HighSensitivity D5000 Screentape (RNA).

Results are shown in FIGS. 17A-C.

### Example 7 (MultiOmics BALI Showing ATAC-Seq and RNA Via RT and In Situ Template Switching)

TABLE 15
(detection probes, template switching oligos and amplification sequences)

| Oligo ID | Oligo sequence (5′ −> 3′) | SEQ ID NO: |
|---|---|---|
| BL374/oBC3620 | TCGTCGGCAGCGTCAGATGTGTATAAGAGACAG | 51 |
| BL375/oBC3631 | GTCTCGTGGGCTCGGAGATGTGTATAAGAGACAG | 54 |
| BL538 | /5Phos/CTGTCTCTTATACACATCTGATCGACT/3Inv | 69 |
| | dT/ | |
| BL366 | /5Cy5/AGACGTGTGCTCTTCCGATCTGTTTAGCTGCTA | 127 |
| | GCT/ideSBioTEG/AGCGCTTTTTTTTTTTTTTTTTTT | (5′ to ideSBioTEG) |
| | TTTTTTTTTTTT | 128 |
| | | (ideSBioTEG to 3′) |
| BL320 | CTAGCTAGTAATACGACTCACTATAGGGAGtacactctt | 71 |
| | tccctacacgacgctcttccgatctrGrGrG | |
| BL449 | AATGATACGGCGACCACCGAGATCTACACTCGTCGGCAG | 72 |
| | CGTCAGATGTG | |
| Ad2.1 | CAAGCAGAAGACGGCATACGAGATTCGCCTTAGTCTCGT | 73 |
| | GGGCTCGGAGATGT | |
| Truseq_Universal_R1 | AATGATACGGCGACCACCGAGATCTACACTCTTTCCCTA | 74 |
| | CACGACGCTCTTCCGATCT | |
| A012_Truseq_R2 oligo | CAAGCAGAAGACGGCATACGAGATtacaagGTGACTGGA | 75 |
| | GTTCAGACGTGTGCTCTTCCGATC | |

/5phos/corresponds to a 5′ phosphate group
/3InvdT/correspond to a 3′ inverted deoxy T group
rG corresponds to a RNA guanine nucleotide
/5cy5/corresponds to a 5′ fluorescent cyanine 5 group
/ideSBioTEG/corresponds to an internal spacer desthiobiotin group linked through a
TEG to a nucleotide

Oligos were all resuspended in H2O to a concentration of 100 uM unless otherwise specified (i.e., BL374, BL375, BL538)

### Cell Preparation

Approx 40,000 4T1 murine cells were plated on 40 mm round coverslips (#1.5 thickness, 170 um+−5 um) in DMEM medium supplemented with 10% fetal bovine serum and cultured until the coverslip was approximately 80% confluent.

### Transposome Assembly (Tn5)

Oligonucleotides BL375, BL538 and BL374 were resuspended to a concentration of 100 μM in tn5 annealing buffer (40 mM Tris-HCl pH8.0, 50 mM NaCl). Then, two oligo mixes were prepared as follows: (i) 5 ul BL374+5 ul BL538, and (ii) 5 ul BL375+5 ul BL538.

The mixes were transferred to thin-walled 200 ul PCR tubes and subjected to the following PCR program to anneal the tn5 adapters: 95° C. for 5 minutes, ramp down 0.1° C./sec to 65° C., 65° C. for 5′, ramp down 0.1° C./sec to 12° C., 12° C. for 5 minutes. The annealed adapters were mixed in equal amount (3 ul+3 ul) and combined with 2 volumes (6 ul) commercial tn5 transposon enzyme (diagenode unloaded tagmentase), and finally incubated at 23° C. for 30′. The resulting transposon complex was stored on ice until used.

### Fixation, Permeabilization and Transposition

All steps were performed at room temperature (21° C.) unless otherwise specified. The coverslip coated with 4T1 cells was transferred to a 6 cm petri dish and washed once in 5 ml PBS, then fixed for 10 minutes in 5 ml 0.5% PFA in 1×PBS. The fixative was then inactivated by a wash in 1M glycine/1×PBS for 5′. The fixed coverslip was washed twice for 5 minutes each in 1×PBS and incubated with 5 ml permeabilization buffer (0.5% Triton X-100, 3 mM MgCl₂, 1:1000 NEB Murine RNase inhibitor, in 1×PBS). After permeabilization, the slide was further washed for three times for 5 minutes each in 5 ml 1×PBS supplemented with 3 mM MgCl2, 0.5% BSA and 1:1000 Murine RNase inhibitor.

The sample was then incubated for 5 minutes in 5 ml freshly prepared transposition buffer (500 ul Tris-Acetate pH8, 5 mM MgCl₂, 0.1% Tween 20, 10% DMF, in 0.3×PBS). 33 ul of transposition mix were prepared by combining 30 ul of transposition buffer (see above) with 0.5 ul RNAsin plus RNase inhibitor (Promega) and 2.5 ul of the assembled transposon complex from the previous step. The 30 ul were transferred to a sheet of parafilm placed on a flat 15 ml petri dish, and the sample coverslip was inverted over it. The sample was then incubated for 30 minutes at 37° C. in a humid chamber to allow DNA tagmentation to happen. After this incubation, the coverslip was moved again to a 6 ml petri dish and washed once for 5 minutes in tn5 stop buffer (10 mM Tris-Acetate pH8, 20 mM EDTA, in H2O).

### Reverse Transcription and In-Situ Template Switching

A post-fixation step was then performed by incubating the sample in 5 ml 4% PFA in 1×PBS for 5 minutes, and the fixative was quenched by a 5′ wash in 5 ml 1M glycine/1×PBS, followed by two washes for 5 minutes each in 1×PBS. The sample was then incubated for 5 minutes in 0.1N HCl (diluted in H₂O), and washed twice more for 5′ each in 1×PBS.

The sample nucleic acids were denatured by incubating the coverslip in 1×PBS pre-heated at 65° C. for 5 minutes in a hybridization oven set at 65° C. 50 ul of pre-hybridization mix were then prepared by diluting 1.5 ul of 100 UM BL366 oligonucleotide in 50 ul H2O, and the coverslip was incubated with this mix at 65° C. for 10 minutes, in a humid chamber, using the same technique described for the transposition step above.

After pre-hybridization, the sample was placed on ice for 5 minutes while a reverse transcription/template switching mix was prepared combining the following: 2.5 ul 10 mM dNTP (Thermo Scientific), 1.5 ul BL366 oligonucleotide (pre-heated to 65 C for 2 minutes and snap-cooled on ice for 1′), 10 ul Superscript IV first-strand buffer 5×(Thermo Scientific), 1 ul RNAsin Plus RNase inhibitor (Promega), 3 ul BL320 oligonucleotide, 5 ul 5M Betaine, 0.5 ul ET-SSB protein (NEB), 2.5 ul 100 mM DTT, 2.5 ul Superscript IV enzyme (Thermo Scientific) and 21.5 ul H2O. The sample was incubated with this mix at 42° C. for 18 h, in a humid chamber, using the same technique described above.

After the 18° C. incubation, reverse transcription was blocked by transferring the coverslip to a 6 cm petri dish and washing it for 10′ in 5 ml of 1×PBS pre-heated at 85° C., in a hybridization oven set to 85° C.

### Lysis, Pull Down and Purification

500 ul RIPA buffer (50 mM tris pH 7.5, 1 mM EDTA, 1% Igepal CA-630, 0.5% Sodium Deoxycholate, 0.1% SDS, 1 mM DTT, 150 mM NaCl) were pipetted over the sample coverslip, and cells were scraped using a silicon blade cell scraper. The scraped cells were collected in a low-retention 1.5 ml Eppendorf tube, homogenized by passing them through a 1 ml syringe fitted with a 26G needle for 10 strokes, and incubated at 60° C. for 1 h with constant 1100 RPM shaking using a thermomixer. In the meanwhile, 20 ul of Dynabeads myOne T1 streptavidin magnetic beads (Thermo Scientific) were transferred to a separate low-retention 1.5 ml Eppendorf tube and washed twice with PBST (1×PBS. 0.1% Tween 20). After the completion of lysis, the sample lysate and the magnetic beads were combined and incubated for 30′ at room temperature with end-to-end rotation to capture the desthiobiotin-labelled cDNA. After this step, beads were collected using a magnet (Dynamag II, Thermo Scientific) and the supernatant containing the DNA transposed molecule was collected into a separate low-retention 1.5 ml Eppendorf tube. The beads were washed twice for 10 minutes each in RIPA buffer, and twice for 5 minutes each in PBST, each time resuspending them in the buffer, incubating them with end-to-end rotation, and immobilizing them on the magnet to remove the buffer. Finally, beads were resuspended in 50 ul elution buffer (50 mM tris pH 7.5, 25 mM D-Biotin, in H2O) and incubated for 18 h at 25° C. with constant shaking at 1100 RPM using a thermomixer, in order to release the desthiobiotin-labelled cDNA.

### DNA Library Preparation

The tube with the supernatant from the previous step, containing the DNA transposed molecules, was purified using a MinElute reaction cleanup kit (Qiagen) according to manufacturer's instructions, eluting the DNA in 30 ul EB buffer (Qiagen). A PCR mix was then produced combining the following: 6 ul eluted DNA, 25 ul 2×Q5 PCR master mix (NEB), 0.625 ul 100 UM BL449 oligo, 0.625 ul 100 uM AD2.1 oligo, and 17.5 ul H2O. The mix was transferred to a thin-walled PCR tube and PCR was run using the following program: 1) 72° C. 5 minutes, 2) 98° C. 3 minutes, 3) 98° C. 15 seconds, 4) 68° C. 20 seconds, 5) 72° C. 20 seconds, 6) go to 3 for 15 cycles, 7) 72° C. 2 minutes.

The PCR was purified using AMPpure SPRI beads (Beckman Coulter) according to manufacturer's instructions, using 0.8× bead/DNA ratio (40 ul of beads). The purified library was eluted in 30 ul H2O.

### RNA Library Preparation

A PCR mix was produced combining the following: 6 ul eluted cDNA from the pull-down step, 25 ul 2×Q5 PCR master mix (NEB), 0.5 ul 100 uM Truseq_Universal_R1 oligo, 0.5 ul 100 μM A012_Truseq_R2 oligo, and 18 ul H2O. The mix was transferred to a thin-walled PCR tube and PCR was run using the following program: 1) 98° C. 3 minutes, 2) 98° C. 20 seconds, 3) 69° C. 20 seconds, 4) 72° C. 20 seconds, 5) go to 2 for 24 cycles, 6) 72° C. 2 minutes.

The PCR was purified using AMPpure SPRI beads (Beckman Coulter) according to manufacturer's instructions, using 0.8× bead/DNA ratio (40 ul of beads). The purified library was eluted in 30 ul H2O.

### Library Sequencing and Analysis

Libraries were quantified using the KAPA library quantification kit for Illumina platforms (Roche) according to supplier's instruction. Libraries were pooled in equal molar amounts and sequenced on a NextSeq550 instrument with a paired-end 150nt protocol. Approximately 10 million reads were generated for both the DNA and cDNA libraries.

The demultiplexed fastQ files corresponding to the DNA library was first trimmed using the cutadapt package using the following parameters: -u -50 -e 0.05, removing the first 50 nucleotides from both the read1 and read2 sequences in order to delete sequencing/integration adapters. The two resulting files were then mapped on the mm³⁹ release of the mouse genome using the bowtie2 package with options —local —very-sensitive-local —no-mixed —no-discordant —phred33 -I 10 -X 700 -p 10. The resulting SAM files were sorted and indexed using the samtools package, and coverage was calculated using the bamCoverage tool from the deeptools package with the following options —normalizeUsing RPKM —smoothLength 75. The final bigwig file was visualized using the UCSC genome browser.

The demultiplexed fastQ files corresponding to the cDNA library were first filtered using the cutadapt package using the following parameters: -G \hat{ }NNNNNGCTGCTAGC -e 0.05 —pair-filter=both —discard-untrimmed —match-read-wildcards. This removed all reads not matching the expected structure at the 5′ end. The two resulting files were then trimmed, again using the cutadapt tool, using parameters -u 3-e 0.05 for read1 and -u 70-e 0.05 for read2, removing sequencing adapters and template switching sequences. The trimmed reads were mapped to the mm39 release of the mouse genome using the STAR package with the following parameters: —outSAMtype BAM Unsorted —quantMode GeneCounts. The resulting BAM files were sorted and indexed using the samtools package, and coverage was calculated using the bamCoverage tool from the deeptools package with the following options —normalizeUsing RPKM —smoothLength 75. The final bigwig file was visualized using the UCSC genome browser.

### Reference Data

The reference RNAseq data was produced from approximately 50 ng of total RNA extracted from 4T1 cells using TriZOL reagent (Thermo Fisher) according to supplier's instruction). The RNA was used to generate RNASeq libraries using the NebNEXT Ultra II directional RNA library prep kit (NEB) with the rRNA depletion workflow, according to supplier's instructions. Processing was performed as above.

The reference ATAC-Seq data was produced by performing the ATAC-Bali protocol (as per Example 1) on 40,000 4T1 murine cells.

Results are shown in FIGS. 18A-B.

### Example 8 (ATAC-BALI on Cells with 3 Ligations)

### Assembling Transposomes (Tn5)

In a PCR tube, adapter pair (detection probe) Tn5ME-A was produced by mixing 9.5 ul of 100 uM oBC3620 (Tn5MEfwd-A) and 9.5 ul of 100 uM oBC3622 (Tn5MErev) oligonucleotides (re-suspended in nuclease-free water) with 1 ul of 20× annealing buffer (800 mM Tris-HCl PH 8.0, 1 M NaCl). In a separate PCR tube, adapter pair (detection probe) Tn5ME-OL1-PC was generated by mixing 9.5 ul of 100 uM oBC3737 (Tn5MEfwd-OL1-PC) and 9.5 ul of 100 μM oBC3622 (Tn5MErev) oligonucleotides (re-suspended in nuclease-free water) were mixed with 1 ul of 20× annealing buffer (800 mM Tris-HCl PH 8.0, 1 M NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed oligos were stored at −20° C. until further use.

Transposomes were assembled with equal amounts of separately pre-annealed Tn5ME-A and Tn5ME-OL1-PC adapters (detection probes). In a PCR tube, 5 ul of annealed Tn5ME-A adapters (oBC3620/oBC3622) and 5 ul of annealed Tn5ME-OL1-PC adapters (oBC3737/oBC3622) were mixed before adding 10 ul of 2 mg/ml Tagmentase (Tn5 Transposase, unloaded) and incubation for 30 minutes at 23° C. in a thermocycler. 20 ul of loaded transposome complexes were mixed with 10 ul of 100% glycerol and stored at −20° C. until further use.

TABLE 16
(detection probes)

| Oligo ID | Oligo sequence (5′ −> 3′) | SEQ ID NO: |
|---|---|---|
| oBC3620 | TCGTCGGCAGCGTCAGATGTGTATAAGAGACAG | 51 |
| oBC3622 | /5 Phos/CTGTCTCTTATACACATCT | 52 |
| oBC3737 | /5SpPC/gtcactccgcctcgtCGTGATAGATGTGTATAAGAGACAG | 77 |

/5Phos/indicates 5′ phosphorylated group.
/5SpPC/indicates 5′ photocaged group that can be cleaved under UV light into a 5′
phosphorylated group. This oligonucleotide was purified using “HPLC Purification”.

### Annealing of Ligation Fragments

Ligation fragments (index sequences) were annealed: 8 ul of oBC3730 (100 uM) and 8 ul of oBC3738 (100 uM) oligonucleotides (OL1 ligation fragments) were mixed with 4 ul water and 20 ul 2× annealing buffer (20 mM Tris, pH7.5-8.0, 100 mM NaCl, 2 mM EDTA) in a PCR tube. 8 ul of oBC3733 (100 uM) and 8 ul of oBC3740 (100 μM) oligonucleotides (OL2 ligation fragments) were mixed with 4 ul water and 20 ul 2× annealing buffer (20 mM Tris, pH7.5-8.0, 100 mM NaCl, 2 mM EDTA) in a PCR tube. 8 ul of oBC3735 (100 uM) and 8 ul of oBC3736 (100 uM) oligonucleotides (OL3 ligation fragments) were mixed with 4 ul water and 20 ul 2× annealing buffer (20 mM Tris, pH7.5-8.0, 100 mM NaCl, 2 mM EDTA) in a PCR tube. Tubes were mixed and incubated in a PCR thermocycler for 5 minutes at 95° C., then cooled down to 4° C. at −0.1° C. per second. Annealed ligation fragments were stored at −20° C. until use.

TABLE 17
(ligation fragments)

| Oligo ID | Oligo sequence (5′ > 3′) | SEQ ID NO: |
|---|---|---|
| oBC3730 | acgaggcggagtgaccgGAGGGTCATCgc | 78 |
| oBC3738 | /5SpPC/GGACGAGAGGACTGCgcGATGACCCTCcg | 79 |
| oBC3733 | GCAGTCCTCTCGTCCcgGCTGTCCCGTgc | 80 |
| oBC3740 | /5SpPC/CACCCAGACTCTCTTgcACGGGACAGCcg | 81 |
| oBC3735 | AAGAGAGTCTGGGTGcgCGGGTGTGACCTGTCTCTTATACACATCTCCGAGCCCACGAGAC | 82 |
| oBC3736 | GTCTCGTGGGCTCGGAGATGTGTATAAGAGACAGGTCACACCCGCG | 64 |

/5SpPC/indicates 5′ photocaged group that can be cleaved under UV light into a 5′
phosphorylated group. This oligonucleotide was purified using ″HPLC Purification″.

### Preparation of Slides and Plating of Cells on Slide

Slide chambers (8-well glass slide) were washed once with 500 ul 1×PBS (137 mM NaCl, 2.7 mM KCl, 10 mM Na₂HPO₄, 1.8 mM KH₂PO₄). Per chamber, 40,000 mouse 4T1 cells in 200 ul growth media (Dulbecco's Modified Eagle Medium supplemented with 10% Fetal Bovine Serum, 1% Penicillin-Streptomycin and 1% L-Glutamine) from a 2.5×10\hat{ }5 cells/ml dilution were plated. The cells were incubated over night at 37° C. in a tissue culture incubator with 5% CO2. Cells were checked under a microscope.

### Fixation

Cells were removed from a tissue culture incubator and the culture media removed. To each chamber, 500 ul of 0.5% formaldehyde diluted in PBS (27 ul of 37% PFA stock diluted in 1973 ul PBS) was added. This was incubated for 5 min at room temperature. Fixation was quenched with glycine (25 ul of 2.5M stock per chamber, corresponding to 1/20th of total sample volume). This was then incubated at room temperature for 5 minutes. The PFA and glycine solution was removed, and the cell chambers were washed twice with 500 ul of 1×PBS. Cells were checked under a microscope.

### Permeabilization

Cell chambers were rinsed once in nuclease-free water. 500 ul lysis buffer (10 mM Tris-HCl pH7.4, 10 mM NaCl, 3 mM MgCl2, 0.01% Tween-20, 0.01% NP-40, 0.001% Digitonin, 1% BSA) were added for permeabilization and then incubated for 15 minutes at room temperature. Lysis buffer was removed and 500 ul wash buffer (10 mM Tris-HCl PH7.4, 10 mM NaCl, 3 mM MgCl₂, 0.1% Tween-20, 1% BSA) was added followed by incubation for 5 minutes at room temperature.

### Tagmentation

Wash buffer was removed and 100 μl of transposition mix (0.5 ul of home-loaded Tn5 transposase complex with Tn5ME-A and Tn5ME-OL1-PC adapters (detection probes) in 10 mM Tris-HCl PH 7.6, 5 mM MgCl2, 10% Dimethyl Formamide, 0.33×PBS, 0.1% Tween-20, 0.01% Digitonin) were added before incubation for 30 minutes at 37° C. in a hybridisation oven. Transposition mix was removed and 200 ul of 40 mM EDTA was added and incubated at room temperature for 5 minutes to stop transposition/inactivate the transposase. EDTA was removed and 200 ul NEBuffer 3.1, was added and then incubated at room temperature for 5 minutes.

### Uncaging 1

Chambers that should not be illuminated (−UV controls) were covered with aluminium foil. Slides were placed under a UV transilluminator (with a mid-wavelength of 302 nm, suitable to uncage the photo-cleavable [5SpPC] modifications containing a photo-liable functional group that is cleavable by UV light of specific wavelength between 300-350 nm) and irradiated for 120-150 seconds.

### Ligation 1

Buffer was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed OL1 ligation fragments at 25 uM concentration) was added, then incubated for 30 minutes at 37° C. in a hybridisation oven. Ligation reaction solution was removed, and cells were washed twice with 500 ul NEBuffer 3.1 at room temperature. The buffer was removed and 200 ul NEBuffer 3.1 was added.

### Uncaging 2

Chambers that should not be illuminated (−UV controls) were covered with aluminium foil. Slides were placed under a UV transilluminator (with a mid-wavelength of 302 nm, suitable to uncage the photo-cleavable [5SpPC] modifications containing a photo-liable functional group that is cleavable by UV light of specific wavelength between 300-350 nm) and irradiated for 120-150 seconds.

### Ligation 2

Buffer was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed OL2 ligation fragments at 25 uM concentration) was added, then incubated for 30 minutes at 37° C. in a hybridisation oven. Ligation reaction solution was removed, and cells were washed twice with 500 ul NEBuffer 3.1 at room temperature. The buffer was removed and 200 ul NEBuffer 3.1 was added.

### Uncaging 3

Chambers that should not be illuminated (−UV controls) were covered with aluminium foil. Slides were placed under a UV transilluminator (with a mid-wavelength of 302 nm, suitable to uncage the photo-cleavable [5SpPC] modifications containing a photo-liable functional group that is cleavable by UV light of specific wavelength between 300-350 nm) and irradiated for 120-150 seconds.

### Ligation 3

Buffer was removed and 60 ul of Ligation reaction solution (24 ul ligation mix [5.6 ul 10× T4 DNA ligase buffer, 2.28 ul of T4 DNA ligase high conc, 1.12 ul 5% Triton X-100, 15.01 ul nuclease-free water], 24 ul of 1× NEBuffer 3.1, 12 ul of annealed OL3 ligation fragments at 25 uM concentration) was added, then incubated for 30 minutes at 37° C. in a hybridisation oven. Ligation reaction solution was removed, and cells were incubated in 500 ul 1×PBS at room temperature.

### Lysis and De-Crosslinking

Chambers were quickly rinsed in 500 ul nuclease-free water, then all liquid was removed. 200 ul of reverse crosslinking solution (50 mM Tris-HCl pH8.0, 1 mM EDTA, 1% SDS, 150 mM NaCl, 0.4 mg/ml proteinase K) were added and incubated at room temperature for 20 minutes, carefully shaking the chambers every 5 minutes. Subsequently, the solution was transferred to an Eppendorf tube and incubated with the samples overnight at 60° C. with shaking (1,000 rpm) in a thermomixer (Eppendorf). Slides were checked under a microscope for residual cells indicative of unsuccessful lysis.

### Library Preparation

DNA was purified using Zymo DNA Clean & Concentrator-5 columns. 1 ml of DNA Binding buffer was added to 200 ul lysate, mixed by pipetting up and down and the mixture added to a DNA column. This was followed by spinning at 16,000×g for 30 sec and discarding the flow-through. Column was washed twice with 200 ul Wash buffer, spinning at 16,000×g for 30 sec each time. DNA was eluted in 20 ul elution buffer.

Two PCRs were set up per sample in PCR tubes: PCR A controls for integration while PCR B generated the actual sequencing library. For PCR A, mixed 9.5 ul eluted DNA with 10.5 ul water, 2.5 ul of 25 uM oBC3657 oligonucleotide, 2.5 ul of 25 uM oBC3766 oligonucleotide, and 25 ul of NEBNext High-Fidelity 2× Master Mix. For PCR B, mixed 9.5 ul eluted DNA with 10.5 ul water, 2.5 ul of 25 uM oBC3657 oligonucleotide, 2.5 ul of 25 uM P7-containing Ad2.xx oligonucleotide (see Table 18), and 25 ul of NEBNext High-Fidelity 2× Master Mix. The reactions were incubated in a PCR cycler for 5 minutes at 72° C. (gap repair), 2 minutes at 98° C., followed by 17 cycles of 10 seconds at 98° C., 10 seconds at 60° C., and 20 seconds at 72° C. Finish reaction by incubation for 1 minute at 72° C., then held at 4° C.

TABLE 18
(amplification sequences)

| Oligo ID | Oligo sequence (5′ > 3′) | SEQ ID NO: |
|---|---|---|
| oBC3657 | AATGATACGGCGACCACCGAGATCTACACTCGTCGGCAGCGTCAG | 56 |
| oBC3766 | CAAGCAGAAGACGGCATACGAGATgtcactccgcctcgt | 83 |
| Ad2.12 | CAAGCAGAAGACGGCATACGAGATTCCTCTACGTCTCGTGGGCTCGGAGATGTG | 58 |
| Ad2.13 | CAAGCAGAAGACGGCATACGAGATCAGATCCAGTCTCGTGGGCTCGGAGATGTG | 59 |
| Ad2.6 | CAAGCAGAAGACGGCATACGAGATCATGCCTAGTCTCGTGGGCTCGGAGATGTG | 67 |
| Ad2.7 | CAAGCAGAAGACGGCATACGAGATGTAGAGAGGTCTCGTGGGCTCGGAGATGTG | 84 |

Adxx oligonucleotides for the library preparation were adapted with slight modifications from Buenrostro et al. 2015 (https://doi.org/10.1033/nature 14590).

PCR clean up reactions were performed using 1.2× volume of Ampure XP beads. 60 ul of Ampure XP beads was added to a 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to the sample, vortexed to mix and incubated at room temperature for 2 minutes. Beads were bound to a magnet at room temperature for 5 minutes and the eluate containing library DNA fragments was collected.

Resulting libraries were checked on a DNA D5000 TapeStation instrument, quantified using the KAPA library quantification kit for Illumina platforms (Roche) according to supplier's instruction and sequenced in-house.

Results are shown in FIG. 22.

### Example 9 (CUT&Tag-BALI on Tissue)

### Assembling Transposomes (Tn5)

In a PCR tube, adapter pair (detection probe) Tn5ME-A was produced by mixing 13 ul of 100 uM oBC3620 (Tn5MEfwd-A) and 6.5 ul of 100 uM oBC3622 (Tn5MErev) oligonucleotides (re-suspended in nuclease-free water) with 1 ul of 20× annealing buffer (800 mM Tris-HCl PH 8.0, 1 M NaCl). In a separate PCR tube, adapter pair (detection probe) Tn5ME-6 mer was generated by mixing 13 ul of 100 uM oBC3952 (Tn5MEfwd-6 mer-P) and 6.5 ul of 100 uM oBC3622 (Tn5MErev) oligonucleotides (re-suspended in nuclease-free water) were mixed with 1 ul of 20× annealing buffer (800 mM Tris-HCl pH 8.0, 1 M NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Annealed oligos were stored at −20° C. until further use.

Transposomes were assembled with equal amounts of separately pre-annealed Tn5ME-A and Tn5ME-6 mer adapters (detection probes). In a PCR tube, 3.13 ul of annealed Tn5ME-A adapters (oBC3620/oBC3622) and 3.13 ul of annealed Tn5ME-6 mer adapters (oBC3952/oBC3622) were mixed before adding 5 ul of 3.68 mg/ml pA-Tn5 (protein A-Tn5) Transposase (unloaded) and incubation for 30 minutes at 23° C. in a thermocycler. The loaded transposome complexes were mixed with 6.25 ul of 100% glycerol and stored at −20° C. until further use.

TABLE 19
(detection probes)

| Oligo ID | Oligo sequence (5′ −> 3′) | SEQ ID NO: |
|---|---|---|
| oBC3620 | TCGTCGGCAGCGTCAGATGTGTATAAGAGACAG | 51 |
| oBC3622 | /5Phos/CTGTCTCTTATACACATCT | 52 |
| oBC3952 | /5Phos/GGTATTATGGAACGTGATAGATGTGTATAAGAGACAG | 85 |

/5Phos/indicates 5′ phosphorylated group.

### Annealing of Ligation Fragments

Ligation fragments (containing index sequences) were annealed: 7.125 ul of BL584 (100 uM) and 7.125 ul of BL585 (100 uM) oligonucleotides (584/585 ligation fragments) were mixed with 0.75 ul 20×SSC (3M NaCl, 0.3M sodium citrate, pH7.0) in a PCR tube. 7.125 ul of BL623 (100 uM) and 7.125 ul of BL583 (100 uM) oligonucleotides (623/583 ligation fragments) were mixed with 0.75 ul 20×SSC (3M NaCl, 0.3M sodium citrate, pH7.0) in a PCR tube. 7.125 ul of BL624 (100 uM) and 7.125 ul of BL601 (100 uM) oligonucleotides (624/601 ligation fragments) were mixed with 0.75 ul 20×SSC (3M NaCl, 0.3M sodium citrate, pH7.0) in a PCR tube. Tubes were mixed and incubated in a PCR thermocycler for 5 minutes at 95° C., then cooled down to 4° C. at −0.1° C. per second. Annealed ligation fragments were stored at −20° C. until use.

TABLE 20
(ligation fragments)

| Oligo ID | Oligo sequence (5′ > 3′) | SEQ ID NO: |
|---|---|---|
| BL584 | AAT/iSpPC/AATAACCGCACGAGTCCCG | 86 |
| | | (iSpPC to 3′) |
| BL585 | AATACCCGGGACTCGTGC | 87 |
| BL623 | /5deSBioTEG/AGACGTGTGCTCTTCCGATCTNNNNNNGCACCTAGATCG | 88 |
| BL583 | GGTTATTCGATCTAGGTGC | 89 |
| BL624 | /5deSBioTEG/AGACGTGTGCCTAGCTTCTTCCGATCTNNNNNNGCACGAGTCCCG | 90 |
| BL601 | GGTTATTCGGGACTCGTGC | 91 |

/iSpPC/indicates an internal photocaged group that can be cleaved under UV light into a 5′
phosphorylated group. This oligonucleotide was purified using “HPLC Purification”.
N indicates any base.
/5deSBioTEG/indicates a 5′ desthiobiotin group linked through a TEG linker to a nucleotide
(Desthiobiotin-TEG). This oligonucleotide was purified using “HPLC Purification”.

### Tissue Sectioning

Brains from E16.5 mouse were collected, and immediately embedded in OCT, snap-frozen in 2-Methylbutane and stored at −80° C. Tissue sections were cut at a thickness of 10 μm using RNase-clean procedures. Sections were mounted on SuperFrost Plus microscope slides and immediately transferred on dry ice, and later stored at −80° C. until use.

### Preparation of Tissue Slides

Tissue slides were taken from −80° C. and equilibrated and air dried at room temperature for 5 minutes. Tissue slice was encircled with a pap-pen and let dry for 5 minutes at room temperature. Tissue was incubated for 5 minutes with 400 ul 1×PBS (137 mM NaCl, 2.7 mM KCl, 10 mM Na₂HPO₄, 1.8 mM KH₂PO₄) at room temperature.

### Fixation

The PBS was removed from the tissue by aspiration (with pump). To each slide, 400 ul of 0.2% formaldehyde diluted in PBS (43.2 ul of 37% PFA stock diluted in 7956.8 ul PBS) was added. This was incubated for 5 min at room temperature. Fixation was quenched with 400 ul 1.25M glycine (0.5 ml of 2.5M stock solution mixed with 0.5 ml water) per slide. This was then incubated at room temperature for 5 minutes. PFA and glycine solution were removed, and slides were washed twice with 400 ul of C&T wash buffer (20 mM HEPES PH 7.5, 150 mM NaCl, 0.5 mM spermidine) containing 1× Roche Complete Mini Protease Inhibitor EDTA-free tablet/10 ml.

### Permeabilization and Antibody Incubation

400 ul C&T permeabilization buffer (20 mM HEPES pH 7.5, 150 mM NaCl, 0.5 mM spermidine, 0.01% NP-40, 0.01% Digitonin) containing 1× Roche Complete Mini Protease Inhibitor EDTA-free tablet/10 ml was added and incubated for 5 minutes at room temperature. Buffer was removed from the tissue by aspiration (with pump). 50 ul C&T primary antibody buffer (20 mM HEPES pH 7.5, 150 mM NaCl, 0.5 mM spermidine, 0.01% NP-40, 0.01% Digitonin, 2 mM EDTA, 0.001% BSA) containing 1× Roche Complete Mini Protease Inhibitor EDTA-free tablet/10 ml and 1 ul of primary antibody [corresponding to 1:50 for rabbit anti-H3K27me3 antibody which detects tri-methylated histone 3 lysine 27, a repressive mark] or no antibody as control was added. A coverslip was added, and the sample was rocked at 4° C. for overnight. Buffer was removed from the tissue by aspiration (with pump). 50 ul C&T permeabilization buffer (20 mM HEPES pH 7.5, 150 mM NaCl, 0.5 mM spermidine, 0.01% NP-40, 0.01% Digitonin) containing 1× Roche Complete Mini Protease Inhibitor EDTA-free tablet/10 ml and 1 ul of secondary antibody (corresponding to 1:50 for anti-rabbit secondary guinea pig antibody), which binds to the primary antibody, was added. A coverslip was added, and the sample was rocked at room temperature for 50 minutes. The liquid was removed, and the tissue sample washed once with 400 ul C&T wash buffer for 5 minutes at room temperature.

### Tagmentation

Buffer was removed and 50 μl of transposition mix (0.4 ul of home-loaded pA-Tn5 transposase complex with Tn5ME-A and Tn5ME-6 mer adapters in 20 mM HEPES pH 7.5, 300 mM NaCl, 0.5 mM spermidine, 0.01% Digitonin) containing 1× Roche Complete Mini Protease Inhibitor EDTA-free tablet/10 ml was added. A coverslip was added, and the sample was rocked at room temperature for 1 hour. Transposition mix was removed, and sample washed thrice with 400 ul C&T-300 wash buffer (20 mM HEPES pH 7.5, 300 mM NaCl, 0.5 mM spermidine) containing 1× Roche Complete Mini Protease Inhibitor EDTA-free tablet/10 ml. For the third wash, the samples were incubated for 5 minutes at room temperature. The liquid was removed, and the hybridization chamber was glued onto the slide. 200 ul C&T Tagmentation buffer (20 mM HEPES PH 7.5, 300 mM NaCl, 0.5 mM spermidine, 10 mM MgCl₂) containing 1× Roche Complete Protease Inhibitor EDTA-free tablet per 50 ml was added and incubated for 50 minutes at 37° C. in a hybridisation oven. Buffer was removed, and 200 ul of 40 mM EDTA was added before incubating at room temperature for 5 minutes to stop transposition/inactivate the transposase. EDTA was removed and the sample washed thrice with 400 ul 1×PBS. For the third wash, the samples were incubated for 5 minutes at room temperature.

### Ligation of Photocleavable Adapter

Samples were washed twice for 5 minutes in 2×SSC buffer at room temperature. Buffer was removed and samples were washed for 5 minutes in 1× T4 DNA Ligase buffer (50 mM Tris-HCl, 10 mM MgCl2, 1 mM ATP, 10 mM DTT, pH7.5). Buffer was removed and 50 ul of Ligation reaction solution (46.7 ul ligation mix [5 ul 10× T4 DNA ligase buffer, 2.5 ul of T4 DNA ligase high conc, 39.2 ul nuclease-free water], 3.3 ul of 45 uM annealed 584/585 ligation fragments) was added to the slide and covered with a flexible coverslip, then incubated for 45 minutes at 25° C. in a hybridisation oven in the dark. Solution was removed and sample washed once for 10 minutes in 400 ul 2×SSC buffer containing a 1:1,000 dilution of DRAQ5 at room temperature in the dark. Buffer was removed and samples washed 1× for 5 minutes in 2×SSC buffer containing 10% EC at room temperature in the dark. Buffer was removed and samples washed 1× for 5 minutes in 2×SSC buffer containing at room temperature in the dark.

### Uncaging Area 1

The buffer was removed and 100 ul of 2×SSC buffer were added to the sample, then covered using a 22×22 mm coverslip. The periaqueductal layer of both sides of the cortex was uncaged using the 405 nm laser (at 100% power) of a Leica SP5 confocal microscope for 5 minutes per field. Slide was transferred to a reservoir containing 2×SSC buffer that allowed the removal of the coverslip.

### Ligation Area 1

Samples were washed for 5 minutes in 2×SSC buffer at room temperature. Buffer was removed and samples were washed for 5 minutes in 1× T4 DNA Ligase buffer (50 mM Tris-HCl, 10 mM MgCl₂, 1 mM ATP, 10 mM DTT, pH7.5). Buffer was removed and 50 ul of Ligation reaction solution (46.7 ul ligation mix [5 ul 10× T4 DNA ligase buffer, 2.5 ul of T4 DNA ligase high conc, 39.2 ul nuclease-free water], 3.3 ul of 45 uM annealed 583/623 ligation fragments) was added to the slide and covered with a flexible coverslip, then incubated for 45 minutes at 25° C. in a hybridisation oven in the dark. Solution was removed and sample washed once for 10 minutes in 2×SSC buffer at room temperature in the dark. Buffer was removed and samples washed 1× for 5 minutes in 2×SSC buffer containing 10% EC at room temperature in the dark. Buffer was removed and samples washed 1× for 5 minutes in 2×SSC buffer containing at room temperature in the dark.

### Uncaging Area 2

The buffer was removed and 100 ul of 2×SSC buffer were added to the sample, then covered using a 22×22 mm coverslip. The cortical layer of both sides of the cortex was uncaged using the 405 nm laser (at 100% power) of a Leica SP5 confocal microscope for 5 minutes per field. Slide was transferred to a reservoir containing 2×SSC buffer that allowed the removal of the coverslip.

### Ligation Area 2

Samples were washed for 5 minutes in 2×SSC buffer at room temperature. Buffer was removed and samples were washed for 5 minutes in 1× T4 DNA Ligase buffer (50 mM Tris-HCl, 10 mM MgCl2, 1 mM ATP, 10 mM DTT, pH7.5). Buffer was removed and 50 ul of Ligation reaction solution (46.7 ul ligation mix [5 ul 10× T4 DNA ligase buffer, 2.5 ul of T4 DNA ligase high conc, 39.2 ul nuclease-free water], 3.3 ul of 45 uM annealed 601/624 ligation fragments) was added to the slide and covered with a flexible coverslip, then incubated for 45 minutes at 25° C. in a hybridisation oven in the dark. Solution was removed and sample washed once for 10 minutes in 2×SSC buffer at room temperature in the dark. Buffer was removed and samples washed 1× for 5 minutes in 2×SSC buffer containing 10% EC at room temperature in the dark. Buffer was removed and samples washed 1× for 5 minutes in 2×SSC buffer containing at room temperature in the dark.

### Lysis and De-Crosslinking

A new hybridization chamber (with adhesive) was added to the top of the slide. All liquid was removed and 200 ul of reverse crosslinking solution (50 mM Tris-HCl PH8.0, 1 mM EDTA, 1% SDS, 150 mM NaCl, 0.4 mg/ml proteinase K) was added and incubated in a wet box at 60° C. overnight.

### Library Preparation

DNA was purified using Zymo DNA Clean & Concentrator-5 columns. 1 ml of DNA Binding buffer was added to 200 ul lysate, mixed by pipetting up and down and added mixture to a DNA column. This was spun at 16,000×g for 30 sec and the flow-through discarded. Column was washed twice with 200 ul Wash buffer, spinning at 16,000×g for 30 sec each time. DNA was eluted in 20 ul elution buffer.

Quantitative PCR (qPCR) was performed to estimate the number of PCR cycles required for optimal library amplification. For each sample, the following reaction were set up in a qPCR plate: 1.9 ul eluted DNA were mixed with 1.2 ul water, 0.4 ul 1×SYBR Green I Nucleic Acid Stain, 1.0 ul of 12.5 uM TruSeq oligonucleotide (see table 21 below), 0.5 ul of 25 uM oBC3657 oligonucleotide, and 5 ul of NEBNext High-Fidelity 2× Master Mix. The reactions were incubated in a Bio-Rad CFX96 qPCR machine for 5 minutes at 72° C. (gap repair), 2 minutes at 98° C., followed by 40 cycles of 10 seconds at 98° C., 10 seconds at 60° C., and 20 seconds at 72° C. SYBR fluorescence was measured following each cycle. Finish reaction by incubation for 1 minute at 72° C., then held at 4° C.

Based on the number of required cycles for library amplification, the following reaction was set up in a PCR tube for each sample: 9.5 ul eluted DNA were mixed with 8.0 ul water, 5.0 ul of 12.5 uM TruSeq oligonucleotide, 2.5 ul of 25 uM oBC3657 oligonucleotide, and 25 ul of NEBNext High-Fidelity 2× Master Mix. The reactions were incubated in a PCR cycler for 5 minutes at 72° C. (gap repair), 2 minutes at 98° C., followed by the estimated cycle number for 10 seconds at 98° C., 10 seconds at 60° C., and 20 seconds at 72° C. Finish reaction by incubation for 1 minute at 72° C., then held at 4° C.

TABLE 21
(amplification sequences)

| Oligo ID | Oligo sequence (5′ > 3′) | SEQ ID NO: |
|---|---|---|
| oBC3657 | AATGATACGGCGACCACCGAGATCTACACTCGTCGGCAGCGTCAG | 56 |
| TruSeq A001 | CAAGCAGAAGACGGCATACGAGATCGTGATGTGACTGGAGTTCAGACGTGTGCTCTTCCGATC | 92 |
| TruSeq A002 | CAAGCAGAAGACGGCATACGAGATacatcgGTGACTGGAGTTCAGACGTGTGCTCTTCCGATC | 93 |
| TruSeq A003 | CAAGCAGAAGACGGCATACGAGATgcctaaGTGACTGGAGTTCAGACGTGTGCTCTTCCGATC | 94 |
| TruSeq A004 | CAAGCAGAAGACGGCATACGAGATtggtcaGTGACTGGAGTTCAGACGTGTGCTCTTCCGATC | 95 |
| TruSeq A005 | CAAGCAGAAGACGGCATACGAGATcactgtGTGACTGGAGTTCAGACGTGTGCTCTTCCGATC | 96 |
| TruSeq A007 | CAAGCAGAAGACGGCATACGAGATatctgGTGACTGGAGTTCAGACGTGTGCTCTTCCGATC | 97 |
| Ad2.14 | CAAGCAGAAGACGGCATACGAGATACAAACGGGTCTCGTGGGCTCGGAGATGTG | 60 |
| Ad2.15 | CAAGCAGAAGACGGCATACGAGATACCCAGCAGTCTCGTGGGCTCGGAGATGTG | 61 |

Adxx oligonucleotides for the library preparation were adapted with slight modifications from Buenrostro et al. 2015 (https://doi.org/10.1038/nature14590).

Clean-up PCR reactions were performed using 1.2× volume of Ampure XP beads. 60 ul of Ampure XP beads were added to 50 ul PCR sample. This was mixed well and incubated at room temperature for 10 minutes. Beads were bound to a magnet at room temperature for 5 minutes. While bound to the magnet, beads were washed twice with 170 ul freshly made 80% EtOH for 30 seconds. Beads were air dried at room temperature for 5 minutes. 20 ul EB buffer was added to sample, vortexed to mix and incubated at room temperature for 2 minutes. Beads were bound to a magnet at room temperature for 5 minutes and the eluate containing library DNA fragments was collected.

Resulting libraries were checked on a High Sensitivity DNA D5000 TapeStation instrument, quantified using the KAPA library quantification kit for Illumina platforms (Roche) according to supplier's instruction and sequenced in-house.

Results are shown in FIG. 23.

### Example 10 (ATAC-BALI on Tissue)

### Assembling Transposomes (Tn5)

In a PCR tube, adapter pair (detection probe) Tn5ME-A was produced by mixing 2.5 ul of 200 UM BL-515 (Tn5MEfwdA) and 2.5 ul of 200 UM BL-538 (ME_rev_p_blocked) oligonucleotides (resuspended in 1× Annealing buffer) with 5 ul of 1× annealing buffer (40 mM Tris-HCl PH 8.0, 50 mM NaCl). In a separate PCR tube, adapter pair (detection probe) Tn5ME-6A was generated by mixing 2.5 ul of 200 uM BL-515 (TA6+6_NEB01_Tn5ME) and 2.5 ul of 200 uM BL-538 (ME_rev_p_blocked) oligonucleotides (resuspended in 1× Annealing buffer) with 5 ul of 1× annealing buffer (40 mM Tris-HCl PH 8.0, 50 mM NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second. Transposomes were assembled with equal amounts of separately pre-annealed Tn5ME-A and Tn5ME-6A adapters (detection probes). In a PCR tube, 3 ul of annealed Tn5ME-A adapters, 3 ul of annealed Tn5ME-6A adapters and 6 μL of unloaded Tn5 transposase were mixed and incubated for 30 minutes at 23° C. in a thermocycler (not exceeding 60 minutes before incubation on tissue).

TABLE 22
(detection probes)

| | | SEQ |
|---|---|---|
| Oligo ID | Oligo sequence (5′ −> 3′) | ID NO: |
| oBC3620 | TCGTCGGCAGCGTCAGATGTGTATAAGAGACAG | 51 |
| BL699 | /5Phos/GGTATTATGGAACGTGATAGATGTGT | 98 |
| | ATAAGAGACAG | |
| BL538 | /5Phos/CTGTCTCTTATACACATCT GATCGA | 69 |
| | CT/3InvdT/ | |

/5Phos/indicates 5′ phosphorylated group./3InvdT/indicates 3′
inverted dT modification.

### Tissue Sectioning

Brains from adult mouse were collected, and immediately embedded in OCT, snap-frozen in 2-Methylbutane and stored at −80° C. Tissue sections were cut at a thickness of 10 μm using RNase clean procedures. Sections were mounted on SuperFrost Plus microscope slides and immediately transferred on dry ice, and later stored at −80° C. until use.

### Tissue Processing

Unless otherwise stated, tissue processing was conducted at room temperature (˜19-20° C.). On day 1 of the experiment, sections were retrieved from storage at −80° C., dried on a heat block at −37° C. for 5 minutes to improve adhesion to the slide, and a hydrophobic well was drawn around the section with a PapPen. The embedding matrix was removed by washing in 1×PBS for 5 minutes. After careful and gentle removal of PBS, sections were fixed for exactly 5 minutes in 0.5% PFA (diluted in 1×PBS) immediately quenched with 1.25M Glycine in 1×PBS (one quick wash and one wash for 5 minutes) and washed twice with 1×PBS for 5 minutes.

### Fixation

After careful removal of 1×PBS, sections were permeabilised in 1× Permeabilization Buffer (10 mM TrisHCl pH 7.4, 10 mM NaCl, 3 mM MgCl2, 0.01% Tween-20, 0.001% digitonin, 1% BSA in H₂O) for 15 minutes, and then washed in Wash Buffer (10 mM TrisHCl PH 7.4, 10 mM NaCl, 3 mM MgCl2, 1% BSA, 0.1% Tween-20) for one quick wash and one wash for 5 minutes, followed by two washes in 1×PBS for 5 minutes each.

### Tagmentation

After PBS was carefully removed from section, a SecureSeal hybridization chamber was applied to cover the sections. For each chamber, 200 μL of tagmentation mix (10 mM Tris HCl pH 7.6, 1M MgCl2, 10% Dimethyl Formamide, 0.33×PBS, 0.01% Tween-20, 0.01% Digitonin in H20) were added and incubated at −37° C. for 35 minutes. The chamber and transposition were removed, and the sections washed once in 40 mM EDTA for 5 minutes and three times in 1×PBS. Sections were stored in 1×PBS at 4° C. overnight.

### Cyclical Ligation and Uncaging

Each sections underwent, as appropriate, cycles of ligations, followed by washes, photorelease, and washes. Each step is described individually below.

### Ligations

Ligation fragments (index sequences) were annealed as follows. 7.125 ul of each forward and reverse oligonucleotide (100 uM in H₂O) were mixed with 1 μL of 20×SSC and annealed in a PCR thermocycler for 5 minutes at 95° C., then cooled down to 65° C. at −0.1° C. per second. A different index sequence was annealed for each bit for the spatial barcode: bit1_bulk (BL680, BL676), bit2_bulk (BL681, 677), bit3_bulk (BL683, BL642), bit4_area1 (BL732, BL644), bit4_area2 (BL733, BL 677), bit1_direct (BL732, BL641). Following incubation for 5 minutes at 65° C., the samples were cooled down to 10° C. at −0.1° C. per second. Annealed ligation fragments were stored at 4° C., light protected until use. Handling of photocleavable reagents was always performed in light protected conditions.

Sections were retrieved from storage and washed once in 1×PBS for 5 minutes, twice in 2×SSC for 5 minutes, and once in 1× Ligation Buffer (NEB) (50 mM TrisHCl, 10 mM MgCl₂, 1 mM ATP, 10 mM DTT, pH 7.5 at 25° C.) for 5 minutes. After careful removal of the ligation buffer, 50 uL Ligation Mix (1× Ligation Buffer (NEB), 100 U/uL T4 Ligase (NEB), 2.85 uM annealed index sequences in H₂O). ligation mix was applied to each section and then covered with 18×18 mm coverslips. Ligation were incubated for 60 minutes (first ligation) or 30 minutes (subsequent ligations) at 25° C. in a humid light protected chamber. The coverslip and ligation mix were removed by dipping the slides in a reservoir containing 2×SSC. Sections were washed once in 2×SSC+1:1000 Draq5 for 10 minutes, once in 2×SSC+10% EC for 5 minutes, and once in 2×SSC for 5 minutes.

TABLE 23
(index sequences)

| Oligo | | SEQ |
|---|---|---|
| ID | Oligo sequence (5′ > 3′) | ID NO: |
| BL680 | AAT/iSpPC/AATAACC GC ACTCAGGT CG | 99 |
| | | (iSpPC |
| | | to 3′) |
| BL676 | AATACC CG ACCTGAGT GC | 100 |
| BL681 | AAT/iSpPC/GGTATT GC ACGGACGA CG | 101 |
| | | (iSpPC |
| | | to 3′) |
| BL677 | GGTTATT CG TCGTCCGT GC | 102 |
| BL683 | AAT/iSpPC/AATAACC GC ACCTAGAT CG | 103 |
| | | (iSpPC |
| | | to 3′) |
| BL642 | AATACC CG ATCTAGGT GC | 104 |
| BL732 | AGACGTGTGCTCTTCCGATCT NNNNNN GC | 105 |
| | ACGAGTCC CG | |
| BL644 | GGTTATT CG GGACTCGT GC | 106 |
| BL733 | AGACGTGTGCTCTTCCGATCT NNNNNN GC | 107 |
| | ACGGACGA CG | |
| BL641 | AATACC CG GGACTCGT GC | 108 |

/iSpPC/indicates an internal photocaged group that can be
cleaved under UV light into a 5′ phosphorylated group. This
oligonucleotide was purified using “HPLC Purification”.
N indicates any base.

### Photorelease

The photocleavable cage was removed from the oligonucleotides by illumination with UV light of wavelengths compatible with the properties of the chemical group. Two different illumination protocols were applied: bulk uncaging refers to illumination of the whole slide after the first, second and third (only for the positive sample) ligation, whereas spatial uncaging refers to illumination of a partial area of the tissue section defined by the user to install spatial specific barcodes after the third ligation. Bulk uncaging was performed at a UV transilluminator fitted with 365 mm bulbs for 15 minutes, while the sections were covered with ˜500 μL of 2×SSC. Spatial specific illumination was performed on a Leica SP5 confocal microscope equipped with a 30 mW 405 nm solid state laser, an argon laser line at 488 and 514 nm, a He—Ne laser at 543 nm, and a solid state647 nm laser. Slides were mounted on the microscope with 200 uL 2×SSC and a 18 mm×18 mm coverslip (1.5 thickness), with the coverslip facing the objective. After imaging the nuclear staining of Draq5 using the 647 nm laser, the area of interest was defined by the user and photorelease was produced by illuminating with 100% power of the 405 nm laser for 10 minutes. After photorelease, the slide was washed once for 5 minutes in 2×SSC.

### Lysis and De-Crosslinking

After 2×SSC was carefully removed from section, a SecureSeal hybridization chamber was applied to cover the sections. For each chamber, 200 μL of Lysis mix (198 uL RIPA buffer+2 uL ProteinaseK, NEB) were added and the slides incubates in a humid sealed chamber at 60° C. overnight. The lysate was collected from the chambers and purified with a Qiagen MinElute Reaction Cleanup kit (1 column/section) according to the manufacturer protocol. Samples were eluted in 22 μL of EB buffer.

### Library Preparation

Libraries were amplified via combined PCR/qPCR to assess the optimal number of cycles to avoid overamplification of the material. Per each sample, a PCR was set up as follows: 5 uL purified lysate in EB buffer, 1.25 uL of 50 uM universal Nextera P5 primer (BL727), 1.25 uL 50 uM Truseq indexed P7 adapter, 25 uL 2× q5 non Hotstart master mix PCR, H20 to 50 uL final. The reactions were incubated in a PCR cycler for 5 minutes at 72° C. (gap repair), 3 minutes at 98° C., followed by 5 cycles of 20 seconds at 98° C., 20 seconds at 69° C., and 20 seconds at 72° C., then samples store on ice for further amplification. In order to assess the optimal number of cycles, a 5 μL aliquot of the PCR reaction was added to 0.25 uL of 50 uM universal Nextera P5 primer (BL727), 0.25 uL 50 uM Truseq indexed P7 adapter, 5 uL 2× q5 non Hotstart master mix PCR, 0.75 uL 20× EvaGreen, H20 to 15 uL final. The reaction was analysed in a CFX-96 RealTime PCR thermocycler with the same conditions as above for 40 cycles. The optimal number of cycles was defined as that yielding ⅓ of the signal at plateau. The remaining 45 μL of PCR reaction were further amplified according to the qPCR results with the same cycle temperatures, and a final incubation of 5 minutes at 72° C., then held at 4° C. PCR reactions were purified with 1.2× volumes of AmpureXP beads, following the manufacturer protocol and eluted in 22 uL EB buffer. Resulting libraries were checked on a DNA D5000 TapeStation instrument, quantified using the KAPA library quantification kit for Illumina platforms (Roche) according to supplier's instruction and sequenced in-house.

Results are shown in FIG. 24.

TABLE 24
(amplification sequences)

| Oligo ID | Oligo sequence (5′ > 3′) | SEQ ID NO: |
|---|---|---|
| BL727 | AATGATACGGCGACCACCGAGATCTACACTCGTCGGCAGCGTCAG | 109 |
| Truseq_A006 | CAAGCAGAAGACGGCATACGAGAT attggc | 110 |
| | GTGACTGGAGTTCAGACGTGTGCTCTTCCGATC | |
| Truseq_A0012 | CAAGCAGAAGACGGCATACGAGATtacaagGTGACTGG | 111 |
| | AGTTCAGACGTGTGCTCTTCCGATC | |
| Truseq_A003 | CAAGCAGAAGACGGCATACGAGAT gcctaa | 112 |
| | GTGACTGGAGTTCAGACGTGTGCTCTTCCGATC | |
| Truseq_A004 | CAAGCAGAAGACGGCATACGAGAT tggtca | 113 |
| | GTGACTGGAGTTCAGACGTGTGCTCTTCCGATC | |
| Truseq_A005 | CAAGCAGAAGACGGCATACGAGAT cactgt | 114 |
| | GTGACTGGAGTTCAGACGTGTGCTCTTCCGATC | |

### Example 11 (MultiOmics BALI Showing ATAC-Seq and RNA on Tissue)

### Assembling Transposomes (Tn5)

In a PCR tube, adapter pair (detection probe) Tn5ME-A was produced by mixing 2.5 ul of 200 uM BL749 (Tn5MEfwdA-Biotin) and 2.5 ul of 200 UM BL538 (ME_rev_p_blocked) oligonucleotides (resuspended in 1× Annealing buffer) with 5 ul of 1× annealing buffer (40 mM Tris-HCl PH 8.0, 50 mM NaCl). In a separate PCR tube, adapter pair (detection probe) Tn5ME-6A was generated by mixing 2.5 ul of 200 UM BL-515 (TA6+6_NEB01_Tn5ME) and 2.5 ul of 200 UM BL538 (ME_rev_p_blocked) oligonucleotides (resuspended in 1× Annealing buffer) with 5 ul of 1× annealing buffer (40 mM Tris-HCl PH 8.0, 50 mM NaCl). Tubes were mixed and incubated in a PCR thermocycler for 5 minutes at 95° C., then cooled down to 65° C. at −0.1° C. per second. Following incubation for 5 minutes at 65° C., the samples were cooled down to 4° C. at −0.1° C. per second.

Transposomes were assembled with equal amounts of separately pre-annealed Tn5ME-A and Tn5ME-6A adapters (detection probes). In a PCR tube, per each sample, 0.5 ul of annealed Tn5ME-A adapters, 0.5 ul of annealed Tn5ME-6A adapters and 1 ul of unloaded Tn5 transposase were mixed and incubated for 30 minutes at 23° C. in a thermocycler (not exceeding 60 minutes before incubation on tissue).

TABLE 25
(detection probes)

| Oligo ID | Oligo sequence (5′ −> 3′) | SEQ ID NO: |
|---|---|---|
| BL749 | /5BiotinTEG/TCGTCGGCAGCGTCAGATGTGTATAAGAGACAG | 115 |
| BL699 | /5Phos/GGTATTATGGAACGTGATAGATGTGTATAAGAGACAG | 98 |
| BL538 | /5Phos/CTGTCTCTTATACACATCT GATCGACT/3InvdT/ | 69 |

/5Phos/indicates 5′ phosphorylated group./3InvdT/indicates 3′ inverted dT
modification.
/5BiotinTEG/indicates a 5′ biotin group linked through a TEG linker to a
nucleotide. This oligonucleotide was purified using “HPLC Purification”.

### Tissue Sectioning

Brains from adult mouse were collected, and immediately embedded in OCT, snap-frozen in 2-Methylbutane and stored at −80° C. Tissue sections were cut at a thickness of 10 μm using RNase clean procedures. Sections were mounted on SuperFrost Plus microscope slides and immediately transferred on dry ice, and later stored at −80° C. until use.

### Tissue Processing

Unless otherwise stated, tissue processing was conducted at room temperature (˜19-20° C.). On day 1 of the experiment, sections were retrieved from storage at −80° C., dried on a heat block at −37° C. for 5 minutes to improve adhesion to the slide, and a hydrophobic well was drawn around the section with a PapPen. The embedding matrix was removed by washing in 1×PBS-RI (1×PBS+1:1000 Murine RNase Inhibitor, NEB) for 5 minutes. After careful and gentle removal of PBS, sections were fixed for exactly 5 minutes in 0.5% PFA (diluted in 1×PBS) immediately quenched with 1.25M Glycine in 1×PBS (one quick wash and one wash for 5 minutes) and washed twice with 1×PBS-RI for 5 minutes.

### Fixation

After careful removal of 1×PBS, sections were permeabilised in 1× Permeabilization Buffer (10 mM TrisHCl PH 7.4, 10 mM NaCl, 3 mM MgCl2, 0.01% Tween-20, 0.001% digitonin, 1% BSA, 1:1000 Murine RNAse Inhibitor in H₂O) for 15 minutes, and then washed in Wash Buffer (10 mM TrisHCl PH 7.4, 10 mM NaCl, 3 mM MgCl2, 1% BSA, 0.1% Tween-20, 1:1000 Murine RNAse Inhibitor in H₂O) for one quick wash and one wash for 5 minutes, followed by two washes in 1×PBS for 5 minutes each.

### Tagmentation

After PBS was carefully removed from section, a SecureSeal hybridization chamber was applied to cover the sections. For each chamber, 200 uL of tagmentation mix (10 mM Tris HCl pH 7.6, 1M MgCl2, 10% Dimethyl Formamide, 0.33×PBS, 0.01% Tween-20, 0.01% Digitonin, 1:1000 Murine RNase Inhibitor in H₂O) were added and incubated at −37° C. for 35 minutes. The chamber and transposition were removed, and the sections washed once in 40 mM EDTA+1:1000 Murine RNase Inhibitor for 5 minutes and three times in 1×PBS.

### Reverse Transcription and Template Switching

In order to resolve potential secondary structures in the RNA, sections were incubated for 5 minutes in pre-heated 1×PBS at 65° C., then immediately placed in ice-cold 1×PBS for 10 minutes. In parallel, a reverse transcription mix was assembled as two separate mixes. In RT-A, mixed 1.5 uL of 100 UM RT oligo, 2.5 uL 10 mM each dNTPs and 10 uL H20. RT-A was incubated at 65° C. for 5 minutes and then placed immediately on ice. In RT-B, mixed 10 uL 5× SuperScript IV First Strand buffer, 2.5 ul SuperScript IV, 1 uL RNAsin Plus, 3 uL 100 uM BL617 (Template switching oligo), 5 uL 5M Betaine, 2.5 uL 100 mM DTT, 14 uL RT-A mix, 11 uL H20. After careful removal of 1×PBS, 50 uL RT-B mix was applied to each section and then covered with 18×18 mm coverslips. The reaction was incubated at 42° C. overnight in a sealed humid chamber. The coverslip and ligation mix were removed by dipping the slides in a reservoir containing 1×PBS. Sections were washed twice in 2×SSC for 5 minutes, once in 2×SSC+10% EC for 10 minutes, and twice in 2×SSC for 5 minutes.

TABLE 26
(template switching oligos)

| Oligo | | SEQ |
|---|---|---|
| ID | Oligo sequence (5′ > 3′) | ID NO: |
| BL730 | /5Phos/GGTATT CTAGACTG | 116 |
| | TTTTTTTTTTTTTTTTTTTTTTTTTTTTTTVN | |
| BL617 | tacactctttccctacacgacgctcttccgat | 117 |
| | ctrGrGrG | |

/5Phos/indicates 5′ phosphorylated group. rG indicates
a ribonucleotide Guanosine.
V indicates any base that is not T (i.e., A, C, G), N
indicates any base.

### Cyclical Ligation and Uncaging

Each sections underwent, as appropriate, cycles of ligations, followed by washes, photorelease, and washes. Each step is described individually below.

### Ligations

Ligation fragments (index sequences) were annealed as follows. 7.125 ul of each forward and reverse oligonucleotide (100 uM in H₂O) were mixed with 1 μL of 20×SSC, and annealed in a PCR thermocycler for 5 minutes at 95° C., then cooled down to 65° C. at −0.1° C. per second. Following incubation for 5 minutes at 65° C., the samples were cooled down to 10° C. at −0.1° C. per second. Annealed ligation fragments were stored at 4° C., light protected until use. Handling of photocleavable reagents was always performed in light protected conditions.

Sections were washed once in 1× Ligation Buffer (NEB) (50 mM TrisHCl, 10 mM MgCl₂, 1 mM ATP, 10 mM DTT, pH 7.5 at 25° C.) for 5 minutes. After careful removal of the ligation buffer, 50 uL Ligation Mix (1× Ligation Buffer (NEB), 100 U/uL T4 Ligase (NEB), 2.85 uM annealed index sequences in H₂O) was applied to each section and then covered with 18×18 mm coverslips. Ligation were incubated for 60 minutes at 25° C. in a humid light protected chamber. The coverslip and ligation mix were removed by dipping the slides in a reservoir containing 2×SSC. Sections were washed once in 2×SSC+1:1000 Draq5 for 10 minutes, once in 2×SSC+10% EC for 5 minutes, and once in 2×SSC for 5 minutes.

### Photorelease

The photocleavable cage was removed from the oligonucleotides by illumination with UV light of wavelengths compatible with the properties of the chemical group. Two different illumination protocols were applied: bulk uncaging refers to illumination of the whole slide after the first, second and third (only for the positive sample) ligation, whereas spatial uncaging refers to illumination of a partial area of the tissue section defined by the user to install spatial specific barcodes after the third ligation. Bulk uncaging was performed at a UV transilluminator fitted with 365 mm bulbs for 15 minutes, while the sections were covered with ˜500 uL of 2×SSC. Spatial specific illumination was performed on a Leica SP5 confocal microscope equipped with a 30 mW 405 nm solid state laser, an argon laser line at 488 and 514 nm, a He—Ne laser at 543 nm, and a solid state647 nm laser. Slides were mounted on the microscope with 200 uL 2×SSC and a 18 mm×18 mm coverslip (1.5 thickness), with the coverslip facing the objective. After imaging the nuclear staining of Draq5 using the 647 nm laser, the area of interest was defined by the user and photorelease was produced by illuminating with 100% power of the 405 nm laser for 15 minutes. After photorelease, the slide was washed once for 5 minutes in 2×SSC.

TABLE 27
(index sequences)

| Oligo | | SEQ |
|---|---|---|
| ID | Oligo sequence (5′ > 3′) | ID NO: |
| BL680 | AAT/iSpPC/AATAACC GC ACTCAGGT CG | 99 |
| | | (iSpPC |
| | | to 3′) |
| BL676 | AATACC CG ACCTGAGT GC | 100 |
| BL681 | AAT/iSpPC/GGTATT GC ACGGACGA CG | 101 |
| | | (iSpPC |
| | | to 3′) |
| BL677 | GGTTATT CG TCGTCCGT GC | 102 |
| BL683 | AAT/iSpPC/AATAACC GC ACCTAGAT CG | 103 |
| | | (iSpPC |
| | | to 3′) |
| BL642 | AATACC CG ATCTAGGT GC | 104 |
| BL732 | AGACGTGTGCTCTTCCGATCT NNNNNN GC | 105 |
| | ACGAGTCC CG | |
| BL644 | GGTTATT CG GGACTCGT GC | 106 |
| BL733 | AGACGTGTGCTCTTCCGATCT NNNNNN GC | 107 |
| | ACGGACGA CG | |
| BL641 | AATACC CG GGACTCGT GC | 108 |

/iSpPC/indicates an internal photocaged group that
can be cleaved under UV light into a 5′ phosphorylated
group. This oligonucleotide was purified using ″HPLC
Purification″.
N indicates any base.

### Lysis and De-Crosslinking

After 2×SSC was carefully removed from section, a SecureSeal hybridization chamber was applied to cover the sections. For each chamber, 225 μL of Lysis mix (222.75 uL RIPA buffer+2.25 uL ProteinaseK, NEB) were added and the slides incubates in a humid sealed chamber at 60° C. overnight. The lysate was collected from the chambers split as follows: 150 uL for Biotin depletion (cDNA libraries) and 75 ul for column clean up using the Monarch PCR & DNA Cleanup Kit (ATAC libraries).

cDNA Library Preparation

cDNA was depleted from the biotinylated fragments generated by Tn5 via Biotin assisted depletion. For each sample, 20 μL of streptavidin-coupled MyOne C1 beads were washed three times in B&W-T buffer (5 mM TrisHCl pH8.0, 1M NaCl, 0.5 mM EDTA, 0.05% Tween-20). In the meantime, 10 μL of PMSF were added to the lysate fraction (150 uL) at Room Temperature (˜19-20° C.) for 10 minutes to block the proteolytic activity of Proteinase K, and then added to the beads previously resuspended in 2× B&W-T (10 mM TrisHCl pH8.0, 2 M NaCl, 1 mM EDTA, 0.1% Tween-20). The beads and lysate were incubated at Room Temperature for 1 hour gently rotating at 20 rpm. The mix was placed on a magnet to separate the beads and the supernatant (containing the cDNA fraction), which was collected and purified using the Monarch PCR & DNA Cleanup Kit (ATAC libraries), then eluted in 12.5 uL EB buffer. The cDNA was then treated with RNaseH (12.5 uL purified cDNA, 1.5 uL 10× RNaseH buffer, 1 uL RNaseH) for 30 minutes at 30° C., and then the enzyme inactivated at 65° C. for 20 minutes. The reaction was directly used as input for PCR pre-amplification of the cDNA fraction as follows: 15 uL RNaseH mix, 0.25 uL of 100 UM BL752, 0.25 uL of 100 uM BL753, 25 uL 2× q5 non Hot-start master mix PCR, H₂O to 50 ul final. The reactions were incubated in a PCR cycler for 3 minutes at 98° C., followed by 5 cycles of 15 seconds at 98° C., 20 seconds at 67° C., and 60 seconds at 72° C., then samples store on ice for further amplification. In order to assess the optimal number of cycles, a 5 ul aliquot of the PCR reaction was added to 0.5 uL of 10 UM BL753, 0.5 uL 10 UM BL753, 5 uL 2× q5 non Hot-start master mix PCR, 0.75 uL 20× EvaGreen, H₂O to 15 ul final. The reaction was analysed in a CFX-96 RealTime PCR thermocycler with the same conditions as above for 40 cycles. The optimal number of cycles was defined as that yielding ⅓ of the signal at plateau. The remaining 45 μL of PCR reaction were further amplified according to the qPCR results with the same cycle temperatures, and a final incubation of 5 minutes at 72° C., then held at 4° C. PCR reactions were purified with 0.8× volumes of AmpureXP beads, following the manufacturer protocol and eluted in 22 uL EB buffer. Resulting libraries were checked on a DNA D5000 TapeStation instrument.

The amplified cDNA was tagmented to reduce its size and introduce adapters to enable a further round of amplification to generate NGS libraries. The transposome complex was assembled as above, with one modification: for each sample, 0.25 ul pre-annealed Tn5ME-A were mixed with 0.25 uL unloaded Tn5 and incubated at 23° C. for 30 minutes. The transposition mix was assembled by mixing 50 ng of pre-amplified cDNA, 0.5 uL loaded Tn5, 25 uL 2× TD buffer (20 mM Tris HCl pH 7.6, 2M MgCl2, 20% Dimethyl Formamide), H₂O up to 50 μL. The reaction was incubated at 37° C. for 30 minutes, then stopped adding 1 volume (50 uL) of 80 mM EDTA, and 2.5 uL of 2% SDS solution for 15 minutes at Room Temperature (˜19-20° C.). Samples were purified with 1.2× volumes of AmpureXP beads, following the manufacturer protocol and eluted in 22 uL EB buffer. The tagmented cDNA was PCR amplified as follows: 20 uL tagmented cDNA, 2.5 uL of 10 uM universal Nextera P5 primer (BL727), 2.5 uL 10 uM Truseq indexed P7 adapter, 25 μL of 2× NEB Next High fidelity Mastermix, H₂O to 50 ul final. The reactions were incubated in a PCR cycler for 5 minutes at 72° C. (gap repair), 1 minute at 98° C., followed by 5 cycles of 10 seconds at 98° C., 20 seconds at 67° C., and 45 seconds at 72° C., then a final amplification at 72° C. for 5 minutes and held at 10° C. The resulting libraries were were purified with 0.8× volumes of AmpureXP beads, following the manufacturer protocol, eluted in 22 uL EB buffer, then checked on a DNA HS5000 Tapestation cartridge.

### ATAC Libraries Preparation

A fraction (75 uL) of the lysate was purified with the Monarch PCR & DNA Cleanup Kit (ATAC libraries) and eluted in 20 uL. Libraries were amplified via combined PCR/qPCR to assess the optimal number of cycles to avoid overamplification of the material. Per each sample, a PCR was set up as follows: 15 uL purified lysate in EB buffer, 2.5 uL of 10 uM universal Nextera P5 primer (BL727), 2.5 uL 10 uM Truseq indexed P7 adapter, 25 ul 2× q5 non Hotstart master mix PCR, H₂O to 50 uL final. The reactions were incubated in a PCR cycler for 5 minutes at 72° C. (gap repair), 3 minutes at 98° C., followed by 5 cycles of 20 seconds at 98° C., 20 seconds at 65° C., and 20 seconds at 72° C., then samples store on ice for further amplification. In order to assess the optimal number of cycles, a 5 uL aliquot of the PCR reaction was added to 0.5 uL of 10 uM universal Nextera P5 primer (BL727), 0.5 uL 10 uM Truseq indexed P7 adapter, 5 uL 2× q5 non Hotstart master mix PCR, 0.75 uL 20× EvaGreen, H₂O to 15 uL final. The reaction was analysed in a CFX-96 RealTime PCR thermocycler with the same conditions as above for 40 cycles. The optimal number of cycles was defined as that yielding ⅓ of the signal at plateau. The remaining 45 uL of PCR reaction were further amplified according to the qPCR results with the same cycle temperatures, and a final incubation of 5 minutes at 72° C., then held at 4° C. PCR reactions were purified with 1.2× volumes of AmpureXP beads, following the manufacturer protocol and eluted in 22 uL EB buffer. Resulting libraries were checked on a DNA D5000 TapeStation instrument.

Results are shown in FIG. 25.

TABLE 28
(amplification sequences)

| Oligo ID | Oligo sequence (5′ > 3′) | SEQ ID NO: |
|---|---|---|
| BL752 | CCTACACGACGCTCTTCCG | 118 |
| BL753 | AGACGTGTGCTCTTCCGATC | 119 |
| BL727 | AATGATACGGCGACCACCGAGATCTACACTCGTCGGCAG | 109 |
| | CGTCAG | |
| Truseq_A001 | CAAGCAGAAGACGGCATACGAGAT CGTGAT | 120 |
| | GTGACTGGAGTTCAGACGTGTGCTCTTCCGATC | |
| Truseq_A002 | CAAGCAGAAGACGGCATACGAGAT acatcg | 121 |
| | GTGACTGGAGTTCAGACGTGTGCTCTTCCGATC | |
| Truseq_A003 | CAAGCAGAAGACGGCATACGAGAT gcctaa | 112 |
| | GTGACTGGAGTTCAGACGTGTGCTCTTCCGATC | |
| Truseq_A004 | CAAGCAGAAGACGGCATACGAGAT tggtca | 113 |
| | GTGACTGGAGTTCAGACGTGTGCTCTTCCGATC | |
| Truseq_A005 | CAAGCAGAAGACGGCATACGAGAT cactgt | 114 |
| | GTGACTGGAGTTCAGACGTGTGCTCTTCCGATC | |
| Truseq_A006 | CAAGCAGAAGACGGCATACGAGAT attggc | 110 |
| | GTGACTGGAGTTCAGACGTGTGCTCTTCCGATC | |
| Truseq_A007 | CAAGCAGAAGACGGCATACGAGAT atctg | 122 |
| | GTGACTGGAGTTCAGACGTGTGCTCTTCCGATC | |
| Truseq_A008 | CAAGCAGAAGACGGCATACGAGAT tcaagt | 123 |
| | GTGACTGGAGTTCAGACGTGTGCTCTTCCGATC | |
| Truseq_A009 | CAAGCAGAAGACGGCATACGAGAT ctgatc | 124 |
| | GTGACTGGAGTTCAGACGTGTGCTCTTCCGATC | |
| Truseq_A010 | CAAGCAGAAGACGGCATACGAGAT aagcta | 125 |
| | GTGACTGGAGTTCAGACGTGTGCTCTTCCGATC | |
| Truseq_A011 | CAAGCAGAAGACGGCATACGAGAT gtagcc | 126 |
| | GTGACTGGAGTTCAGACGTGTGCTCTTCCGATC | |
| Truseq_A0012 | CAAGCAGAAGACGGCATACGAGAT tacaag | 111 |
| | GTGACTGGAGTTCAGACGTGTGCTCTTCCGATC | |

1. (canceled)

2. A method of spatially barcoding (i) open chromatin and/or chromatin features, and (ii) RNA located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecules of step (a), wherein the or each detection probe optionally comprises a photocleavable group;
(c) Contacting the substrate with one or more detection probes to allow the or each detection probe to bind to an RNA of interest, wherein the or each detection probe optionally comprises a photocleavable group;
(d) Optionally, if the or each detection probe does not comprise a photocleavable group, adding a photocleavable group to the or each detection probe;
(e) Illuminating a location of interest within the tissue to be spatially barcoded, wherein the illumination cleaves or alters the photocleavable group of the or each detection probe within the location;
(f) Adding an index sequence of the spatial barcode to the or each detection probe within the location illuminated in step (e), wherein the index sequence comprises a photocleavable group;
(g) Repeating steps (e) and (f) until the desired index sequences are added to form a spatial barcode attached to the or each detection probe within the location of interest;
(h) Optionally sequencing the one or more spatially barcoded detection probes of step (g) or a derivative thereof.

3. The method according to claim 2, wherein the chromatin feature of interest is a histone mark, a transcription factor, a chromatin factor, a DNA modification site, and/or a G-quadruplex.

4. The method of claim 2, wherein the one or more chromatin feature binding molecules is an antibody or antigen binding fragment thereof, and wherein the one or more secondary binding molecules is an antibody binding protein.

5. A method of spatially barcoding (i) open chromatin and/or chromatin features, and (ii) RNA located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecules of step (a), wherein the or each detection probe optionally comprises a photocleavable group;
(c) Contacting the substrate with a detection probe that binds to one or more RNA molecules of interest;
(d) Performing in situ reverse transcription to generate RNA:DNA hybrids from the RNA molecules of interest;
(e) Optionally, contacting the substrate with one or more additional transposase complexes, wherein each additional transposase complex comprises a transposase bound to at least one detection probe and optionally comprises one or more secondary binding molecules, to allow the or each additional transposase complex to attach the detection probe to an RNA:DNA hybrid of interest, wherein the or each detection probe optionally comprises a photocleavable group; or optionally performing template switching;
(f) Optionally, if the or each detection probe does not comprise a photocleavable group, adding a photocleavable group to the or each detection probe;
(g) Illuminating a location of interest within the tissue to be spatially barcoded, wherein the illumination cleaves or alters the photocleavable group of the or each detection probe within the location;
(h) Adding an index sequence of the spatial barcode to the or each detection probe within the location illuminated in step (g), wherein the index sequence comprises a photocleavable group;
(i) Repeating steps (g) and (h) until the desired index sequences are added to form a spatial barcode attached to the or each detection probe within the location of interest; and
(j) Optionally sequencing the one or more spatially barcoded detection probes of step (i) or a derivative thereof.

6. The method according to claim 5, wherein the chromatin feature of interest is a histone mark, a transcription factor, a chromatin factor, a DNA modification site, and/or a G-quadruplex.

7. The method according to claim 5, wherein, in step (e) template switching takes place, and wherein in step (d) a template switching oligonucleotide is annealed to the RNA:DNA hybrid, and preferably the RNA:DNA hybrid is labelled with a sequence complementary to the template switching oligonucleotide allowing the subsequent amplification of the molecule by enzymatic DNA synthesis, preferably wherein the sequence comprises a PCR primer binding sequence and/or a T7 polymerase promoter sequence, more preferably wherein the sequence comprises a DNA oligonucleotide and optionally a chemical additive selected from Polyethylene glycol (PEG), betaine, Extreme Thermostable Single-Stranded DNA Binding Protein (ET-SSB), and manganese ions.

8. The method of claim 5, wherein the one or more chromatin feature binding molecules is an antibody or binding fragment thereof, and wherein the one or more secondary binding molecules is an antibody binding protein.

9. The method of claim 5, wherein the detection probe that binds to one or more RNA molecules of interest is an RNA detection probe, preferably the RNA detection probe comprises a nucleic acid comprising an extendable 3′-OH end, a modality barcode, and a photocleavable group.

10. The method of claim 5, wherein the method further comprises an amplification step.

11. (canceled)

12. (canceled)

13. (canceled)

14. (canceled)

15. A method of spatially barcoding open chromatin and/or chromatin features located on or within a substrate, comprising:
(a) Optionally, contacting the substrate with one or more chromatin feature binding molecules, wherein each chromatin feature binding molecule binds to a chromatin feature of interest;
(b) Contacting the substrate with one or more transposase complexes, wherein each transposase complex comprises a transposase bound to at least one detection probe and optionally comprises one or more secondary binding molecules, to allow the or each transposase complex to attach the detection probe to DNA at an area of open chromatin and/or to DNA in proximity of the chromatin feature binding molecule of step (a), wherein the or each detection probe optionally comprises a photocleavable group;
(c) Optionally, if the or each detection probe does not comprise a photocleavable group, adding a photocleavable group to the or each detection probe;
(d) Illuminating a location of interest within the tissue to be spatially barcoded, wherein the illumination cleaves or alters the photocleavable group of the or each detection probe within the location;
(e) Adding an index sequence of the spatial barcode to the or each detection probe within the location illuminated in step (d), wherein the index sequence comprises a photocleavable group;
(f) Repeating steps (d) and (e) until the desired index sequences are added to form a spatial barcode attached to the or each detection probe within the location of interest;
(g) Optionally sequencing the one or more spatially barcoded detection probes of step (f) or a derivative thereof.

16. The method of claim 15 wherein the chromatin feature is of interest is a histone mark, a transcription factor, a chromatin factor, a DNA modification site, and/or a G-quadruplex.

17. The method of claim 15, wherein the one or more chromatin feature binding molecules is an antibody or binding fragment thereof, and wherein the one or more secondary binding molecules is an antibody binding protein.

18. (canceled)

19. (canceled)

20. A transposase complex comprising a dimer of transposases, each transposase comprising a detection probe, each detection probe having a first binding region for binding to the transposase, wherein at least one of the detection probes further comprises a second binding region for binding to spatial barcode, and at least one of the detection probes optionally further comprises a photocleavable group.

21. A transposase complex according to claim 20 wherein the dimer comprises a first transposase and a second transposase, each comprising a first detection probe and a second detection probe respectively, preferably wherein the first detection probe comprises the second binding region and the photocleavable group.

22. A transposase complex according to claim 20, wherein the second binding region is capable of binding to an index sequence, preferably an index sequence comprised in a spatial barcode.

23. A transposase complex according to claim 20, further comprising one or more secondary binding molecules linked to the or each transposase, preferably the or each secondary binding molecule is an antibody binding protein.


SEQUENCE LISTING (128 sequences)

SEQ ID NO: 1 - protein, 476 aa, unidentified
  source 1..476: note = wild type tn5
MITSALHRAADWAKSVFSSAALGDPRRTARLVNVAAQLAKYSGKSITISSEGSLAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 2 - protein, 476 aa, synthetic construct
  source 1..476: note = E54K L372P hyperactive tn5
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 3 - protein, 476 aa, synthetic construct
  source 1..476: note = E54K L372P M56A hyperactive tn5
MITSALHRAADWAKSVFSSAALGDPRRTARLVNVAAQLAKYSGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 4 - protein, 476 aa, synthetic construct
  source 1..476: note = E54K L372P M56A Y41A hyperactive tn5
MITSALHRAADWAKSVFSSAALGDPRRTARLVNVAAQLAKASGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 5 - protein, 476 aa, synthetic construct
  source 1..476: note = E54K L372P M56A S42A hyperactive tn5
MITSALHRAADWAKSVFSSAALGDPRRTARLVNVAAQLAKYAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 6 - protein, 476 aa, synthetic construct
  source 1..476: note = E54K L372P M56A Y41C C187A C402A hyperactive tn5
MITSALHRAADWAKSVFSSAALGDPRRTARLVNVAAQLAKCAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVADREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDEAQLLGYLDKGKRKRKEKAGSLQWAYMAIARLGGFMDSKRTGIASWGALWEGWEALQSKLDGFLAAKDLMAQGIKI

SEQ ID NO: 7 - protein, 476 aa, synthetic construct
  source 1..476: note = E54K L372P M56A W450C C187A C402A hyperactive tn5
MITSALHRAADWAKSVFSSAALGDPRRTARLVNVAAQLAKYAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVADREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDEAQLLGYLDKGKRKRKEKAGSLQWAYMAIARLGGFMDSKRTGIASWGALCEGWEALQSKLDGFLAAKDLMAQGIKI

SEQ ID NO: 8 - protein, 476 aa, synthetic construct
  source 1..476: note = E54K L372P M56A E451C C187A C402A hyperactive tn5
MITSALHRAADWAKSVFSSAALGDPRRTARLVNVAAQLAKYAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVADREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDEAQLLGYLDKGKRKRKEKAGSLQWAYMAIARLGGFMDSKRTGIASWGALWCGWEALQSKLDGFLAAKDLMAQGIKI

SEQ ID NO: 9 - protein, 476 aa, synthetic construct
  source 1..476: note = E54K L372P M56A E454C C187A C402A hyperactive tn5
MITSALHRAADWAKSVFSSAALGDPRRTARLVNVAAQLAKYAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVADREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDEAQLLGYLDKGKRKRKEKAGSLQWAYMAIARLGGFMDSKRTGIASWGALWEGWCALQSKLDGFLAAKDLMAQGIKI

SEQ ID NO: 10 - protein, 476 aa, synthetic construct
  source 1..476: note = and E54K L372P M56A W450C Y41C C187A C402A hyperactive tn5
MITSALHRAADWAKSVFSSAALGDPRRTARLVNVAAQLAKCAGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVADREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDEAQLLGYLDKGKRKRKEKAGSLQWAYMAIARLGGFMDSKRTGIASWGALCEGWCALQSKLDGFLAAKDLMAQGIKI

SEQ ID NO: 11 - DNA, 102 bp, synthetic construct
  source 1..102: mol_type = other DNA; note = BALI_09
  modified_base 102: mod_base = OTHER; note = Aminolink C6
ggtagtatatgtcgagagctagccgcgccttaagataccaattagagtagatcggaagagcgtcgtgtagctccctatagtgagtcgtattactagctagcg

SEQ ID NO: 12 - DNA, 15 bp, synthetic construct
  source 1..15: mol_type = other DNA; note = BALI_10
gctagctctcgacat

SEQ ID NO: 13 - DNA, 19 bp, synthetic construct
  source 1..19: mol_type = other DNA; note = BALI_11
  misc_feature 1: note = Atto488 green fluorescent group bound to the 5 prime of the molecule via the photocleavable spacer oligo modification shown in figure 1
gtacctgtcagctaccatg

SEQ ID NO: 14 - DNA, 19 bp, synthetic construct
  source 1..19: mol_type = other DNA; note = BALI_12
  modified_base 1: mod_base = OTHER; note = 5' phosphate
atactacccatggtagctg

SEQ ID NO: 15 - DNA, 18 bp, synthetic construct
  source 1..18: mol_type = other DNA; note = BALI_13
  misc_feature 1: note = cyanine 5 (Cy5) fluorescent group bound to the 5 prime end of a molecule via the photocleavable spacer oligo modification shown in figure 1
gtatcgagctctatacac

SEQ ID NO: 16 - DNA, 18 bp, synthetic construct
  source 1..18: mol_type = other DNA; note = BALI_14
  modified_base 1: mod_base = OTHER; note = 5' phosphate
acaggtacgtgtatagag

SEQ ID NO: 17 - DNA, 19 bp, synthetic construct
  source 1..19: mol_type = other DNA; note = BALI_15
  misc_feature 1: note = Atto565 red fluorescent group bound to the 5 prime end of a molecule via the photocleavable spacer oligo modification shown in figure 1
gtgagcgtcggacacctac

SEQ ID NO: 18 - DNA, 19 bp, synthetic construct
  source 1..19: mol_type = other DNA; note = BALI_16
  modified_base 1: mod_base = OTHER; note = 5' phosphate
ctcgatacgtaggtgtccg

SEQ ID NO: 19 - DNA, 136 bp, synthetic construct
  source 1..136: mol_type = other DNA; note = BALI_77
  misc_feature 1: note = The photocleavable spacer modification as shown in figure 1
gtgaccatgtcgagagctagcnnnnnnnnnnnnnnncatcgtgagtagatcggaagagcgtcgtgtagctccctatagtgagtcgtattactagctagcgttgaagaagatggtgcgctcctggacgtagccttcg

SEQ ID NO: 20 - DNA, 136 bp, synthetic construct
  source 1..136: mol_type = other DNA; note = BALI_78
  misc_feature 1: note = The photocleavable spacer modification as shown in figure 1
gtgaccatgtcgagagctagcnnnnnnnnnnnnnnncatcgtgagtagatcggaagagcgtcgtgtagctccctatagtgagtcgtattactagctagcgcttgaagtcgatgcccttcagctcgatgcggttcac

SEQ ID NO: 21 - DNA, 136 bp, synthetic construct
  source 1..136: mol_type = other DNA; note = BALI_79
  misc_feature 1: note = The photocleavable spacer modification as shown in figure 1
gtgaccatgtcgagagctagcnnnnnnnnnnnnnnncatcgtgagtagatcggaagagcgtcgtgtagctccctatagtgagtcgtattactagctagcgtgcttgtcggccatgatatagacgttgtggctgttg

SEQ ID NO: 22 - DNA, 136 bp, synthetic construct
  source 1..136: mol_type = other DNA; note = BALI_80
  misc_feature 1: note = The photocleavable spacer modification as shown in figure 1
gtgaccatgtcgagagctagcnnnnnnnnnnnnnnncatcgtgagtagatcggaagagcgtcgtgtagctccctatagtgagtcgtattactagctagcgcatgtgatcgcgcttctcgttggggtctttgctcag

SEQ ID NO: 23 - DNA, 136 bp, synthetic construct
  source 1..136: mol_type = other DNA; note = BALI_81
  misc_feature 1: note = The photocleavable spacer modification as shown in figure 1
gtgaccatgtcgagagctagcnnnnnnnnnnnnnnnggtacagatgagatcggaagagcgtcgtgtagctccctatagtgagtcgtattactagctagcggatggccatgttatcctcctcgcccttgctcaccat

SEQ ID NO: 24 - DNA, 136 bp, synthetic construct
  source 1..136: mol_type = other DNA; note = BALI_82
  misc_feature 1: note = The photocleavable spacer modification as shown in figure 1
gtgaccatgtcgagagctagcnnnnnnnnnnnnnnnggtacagatgagatcggaagagcgtcgtgtagctccctatagtgagtcgtattactagctagcgctcggggaaggacagcttcaagtagtcggggatgtc

SEQ ID NO: 25 - DNA, 136 bp, synthetic construct
  source 1..136: mol_type = other DNA; note = BALI_83
  misc_feature 1: note = The photocleavable spacer modification as shown in figure 1
gtgaccatgtcgagagctagcnnnnnnnnnnnnnnnggtacagatgagatcggaagagcgtcgtgtagctccctatagtgagtcgtattactagctagcgcttgtaggtggtcttgacctcagcgtcgtagtggcc

SEQ ID NO: 26 - DNA, 136 bp, synthetic construct
  source 1..136: mol_type = other DNA; note = BALI_84
  misc_feature 1: note = The photocleavable spacer modification as shown in figure 1
gtgaccatgtcgagagctagcnnnnnnnnnnnnnnnggtacagatgagatcggaagagcgtcgtgtagctccctatagtgagtcgtattactagctagcgctgttccacgatggtgtagtcctcgttgtgggaggt

SEQ ID NO: 27 - DNA, 15 bp, synthetic construct
  source 1..15: mol_type = other DNA; note = BALI_85
gctagctctcgacat

SEQ ID NO: 28 - DNA, 32 bp, synthetic construct
  source 1..32: mol_type = other DNA; note = BALI_86
agacgtgtgctcttccgatctcagctaccatg

SEQ ID NO: 29 - DNA, 38 bp, synthetic construct
  source 1..38: mol_type = other DNA; note = BALI_87
ggtcaccatggtagctgagatcggaagagcacacgtct

SEQ ID NO: 30 - DNA, 32 bp, synthetic construct
  source 1..32: mol_type = other DNA; note = BALI_88
agacgtgtgctcttccgatctgacaatgaggc

SEQ ID NO: 31 - DNA, 38 bp, synthetic construct
  source 1..38: mol_type = other DNA; note = BALI_89
ggtcacgcctcattgtcagatcggaagagcacacgtct

SEQ ID NO: 32 - DNA, 21 bp, synthetic construct
  source 1..21: mol_type = other DNA; note = BALI_90
agacgtgtgctcttccgatct

SEQ ID NO: 33 - DNA, 28 bp, synthetic construct
  source 1..28: mol_type = other DNA; note = BALI_91
taatacgactcactatagggagctacac

SEQ ID NO: 34 - DNA, 58 bp, synthetic construct
  source 1..58: mol_type = other DNA; note = Illumina universal FW Truseq
aatgatacggcgaccaccgagatctacactctttccctacacgacgctcttccgatct

SEQ ID NO: 35 - DNA, 63 bp, synthetic construct
  source 1..63: mol_type = other DNA; note = Illumina Truseq LT rev. index 006
caagcagaagacggcatacgagatattggcgtgactggagttcagacgtgtgctcttccgatc

SEQ ID NO: 36 - DNA, 63 bp, synthetic construct
  source 1..63: mol_type = other DNA; note = Illumina Truseq LT rev. index 012
caagcagaagacggcatacgagattacaaggtgactggagttcagacgtgtgctcttccgatc

SEQ ID NO: 37 - DNA, 90 bp, synthetic construct
  source 1..90: mol_type = other DNA; note = BALI_92
  modified_base 1: mod_base = OTHER; note = 5' acrydite group
cgacatcggcaagctactacggctagcctaatacgactcactatagggagactctaattggtatagaattccgcggctagctctcgacat

SEQ ID NO: 38 - DNA, 72 bp, synthetic construct
  source 1..72: mol_type = other DNA; note = BALI_93
  modified_base 1: mod_base = OTHER; note = Phosphate
gtgacgtatgtcgagagctagccgcggaattctataccaattagagtctccctatagtgagtcgtattaggc

SEQ ID NO: 39 - DNA, 18 bp, synthetic construct
  source 1..18: mol_type = other DNA; note = BALI_94
  misc_feature 1: note = Cyanine 5 (Cy5) fluorescent group bound to the 5 prime end of a molecule via the photocleavable spacer modification shown in figure 1
gagcgtgcaccacagtcg

SEQ ID NO: 40 - DNA, 19 bp, synthetic construct
  source 1..19: mol_type = other DNA; note = BALI_95
acgtcaccgactgtggtgc

SEQ ID NO: 41 - DNA, 19 bp, synthetic construct
  source 1..19: mol_type = other DNA; note = BALI_96
  misc_feature 1: note = Cyanine 3 (Cy3) fluorescent group bound to the 5 prime of the molecule via the photocleavable spacer modification as shown in figure 1
gtggaccgcacgtatggcg

SEQ ID NO: 42 - DNA, 18 bp, synthetic construct
  source 1..18: mol_type = other DNA; note = BALI_97
acgctccgccatacgtgc

SEQ ID NO: 43 - DNA, 19 bp, synthetic construct
  source 1..19: mol_type = other DNA; note = BALI_98
  misc_feature 1: note = Cyanine 3 (Cy3) fluorescent group bound to the 5 prime of the molecule via the photocleavable spacer modification as shown in figure 1
gtggaccgcacgagtcccg

SEQ ID NO: 44 - DNA, 18 bp, synthetic construct
  source 1..18: mol_type = other DNA; note = BALI_99
acgctccgggactcgtgc

SEQ ID NO: 45 - DNA, 33 bp, synthetic construct
  source 1..33: mol_type = other DNA; note = BALI_100
agacgtgtgctcttccgatctactgaggtgagc

SEQ ID NO: 46 - DNA, 40 bp, synthetic construct
  source 1..40: mol_type = other DNA; note = BALI_101
ggtccacgctcacctcagtagatcggaagagcacacgtct

SEQ ID NO: 47 - DNA, 33 bp, synthetic construct
  source 1..33: mol_type = other DNA; note = BALI_102
agacgtgtgctcttccgatctcgacgatctagc

SEQ ID NO: 48 - DNA, 40 bp, synthetic construct
  source 1..40: mol_type = other DNA; note = BALI_103
ggtccacgctagatcgtcgagatcggaagagcacacgtct

SEQ ID NO: 49 - DNA, 21 bp, synthetic construct
  source 1..21: mol_type = other DNA; note = BALI_104
agacgtgtgctcttccgatct

SEQ ID NO: 50 - DNA, 82 bp, synthetic construct
  source 1..82: mol_type = other DNA; note = BALI_105
aatgatacggcgaccaccgagatctacactctttccctacacgacgctcttccgatctnnnnnngcggctagctctcgacat

SEQ ID NO: 51 - DNA, 33 bp, synthetic construct
  source 1..33: mol_type = other DNA; note = oBC3620
tcgtcggcagcgtcagatgtgtataagagacag

SEQ ID NO: 52 - DNA, 19 bp, synthetic construct
  source 1..19: mol_type = other DNA; note = oBC3622
  modified_base 1: mod_base = OTHER; note = 5' phosphorylation
ctgtctcttatacacatct

SEQ ID NO: 53 - DNA, 34 bp, synthetic construct
  source 1..34: mol_type = other DNA; note = oBC3656
  misc_feature 1: note = 5 prime photocaged group called /5SpPC/ that can be cleaved under UV light into a 5 prime phosphorylated group
gtcactccgcctcgtagatgtgtataagagacag

SEQ ID NO: 54 - DNA, 34 bp, synthetic construct
  source 1..34: mol_type = other DNA; note = oBC3631
gtctcgtgggctcggagatgtgtataagagacag

SEQ ID NO: 55 - DNA, 35 bp, synthetic construct
  source 1..35: mol_type = other DNA; note = oBC3650
acgaggcggagtgacctgtctcttatacacatctc

SEQ ID NO: 56 - DNA, 45 bp, synthetic construct
  source 1..45: mol_type = other DNA; note = oBC3657
aatgatacggcgaccaccgagatctacactcgtcggcagcgtcag

SEQ ID NO: 57 - DNA, 41 bp, synthetic construct
  source 1..41: mol_type = other DNA; note = oBC3716
caagcagaagacggcatacgagatgtcactccgcctcgtag

SEQ ID NO: 58 - DNA, 54 bp, synthetic construct
  source 1..54: mol_type = other DNA; note = Ad2.12
caagcagaagacggcatacgagattcctctacgtctcgtgggctcggagatgtg

SEQ ID NO: 59 - DNA, 54 bp, synthetic construct
  source 1..54: mol_type = other DNA; note = Ad2.13
caagcagaagacggcatacgagatcagatccagtctcgtgggctcggagatgtg

SEQ ID NO: 60 - DNA, 54 bp, synthetic construct
  source 1..54: mol_type = other DNA; note = Ad2.14
caagcagaagacggcatacgagatacaaacgggtctcgtgggctcggagatgtg

SEQ ID NO: 61 - DNA, 54 bp, synthetic construct
  source 1..54: mol_type = other DNA; note = Ad2.15
caagcagaagacggcatacgagatacccagcagtctcgtgggctcggagatgtg

SEQ ID NO: 62 - DNA, 40 bp, synthetic construct
  source 1..40: mol_type = other DNA; note = oBC3729
  modified_base 1: mod_base = OTHER; note = 5' phosphorylation
gtcactccgcctcgtcgtgatagatgtgtataagagacag

SEQ ID NO: 63 - DNA, 41 bp, synthetic construct
  source 1..41: mol_type = other DNA; note = oBC3751
  modified_base 1: mod_base = OTHER; note = 5' phosphorylation
  modified_base 22: mod_base = OTHER; note = biotinylated deoxythymidine
gtcactccgcctcgtacatcgttttttttttttttttttvn

SEQ ID NO: 64 - DNA, 46 bp, synthetic construct
  source 1..46: mol_type = other DNA; note = oBC3736
gtctcgtgggctcggagatgtgtataagagacaggtcacacccgcg

SEQ ID NO: 65 - DNA, 48 bp, synthetic construct
  source 1..48: mol_type = other DNA; note = oBC3758
acgaggcggagtgaccgcgggtgtgacctgtctcttatacacatctcc

SEQ ID NO: 66 - DNA, 54 bp, synthetic construct
  source 1..54: mol_type = other DNA; note = Ad2.5
caagcagaagacggcatacgagataggagtccgtctcgtgggctcggagatgtg

SEQ ID NO: 67 - DNA, 54 bp, synthetic construct
  source 1..54: mol_type = other DNA; note = Ad2.6
caagcagaagacggcatacgagatcatgcctagtctcgtgggctcggagatgtg

SEQ ID NO: 68 - DNA, 36 bp, synthetic construct
  misc_feature 1..33: note = DNA
  source 1..36: mol_type = other DNA; note = oBC3763
  misc_feature 34..35: note = RNA
  misc_feature 36: note = DNA
  modified_base 36: mod_base = OTHER; note = locked nucleic acid (LNA)
tcgtcggcagcgtcagatgtgtataagagacagggg

SEQ ID NO: 69 - DNA, 28 bp, synthetic construct
  source 1..28: mol_type = other DNA; note = BL538
  modified_base 1: mod_base = OTHER; note = 5 prime phosphate
  modified_base 28: mod_base = OTHER; note = inverted deoxythymidine
ctgtctcttatacacatctgatcgactt

SEQ ID NO: 70 - skipped

SEQ ID NO: 71 - DNA, 67 bp, synthetic construct
  misc_feature 1..64: note = DNA
  misc_feature 65..67: note = RNA
  source 1..67: mol_type = other DNA; note = BL320
ctagctagtaatacgactcactatagggagtacactctttccctacacgacgctcttccgatctggg

SEQ ID NO: 72 - DNA, 50 bp, synthetic construct
  source 1..50: mol_type = other DNA; note = BL449
aatgatacggcgaccaccgagatctacactcgtcggcagcgtcagatgtg

SEQ ID NO: 73 - DNA, 53 bp, synthetic construct
  source 1..53: mol_type = other DNA; note = Ad2.1
caagcagaagacggcatacgagattcgccttagtctcgtgggctcggagatgt

SEQ ID NO: 74 - DNA, 58 bp, synthetic construct; identical to SEQ ID NO: 34
  source 1..58: mol_type = other DNA; note = Truseq_Universal_R1

SEQ ID NO: 75 - DNA, 63 bp, synthetic construct; identical to SEQ ID NO: 36
  source 1..63: mol_type = other DNA; note = A012_Truseq_R2 oligo

SEQ ID NO: 76 - DNA, 19 bp, synthetic construct
  source 1..19: mol_type = other DNA
  modified_base 1: mod_base = OTHER; note = 5' phosphorylation
ctgtctcttatacacatct

SEQ ID NO: 77 - DNA, 40 bp, synthetic construct; identical to SEQ ID NO: 62
  source 1..40: mol_type = other DNA; note = oBC3737
  misc_feature 1: note = 5 prime photocaged group called /5SpPC/ that can be cleaved under UV light into a 5 prime phosphorylated group

SEQ ID NO: 78 - DNA, 29 bp, synthetic construct
  source 1..29: mol_type = other DNA; note = oBC3730
acgaggcggagtgaccggagggtcatcgc

SEQ ID NO: 79 - DNA, 29 bp, synthetic construct
  source 1..29: mol_type = other DNA; note = oBC3738
  misc_feature 1: note = 5 prime photocaged group called /5SpPC/ that can be cleaved under UV light into a 5 prime phosphorylated group
ggacgagaggactgcgcgatgaccctccg

SEQ ID NO: 80 - DNA, 29 bp, synthetic construct
  source 1..29: mol_type = other DNA; note = oBC3733
gcagtcctctcgtcccggctgtcccgtgc

SEQ ID NO: 81 - DNA, 29 bp, synthetic construct
  source 1..29: mol_type = other DNA; note = oBC3740
  misc_feature 1: note = 5 prime photocaged group called /5SpPC/ that can be cleaved under UV light into a 5 prime phosphorylated group
cacccagactctcttgcacgggacagccg

SEQ ID NO: 82 - DNA, 61 bp, synthetic construct
  source 1..61: mol_type = other DNA; note = oBC3735
aagagagtctgggtgcgcgggtgtgacctgtctcttatacacatctccgagcccacgagac

SEQ ID NO: 83 - DNA, 39 bp, synthetic construct
  source 1..39: mol_type = other DNA; note = oBC3766
caagcagaagacggcatacgagatgtcactccgcctcgt

SEQ ID NO: 84 - DNA, 54 bp, synthetic construct
  source 1..54: mol_type = other DNA; note = Ad2.7
caagcagaagacggcatacgagatgtagagaggtctcgtgggctcggagatgtg

SEQ ID NO: 85 - DNA, 37 bp, synthetic construct
  source 1..37: mol_type = other DNA; note = oBC3952
  modified_base 1: mod_base = OTHER; note = 5' phosphorylated group
ggtattatggaacgtgatagatgtgtataagagacag

SEQ ID NO: 86 - DNA, 19 bp, synthetic construct
  source 1..19: mol_type = other DNA; note = BL584
  misc_feature 1: note = the 5' end is attached to another sequence (5'-AAT-3') through an internal photocaged group called /iSpPC/ that can be cleaved under UV light into a 5 prime phosphorylated group
aataaccgcacgagtcccg

SEQ ID NO: 87 - DNA, 18 bp, synthetic construct
  source 1..18: mol_type = other DNA; note = BL585
aatacccgggactcgtgc

SEQ ID NO: 88 - DNA, 39 bp, synthetic construct
  source 1..39: mol_type = other DNA; note = BL623
  misc_feature 1: note = 5' desthiobiotin group called /5deSBioTEG/ linked through a TEG linker to a nucleotide
agacgtgtgctcttccgatctnnnnnngcacctagatcg

SEQ ID NO: 89 - DNA, 19 bp, synthetic construct
  source 1..19: mol_type = other DNA; note = BL583
ggttattcgatctaggtgc

SEQ ID NO: 90 - DNA, 45 bp, synthetic construct
  source 1..45: mol_type = other DNA; note = BL624
  misc_feature 1: note = 5' desthiobiotin group called /5deSBioTEG/ linked through a TEG linker to a nucleotide (Desthiobiotin-TEG)
agacgtgtgcctagcttcttccgatctnnnnnngcacgagtcccg

SEQ ID NO: 91 - DNA, 19 bp, synthetic construct
  source 1..19: mol_type = other DNA; note = BL601
ggttattcgggactcgtgc

SEQ ID NO: 92 - DNA, 63 bp, synthetic construct
  source 1..63: mol_type = other DNA; note = TruSeq A001
caagcagaagacggcatacgagatcgtgatgtgactggagttcagacgtgtgctcttccgatc

SEQ ID NO: 93 - DNA, 63 bp, synthetic construct
  source 1..63: mol_type = other DNA; note = TruSeq A002
caagcagaagacggcatacgagatacatcggtgactggagttcagacgtgtgctcttccgatc

SEQ ID NO: 94 - DNA, 63 bp, synthetic construct
  source 1..63: mol_type = other DNA; note = TruSeq A003
caagcagaagacggcatacgagatgcctaagtgactggagttcagacgtgtgctcttccgatc

SEQ ID NO: 95 - DNA, 63 bp, synthetic construct
  source 1..63: mol_type = other DNA; note = TruSeq A004
caagcagaagacggcatacgagattggtcagtgactggagttcagacgtgtgctcttccgatc

SEQ ID NO: 96 - DNA, 63 bp, synthetic construct
  source 1..63: mol_type = other DNA; note = TruSeq A005
caagcagaagacggcatacgagatcactgtgtgactggagttcagacgtgtgctcttccgatc

SEQ ID NO: 97 - DNA, 62 bp, synthetic construct
  source 1..62: mol_type = other DNA; note = TruSeq A007
caagcagaagacggcatacgagatatctggtgactggagttcagacgtgtgctcttccgatc

SEQ ID NO: 98 - DNA, 37 bp, synthetic construct; identical to SEQ ID NO: 85
  source 1..37: mol_type = other DNA; note = BL699
  modified_base 1: mod_base = OTHER; note = 5 prime phosphorylated group

SEQ ID NO: 99 - DNA, 19 bp, synthetic construct
  source 1..19: mol_type = other DNA; note = BL680
  misc_feature 1: note = the 5' end is attached to another sequence (5'-AAT-3') through an internal photocaged group called /iSpPC/ that can be cleaved under UV light into a 5 prime phosphorylated group
aataaccgcactcaggtcg

SEQ ID NO: 100 - DNA, 18 bp, synthetic construct
  source 1..18: mol_type = other DNA; note = BL676
aatacccgacctgagtgc

SEQ ID NO: 101 - DNA, 18 bp, synthetic construct
  source 1..18: mol_type = other DNA; note = BL681
  misc_feature 1: note = the 5' end is attached to another sequence (5'-AAT-3') through an internal photocaged group called /iSpPC/ that can be cleaved under UV light into a 5 prime phosphorylated group
ggtattgcacggacgacg

SEQ ID NO: 102 - DNA, 19 bp, synthetic construct
  source 1..19: mol_type = other DNA; note = BL677
ggttattcgtcgtccgtgc

SEQ ID NO: 103 - DNA, 19 bp, synthetic construct
  source 1..19: mol_type = other DNA; note = BL683
  misc_feature 1: note = the 5' end is attached to another sequence (5'-AAT-3') through an internal photocaged group called /iSpPC/ that can be cleaved under UV light into a 5 prime phosphorylated group
aataaccgcacctagatcg

SEQ ID NO: 104 - DNA, 18 bp, synthetic construct
  source 1..18: mol_type = other DNA; note = BL642
aatacccgatctaggtgc

SEQ ID NO: 105 - DNA, 39 bp, synthetic construct
  source 1..39: mol_type = other DNA; note = BL732
agacgtgtgctcttccgatctnnnnnngcacgagtcccg

SEQ ID NO: 106 - DNA, 19 bp, synthetic construct
  source 1..19: mol_type = other DNA; note = BL644
ggttattcgggactcgtgc

SEQ ID NO: 107 - DNA, 39 bp, synthetic construct
  source 1..39: mol_type = other DNA; note = BL733
agacgtgtgctcttccgatctnnnnnngcacggacgacg

SEQ ID NO: 108 - DNA, 18 bp, synthetic construct
  source 1..18: mol_type = other DNA; note = BL641
aatacccgggactcgtgc

SEQ ID NO: 109 - DNA, 45 bp, synthetic construct; identical to SEQ ID NO: 56
  source 1..45: mol_type = other DNA; note = BL727

SEQ ID NO: 110 - DNA, 63 bp, synthetic construct; identical to SEQ ID NO: 35
  source 1..63: mol_type = other DNA; note = Truseq_A006

SEQ ID NO: 111 - DNA, 63 bp, synthetic construct; identical to SEQ ID NO: 36
  source 1..63: mol_type = other DNA; note = Truseq_A0012

SEQ ID NO: 112 - DNA, 63 bp, synthetic construct; identical to SEQ ID NO: 94
  source 1..63: mol_type = other DNA; note = Truseq_A003

SEQ ID NO: 113 - DNA, 63 bp, synthetic construct; identical to SEQ ID NO: 95
  source 1..63: mol_type = other DNA; note = Truseq_A004

SEQ ID NO: 114 - DNA, 63 bp, synthetic construct; identical to SEQ ID NO: 96
  source 1..63: mol_type = other DNA; note = Truseq_A005

SEQ ID NO: 115 - DNA, 33 bp, synthetic construct; identical to SEQ ID NO: 51
  source 1..33: mol_type = other DNA; note = BL749
  misc_feature 1: note = 5 prime biotin group called /5BiotinTEG/ linked through a TEG linker to a nucleotide

SEQ ID NO: 116 - DNA, 46 bp, synthetic construct
  source 1..46: mol_type = other DNA; note = BL730
  modified_base 1: mod_base = OTHER; note = 5 prime phosphorylated group
ggtattctagactgttttttttttttttttttttttttttttttvn

SEQ ID NO: 117 - DNA, 37 bp, synthetic construct
  misc_feature 1..34: note = DNA
  misc_feature 35..37: note = RNA
  source 1..37: mol_type = other DNA; note = BL617
tacactctttccctacacgacgctcttccgatctggg

SEQ ID NO: 118 - DNA, 19 bp, synthetic construct
  source 1..19: mol_type = other DNA; note = BL752
cctacacgacgctcttccg

SEQ ID NO: 119 - DNA, 20 bp, synthetic construct
  source 1..20: mol_type = other DNA; note = BL753
agacgtgtgctcttccgatc

SEQ ID NO: 120 - DNA, 63 bp, synthetic construct; identical to SEQ ID NO: 92
  source 1..63: mol_type = other DNA; note = Truseq_A001

SEQ ID NO: 121 - DNA, 63 bp, synthetic construct; identical to SEQ ID NO: 93
  source 1..63: mol_type = other DNA; note = Truseq_A002

SEQ ID NO: 122 - DNA, 62 bp, synthetic construct; identical to SEQ ID NO: 97
  source 1..62: mol_type = other DNA; note = Truseq_A007

SEQ ID NO: 123 - DNA, 63 bp, synthetic construct
  source 1..63: mol_type = other DNA; note = Truseq_A008
caagcagaagacggcatacgagattcaagtgtgactggagttcagacgtgtgctcttccgatc

SEQ ID NO: 124 - DNA, 63 bp, synthetic construct
  source 1..63: mol_type = other DNA; note = Truseq_A009
caagcagaagacggcatacgagatctgatcgtgactggagttcagacgtgtgctcttccgatc

SEQ ID NO: 125 - DNA, 63 bp, synthetic construct
  source 1..63: mol_type = other DNA; note = Truseq_A010
caagcagaagacggcatacgagataagctagtgactggagttcagacgtgtgctcttccgatc

SEQ ID NO: 126 - DNA, 63 bp, synthetic construct
  source 1..63: mol_type = other DNA; note = Truseq_A011
caagcagaagacggcatacgagatgtagccgtgactggagttcagacgtgtgctcttccgatc

SEQ ID NO: 127 - DNA, 36 bp, synthetic construct
  source 1..36: mol_type = other DNA
  misc_feature 1: note = 5 prime fluorescent cyanine 5 (Cy5) group
  misc_feature 36: note = the 3' end is attached to another nucleotide sequence through an internal spacer desthiobiotin group called /ideSBioTEG/ linked through a TEG to a nucleotide
agacgtgtgctcttccgatctgtttagctgctagct

SEQ ID NO: 128 - DNA, 36 bp, synthetic construct
  source 1..36: mol_type = other DNA
  misc_feature 1: note = the 5' end is attached to another nucleotide sequence through an internal spacer desthiobiotin group called /ideSBioTEG/ linked through a TEG to a nucleotide
agcgcttttttttttttttttttttttttttttttt